SEQUENCE LISTING (5 sequences)

SEQ ID NO: 1 - protein, 4 aa, Artificial Sequence
  Synthetic: peptide linker
GFLG

SEQ ID NO: 2 - protein, 4 aa, Artificial Sequence
  Synthetic: peptide linker
KPLR

SEQ ID NO: 3-4 - skipped (2 entries)

SEQ ID NO: 5 - protein, 4 aa, Artificial Sequence
  Synthetic: peptide linker
YLLL

(19) United States
(12) Patent Application Publication
(10) Pub. No.: US 2023/0390406 A1
(43) Pub. Date: Dec. 7, 2023

(54) Star Polymer Drug Conjugates

(71) Applicants: Vaccitech North America, Inc., Baltimore, MD (US); The United States of America, As Represented By The Secretary, Department Of Health and Human Servi, Bethesda, MD (US)

(72) Inventors: Geoffrey Martin Lynn, Baltimore, MD (US); Yaling Zhu, Baltimore, MD (US); David Robert Wilson, Baltimore, MD (US); Jake Holechek, Baltimore, MD (US); Bridget Duvall, Baltimore, MD (US); Andrew Scott Ishizuka, Washington, DC (US); Qiuyin Ren, Bethesda, MD (US)

(21) Appl. No.: 18/032,538

(22) PCT Filed: Oct. 18, 2021
(86) PCT No.: PCT/US2021/055414

§ 371 Date: Apr. 18, 2023

Related U.S. Application Data
(60) Provisional application No. 63/093,445, filed on Oct. 19, 2020.

(51) Int. Cl. A61K 47/64; A61K 47/65; A61K 47/59; A61P 35/00

(52) U.S. Cl. CPC A61K 47/641; A61K 47/65; A61K 47/645; A61K 47/593; A61K 47/595; A61P 35/00

(57) ABSTRACT

A star polymer of formula O[D1]-([X]-A(D2)-[Z]-[D3])n where O is a core; A is a polymer arm that comprises reactive monomers, hydrophilic monomers and/or charged monomers and is attached to the core; X is a linker molecule between the core and the polymer arm; Z is a linker molecule between the end of the polymer arm and D3; D1 is a drug molecule linked to the core; D2 is a drug molecule linked to reactive monomers distributed along the polymer arm; D3 is a drug molecule linked to the ends of the polymer arms; n is an integer number; [ ] denotes that the group is optional; and D2 is linked to the reactive monomers distributed along the polymer arm at a density of between 1 mol % and 80 mol %.

### CROSS-REFERENCE TO RELATED APPLICATIONS

This application claims priority to U.S. Provisional Application No. 63/093,445, filed on Oct. 19, 2020, the disclosure of which is hereby incorporated by reference in its entirety.

This invention was created in the performance of a Cooperative Research and Development Agreement with the National Institutes of Health, an Agency of the Department of Health and Human Services. The Government of the United States has certain rights in this invention.

### INTRODUCTION

The present disclosure relates to compositions and methods of manufacturing star polymers as systems for delivering pharmaceutically active compounds for use in different biomedical applications, particularly for delivering pharmaceutically active compounds by the intravenous route for cancer treatment.

### BACKGROUND

Drug delivery systems can be used to modulate the pharmacokinetics of pharmaceutically active compounds used for a variety of applications. For example, drug delivery systems based on liposomes, micelles and linear polymers have been used to package cytotoxic drugs used for cancer treatment. Drug delivery systems have been used to perform any one or all of the following functions: (i) improve drug solubility; (ii) limit distribution and passively or actively target drug molecules to specific tissues; (iii) control the release of drug into specific tissues or cellular compartments; and (iv) protect drug molecules from degradation.

In addition to the aforementioned functions, drug delivery systems used with drugs that bind to extracellular receptors may also perform the function of providing a scaffold for arraying the drug molecules to optimally engage its cognate extracellular receptor. Applications of drug delivery systems for arraying drugs for binding extracellular receptors include the use of delivery systems to array checkpoint inhibitors as a means for reversing immune suppression for cancer treatment. Other applications include the array of targeting molecules that bind to extracellular and/or transmembrane proteins. Another application includes the use of drug delivery systems to array therapeutic monoclonal antibodies or antibody fragments that can be used for the treatment of variety of diseases that rely on recombinant antibody technologies.

There are a variety of challenges that presently limit the utility of drug delivery systems. Many drug delivery systems are often limited by relatively low loading of pharmaceutically active compounds, i.e., low mass ratio of compound to carrier (e.g., polymer carrier), which limits the concentration of active compound that can reach tissues where it is needed. Therefore, next generation delivery systems should be developed to maximize loading of pharmaceutically active compounds.

Another challenge is that many drug delivery systems, such as liposomes and PLGA particles, are often larger than >100 nm or may form aggregates that may be too large for the intended application. In this regard, particles between 10-100 nm in size have been proposed to be an optimal size range for use in a variety of applications, especially for the intravenous delivery of chemotherapeutics and/or immunostimulants to cancers.

A further challenge is that drug delivery systems based on amphiphilic materials often require high net charge (i.e., positive or negative zeta potential) to keep the particles from aggregating. This high net charge can lead to unwanted interactions of the materials with certain tissues, such as non-specific interactions of positively charged particles with cell surfaces. Therefore, novel delivery systems that have optimal charge and surface properties are needed for improving delivery of pharmaceutically active compounds to target tissues by avoiding non-specific interactions with other tissues and/or proteins.

An especially pronounced challenge that has not been adequately addressed by contemporary technologies is the induction of unwanted antibodies against the delivery system or cargo that can lead to rapid clearance of the delivery system from the blood following two or more injections, referred to as “accelerated blood clearance.” The utility of any delivery system of pharmaceutically active compounds may be limited by the induction of unwanted antibody responses. Therefore, approaches for limiting the induction of antibodies that lead to accelerated blood clearance are needed.

Finally, manufacturability remains a major challenge to the translation of drug delivery systems. Drug delivery systems based on emulsions often have high and variable loading as well as broad ranges of particle sizes. Additionally, many drug delivery systems also face major challenges during sterile filtration required by the FDA for injectable drug products. Therefore, chemically defined approaches to achieving precise and reproducible loading on narrow range sizes of particles that are amenable to sterile filtration are needed.

Thus, there is a need for improved drug delivery systems that address one or more of the aforementioned challenges. The present disclosure described novel compositions and methods of manufacturing star polymer drug conjugates that address one or more of these challenges.

### SUMMARY

Embodiment 1 is a star polymer having the formula O[D1]-([X]-A(D2)-[Z]-[D3])ₙ where O is a core; each A is a polymer arm attached to the core; each X is a linker molecule between the core and the polymer arm; each Z is a linker molecule between an end of the polymer arm and D3; D1 is a drug molecule linked to the core; each D2 is a drug molecule linked to reactive monomers distributed along the backbone of the polymer arm; each D3 is a drug molecule linked to the ends of the polymer arms; n is an integer from 5 to 60; wherein each A, X, Z, D2 and D3 may be the same or different; [ ] denotes that the group is optional; wherein the polymer arm, A, comprises reactive monomers, hydrophilic monomers, charged monomers, or any combination thereof, and D2 is linked to the reactive monomers distributed along the polymer arm at a density of between 1 mol % and 80 mol %.

Embodiment 2 is the star polymer of embodiment 1, wherein each D2 is independently selected from amphiphilic or hydrophobic drug molecules, and D2 is linked to the polymer arms at a density of between about 1 mol % and about 40 mol %, or between about 5 mol % and 20 mol %, or between about 7.5 mol % and 15 mol %.

Embodiment 3 is the star polymer of embodiment 1 or 2, wherein the polymer arm comprises charged monomers that are negatively charged at pH 7.4.

Embodiment 4 is the star polymer of any one of embodiments 1 to 3, wherein the charged monomers are distributed along the polymer arm at a density of between about 0.125 to 2.0 times the density at which D2 is linked to reactive monomers distributed along the backbone of the polymer arm.

Embodiment 5 is the star polymer of any one of embodiments 1 to 4, wherein the charged monomers comprise carboxylic acids and/or carboxylic acid salts.

Embodiment 6 is the star polymer of any one of embodiments 1 to 5, wherein the charged monomer comprises beta-alanine, butanoic acid, methyl butanoic acid, dimethylbutanoic acid, 3,3′-((2-(6-aminohexanamido)propane-1,3-diyl)bis(oxy))dipropionic acid, or 13-(6-aminohexanamido)-6,20-bis((2-carboxyethoxy)methyl)-8,18-dioxo-4,11,15,22-tetraoxa-7,19-diazapentacosanedioic acid.

Embodiment 7 is the star polymer of any one of embodiments 1 to 6, wherein the charged monomers are selected from (meth)acrylates and (meth)acrylamides having the chemical formula CH₂═CR₅—C(O)—R₄; wherein R₄ is independently selected from —OR₆, —NHR₆ or —N(CH₃)R₆; R₅ is independently selected from H or CH₃; and R₆ is selected from OH (except for NHR₆ or —N(CH₃)R₆), (CH₂)ⱼCH(NH₂)COOH, (CH₂)ⱼCOOH, (CH₂)ⱼCH(CH₃)COOH, (CH₂)ⱼC(CH₃)₂COOH, CH(COOH)CHCH₂COOH, (CH₂)ⱼNH(CH₂)ⱼCOOH, (CH₂)ⱼN(CH₃)(CH₂)ⱼCOOH, (CH₂)ⱼN⁺(CH₃)₂(CH₂)ⱼCOOH, (CH₂)ⱼN⁺(CH₂—CH₃)₂(CH₂)ⱼCOOH, (CH₂)ₜ—C(O)—NH—(CH₂)ⱼCH(NH₂)COOH, (CH₂)ₜ—C(O)—NH—(CH₂)ⱼCOOH, (CH₂)ₜ—C(O)—NH—(CH₂)ⱼCH(CH₃)COOH, (CH₂)ₜ—C(O)—NH—(CH₂)ⱼC(CH₃)₂COOH, (CH₂)ₜ—C(O)—NH—CH(COOH)CH—CH₂COOH, (CH₂)ₜ—C(O)—NH—(CH₂)ⱼNH(CH₂)ⱼCOOH, (CH₂)ₜ—C(O)—NH—(CH₂)ⱼN(CH₃)(CH₂)ⱼCOOH, (CH₂)ₜ—C(O)—NH—(CH₂)ⱼN⁺(CH₃)₂(CH₂)ⱼCOOH, (CH₂)ₜ—C(O)—NH—(CH₂)ⱼN⁺(CH₂—CH₃)₂(CH₂)ⱼCOOH, (CH₂CH₂O)ₜCH₂CH₂C(O)—NH—(CH₂)ⱼCH(NH₂)COOH, (CH₂CH₂O)ₜCH₂CH₂C(O)—NH—(CH₂)ⱼCOOH, (CH₂CH₂O)ₜCH₂CH₂C(O)—NH—(CH₂)ⱼCH(CH₃)COOH, (CH₂CH₂O)ₜCH₂CH₂C(O)—NH—(CH₂)ⱼC(CH₃)₂COOH, (CH₂CH₂O)ₜCH₂CH₂C(O)—NH—CH(COOH)CHCH₂COOH, (CH₂CH₂O)ₜCH₂CH₂C(O)—NH—(CH₂)ⱼNH(CH₂)ⱼCOOH, (CH₂CH₂O)ₜCH₂CH₂C(O)—NH—(CH₂)ⱼN(CH₃)(CH₂)ⱼCOOH, (CH₂CH₂O)ₜCH₂CH₂C(O)—NH—(CH₂)ⱼN⁺(CH₃)₂(CH₂)ⱼCOOH, (CH₂CH₂O)ₜCH₂CH₂C(O)—NH—(CH₂)ⱼN⁺(CH₂—CH₃)₂(CH₂)ⱼCOOH, wherein t and j are each an integer number of repeating units, each independently selected from between 1 to 6, such as 1, 2, 3, 4, 5 or 6.

Embodiment 8 is the star polymer of embodiment 7, wherein R₄ is independently selected from —NHR₆ or —N(CH₃)R₆; R₅ is independently selected from H or CH₃; and R₆ is selected from (CH₂)₂COOH, (CH₂)₃COOH, (CH₂)₂CH(CH₃)COOH, (CH₂)₂C(CH₃)₂COOH, (CH₂)ₜ—C(O)—NH—(CH₂)₂COOH, (CH₂)ₜ—C(O)—NH—(CH₂)₃COOH, (CH₂)ₜ—C(O)—NH—(CH₂)₂CH(CH₃)COOH or (CH₂)ₜ—C(O)—NH—(CH₂)₂C(CH₃)₂COOH, (CH₂CH₂O)ₜCH₂CH₂C(O)—(CH₂)₂COOH, (CH₂CH₂O)ₜCH₂CH₂C(O)—(CH₂)₃COOH, (CH₂CH₂O)ₜCH₂CH₂C(O)—(CH₂)₂CH(CH₃)COOH or (CH₂CH₂O)ₜCH₂CH₂C(O)—(CH₂)₂C(CH₃)₂COOH, wherein t is an integer number of repeating units selected from between 1 to 6, such as 1, 2, 3, 4, 5 or 6.

Embodiment 9 is the star polymer of any one of embodiments 5 to 8, wherein the carboxylic acid is in the form of an alkylammonium salt.

Embodiment 10 is the star polymer of any one of embodiments 1 to 9, wherein D2 is linked to reactive monomers distributed along the polymer arm at a density of between about 1 mol % and about 8 mol % or between about 3 mol % and about 7 mol % and the polymer arm comprises charged monomers that comprise a nitrogen base selected from primary amines, secondary amines, tertiary amines, aromatic amines, and nitrogen heterocycles that are distributed along the polymer arm at a density of between about 3 mol % and about 30 mol % or about 5 mol % and about 20 mol %.

Embodiment 11 is the star polymer of embodiment 10, wherein the nitrogen base is selected from groups comprising pyrrole, imidazole, pyridine, pyrimidine, pyrazine, diazepine, indole, quinoline, amino quinoline, amino pyridine, purine, pteridine, aniline, or naphthalene amine rings.

Embodiment 12 is the star polymer of any one of embodiments 10 to 11, wherein the charged monomer is selected from (meth)acrylates and (meth)acrylamides with chemical formula CH₂═CR₅—C(O)—R₄ (“Formula II”), wherein R₄ is independently selected from —OR₆, —NHR₆ or —N(CH₃)R₆; R₅ is independently selected from H or CH₃; and R₆ is selected from (CH₂)ⱼ-imidazole, (CH₂)ⱼ-pyridine amine, (CH₂)ⱼ-quinoline amine, (CH₂)ⱼ-naphthalene amine, (CH₂)ⱼN(CH₃)₂, CH₂N(CH₃)₂, CH₂CH₂N(CH₃)₂, CH₂CH₂CH₂N(CH₃)₂, CH₂N(CH₂CH₃)₂, (CH₂)ⱼN(CH₂CH₃)₂, CH₂CH₂N(CH₂CH₃)₂, CH₂CH₂CH₂N(CH₂CH₃)₂, CH₂N(CH(CH₃)₂)₂, (CH₂)ⱼN((CH(CH₃)₂)₂, CH₂CH₂N((CH(CH₃)₂)₂, CH₂CH₂CH₂N(CH(CH₃)₂)₂, (CH₂)ₜ—C(O)—NH—(CH₂)ⱼ-imidazole, (CH₂)ₜ—C(O)—NH—(CH₂)ⱼ-pyridine amine, (CH₂)ₜ—C(O)—NH—(CH₂)ⱼ-quinoline amine, (CH₂)ₜ—C(O)—NH—(CH₂)ⱼ-naphthalene amine, (CH₂)ₜ—C(O)—NH—(CH₂)ⱼN(CH₃)₂, CH₂N(CH₃)₂, (CH₂)ₜ—C(O)—NH—CH₂CH₂N(CH₃)₂, (CH₂)ₜ—C(O)—NH—CH₂CH₂CH₂N(CH₃)₂, (CH₂)ₜ—C(O)—NH—CH₂N(CH₂CH₃)₂, (CH₂)ₜ—C(O)—NH—(CH₂)ⱼN(CH₂CH₃)₂, (CH₂)ₜ—C(O)—NH—CH₂CH₂N(CH₂CH₃)₂, CH₂CH₂CH₂N(CH₂CH₃)₂, CH₂N(CH(CH₃)₂)₂, (CH₂)ₜ—C(O)—NH—(CH₂)ⱼN((CH(CH₃)₂)₂, (CH₂)ₜ—C(O)—NH—CH₂CH₂N((CH(CH₃)₂)₂, (CH₂)ₜ—C(O)—NH—CH₂CH₂CH₂N(CH(CH₃)₂)₂, (CH₂CH₂O)ₜCH₂CH₂(O)—NH—(CH₂)ⱼ-imidazole, (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-quinoline amine, (CH₂CH₂O)ₜCH₂CH₂C(O)—NH—(CH₂)ⱼ-naphthalene amine, (CH₂CH₂O)ₜCH₂CH₂C(O)—NH—(CH₂)ⱼN(CH₃)₂, CH₂N(CH₃)₂, (CH₂CH₂O)ₜCH₂CH₂C(O)—NH—CH₂CH₂N(CH₃)₂, (CH₂CH₂O)ₜCH₂CH₂C(O)—NH—CH₂CH₂CH₂N(CH₃)₂, (CH₂)ₜ—C(O)—NH—CH₂N(CH₂CH₃)₂, (CH₂CH₂O)ₜCH₂CH₂C(O)—NH—(CH₂)ⱼN(CH₂CH₃)₂, (CH₂CH₂O)ₜCH₂CH₂C(O)—NH—CH₂CH₂N(CH₂CH₃)₂, CH₂CH₂CH₂N(CH₂CH₃)₂, CH₂N(CH(CH₃)₂)₂, (CH₂CH₂O)ₜCH₂CH₂C(O)—NH—(CH₂)ⱼN((CH(CH₃)₂)₂, (CH₂CH₂O)ₜCH₂CH₂C(O)—NH—CH₂CH₂N((CH(CH₃)₂)₂, or (CH₂CH₂O)ₜCH₂CH₂C(O)—NH—CH₂CH₂CH₂N(CH(CH₃)₂)₂, wherein t and j are each an integer number of repeating units, each independently selected from between 1 to 6, such as 1, 2, 3, 4, 5 or 6.

Embodiment 13 is the star polymer of any one of embodiments 2 to 12, wherein the amphiphilic or hydrophobic drug molecule is selected from immunostimulants or chemotherapeutics.

Embodiment 14 is the star polymer of embodiment 13, wherein the immunostimulants are selected from pyrimidoindole or lipid-based TLR-4 agonists; adenine-, imdazoquinoline-, or benzonaphthyridine-based TLR-7, TLR-8 or TLR-7/8 agonists; xanthonoid-, amidobenzimidazole-based agonists of STING; and, peptide or 3-(2,3-dihydro-1,4-benzodioxin-6-yl)-2-methylphenyl]methanol based inhibitors of PD1/PDL1.

Embodiment 15 is the star polymer of embodiment 14, wherein the imidazoquinoline-based TLR-7, TLR-8 or TLR-7/8a has the structure:

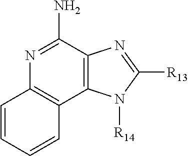

wherein R₁₃ is selected from one of hydrogen, optionally substituted lower alkyl, or optionally substituted lower alkyl ether; and R₁₄ is selected from one of optionally substituted arylalkylamine, or optionally substituted lower alkylamine, wherein the amine provides a reactive handle for attachment to the reactive monomer either directly or via a linker.

Embodiment 16 is the star polymer of embodiment 14, wherein the amidobenzimidazole-based STINGa has the following structure:

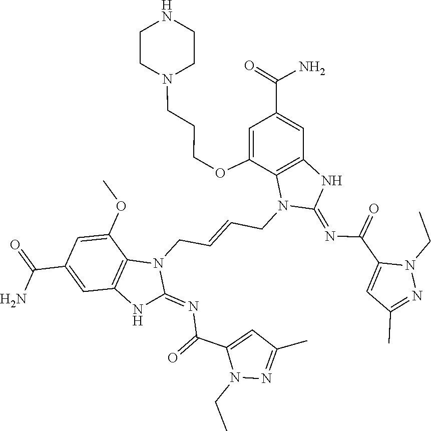 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

Embodiment 17 is the star polymer of embodiment 13, wherein the chemotherapeutics are selected from alkylating agents, antibiotics, antimetabolites, topoisomerase inhibitors, mitotic inhibitors, receptor tyrosine kinase inhibitors, angiogenesis inhibitors, steroids and anti-hormonal agents.

Embodiment 18 is the star polymer of embodiment 1, wherein each D2 is independently selected from hydrophilic drug molecules and D2 is linked to the polymer arms at a density of between about 1 mol % and about 40 mol %, and the hydrophilic monomer is distributed along the polymer arms at a density of between about 60 mol % to about 99 mol %.

Embodiment 19 is the star polymer of embodiment 18, wherein each D2 is independently selected from hydrophilic immunostimulants or hydrophilic chemotherapeutics.

Embodiment 20 is the star polymer of embodiment 19, wherein the hydrophilic immunostimulants are selected from ssRNA-based agonists of TLR-3, hydroxy-adenine based TLR-7 agonists, oligonucleotide-based agonists of TLR-9 and/or cyclic dinucleotide-based STING agonists.

Embodiment 21 is the star polymer of embodiment 20, wherein the cyclic dinucleotide-based STING agonists has the structure:

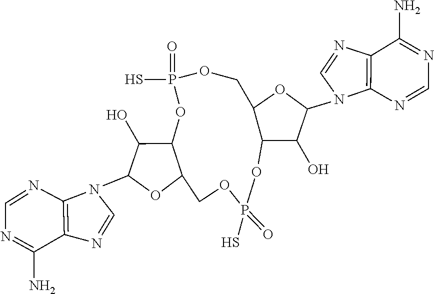 [SMILES: Nc1ncnc2c1ncn2C1OC2COP(=O)(S)OC3C(COP(=O)(S)OC2C1O)OC(n1cnc2c(N)ncnc21)C3O]

Embodiment 22 is the star polymer of embodiment 21, wherein the cyclic dinucleotide-based STING agonist has R or S stereochemistry at the phosphorous stereocenter.

Embodiment 23 is a star polymer of formula O[D1]-([X]-A1(D2)-b-A2-[Z]-[D3])n where O is a core; A1 and A2 collectively form a polymer arm (A) attached to the core, wherein each polymer arm comprises a first block A1 and a second block A2, which are proximal and distal to the core, respectively; each X is a linker molecule between the core and the polymer arm; each Z is a linker molecule between the end of the polymer arm and D3; D1 is a drug molecule linked to the core; each D2 is a drug molecule linked to reactive monomers distributed along the backbone of the polymer arm; each D3 is a drug molecule linked to the ends of the polymer arms; n is an integer number from 5 to 60; wherein each A, A1, A2, X, Z, D2 and D3 may be the same or different; [ ] denotes that the group is optional; the polymer arm comprises reactive monomers, hydrophilic monomers, charged monomers, or any combination thereof; and, D2 is linked to the reactive monomers distributed along the first block of the polymer arm at a density of between 1 mol % and 80 mol %.

Embodiment 24 is the star polymer of embodiment 23, wherein the second block comprises charged monomers that comprise a nitrogen base selected from primary amines, secondary amines, tertiary amines, aromatic amines and nitrogen heterocycles that are distributed along the backbone of the polymer arm at a density of between about 3 mol % and about 30 mol % or about 5 mol % and about 20 mol %.

Embodiment 25 is the star polymer of embodiment 24, wherein the nitrogen base is selected from groups comprising pyrrole, imidazole, pyridine, pyrimidine, pyrazine, diazepine, indole, quinoline, amino quinoline, amino pyridine, purine, pteridine, aniline, and naphthalene amine rings.

Embodiment 26 is the star polymer of embodiment 24 or 25, wherein the charged monomer is selected from (meth)acrylates and (meth)acrylamides with chemical formula CH₂═CR₅—C(O)—R₄ (“Formula II”), wherein R₄ is independently selected from —OR₆, —NHR₆ or —N(CH₃)R₆; R₅ is independently selected from H or CH₃; and R₆ is selected from (CH₂)ⱼ-imidazole, (CH₂)ⱼ-pyridine amine, (CH₂)ⱼ-quinoline amine, (CH₂)ⱼ-naphthalene amine, (CH₂)ⱼN(CH₃)₂, CH₂N(CH₃)₂, CH₂CH₂N(CH₃)₂, CH₂CH₂CH₂N(CH₃)₂, CH₂N(CH₂CH₃)₂, (CH₂)ⱼN(CH₂CH₃)₂, CH₂CH₂N(CH₂CH₃)₂, CH₂CH₂CH₂N(CH₂CH₃)₂, CH₂N(CH(CH₃)₂)₂, (CH₂)ⱼN((CH(CH₃)₂)₂, CH₂CH₂N((CH(CH₃)₂)₂, CH₂CH₂CH₂N(CH(CH₃)₂)₂, (CH₂)ₜ—C(O)—NH—(CH₂)ⱼ-imidazole, (CH₂)ₜ—C(O)—NH—(CH₂)ⱼ-pyridine amine, (CH₂)ₜ—C(O)—NH—(CH₂)ⱼ-quinoline amine, (CH₂)ₜ—C(O)—NH—(CH₂)ⱼ-naphthalene amine, (CH₂)ₜ—C(O)—NH—(CH₂)ⱼN(CH₃)₂, CH₂N(CH₃)₂, (CH₂)ₜ—C(O)—NH—CH₂CH₂N(CH₃)₂, (CH₂)ₜ—C(O)—NH—CH₂CH₂CH₂N(CH₃)₂, (CH₂)ₜ—C(O)—NH—CH₂N(CH₂CH₃)₂, (CH₂)ₜ—C(O)—NH—(CH₂)ⱼN(CH₂CH₃)₂, (CH₂)ₜ—C(O)—NH—CH₂CH₂N(CH₂CH₃)₂, CH₂CH₂CH₂N(CH₂CH₃)₂, CH₂N(CH(CH₃)₂)₂, (CH₂)ₜ—C(O)—NH—(CH₂)ⱼN((CH(CH₃)₂)₂, (CH₂)ₜ—C(O)—NH—CH₂CH₂N((CH(CH₃)₂)₂, (CH₂)ₜ—C(O)—NH—CH₂CH₂CH₂N(CH(CH₃)₂)₂, (CH₂CH₂O)ₜCH₂CH₂(O)—NH—(CH₂)ⱼ-imidazole, (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-quinoline amine, (CH₂CH₂O)ₜCH₂CH₂C(O)—NH—(CH₂)ⱼ-naphthalene amine, (CH₂CH₂O)ₜCH₂CH₂C(O)—NH—(CH₂)ⱼN(CH₃)₂, CH₂N(CH₃)₂, (CH₂CH₂O)ₜCH₂CH₂C(O)—NH—CH₂CH₂N(CH₃)₂, (CH₂CH₂O)ₜCH₂CH₂C(O)—NH—CH₂CH₂CH₂N(CH₃)₂, (CH₂)ₜ—C(O)—NH—CH₂N(CH₂CH₃)₂, (CH₂CH₂O)ₜCH₂CH₂C(O)—NH—(CH₂)ⱼN(CH₂CH₃)₂, (CH₂CH₂O)ₜCH₂CH₂C(O)—NH—CH₂CH₂N(CH₂CH₃)₂, CH₂CH₂CH₂N(CH₂CH₃)₂, CH₂N(CH(CH₃)₂)₂, (CH₂CH₂O)ₜCH₂CH₂C(O)—NH—(CH₂)ⱼN((CH(CH₃)₂)₂, (CH₂CH₂O)ₜCH₂CH₂C(O)—NH—CH₂CH₂N((CH(CH₃)₂)₂, or (CH₂CH₂O)ₜCH₂CH₂C(O)—NH—CH₂CH₂CH₂N(CH(CH₃)₂)₂, wherein t and j are each an integer number of repeating units, each independently selected from between 1 to 6, such as 1, 2, 3, 4, 5 or 6.

Embodiment 27 is the star polymer of any of embodiment 23 to 26, wherein each D2 is independently selected from amphiphilic or hydrophobic drug molecules linked to the first block of the polymer arm at a density of between about 1 mol % to about 80 mol %, or between about 5 mol % to about 40 mol %, or between about 10 mol % to about 30 mol %.

Embodiment 28 is the star polymer of any one of embodiments 23 to 27, wherein the first block is linked to the second block through a pH-sensitive bond selected from hydrazone, silyl-ether and ketal linkages.

Embodiment 29 is the star polymer of any one of embodiments 23 to 28, wherein the degree of polymerization block ratio of the first block to the second block is about 1:5 to about 2:1.

Embodiment 30 is the star polymer of any one of embodiments 1 to 29, wherein D2 is linked to reactive monomers selected from (meth)acrylates and (meth)acrylamides of chemical formula CH₂═CR₈—C(O)—R₇ (“Formula III”), wherein R₇ is an acryl side group comprising a linker molecule for the attachment of D2.

Embodiment 31 is the star polymer of any one of embodiments 1 to 29, wherein D2 is linked to the reactive monomers through a pH-sensitive bond selected from hydrazone, silyl ether and ketal linkages.

Embodiment 32 is the star polymer of embodiment 31, wherein the pH-sensitive bond is a carbohydrazone.

Embodiment 33 is the star polymer of any one of embodiments 1 to 29, wherein D2 is linked to reactive monomers through an enzyme degradable peptide or a sulfatase cleavable linker.

Embodiment 34 is the star polymer of any one of embodiments 1 to 33, wherein each polymer arm independently has a number average molecular weight between about 5 kDa to about 60 kDa, or about 15 kDa to about 50 kDa or about 20 kDa to 40 kDa or about 25 to about 35 kDa.

Embodiment 35 is the star polymer of any one of embodiments 1 to 34, wherein the core (O) has greater than 5 points of attachment for polymer arms (A).

Embodiment 36 is the star polymer of any one of embodiments 1 to 35, wherein the core (O) comprises a branched polymer or dendrimer.

Embodiment 37 is the star polymer of any one of embodiments 1 to 36, wherein the dendrimer or branched polymer that is used to form the core (O) has surface amine groups used for the attachment of polymer arms (A) either directly or via a linker X.

Embodiment 38 is the star polymer of any one of embodiments 1 to 37, wherein the core (O) is a dendrimer selected from PAMAM, bis(MPA), or poly(L-lysine) (PLL).

Embodiment 39 is the star polymer of any one of embodiments 1 to 38, wherein n is greater than or equal to 5 and less than or equal to 60, or n is greater than or equal to 10 and less than or equal to 45, or n is greater than or equal to 20 and less than or equal to 35.

Embodiment 40 is the star polymer of any one of embodiments 1 to 39 comprising a second polymer arm that is linked to the core through an amide linker or pH-sensitive linkage selected from hydrazone, ketal and silyl ether linkages, wherein the second polymer arm comprises hydrophilic monomers, charged monomers, or any combination thereof, additionally wherein the second polymer arm has a number average molecular weight that is equal to or higher than the number average molecular weight of first the polymer arm.

Embodiment 41 is the star polymer of embodiment 40, wherein the polymer arm, A, is 5% to 100% of the polymer arms, and the second polymer arm is 0% to 95% of the polymer arms, or wherein the polymer arm, A, is 50% to 100% of the polymer arms, and the second polymer arm is 0% to 50% of the polymer arms, or wherein the polymer arm, A, is 80% to 100% of the polymer arms, and the second polymer arm is 0% to 20% of the polymer arms.

Embodiment 42 is the star polymer of any one of embodiments 1 to 41, wherein the hydrophilic monomer is selected from acrylates, (meth)acrylates, acrylamides, (meth)acrylamides, allyl ethers, vinyl acetates, vinyl amides, substituted styrenes, amino acids, acrylonitrile, heterocyclic monomers, saccharides, phosphoesters, phosphonamides, sulfonate esters, sulfonamides, or combinations thereof.

Embodiment 43 is the star polymer of embodiment 42, wherein the hydrophilic monomer is selected from (meth)acrylates or (meth)acrylamides of the chemical formula CH₂═CR₂—C(O)—R₁ (“Formula I”), wherein R₁ is independently selected from —OR₃, —NHR₃ or —N(CH₃)R₃; R₂ is independently selected from H and CH₃; and R₃ is independently selected from a neutral hydrophilic substituent, such as H (except for OR₃), CH₃, CH₂CH₃, CH₂CH₂OH, CH₂(CH₂)₂OH, CH₂CH(OH)CH₃, CHCH₃CH₂OH or (CH₂CH₂O)ᵢH, where i is an integer number of repeating units selected from 1, 2, 3, 4, 5 or 6.

Embodiment 44 is the star polymer of any one of embodiments 1 to 43, wherein each D3 is independently selected from targeting molecules.

Embodiment 45 is the star polymer of any one of embodiments 1 to 44, wherein X comprises a triazole, or wherein X comprises between 4 and 24 ethylene oxide units, or wherein X comprises an enzyme degradable linker.

Embodiment 46 is the star polymer of embodiment 45, wherein Z comprises a triazole, or wherein Z comprises between 4 and 24 ethylene oxide units, or wherein Z comprises an enzyme degradable linker.

Embodiment 47 is the star polymer of any one of embodiments 1 to 46, wherein enzyme degradable linker comprises single amino acids, or dipeptides, tripeptides, or tetrapeptides, or combinations thereof.

Embodiment 48 is the star polymer of any one of embodiments 1 to 47, wherein when D3 is absent and the ends of the polymer arms are capped.

Embodiment 49 is the star polymer of embodiment 48, wherein the cap is isobutyronitrile.

Embodiment 50 is the star polymer of any one of embodiments 1 to 49, wherein n is an integer from 20 to 35 and each A, X, and Z is the same.

Embodiment 51 is the star polymer of any one of embodiments 1 to 49, wherein n is an integer from 20 to 35 and each A, X, and Z are chosen to provide at least two different combinations of polymer arm and linkers.

Embodiment 52 is the star polymer of any one of embodiments 1 to 51, wherein the density of charged monomers with a single charged functional group is selected based on the density of attached drug molecule according to Table 1.

Embodiment 53 is the star polymer of embodiment 52, wherein the density of amphiphilic or hydrophobic drug molecules linked to reactive monomers is about 7 mol % to about 15 mol %; and wherein the charged monomers comprise about 5 mol % to about 23 mol % of the monomers in the star polymer.

Embodiment 54 is the star polymer of any one of embodiments 1 to 51, wherein the density of charged monomers with two charged functional groups is selected based on the density of attached drug molecule according to Table 2.

Embodiment 55 is the star polymer of embodiment 54, wherein the density of amphiphilic or hydrophobic drug molecules linked to reactive monomers is about 7 mol % to about 15 mol %; and wherein the bifunctional charged monomers comprises about 3 mol % to about 11 mol % of the monomers in the star polymer.

Embodiment 56 is the star polymer of any one of embodiments 1 to 51, wherein the density of charged monomers with three or four charged functional groups is selected based on the density of attached drug molecule according to Table 3.

Embodiment 57 is the star polymer of embodiment 56, wherein the density of amphiphilic or hydrophobic drug molecules linked to reactive monomers is about 7 mol % to about 15 mol %; and the trifunctional or tetrafunctional charged monomers comprise about 3 mol % to about 11 mol % of the monomers in the star polymer.

Embodiment 58 is a process for preparing a star polymer according to any one of embodiments 1 to 57, the process comprising: producing the polymer arm comprising reactive monomers by RAFT polymerization, reacting the polymer arm comprising the reactive monomers with D2 to link D2 to the reactive monomer, and grafting the polymer arm to the core by reacting X1 with X2 to form the linker X, which links the polymer arm to the core.

Embodiment 59 is the process according to embodiment 58, wherein X1 comprises a strained alkyne and X2 comprises an azide.

Embodiment 60 is the process according to embodiment 59, wherein the strained alkyne is linked to the core via a linker comprising between 4 and 24 ethylene oxide units.

Embodiment 61 is a star polymer having the formula O[D1]-([X]-A-[Z]-D3)n where O is a core; each A is a polymer arm attached to the core; each X is a linker molecule between the core and the polymer arm; each Z is a linker molecule between an end of the polymer arm and D3; D1 is a drug molecule linked to the core; each D3 is a drug molecule linked to the ends of the polymer arms; n is an integer number from 1 to 60; wherein each A, X, Z, and D3 may be the same or different; [ ] denotes that the group is optional, wherein the polymer arm comprises reactive monomers, hydrophilic monomers, charged monomers, or any combination thereof, the polymer arm has a number average molecular weight between about 5 kDa to about 60 kDa, or about 15 kDa to about 50 kDa, or about 20 kDa to about 40 kDa.

Embodiment 62 is the star polymer of any one of embodiments 1 to 57 or 61, wherein D3 is selected from peptide-based CPIs.

Embodiment 63 is the star polymer of embodiment 62, wherein the peptide-based CPI has the structure:

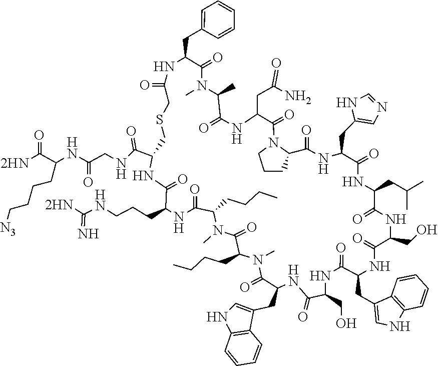

wherein the azide provides a reactive handle for attachment to a polymer arm either directly or via a linker.

Embodiment 64 is the use of the star polymer of any one of embodiments 1 to 63 as a medicament.

Embodiment 65 is a pharmaceutical composition comprising the star polymer of any one of embodiments 1 to 63 and a pharmaceutically acceptable carrier.

Embodiment 66 is the pharmaceutical composition of embodiment 65 for use in the treatment or prophylaxis of cancer.

Embodiment 67 is the pharmaceutical composition of embodiment 65 when used in the treatment or prophylaxis of cancer.

Embodiment 68 is the use of the pharmaceutical composition of embodiment 65 for the treatment or prophylaxis of cancer.

Embodiment 69 is a method of treating cancer in a subject in need of treatment, the method comprising administering the pharmaceutical composition of embodiment 65 to the subject.

Embodiment 70 is the use of the star polymer of any one of embodiments 1 to 63 in the preparation of a medicament for the treatment or prophylaxis of cancer.

Embodiment 71 is the pharmaceutical composition of any one of embodiments 65 to 67, the use of embodiment 68 or the method of embodiment 69 wherein the star polymer is administered by intravenous, intratumoral, intramuscular or subcutaneous routes of administration.

Embodiment 72 is the pharmaceutical composition of any one of embodiments 65 to 67, the use of embodiment 68, the method of embodiment 69 or the use of embodiment 70 wherein the cancer is selected from hematological tumors, such as leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia; solid tumors, such as sarcomas and carcinomas, including fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers (including adenocarcinoma, a bronchiolaveolar carcinoma, a large cell carcinoma, or a small cell carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma); skin cancer, such as a basal cell carcinoma, a squamous cell carcinoma, a Kaposi's sarcoma, or a melanoma; and, premalignant conditions, such as variants of carcinoma in situ, or vulvar intraepithelial neoplasia, cervical intraepithelial neoplasia, or vaginal intraepithelial neoplasia.

### BRIEF DESCRIPTION OF DRAWINGS

FIG. 1 is a generic structure of a star polymer of the present disclosure used for ligand array, wherein a dendrimer core (O) is linked through a linker X to an integer number (n) of polymer arms (A) that are linked to a drug molecule (D3) through a linker Z.

FIG. 2 is a plot of the particle size (number percent) distribution of Compound 87 (darker line, right shifted; mean diameter=26.6 nm), which is a star polymer displaying a peptide-based checkpoint inhibitor (CPI), and the star polymer without the CPI attached (Compound 82, lighter line). Samples were suspended at 0.5 mg/mL in PBS pH 7.4 and particle size was determined using dynamic light scattering (Malvern ZetaSizer Ultra). For Compound 87, n=24 (i.e., 24 polymer arms), each linked to D3, which, in this example, is a peptide-based CPI.

FIG. 3 shows dose-response curves for in vitro inhibition of PD-1/PD-L1 interactions by different PD-1 antagonists, including Compound 87. Inhibition was determined by measuring fluorescence, which is proportional to luciferase expression downstream of T cell receptor signaling. Compound Q conjugated to a star polymer (i.e., Compound 87) demonstrated similar levels of PD-1 inhibition with an EC50 as compared with Nivolumab.

FIG. 4 shows the impact that polymer arm molecular weight and dendrimer core generation have on the size (Rg) of star polymers. These results demonstrate that star polymer size, including hydrodynamic size, can be precisely tuned principally by varying the molecular weight of the polymer arms.

FIG. 5 shows the impact that polymer arm length (expressed as molecular weight; see Table 4) and D3 density have on star polymer hydrodynamic radius (Rh). Note: Polymer arm length principally determined Rh, independent on arm density or D3 density.

FIG. 6 shows that the synthetic route used to synthesize polymer arms (A) can impact the propensity of star polymers to cross-link, which results in increased molecular weight and polydispersity index (PDI) determined by gel permeation chromatography (GPC) in tandem with multi-angle light scattering (MALS) and refractive index (RI) detectors. The figure shows polydispersity index (PDI=Mw/Mn) change over time for star polymers produced using polymer arms with the linker precursor X2 added to the polymer arm either (i) during polymerization or (ii) during the capping step.

FIGS. 7 and 8 show turbidity for different polymer arms in PBS buffer over a pH range of 5.5 to 7.5. Note: Turbidity (OD at 490 nm)>0.05 indicates that the polymer arms are precipitating from solution, i.e., forming aggregates.

FIG. 9 shows survival curves for C57BL/6 mice that were implanted subcutaneously with MC38 tumors, randomized to groups and then provided the indicated treatment (normalized to 50 nmol of TLR-7/8a, 2BXy) by direct intratumoral injection between days 7-10 after tumor implantation.

FIG. 10 shows lymph node cytokine production induced by different compositions of the TLR-7/8a, Compound A (“2BXy”). Each of the TLR-7/8a compositions (normalized to 25 nmol TLR-7/8a dose) were injected subcutaneously at time 0 and lymph nodes were harvested at 4 days and cultured ex vivo, as summarized in the schematic shown at the top of FIG. 10. IL-12 concentrations in the culture supernatant were assessed by ELISA, and the results for each replicate (each lymph node) are shown.

FIG. 11 shows tumor volume and survival curves for tumor bearing mice treated with different compositions of a STINGa. As depicted in FIG. 11A, BALB/c mice were implanted subcutaneously with CT26 tumors, randomized to groups and then provided the indicated treatment (normalized to 35 nmol of STINGa, diABZI) on day 11. Tumor size was measured by digital calipers (FIG. 11B) and survival (FIG. 11C) were assessed up to 80 days after tumor implantation.

FIG. 12 shows tumor volume and survival curves for tumor bearing mice treated with different compositions of a STINGa. As depicted in FIG. 12A, BALB/c mice were implanted subcutaneously with CT26 tumors, randomized to groups and then provided the indicated treatment (normalized to 7 nmol of STINGa, diABZI) on day 11. Tumor size was measured by digital calipers for up to 30 days after tumor implantation (FIG. 12 B & C). To assess acute toxicity, mice were bled 4 hours after treatment, and blood IP-10 concentration was assessed by ELISA (FIG. 12D).

FIG. 13 shows zeta potential for Compounds 99, 103 and 104 in PBS buffer over a pH range from 5.5 to 8.0.

FIG. 14 shows turbidity of Compounds 100 and 105-109 in PBS buffer over a pH range from 5.0 to 8.0.

FIG. 15 shows zeta potential (FIG. 15A) and turbidity (OD 490 nm) (FIG. 15B) for Compounds 110-131 containing different D2 (circle for drug-free polymer arms, square for Naph, triangle for 2BXy and down-pointing triable for diABZI) and varied mol % DMBA (x axis) in PBS buffer at physiologic pH 7.4.

FIG. 16 shows turbidity (OD 490 nm) for Compounds 110-131 containing 10 mol % of D2 (Naph, 2BXy and diABZI) and varied mol % DMBA (0-20 mol %) in PBS buffer at pH ranging from 5.5 to 7.4.

FIG. 17 shows turbidity (OD 490 nm) for star polymer Compounds 132-137 containing 10 mol % diABZI and varied mol % DMBA in PBS buffer at pH ranging from 5.5 to 7.4.

FIG. 18 shows zeta potential for Compounds 135 and Compound 137 containing 10 mol % diABZI and 12.5 or 20 mol % DMBA in PBS buffer at pH ranging from 5.5 to 7.4.

FIG. 19 shows THP1-NF-kB cell uptake of cationic and anionic SRCs bearing diABZI drug molecules at diABZI concentration ranging from 1 to 1000 mM.

FIG. 20 shows THP-1 NF-kB cell uptake of cationic and anionic SRCs bearing diABZI drug molecules at drug concentration ranging from 1 to 1000 mM.

FIG. 21 shows uptake of star polymers after 2 hr incubation with mouse splenocytes at different pH conditions. In FIG. 21A, values are normalized to percent uptake at pH 7.4 for each construct. In FIG. 21B, Mean Fluorescent Intensity (MFI) is graphed to show average uptake of each construct at pH 6.0.

FIG. 22 shows tumor volume and survival curves for tumor bearing mice treated with different compositions of a STINGa. As depicted in FIG. 22A, C57BL/6 mice were implanted subcutaneously with MC38 tumors, randomized to groups, and then provided the indicated treatment (normalized to 35 nmol of STINGa, diABZI) on day 10. Tumor sizes were measured by digital calipers (FIG. 22B) and survival (FIG. 22C) were assessed up to 60 days after tumor implantation. Tumor growth curves are stopped after one mouse/group is euthanized for tumor size. Mice euthanized for reasons other than tumor size are censored.

FIG. 23 shows uptake of star polymers after 2 h incubation with mouse splenocytes at pH 7.4. Mean Fluorescent Intensity (MFI) is graphed to show average uptake of each construct.

FIG. 24 shows tumor volume and survival curves for tumor bearing mice treated with different compositions of a STINGa. As depicted in FIG. 24A, C57BL/6 mice were implanted subcutaneously with MC38 tumors, randomized to groups and then provided the indicated treatment (normalized to 35 nmol of STINGa, diABZI) on day 10. Tumor sizes were measured by digital calipers (FIGS. 24B & C) and survival (FIGS. 24D & E) were assessed up to 60 days after tumor implantation. Tumor growth curves are stopped after one mouse/group is euthanized for tumor size. Mice euthanized for reasons other than tumor size are censored. Body weight was measured at the same time on days d0-3, d5, d7, and d9 after vaccination (FIGS. 24F & G). Values are presented as percent of body weight on the day of vaccination.

FIG. 25. Experiment timecourse using five C57BL/6 per group implanted with B16 tumors, randomized and treated intratumorally (IT) with polymer drug conjugates (diABZI) at a dose of 7 nmol per animal on day 11. Body weight was then assessed on days 11, 12, 13, 15 and 17.

FIG. 26. Tumor growth kinetics, shown as the change in tumor volume (mm³) over time, following intratumoral treatment of B16 tumor (timeline shown in FIG. 25) with SRC Compounds 150 and 166.

FIG. 27. Tumor growth kinetics, shown as the change in tumor volume (mm³) over time, following intratumoral treatment of B116 tumor (timeline shown in FIG. 25) with SDB Compounds 168 and 169.

FIG. 28. Mouse survival Kaplan-Meier curve, shown as the percentage of animals that survived over time, following intratumoral treatment of B16 tumor (timeline shown in FIG. 25 with SRC Compounds 150 and 166.

FIG. 29. Mouse survival Kaplan-Meier curve, shown as the percentage of animals that survived over time, following intratumoral treatment of B16 tumor (timeline shown in FIG. 25) with SDB Compounds 168 and 169.

FIG. 30. Mouse body weight, shown as the change in body weight percentage as measured by time from vaccination, following intratumoral treatment of B16 tumor (timeline shown in FIG. 25) with SRC compounds 150 and 166.

FIG. 31. Mouse body weight following, shown as the change in body weight percentage as measured by time from vaccination, intratumoral treatment of B16 tumor (timeline shown in FIG. 25) with SDB compounds 168 and 169.

FIG. 32 shows the assay diagram for evaluation of AMC-peptides in PBS buffer and in cathepsin B. Peptide linker stock solutions (10 mM in DMSO) are diluted to 1 mM and then incubated with either PBS buffer (negative control) or Cathepsin B in 25 mM 2-ethanesulfonic acid (MES), 1 mM DTT at pH 5, 37° C.; aliquots are removed and analyzed by HPLC at 5 min, 1 hour and 6 hours.

FIG. 33 shows the assay diagram for evaluation of AMC-peptides in mouse plasma. Peptide linker stock solutions (10 mM in DMSO) are diluted to 1 mM and then incubated with mouse plasma; aliquots are removed, blood proteins are precipitated with cold acetonitrile, pelleted with centrifugation, and the supernatant analyzed by HPLC.

FIG. 34 shows the percent cleaved (% cleaved) of AMC-peptides in both cathepsin B and plasma, quantified by monitoring the UV absorbance (350 nm) of AMC compounds after 6 hrs of incubation.

### DESCRIPTION OF EMBODIMENTS

Details of terms and methods are given below to provide greater clarity concerning compounds, compositions, methods and the use(s) thereof for the purpose of guiding those of ordinary skill in the art in the practice of the present disclosure. The terminology in this disclosure is understood to be useful for the purpose of providing a better description of particular embodiments and should not be considered limiting.

About: In the context of the present disclosure, “about” when referring to a measurable value such as an amount, a temporal duration, and the like, is meant to encompass variations of ±20%, 10%, 5%, 1%, or +0.1% from the specified value, as such variations are appropriate to perform the disclosed methods.

Administration: To provide or give to a subject an agent, for example, an immunogenic composition comprising a star polymer as described herein, by any effective route. Exemplary routes of administration include, but are not limited to, oral, injection (such as subcutaneous, intramuscular, intradermal, intraperitoneal, and intravenous), transdermal (for example, topical), intranasal, vaginal, and inhalation routes.

“Administration of” and “administering a” compound should be understood to mean providing a compound, a prodrug of a compound, a star polymer composition or a pharmaceutical composition as described herein. The compound or composition can be administered by another person to the subject or it can be self-administered by the subject.

Antigen-presenting cell (APC): Any cell that presents antigen bound to MHC class I or class II molecules to T cells, including but not limited to monocytes, macrophages, dendritic cells, B cells, T cells and Langerhans cells.

Antigen: Any molecule that contains an epitope that binds to a T cell or B cell receptor and can stimulate an immune response, in particular, a B cell response and/or a T cell response in a subject. The epitopes may be comprised of peptides, glycopeptides, lipids or any suitable molecules that contain an epitope that can interact with components of specific B cell or T cell proteins. Such interactions may generate a response by the immune cell. “Epitope” refers to the region of a peptide antigen to which B and/or T cell proteins, i.e., B-cell receptors and T-cell receptors, interact.

Amphiphilic: The term “amphiphilic” is used herein to describe the properties of a substance containing both hydrophilic or polar (water-soluble) and hydrophobic or non-polar (water-insoluble) groups. Substances with amphiphilic properties may be referred to generically as amphiphiles. Amphiphiles include polymers that are comprised of both a hydrophilic region and a hydrophobic region, such as certain amphiphilic block copolymers described herein that comprise hydrophilic blocks and hydrophobic blocks.

CD4: Cluster of differentiation 4, a surface glycoprotein that interacts with MHC Class II molecules present on the surface of other cells. A subset of T cells that express CD4 are commonly referred to as helper T cells.

CD8: Cluster of differentiation 8, a surface glycoprotein that interacts with MHC Class I molecules present on the surface of other cells. A subset of T cells that express CD8 are commonly referred to as cytotoxic T cells or killer T cells.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units.

Charged monomers: Refers to monomers that have one or more functional groups that are or can be positively or negatively charged (under certain conditions). The functional groups comprising the charged monomers may be partial or full integer values of charge. A charged monomer may have a single charged functional group or multiple charged functional groups, which may be the same or different. Functional groups may be permanently charged or the functional groups comprising the charged molecule may have charge depending on the pH. The charged monomer may be comprised of positive functional groups, negative functional groups or both positive and negative functional groups. The net charge of the charged monomer may be positive, negative or neutral. The charge of a molecule, such as a charged monomer, can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom may be considered to have a full integer value of charge when the number of electrons assigned to that atom is less than or equal to the atomic number of the atom. The charge of a functional group is determined by summing the charge of each atom comprising the functional group. The net charge of the charged monomer is determined by summing the charge of each atom comprising the molecule. Those skilled in the art are familiar with the process of estimating charge of a molecule, or individual functional groups, by summing the formal charge of each atom in a molecule or functional group, respectively.

Charged monomers may comprise negatively charged functional groups such as those that occur as the conjugate base of an acid at physiologic pH (e.g., functional groups with a pKa less than about 6.5), e.g., at a pH of about 7.4. These include but are not limited to molecules bearing carboxylates, sulfates, sulfonates, phosphates, phosphoramidates, and phosphonates. Charged monomers may comprise positively charged functional groups such as those that occur as the conjugate acid of a base at physiologic pH (e.g., functional groups wherein the pKa of the conjugate acid of a base is greater than about 8.5). These include but are not limited to molecules bearing primary, secondary and tertiary amines, as well as ammonium and guanidinium. Charged monomers may comprise functional groups with charge that is pH independent, including quaternary ammonium, phosphonium and sulfonium functional groups. Charged monomers may comprise zwitterions comprising both negative and positive functional groups. Charged monomers useful for the practice of the invention of the present disclosure are disclosed herein. Charged monomers on a copolymer are sometimes referred to as charged comonomers.

For star polymers with polymer arms comprising charged monomers that are pH-responsive, the charge of the average charged monomer and therefore the star polymer as a whole depends on the pH of the aqueous solution in which the star polymer is suspended. For simplicity of discussions herein, charged monomers comprising acids (e.g., carboxylic acids) are said to be negative (or negatively charged, i.e., they exist as the conjugate base of the acid) at pH values greater than or equal to the pKa of the acid (i.e., the pKa of the acid as a polymer) and are described as neutral at pH values less than the pKa. For example, a star polymer with polymer arms comprising charged monomers further comprising a carboxylic acid with pKa of ˜7 would be described as negative at pH of 7.0 or higher, but neutral at 6.9 or less, e.g., 6.5. Similarly, charged monomers comprising bases that are positive upon protonation are said to be positive (or positively charged, i.e., they exist as the conjugate acid of the base) at pH values less than or equal to the pKa (i.e., the pKa of the conjugate acid as a polymer) and are described as neutral at pH values greater than the pKa. For example, a star polymer with polymer arms comprising charged monomers further comprising a tertiary amine with pKa of 7.0 for the conjugate acid of the base would be described as neutral at pH higher than 7.0, but positive at 7.0 or less, e.g., 6.5. Note: Charged monomers that are pH-responsive are still described in chemical formulae and in descriptions as charged monomers, independent of the pH and state of charge of the molecule.

Chemotherapeutic: As defined herein broadly refers to pharmaceutically active molecules useful in the treatment of cancer and include growth inhibitory agents or cytotoxic agents, including alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL® docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA™), BIBW 2992 (TOVOK™), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al, eds.), Chapter 10, McMillan Publishing, are also suitable chemotherapeutic agents.

Chemotherapeutics (also referred to as chemotherapeutic agents) are pharmaceutically active compounds and may therefore be referred to herein generally as drugs or drug molecules, or “D” in formulae, e.g., D2 when linked to reactive monomers distributed along polymer arms. For clarity, the terms chemotherapeutic(s) and chemotherapeutic agent(s) are used herein to describe any synthetic or naturally occurring molecules useful for cancer treatment, though, certain classes of drug molecules may alternatively be described by their mechanism of action, e.g., angiogenesis inhibitors are a type of chemotherapeutic drug that inhibit angiogenesis. While certain immunomodulators, e.g., immunostimulants, may be useful for cancer treatment, immunomodulators, inclusive of immunostimulants and immunosuppressants are not referred to as chemotherapeutics.

Click chemistry reaction: A bio-orthogonal reaction that joins two compounds together under mild conditions in a high yield reaction that generates minimal, biocompatible and/or inoffensive byproducts. An exemplary click chemistry reaction used in the present disclosure is the reaction of a strained-alkyne group provided on a linker precursor X1 with an azide provided on a linker precursor X2 that forms a linker X comprising a triazole through strain-promoted [3+2] azide-alkyne cyclo-addition.

Copolymer: A polymer derived from two (or more) different monomers, as opposed to a homopolymer where only one monomer is used. Since a copolymer includes at least two types of constituent units (also structural units), copolymers may be classified based on how these units are arranged along the chain. A copolymer may be a statistical copolymer (also referred to as a random copolymer) wherein the two or monomer units are distributed randomly; or, the copolymer may be an alternating copolymer wherein the two or more monomer units are distributed in an alternating sequence. The term “block copolymer” refers generically to a polymer composed of two or more contiguous blocks of different constituent monomers or comonomers (if a block comprises two or more different monomers). Block copolymer may be used herein to refer to a copolymer that comprises two or more homopolymer subunits, two or more copolymer subunits or one or more homopolymer subunits and one or more copolymer subunits, wherein the subunits may be linked directly by covalent bonds or the subunits may be linked indirectly via an intermediate non-repeating subunit, such as a junction block or linker. Blocks may be based on linear and/or brush architectures. Block copolymers with two or three distinct blocks are referred to herein as “diblock copolymers” and “triblock copolymers,” respectively. Note: copolymers may be referred to generically as polymers, e.g., a statistical copolymer may be referred to as a polymer or copolymer; and, polymers comprising three distinct units may be referred to as terpolymers, though, polymers comprising four or more units are typically referred to generically as copolymers or polymers. Similarly, a block copolymer may be referred to generically as a polymer. For example, star polymers of the present disclosure may comprise homopolymer, copolymer and/or terpolymer arms, which may be referred to generically as polymers or polymer arms.

Drug: Refers to any pharmaceutically active molecule—including, without limitation, proteins, peptides, sugars, saccharides, nucleosides, inorganic compounds, lipids, nucleic acids, small synthetic chemical compounds, macrocycles, etc.—that has a physiological effect when ingested or otherwise introduced into the body. Pharmaceutically active compounds can be selected from a variety of known classes of compounds, including, for example, analgesics, anesthetics, anti-inflammatory agents, anthelmintics, anti-arrhythmic agents, antiasthma agents, antibiotics (including penicillins), anticancer agents, anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, such as therapeutic antibodies and antibody fragments, MHC-peptide complexes, cytokines and growth factors, glycoproteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines. Drugs may also be referred to as pharmaceutically active agents, pharmaceutically active substances or biologically active compounds or bioactive molecules. Note: Targeting molecules are also considered drugs herein due to their direct physiological effects, as well as indirect affect on PK, distribution and subcellular trafficking of other drugs. Note: Small molecule drugs, as used herein, refers to pharmaceutically active molecules, that are often produced by synthetic means and have molecular weight less than or equal to about 2,500 Daltons, though, more typically, less than or equal to about 1,000 Daltons.

Graft polymer: May be described as a polymer that results from the linkage of a polymer of one composition to the side chains of a second polymer of a different composition. A first polymer linked through co-monomers to a second polymer is a graft copolymer. A first polymer linked through an end group to a second polymer may be described as a block polymer (e.g., A-B type di-block) or an end-grafted polymer. Polymer arms linked (or ‘grafted’) to cores (O) based on branched polymers or dendrimers may be referred to as graft polymers or, more specifically, star polymers.

Hydrophilic: Refers to the tendency of a material to disperse freely in aqueous solutions (sometimes referred to as aqueous media). A material is considered hydrophilic if it prefers interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble up to 0.1 mg/mL or more. Neutral hydrophilic monomers (sometimes referred to as “hydrophilic monomers”) are monomers that form water-soluble polymers. For example, a HPMA monomer may be referred to as a hydrophilic monomer because poly(HPMA) is a water-soluble polymer. Note: Charged monomers may be hydrophilic but are typically charged at physiologically relevant pH values and so are referred to as charge monomers herein, whereas hydrophilic monomers that are not charged at physiologically relevant pH values are referred to as neutral hydrophilic monomers or just hydrophilic monomers. Hydrophilic block refers to the portion of a block copolymer that is water soluble.

Hydrophobic: Refers to the tendency of a material to avoid contact with water. A material is considered hydrophobic if it prefers interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble down to 0.1 mg/mL or less. Hydrophobic monomers are monomers that form polymers that are insoluble in water or insoluble in water at certain temperatures, pH and concentration. For example, a styrene monomer may be referred to as a hydrophobic monomer because poly(styrene) is a water insoluble polymer. Hydrophobic block refers to the portion of a block copolymer that is insoluble in water at certain temperature, pH and concentrations. Hydrophobic drugs (or sometimes “hydrophobic drug molecules”) refer to drug molecules that are insoluble down to about 0.1 mg/mL or less in aqueous solutions at pH of about pH 7.4. Amphiphilic drugs (or sometimes “amphiphilic drug molecules”) are drug molecules that have the tendency to assemble into supramolecular structures, e.g., micelles, in aqueous solutions and/or have limited solubility in aqueous solutions at pH of about pH 7.4. Hydrophobic drug molecules and amphiphilic drug molecules may also be described as amphiphilic or hydrophobic drug molecules, hydrophobic or amphiphilic drug molecules, amphiphilic or hydrophobic drugs, or hydrophobic or amphiphilic drugs.

Immune response: A change in the activity of a cell of the immune system, such as a B cell, T cell, or monocyte, as a result of a stimulus, either directly or indirectly, such as through a cellular or cytokine intermediary. In one embodiment, the response is specific for a particular antigen (an “antigen-specific response”). In one embodiment, an immune response is a T cell response, such as a CD4 T cell response or a CD8 T cell response. In one embodiment, an immune response results in the production of additional T cell progeny. In one embodiment, an immune response results in the movement of T cells. In another embodiment, the response is a B cell response, and results in the production of specific antibodies or the production of additional B cell progeny. In other embodiments, the response is an antigen-presenting cell response. “Enhancing an immune response” refers to co-administration of an adjuvant and an immunogenic agent, such as a peptide antigen, as part of a peptide antigen conjugate, wherein the adjuvant increases the desired immune response to the immunogenic agent compared to administration of the immunogenic agent to the subject in the absence of the adjuvant. In some embodiments, an antigen is used to stimulate an immune response leading to the activation of cytotoxic T cells that kills virally infected cells or cancerous cells. In some embodiments, an antigen is used to induce tolerance or immune suppression. A tolerogenic response may result from the unresponsiveness of a T cell or B cell to an antigen. A suppressive immune response may result from the activation of regulatory cells, such as regulatory T cells that downregulate the immune response, i.e., dampen then immune, response. Antigens administered to a patient in the absence of an adjuvant are generally tolerogenic or suppressive and antigens administered with an adjuvant are generally stimulatory and lead to the recruitment, expansion and activation of immune cells.

Immunomodulators: Refers to a type of drug (i.e., pharmaceutically active substance) that modulates the activity of cells of the immune system, which includes immunostimulants and immunosuppressants.

Immunostimulants: Refers to any synthetic or naturally occurring drugs that promote pro-inflammatory and/or cytotoxic activity by immune cells. Exemplary immunostimulants include pattern recognition receptor (PRR) agonists, such as synthetic or naturally occurring agonists of Toll-like receptors (TLRs), stimulator of interferon gene agonists (STINGa), nucleotide-binding oligomerization domain-like receptor (NLR) agonists, retinoic acid-inducible gene-I-like receptors (RLR) agonists or certain C-type lectin receptor (CLR) agonists, as well as certain cytokines (e.g., certain interleukins), such as IL-2; certain chemokines or small molecules that bind chemokine receptors; certain antibodies, antibody fragments or synthetic peptides that activate immune cells, e.g., through binding to stimulatory receptors, e.g., anti-CD40, or, e.g., by blocking inhibitory receptors, e.g., anti-CTLA4, anti-PD1, etc. Various immunostimulants for the practice of the present disclosure are described throughout the specification. For clarity, certain pharmaceutically active compounds that stimulate the immune system may be referred to as immunostimulants or more generally as drug molecules (abbreviated “D” in formulae).

Linked or coupled: The terms “linked” and “coupled” mean joined together, either directly or indirectly. A first moiety may be covalently or noncovalently linked to a second moiety. In some embodiments, a first molecule is linked by a covalent bond to another molecule. In some embodiments, a first molecule is linked by electrostatic attraction to another molecule. In some embodiments, a first molecule is linked by dipole-dipole forces (for example, hydrogen bonding) to another molecule. In some embodiments, a first molecule is linked by van der Waals forces (also known as London forces) to another molecule. A first molecule may be linked by any and all combinations of such couplings to another molecule. The molecules may be linked indirectly, such as by using a linker (sometimes referred to as linker molecule). The molecules may be linked indirectly by interposition of a component that binds non-covalently to both molecules independently. The term “Linker” used in chemical formula means any suitable linker molecule.

Net charge: The sum of electrostatic charges carried by a molecule or, if specified, a section of a molecule.

Mol %: Refers to the percentage of a particular type of monomeric unit (or “monomer”) that is present in a copolymer (sometimes just referred to as a polymer). For example, a copolymer comprised of 100 monomeric units of A and B with a density (or “mol %”) of monomer A equal to 10 mol % would have 10 monomeric units of A, and the remaining 90 monomeric units (or “monomers”) may be monomer B or another monomer unless otherwise specified.

Monomeric unit: The term “monomeric unit” is used herein to mean a unit of polymer molecule containing the same or similar number of atoms as one of the monomers. Monomeric units, as used in this specification, may be of a single type (homogeneous) or a variety of types (heterogeneous). For example, poly(amino acids) are comprised of amino acid monomeric units; and poly((meth)acrylamides) are comprised of (meth)acrylamide monomeric units. Monomeric units may also be referred to as monomers or monomer units or the like.

Particle: Typically refers to a nano- or micro-sized supramolecular structure comprised of an assembly of molecules, but may also refer to nano-sized macromolecules, e.g., star polymers that are within 1 to 100 nm diameter size range.

Pattern recognition receptors (PRRs): Receptors expressed by various cell populations, particularly innate immune cells that bind to a diverse group of synthetic and naturally occurring molecules referred to as pathogen-associated molecular patterns (PAMPS) as well as damage associated molecular patterns (DAMPs). PAMPs are conserved molecular motifs present on certain microbial organisms and viruses. DAMPs are cellular components that are released or expressed during cell death or damage. PAMP or DAMP activation of pattern recognition receptors induces an intracellular signaling cascade resulting in the alteration of the host cell's physiology. Such physiological changes can include changes in the transcriptional profile of the cell to induce expression of a range of pro-inflammatory and pro-survival genes. The coordinated expression of these genes may enhance adaptive immunity.

There are several classes of PRRs. Non-limiting examples of PRRs include Toll-like receptors (TLRs), RIG-1-like receptors (RLRs), NOD-like receptors (NLRs), Stimulator of Interferon Genes receptor (STING), and C-type lectin receptors (CLRs). Agonists of such PRRs can be used as immunostimulants. For more information on pattern recognition receptors, see Wales et al., Biochem Soc Trans., 35:1501-1503, 2007.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, such as one or more therapeutic cancer vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Physiologic: Refers to a condition or conditions that are representative of the conditions in a subject. A physiologic buffer refers to a buffer that has similar salt and pH to fluids in the body of a subject, such as serum. Physiologic pH is about pH 7.4.

Plurality: The word “plurality” is used herein to mean more than one.

Polar: A description of the properties of matter. Polar is a relative term and may describe a molecule or a portion of a molecule that has partial charge that arises from differences in electronegativity between atoms bonded together in a molecule, such as the bond between nitrogen and hydrogen. Polar molecules have a preference for interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may have a preference for interacting with a polar solvent such as water. In specific, non-limiting cases, introduction of additional polar groups may increase the solubility of a portion of a molecule.

Polymer: A molecule containing repeating structural units (monomers). Polymers linked to cores (O) are referred to as polymer arms (A). Star polymers refers to macromolecules comprising one or more polymer arms (A) grafted to a core (O).

Polymerization: A chemical reaction, usually carried out with a catalyst, heat or light, in which monomers combine to form a chainlike, branched or cross-linked macromolecule (a polymer). The chains, branches or cross-linked macromolecules can be further modified by additional chemical synthesis using the appropriate substituent groups and chemical reactions. The monomers may contain reactive substances. Polymerization commonly occurs by addition or condensation. Addition polymerization occurs when an initiator, usually a free radical, reacts with a double bond in the monomer. The free radical adds to one side of the double bond, producing a free electron on the other side. This free electron then reacts with another monomer, and the chain becomes self-propagating, thus adding one monomer unit at a time to the end of a growing chain. Condensation polymerization involves the reaction of two monomers resulting in the splitting out of a water molecule. In other forms of polymerization, a monomer is added one at a time to a growing chain through the staged introduction of activated monomers, such as during solid phase peptide synthesis.

Purified: Having a composition that is relatively free of impurities or substances that adulterate or contaminate a substance. The term purified is a relative term and does not require absolute purity. Thus, for example, a purified peptide preparation is one in which the peptide or protein is more enriched than the peptide or protein is in its natural environment, for example, within a cell. In one embodiment, a preparation is purified such that the peptide antigen conjugate represents at least 50% of the total content of the preparation. Substantial purification denotes purification from other proteins or cellular components. A substantially purified protein is at least 60%, 70%, 80%, 90%, 95%, 98%, or 99% pure. Thus, in one specific, non-limiting example, a substantially purified protein is 90% free of other proteins or cellular components or contaminating peptides.

Reactive: As used herein describes the stability of a molecule or functional group of a molecule and its propensity to undergo a chemical reaction in the presence of another functional group or molecule. For example, amines have the tendency to react with electrophiles under certain conditions, and therefore molecules comprising amines may be referred to as reactive. Reactive monomers refer to monomers with one or more functional groups that are reactive. Various examples of reactive monomers are described in greater detail elsewhere.

Soluble: Capable of becoming molecularly or ionically dispersed in a solvent to form a homogeneous solution. A soluble molecule is understood to be freely dispersed as single molecules in solution and does not assemble into multimers or other supramolecular structures through interactions. Solubility can be determined by visual inspection, by turbidity measurements or by dynamic light scattering.

Subject and patient: These terms may be used interchangeably herein to refer to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

Targeting molecules: Are broadly defined as molecules that direct therapy to a specific tissue or cell population. Targeting molecules are defined by their intended use and therefore include structurally diverse molecules including without limitation antibodies, Fabs, peptides, aptamers, saccharides (e.g., saccharides that bind to lectin receptors and/or are recognized by cellular transporters), amino acids, neurotransmitters, etc. As targeting molecules are often selected from molecules that bind cellular receptors that can activate downstream signaling cascades and/or impact the activity of other linked molecules, targeting molecules are classified as drug molecules in the present disclosure. In preferred embodiments, targeting molecules are often linked to the ends or proximal to the ends of star polymers. In preferred embodiments of star polymers used for cancer treatment, D3 (i.e., drug molecules linked to the end of the polymer arms) is selected from targeting molecules that bind to tumor vasculature, tumor cells and/or other cells in the tumor microenvironment.

T Cell: A type of white blood cell that is part of the immune system and may participate in an immune response. T cells include, but are not limited to, CD4 T cells and CD8 T cells. A CD4 T cell displays the CD4 glycoprotein on its surface and these cells are often referred to as helper T cells. These cells often coordinate immune responses, including antibody responses and cytotoxic T cell responses, however, CD4 T cells can also suppress immune responses or CD4 T cells may act as cytotoxic T cells. A CD8 T cell displays the CD8 glycoprotein on its surface and these cells are often referred to as cytotoxic or killer T cells, however, CD8 T cells can also suppress immune responses.

Telechelic: Is used to describe a polymer that has one or two reactive ends that may be the same or different. The word is derived from telos and chele, the Greek words for end and claw, respectively. A semi-telechelic polymer describes a polymer with only a single end group, such as a reactive functional group that may undergo additional reactions, such as polymerization. A hetero-telechelic polymer describes a polymer with two end groups, such as reactive functional groups, that have different reactive properties. Herein, polymer arms (A) with different linkers precursors at each end, i.e., X2 and Z1, are heterotelechelic polymers.

Treating, preventing, or ameliorating a disease: “Treating” refers to an intervention that reduces a sign or symptom or marker of a disease or pathological condition after it has begun to develop. For example, treating a disease may result in a reduction in tumor burden, meaning a decrease in the number or size of tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Reducing a sign or symptom or marker of a disease or pathological condition related to a disease, refers to any observable beneficial effect of the treatment and/or any observable effect on a proximal, surrogate endpoint, for example, tumor volume, whether symptomatic or not. Reducing a sign or symptom associated with a tumor or viral infection can be evidenced, for example, by a delayed onset of clinical symptoms of the disease in a susceptible subject (such as a subject having a tumor which has not yet metastasized, or a subject that may be exposed to a viral infection), a reduction in severity of some or all clinical symptoms of the disease, a slower progression of the disease (for example by prolonging the life of a subject having a tumor or viral infection), a reduction in the number of relapses of the disease, an improvement in the overall health or well-being of the subject, or by other parameters well known in the art (e.g., that are specific to a particular tumor or viral infection). A “prophylactic” treatment is a treatment administered to a subject who does not exhibit signs of a disease or exhibits only early signs for the purpose of decreasing the risk or severity of developing pathology.

Tumor or cancer or neoplastic: An abnormal growth of cells, which can be benign or malignant, often but not always causing clinical symptoms. “Neoplastic” cell growth refers to cell growth that is not responsive to physiologic cues, such as growth and inhibitory factors.

A “tumor” is a collection of neoplastic cells. In most cases, tumor refers to a collection of neoplastic cells that forms a solid mass. Such tumors may be referred to as solid tumors. In some cases, neoplastic cells may not form a solid mass, such as the case with some leukemias. In such cases, the collection of neoplastic cells may be referred to as a liquid cancer.

Cancer refers to a malignant growth of neoplastic cells, being either solid or liquid. Features of a cancer that define it as malignant include metastasis, interference with the normal functioning of neighboring cells, release of cytokines or other secretory products at abnormal levels and suppression or aggravation of inflammatory or immunological response(s), invasion of surrounding or distant tissues or organs, such as lymph nodes, etc.

A tumor that does not present substantial adverse clinical symptoms and/or is slow growing is referred to as “benign.”

“Malignant” means causing, or likely to cause in the future, significant clinical symptoms. A tumor that invades the surrounding tissue and/or metastasizes and/or produces substantial clinical symptoms through production and secretion of chemical mediators having an effect on nearby or distant body systems is referred to as “malignant.”

“Metastatic disease” refers to cancer cells that have left the original tumor site and migrated to other parts of the body, e.g., via the bloodstream, via the lymphatic system, or via body cavities, such as the peritoneal cavity or thoracic cavity.

The amount of a tumor in an individual is the “tumor burden”. The tumor burden can be measured as the number, volume, or mass of the tumor, and is often assessed by physical examination, radiological imaging, or pathological examination.

An “established” or “existing” tumor is a tumor that exists at the time a therapy is initiated. Often, an established tumor can be discerned by diagnostic tests. In some embodiments, an established tumor can be palpated. In some embodiments, an established tumor is at least 500 mm³, such as at least 600 mm³, at least 700 mm³, or at least 800 mm³ in size. In other embodiments, the tumor is at least 1 cm long. With regard to a solid tumor, an established tumor generally has a newly established and robust blood supply and may have induced the regulatory T cells (Tregs) and myeloid derived suppressor cells (MDSC).

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Chemical structures may be presented with implicit carbons and/or hydrogens or a combination of carbons and/or hydrogens shown in some parts of a structure with implicit carbons and/or hydrogens shown in other parts of a structure. Chemical structures may also be shown with bond angles and/or stereochemistry when such details are important to convey, or chemical structures may not show bond angles and/or stereochemistry when such details are not needed. Any functional group disclosed herein and/or defined above can be substituted or unsubstituted, unless otherwise indicated herein. Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting.

The present disclosure arises from the inventors' development of novel compositions of matter and methods of manufacturing star polymers having linear polymer arms radiating from branched core structures. The branched core serves as a scaffold for arraying two or more polymer arms to create a star polymer. The star polymer serves as a scaffold for arraying various types of pharmaceutically active compounds.

When the star polymers of the present disclosure are used for delivery of pharmaceutically active compounds, referred to herein as drug molecule(s) or drug(s), selected from chemotherapeutic and/or immunostimulant drugs for cancer treatment, the present inventors have found: (i) a range of hydrodynamic sizes of star polymers that lead to optimal tumor uptake following intravenous administration; (ii) the location and density of drug attachment on polymer arms needed to maximize drug loading; (iii) compositions and architecture of polymer arms that allows for high drug loading; (iv) compositions and synthetic routes that lead to the optimal ranges of star polymer hydrodynamic size and drug density required for intravenous delivery; (iv) compositions of star polymers that prevent unwanted antibody responses that lead to accelerated blood clearance; and (v) compositions of stimuli-responsive star polymers that lead to increased accumulation in tumors; and, (vi) optimal combinations of star polymer architecture and composition, drug molecules and linkers that lead to enhanced tumor regression.

When the star polymers of the present disclosure are used for array of drug molecules that act extracellularly, the present inventors have found: (i) a range of hydrodynamic sizes of star polymers that are suitable for applications for delivery of extracellular receptor binding partners, such as checkpoint inhibitors, as well as for delivering therapeutic biologics molecules, including antibodies, to specific tissues; (ii) a range of polymer arms and drug densities needed to optimally engage cognate receptors; (iii) the compositions and synthetic routes that lead to the optimal ranges of star polymer hydrodynamic size and ligand density; and (iv) compositions of star polymers that prevent unwanted antibody responses that can lead to accelerated blood clearance.

Disclosed herein is a star polymer of formula O[D1]-([X]-A[(D2)]-[Z]-[D3])n where O is a core; A is a polymer arm attached to the core; X is a linker molecule between the core and the polymer arm; Z is a linker molecule between the end of the polymer arm and D3; D1 is one or more drug molecules which may the same or different that are attached to the core; D2 is one or more drug molecules which may be the same or different linked to monomer units distributed along the polymer arm; and D3 is one or more drug molecules which may the same or different linked to the ends of the polymer arms; n is an integer number; [ ] denotes that the group is optional; and at least one of D1, D2 or D3 is present.

In another embodiment, disclosed herein is a star polymer having the formula O[D1]-([X]-A(D2)-[Z]-[D3])ₙ where O is a core; each A is a polymer arm attached to the core; each X is a linker molecule between the core and the polymer arm; each Z is a linker molecule between an end of the polymer arm and D3; D1 is a drug molecule linked to the core; each D2 is a drug molecule linked to reactive monomers distributed along the backbone of the polymer arm; each D3 is a drug molecule linked to the ends of the polymer arms; n is an integer from 5 to 60; wherein each A, X, Z, D2 and D3 may be the same or different; [ ] denotes that the group is optional; wherein the polymer arm comprises reactive monomers, hydrophilic monomers, charged monomers, or any combination thereof, and D2 is linked to the reactive monomers distributed along the polymer arm at a density of between 1 mol % and 80 mol %.

In the foregoing discussion and elsewhere in this specification, the designation -A(D2)- is intended to mean that the drug (D) is linked to monomer units distributed along the polymer arms (A). Similarly, the designation —O(D1)- is intended to mean that the drug (D) is linked to functional groups attached to the core (O).

In preferred embodiments of star polymers comprising amphiphilic or hydrophobic drug molecules use for cancer treatment, the amphiphilic or hydrophobic drug molecule is linked to monomer units distributed along the polymer arm, and the star polymer has the formula 0-([X]-A(D2))n. In other embodiments of star polymers comprising amphiphilic or hydrophobic drug molecules use for cancer treatment, wherein the star polymer includes D3, preferably selected from targeting molecules and/or drug molecules that block B cell receptor signaling (e.g., CD22 agonists), and the amphiphilic or hydrophobic drug molecule is linked to monomer units distributed along the polymer arm, the star polymer has the formula O—([X]-A(D2)-[Z]-D3)n.

In some embodiments, the star polymer comprises diblock copolymer arms comprising D2 that are attached to a first block attached to the core and the star polymer has the formula: —O[D2]-([X]-A1(D2)-b-A2-[Z]-[D3])n, wherein A1 is the first block of the polymer arm, A2 is the second block of the polymer arm and b (italicized) delineates the two blocks.

The following sections describe each of the components of star polymers as well as preferred compositions and combinations of each of the components that lead to unexpected improvements in activity for different biomedical applications, particularly intravenous delivery of pharmaceutically active compounds for cancer treatment.

### Core (O)

Any suitable material can be used for the core (O) with the proviso that the core should be selected to ensure that a sufficient number of polymer arms (A) can be attached for the intended application. In certain embodiments, the core (O) is selected so that five or more polymer arms (A) can be attached to enable sufficient surface coverage. In other embodiments, the number of polymer arm (A) attachment points on the core (O) is increased through the use of an amplifying linker, such that a core (O) with an integer number of attachment points is increased by an integer multiple, e.g., 2, 3, 4, 5, 6, 7, 8, 9 or 10, through the use of an amplifying linker. Suitable amplifying linkers are described elsewhere.

Herein, we describe methods of designing and manufacturing star polymers to maximize loading of polymer arms (A) on cores (O). For some compositions of cores (O) and polymer arms (A), the loading of polymer arms (A) on the core (O) may be complete, i.e., all reactive groups on the core (O) are linked to a polymer arm (A). For certain other compositions of cores (O) and polymer arms (A), polymer arm (A) loading on the core may be incomplete. Thus, for the assembly of certain compositions of star polymers, cores may be selected to include more attachment points than needed. In a non-limiting example of a star polymer comprising immunostimulatory and/or chemotherapeutic drugs with 15 or more arms, a core with 30 or more attachment points is used, such as between 30 and 512 attachment points. In preferred embodiments, the core (O) has between 16 and 256 functional groups on the surface suitable for polymer arm attachment, such as between 32 and 128 attachment points, or between 30 and 150 attachment points. In other preferred embodiments, the core (O) has between 10 and 256 functional groups on the surface suitable for polymer arm attachment, such as between 20 and 128 attachment points.

In some embodiments, the core (O) is based on a dendron or dendrimer. Dendrons and dendrimers are a class of highly branched, chemically defined and monodisperse macromolecules (precise composition and architecture). Dendrimers are typically core-shell structures that are symmetric around the core. In dendrons, the core is usually a chemically addressable group called the focal point. The core of a dendrimer affects its three-dimensional shape, i.e., spheric, ellipsoidic, or cylindric. The surface of a dendrimer is densely packed with functional groups, with the number of functional groups dictated by the generation of the dendrimer. The surface functional groups can be directly used or further modified for the attachment of other components, such as polymer arms (A) or drugs (D). Dendrimers include but are not limited to polyamidoamine (PAMAM), amino acid-based dendrimers, e.g., poly(L-lysine) (PLL), polyamide, polyester, polypropylenimine (PPI), and poly(2,2-bis(hydroxylmethyl)propionic acid) (bis-MPA).

In certain embodiments, the core (O) comprises a polyamidoamine (PAMAM) dendrimer with amine functional groups on the surface. In these embodiments, the polyamidoamine dendrimer has surface amine groups, referred to as X1, that react with the linker precursors X2 attached to the polymer arm (A) to link the polymer arm (A) to the core (O) via the linker (X). In other embodiments, PAMAM dendrimer with amine functional groups on the surface are reacted with a functional linker, e.g., NHS activated ester linked to alkyne through a linker, to yield a PAMAM dendrimer with alkyne functional groups on the surface, wherein the alkyne functional groups (X1) are reacted with azide functional groups (X2) on polymer arms to link the polymer arm to the core via the linker X comprising a triazole. In certain embodiments, the polyamidoamine dendrimer is a fifth-generation dendrimer with 128 functional groups on the surface. In preferred embodiments, the functional groups on the polyamidoamine dendrimer are amines or alkynes, particularly strained alkynes.

Cores (O) may also be selected from hyperbranched polymers, which can have similar properties to dendrimers and dendrons. Unlike chemically defined dendrimers or dendrons, however, hyperbranched polymers are often constructed based on one-pot reactions of AB₂ or AB₃ monomers, requiring essentially no work-up.

A challenge with hyperbranched polymers is that they can have wide molecular weight distributions (and high polydispersity) and therefore can be challenging to characterize. Thus, with the exception of hyperbranched polymers produced by solid-phase synthesis, such as hyperbranched poly(amino acids) produced by solid-phase peptide synthesis, cores (O) based on dendrons and dendrimers are preferred.

### Polymer Arm (A)

The polymer arm (A) is linked to the core (O) through any suitable means, either directly (i.e., X is not present) or indirectly (i.e., via linker molecule (X)). The number of polymer arms is an integer value, n.

The polymer arms (A) radiating from the core (O) are typically water-soluble under physiologic pH and salt concentrations in circulation (i.e., in the blood) and principally serve to increase the hydrodynamic radius of the star polymer and/or provide shielding in circulation, i.e., prevent blood protein binding and/or recognition by antigen presenting cells comprising the reticuloendothelial system. In some embodiments, when two or more different compositions of polymer arm are present on a star polymer, at least one of the polymer compositions is water-soluble at blood pH (˜pH 7.4).

The polymer arms (A) of star polymers used for the delivery of chemotherapeutic and/or immunostimulant drugs for cancer treatment, should be selected to increase drug solubility, reduce/prevent drug degradation and provide a stealth coating to prevent the uptake of the star polymer by cells of the reticuloendothelial system. Polymer arms comprising star polymers used for chemotherapeutic and/or immunostimulant delivery principally function to prevent star polymer uptake by phagocytic cells and therefore should be flexible, non-rigid and non-reactive for serum proteins. Unexpectedly, the inventors of the present disclosure found that hydrophilic arms comprised of anionic monomers can function to improve solubility of star polymers carrying high densities of hydrophobic or amphiphilic small molecule drugs; extend the polymer arm (A) to increase the star polymer hydrodynamic size; prevent antibody responses, which was found to reduce accelerated blood clearance upon repeat dosing; and, improve tumor accumulation.

Polymer arms (A) used for star polymers can be derived from either natural or synthetic sources and may be prepared by any suitable means. Polymer arms (A) are typically prepared by polymerization, which is a chemical reaction usually carried out with a catalyst, heat, or light, in which monomers combine to form a chainlike or cross-linked macromolecule (a polymer). Synthetic polymers may be produced by step-growth (i.e., condensation) polymerization or chain-growth (i.e., free radical, anionic, or cationic) polymerization. In terms of polymerization process, solution polymerization, bulk polymerization, dispersion polymerization and emulsion polymerization are available.

In certain embodiments, polymer arms (A) are prepared by controlled “living” radical polymerization methods to minimize premature termination and enable more precise control over the polymer composition, molecular weight, polydispersity and functionality. In the context of controlled living radical polymerization, highly reactive free radicals generated from the decomposition of an initiator (radical source) are capable of initiating the polymerization of monomers. Chain propagation proceeds as the radical center continues to add monomers; however, for controlled living radical polymerization, the reversible deactivation of radicals occurs either by metal complex via atom transfer radical polymerization (ATRP) mechanism, dithioester or trithioester chain transfer agent (CTA) via reversible addition-fragmentation chain-transfer (RAFT) polymerization mechanism, or nitroxide radical via nitroxide-mediated polymerization (NMP) mechanism. These mechanisms lower the effective concentration of active radicals at any moment during the polymerization process, which prevents potential premature chain termination. The fast and reversible radical activation-deactivation process allows all propagating chains equal opportunity to grow resulting in polymers with very narrow molecular weight distribution and low polydispersity.

Controlled radical polymerization allows polymer arms (A) with a wide range of different polymer functionalities, either introduced through monomer selection, the initiation or quenching of the propagating polymer chain, or post-polymerization modification, sometimes referred to as polymer analogous reaction. While functional groups distributed along the backbones of polymers arms (A) can be modulated through choice of monomer, both end groups of polymer arms (A) can be modulated by selecting suitable initiators and CTAs used for RAFT polymerization.

Accordingly, an initiator comprising a functional group (FG) or drug (D) used to initiate polymerization of monomers in the presence of dithioester- or trithioester-based CTA that is also functionalized with the FG or drug (D) will lead to polymer arms (A) with one end functionalized with the FG or drug (D) and the other end will comprise a dithioester or trithioester that is introduced by the CTA. Polymers capped with a CTA are referred to as “macro-CTAs” and may be used to induce the RAFT polymerization of other monomers, thus providing a simple route for the preparation of block copolymers, such as A-B type di-block copolymers. Alternatively, the dithioester or trithioester may be reduced (to a thiol) and capped with a thiol-reactive moiety or may be capped using an initiator comprising a functional group (FG) or drug (D).

In certain embodiments, X2, Z1 or a drug (D) are introduced to polymer arms by reacting an initiator functionalized with the X2 or Z1 linker precursor or drug (D) with monomers in the presence of CTA to produce a polymer arm intermediate, X2-polymer-CTA, Z1-polymer-CTA or D-polymer-CTA, which is capped using an initiator or thiol-reactive compounds functionalized with an X2 or Z1 linker precursor or drug (D) to obtain a heterotelechelic polymer arm, e.g., X2-polymer-Z1, Z1-polymer-X2 or X2-polymer-D3. Specific examples of polymer arms (A) produced in this manner are described later.

In some embodiments, (meth)acrylamide- and (meth)acrylate-based polymers are synthesized by reversible addition-fragmentation chain-transfer (RAFT) polymerization. In additional embodiments, poly(amino acids) and poly(phosphoesters) are synthesized by ring opening polymerization. For polymers produced by ring opening polymerization, the compounds used for initiating polymerization can be used to introduce functionalities at one end and the other end of the resulting polymer can be capped by any suitable means to introduce the desired functionality. In still other embodiments, peptides (or “poly(amino acids)) are synthesized by solid-phase peptide synthesis (SPPS).

The architecture of the polymer arm (A) is selected to address the specific demands of the application. In some embodiments, linear polymer arms (A) are used to link drugs indirectly via the polymer arm (A) to the core (O) of the star polymer. In other embodiments, the polymer arm (A) is a brush polymer that is used as an amplifying linker and/or to provide additional surface area coverage of the star polymer. In some embodiments, polymer arms (A) with brush architecture are used on star polymer carriers of small molecule immunostimulant and/or chemotherapeutic drugs. For such embodiments, coating star polymers with polymer arms with brush architecture was associated with increased tumor uptake as compared with star polymers comprising linear polymer arms (A). A non-binding explanation is that increased surface area coverage by the hydrophilic polymer arm (A) reduced blood protein binding and/or reduced uptake by phagocytic cells, thereby increasing circulation time and star polymer uptake into tumors.

In other embodiments, polymer arms with diblock architecture are used to segregate different components comprising the star polymer. In some embodiments, diblock copolymers are used to segregate drugs (D), such as small molecule chemotherapeutics and/or immunostimulant drugs, to one block of the di-block polymer. In other embodiments, diblock polymers are used to segregate charged monomers, i.e., charged monomers are only placed on one block of the diblock polymer. In still other embodiments, diblock polymers are used to segregate two or more different components, such as drugs (D) and charged monomers.

Each of the monomer units comprising the polymer arm (A) is selected to meet the demands of the application. Suitable polymer arms may comprise an integer number, b, of hydrophilic monomer units (B), an integer number, c, of charged monomer units (C) and/or an integer number, e, of reactive monomer units (E) that comprise a functional group enabling attachment of drugs (D).

In certain preferred embodiments of star polymers, the polymers arms (A) comprise neutral hydrophilic monomers (B), and optionally one or any combination of a charged monomer (C) or a reactive monomer (E), which may be represented as (B)b-[(C)c]-[(E)e], wherein b is equal to an integer number of repeating units of a neutral, hydrophilic co-monomer, B; c is an integer number of a repeating units of a charged co-monomer, C; e is equal to an integer number of repeating units of a reactive co-monomer, E, used for drug (D) attachment; and, [ ] denotes that the monomer unit is optional.

In some embodiments, the polymer arm (A) is a terpolymer (sometimes referred to as copolymer) comprising neutral hydrophilic monomers, charged monomers and reactive monomers linked to drug (D), which may be represented schematically:

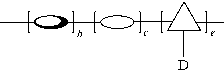

In some embodiments, the polymer arm (A) is a copolymer comprising hydrophilic monomers and charged monomers, which may be represented schematically:

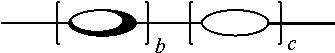

In some embodiments, the polymer arm (A) is a copolymer comprising hydrophilic monomers and reactive monomers linked to drug (D), which may be represented schematically:

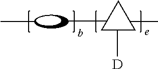

In some embodiments, the polymer arm (A) is a copolymer comprising charged monomers and reactive monomers linked to drug (D), which may be represented schematically:

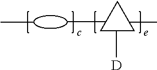

In some embodiments, the polymer arm (A) is a homopolymer comprising only hydrophilic or charged monomers and the polymer arm may be represented schematically:

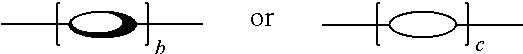

Note: For diblock polymer arms of star polymers described herein, the first block is defined as the block that is proximal to the core and the second block is distal to the core.

In some embodiments, the polymer arm (A) is a diblock copolymer that comprises reactive monomers linked to drug molecules on a first block and only hydrophilic monomers on the second block, which may be represented schematically:

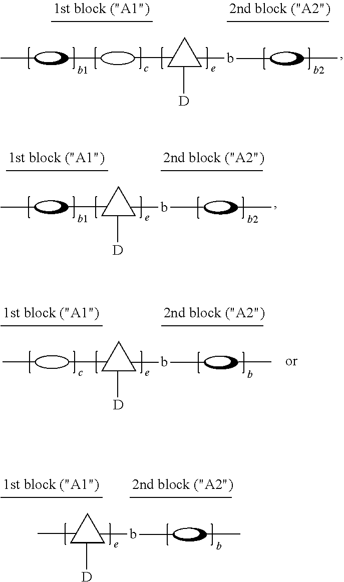

For star polymers comprising diblock polymer arms (A) with monomers (E) linked to amphiphilic or hydrophobic small molecule drugs and hydrophilic monomers (B) on one block and only hydrophilic monomers (B) on the other block, it was found that placing the monomers linked to the amphiphilic or hydrophobic small molecule drugs (D) on the block of the diblock polymer arms (A) proximal to the core of the star polymers resulted in improved stability, i.e., reduced propensity of the star polymers to aggregate.

In some embodiments, the polymer arm (A) is a diblock polymer, and includes reactive monomers linked to drug (D) on the first block and charged monomers on opposite blocks, which may be represented schematically:

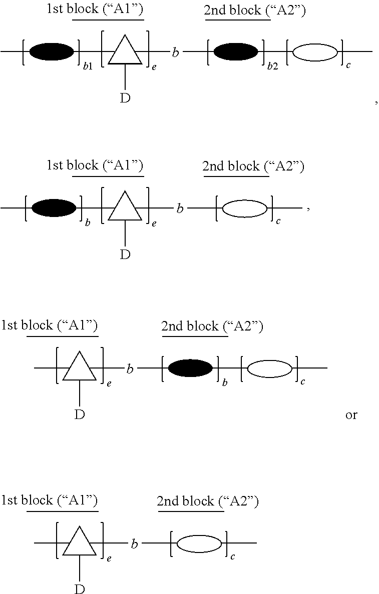

For star polymers comprising diblock polymer arms (A) with reactive monomers linked to amphiphilic or hydrophobic small molecule drugs and hydrophilic monomers on the first block and hydrophilic monomers and charged monomers on the second block, it was found that placement of the monomers linked to the amphiphilic or hydrophobic small molecule drugs (D) proximal to the core and the charged monomers on the opposite block of polymer arms (A) distal to the core led to improved stability of the resulting star polymers. A non-binding explanation for this finding is that the charged block, i.e., the polymer block comprising charged monomers, allows improved solubility and shields the block bearing the amphiphilic or hydrophobic small molecule drug (D).

In some embodiments, the polymer arm is a diblock polymer, and includes charged monomers and drugs (D) on the first block, which may be represented schematically:

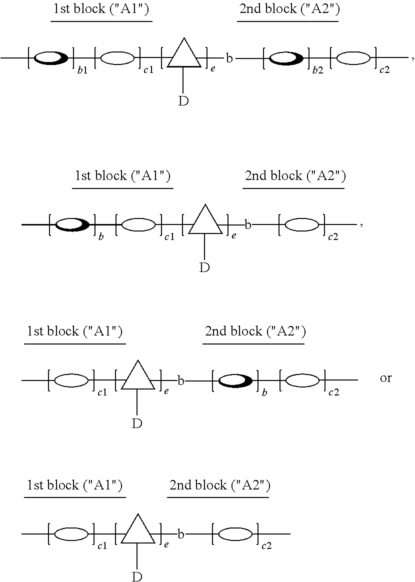

In some embodiments, the polymer arm (A) includes monomers selected from acrylates, (meth)acrylates, acrylamides, (meth)acrylamides, allyl ethers, vinyl acetates, vinyl amides, substituted styrenes, amino acids, acrylonitrile, heterocyclic monomers (i.e., ethylene oxide), saccharides, phosphoesters, phosphonamides, sulfonate esters, sulfonamides, or combinations thereof.

In preferred embodiments of star polymers, the polymer arms (A) comprise neutral hydrophilic monomers, which may be described generically as hydrophilic monomers. In some embodiments, neutral hydrophilic monomers (or hydrophilic monomers) are selected from (meth)acrylates or (meth)acrylamides (inclusive of acrylates, methacrylates, acrylamides and methacrylamides) of the chemical formula CH₂═CR₂—C(O)—R₁ (“Formula I”), wherein the acryl side group R₁ may be selected from one or more of the groups consisting of —OR₃, —NHR₃ or —N(CH₃)R₃, where R₂ can be H or CH₃, and R₃ is independently selected from any hydrophilic substituent. Non-limiting examples of R₃ include but are not limited to H (except for OR₃), CH₃, CH₂CH₃, CH₂CH₂OH, CH₂(CH₂)₂OH, CH₂CH(OH)CH₃, CHCH₃CH₂OH or (CH₂CH₂O)ᵢH, where i is an integer number of repeating units, typically 1 to 6, such as 1, 2, 3, 4, 5 or 6.

A non-limiting example of a neutral hydrophilic monomer of Formula I wherein R₁=NHR₃, R₂=CH₃, and R₃=CH₂CH(OH)CH₃ is N-2-hydroxypropyl(methacrylamide) (HPMA):

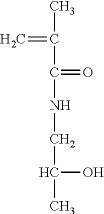

The above example, N-(2-hydroxpropyl(methacrylamide)) (HPMA), is an example of a neutral hydrophilic monomer of Formula I.

In some embodiments of star polymers, the polymer arm (A) comprises charged monomers that contain one or more functional groups (“charged functional group”) that either have a fixed charge or have net charge under certain physiological conditions. Non-limiting examples of charged monomers include (meth)acrylamides and (meth)acrylates that comprise amine, quaternary ammonium, sulfonic acid, sulfuric acid, sulfonium, phosphoric acid, phosphonic acid, phosphonium, carboxylic acid and/or boronic acid functional groups, as well as any combinations or salt forms thereof. Non-limiting examples of salts include e.g., positively charged functional groups, e.g., ammonium ions paired with halide (e.g., chloride) ions. Other non-limiting examples of suitable salts of charged amino acids include conjugate bases of carboxylic, sulfonic and phosphonic acids, paired with group 1 metals, such as sodium, or ammonium or guanidinium ions. In preferred embodiments of polymer arms comprising conjugate bases of acids, the counterion is an ammonium salt, such as the ammonium salt of tris(hydoxymethyl)aminomethane (cas: 77-86-1).

In some embodiments, charged monomers are selected from (meth)acrylates and (meth)acrylamides with chemical formula CH₂═CR₅—C(O)—R₄ (“Formula II”). The acryl side group R₄ may be selected from one or more of the groups consisting of —OR₆, —NHR₆ or —N(CH₃)R₆, where R₅ can be H or CH₃ and R₆ can be selected from, but is not limited to, OH, linear alkyl structures such as (CH₂)ⱼNH₂, (CH₂)ⱼ-imidazole, (CH₂)ⱼ-pyridine amine, (CH₂)ⱼ-(quinoline-amine), (CH₂)ⱼ-pyridine amine, (CH₂)ⱼ-naphthalene amine, (CH₂)ⱼCH(NH₂)COOH, (CH₂)ⱼCOOH, (CH₂)ⱼCH(CH₃)COOH, (CH₂)ⱼC(CH₃)₂COOH, (CH₂)ⱼPO₃H₂, (CH₂)ⱼOPO₃H₂, (CH₂)ⱼSO₃H, (CH₂)ⱼOSO₃H, (CH₂)ⱼB(OH)₂, CH₂N(CH₃)₂, CH₂CH₂N(CH₃)₂, CH₂CH₂CH₂N(CH₃)₂, CH₂N(CH₂CH₃)₂, CH₂CH₂N(CH₂CH₃)₂, CH₂CH₂CH₂N(CH₂CH₃)₂, CH₂N(CH(CH₃)₂), CH₂CH₂N((CH(CH₃)₂), CH₂CH₂CH₂N(CH(CH₃)₂), CH[CH₂N(CH₃)₂]₂, CH(COOH)CHCH₂COOH, (CH₂)ⱼNH(CH₂)ⱼCOOH, (CH₂)ⱼN(CH₃)(CH₂)ⱼCOOH, (CH₂)ⱼN⁺(CH₃)₂(CH₂)ⱼCOOH, (CH₂)ⱼN⁺(CH₂—CH₃)₂(CH₂)ⱼCOOH, [CH₂CH(CH₃)O]₅PO₃H₂, C(CH₃)₂CH₂SO₃H, and C₆H₄B(OH)₂ where j is an integer number of a repeating units, typically between 1 to 6, such as 1, 2, 3, 4, 5 or 6. In some embodiments of (meth)acrylates and (meth)acrylamides of Formula II, the acryl side group comprises tetraalkyl ammonium salts, nitrogen heterocycles or aromatic amines, which may be linked to the monomer through any suitable means either directly or via a linker. Non-limiting examples of nitrogen heterocycles and/or aromatic amines include pyrrole, imidazole, pyridine, pyrimidine, pyrazine, diazepine, indole, quinoline, amino quinoline, amino pyridine, purine, pteridine, aniline, naphthalene amine or the like. In certain preferred embodiments, of (meth)acrylates and (meth)acrylamides of Formula II, the acryl the acryl side group comprises carboxylic acid(s), which may be linked to the monomer through any suitable means either directly or via a linker.

In some embodiments, the acryl side group R₄ may additionally comprise a linker, which is typically selected from short alkyl chains and/or PEG linkers between the charged functional group and the acryl group. Non-limiting examples of monomers of Formula II, wherein R₄ comprises a linker between the acryl side group and the charged functional group include R₄ selected from one or more of the groups consisting of —OR₆, —NHR₆ or —N(CH₃)R₆, where R₆ can be selected from, but is not limited to (CH₂)ₜ—C(O)—NH—(CH₂)ⱼNH₂, (CH₂)ₜ—C(O)—NH—(CH₂)ⱼ-imidazole, (CH₂)ₜ—C(O)—NH—(CH₂)ⱼ-pyridine amine, (CH₂)ₜ—C(O)—NH—(CH₂)ⱼ-(quinoline-amine), (CH₂)ₜ—C(O)—NH—(CH₂)ⱼ-pyridine amine, (CH₂)ₜ—C(O)—NH—(CH₂)ⱼ-naphthalene amine, (CH₂)ₜ—C(O)—NH—(CH₂)ⱼCH(NH₂)COOH, (CH₂)ₜ—C(O)—NH—(CH₂)ⱼCOOH, (CH₂)ₜ—C(O)—NH—(CH₂)ⱼCH(CH₃)COOH, (CH₂)ₜ—C(O)—NH—(CH₂)ⱼC(CH₃)₂COOH, (CH₂)ₜ—C(O)—NH—(CH₂)ⱼPO₃H₂, (CH₂)ₜ—C(O)—NH—(CH₂)ⱼOPO₃H₂, (CH₂)ₜ—C(O)—NH—(CH₂)ⱼSO₃H, (CH₂)ₜ—C(O)—NH—(CH₂)ⱼOSO₃H, (CH₂)ₜ—C(O)—NH—(CH₂)ⱼB(OH)₂, (CH₂)ₜ—C(O)—NH—CH₂N(CH₃)₂, (CH₂)ₜ—C(O)—NH—CH₂CH₂N(CH₃)₂, (CH₂)ₜ—C(O)—NH—CH₂CH₂CH₂N(CH₃)₂, (CH₂)ₜ—C(O)—NH—CH₂N(CH₂CH₃)₂, (CH₂)ₜ—C(O)—NH—CH₂CH₂N(CH₂CH₃)₂, (CH₂)ₜ—C(O)—NH—CH₂CH₂CH₂N(CH₂CH₃)₂, (CH₂)ₜ—C(O)—NH—CH₂N(CH(CH₃)₂), (CH₂)ₜ—C(O)—NH—CH₂CH₂N((CH(CH₃)₂), (CH₂)ₜ—C(O)—NH—CH₂CH₂CH₂N(CH(CH₃)₂), (CH₂)ₜ—C(O)—NH—CH[CH₂N(CH₃)₂]₂, (CH₂)ₜ—C(O)—NH—CH(COOH)CHCH₂COOH, (CH₂)ₜ—C(O)—NH—(CH₂)ₜNH(CH₂)ⱼCOOH, (CH₂)ₜ—C(O)—NH—(CH₂)ⱼN(CH₃)(CH₂)ⱼCOOH, (CH₂)ₜ—C(O)—NH—(CH₂)ⱼN⁺(CH₃)₂(CH₂)ⱼCOOH, (CH₂)ₜ—C(O)—NH—(CH₂)ⱼN⁺(CH₂—CH₃)₂(CH₂)ⱼCOOH, (CH₂)ₜ—C(O)—NH—[CH₂CH(CH₃)O]₅PO₃H₂, (CH₂)ₜ—C(O)—NH—C(CH₃)₂CH₂SO₃H, (CH₂)ₜ—C(O)—NH—C₆H₄B(OH)₂, (CH₂CH₂O)ₜCH₂CH₂C(O)—NH—(CH₂)ⱼNH₂, (CH₂CH₂O)ₜCH₂CH₂C(O)—NH—(CH₂)ⱼ-imidazole, (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-(quinoline-amine), (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-naphthalene amine, (CH₂CH₂O)ₜCH₂CH₂C(O)—NH—(CH₂)ⱼCH(NH₂)COOH, (CH₂CH₂O)ₜCH₂CH₂C(O)—NH—(CH₂)ⱼCOOH, (CH₂CH₂O)ₜCH₂CH₂C(O)—NH—(CH₂)ⱼCH(CH₃)COOH, (CH₂CH₂O)ₜCH₂CH₂C(O)—NH—(CH₂)ⱼC(CH₃)₂COOH, (CH₂CH₂O)ₜCH₂CH₂C(O)—NH—(CH₂)ⱼPO₃H₂, (CH₂CH₂O)ₜCH₂CH₂C(O)—NH—(CH₂)ⱼOPO₃H₂, (CH₂CH₂O)ₜCH₂CH₂C(O)—NH—(CH₂)ⱼSO₃H, (CH₂CH₂O)ₜCH₂CH₂C(O)—NH—(CH₂)ⱼOSO₃H, (CH₂CH₂O)ₜCH₂CH₂C(O)—NH—(CH₂)ⱼB(OH)₂, (CH₂CH₂O)ₜCH₂CH₂C(O)—NH—CH₂N(CH₃)₂, (CH₂CH₂O)ₜCH₂CH₂C(O)—NH—CH₂CH₂N(CH₃)₂, (CH₂CH₂O)ₜCH₂CH₂C(O)—NH—CH₂CH₂CH₂N(CH₃)₂, (CH₂CH₂O)ₜCH₂CH₂C(O)—NH—CH₂N(CH₂CH₃)₂, (CH₂CH₂O)ₜCH₂CH₂C(O)—NH—CH₂CH₂N(CH₂CH₃)₂, (CH₂CH₂O)ₜCH₂CH₂C(O)—NH—CH₂CH₂CH₂N(CH₂CH₃)₂, (CH₂CH₂O)ₜCH₂CH₂C(O)—NH—CH₂N(CH(CH₃)₂), (CH₂CH₂O)ₜCH₂CH₂C(O)—NH—CH₂CH₂N((CH(CH₃)₂), (CH₂CH₂O)ₜCH₂CH₂C(O)—NH—CH₂CH₂CH₂N(CH(CH₃)₂), (CH₂CH₂O)ₜCH₂CH₂C(O)—NH—CH[CH₂N(CH₃)₂]₂, (CH₂CH₂O)ₜCH₂CH₂C(O)—NH—CH(COOH)CH—CH₂COOH, (CH₂CH₂O)ₜCH₂CH₂C(O)—NH—(CH₂)ₜNH(CH₂)ⱼCOOH, (CH₂CH₂O)ₜCH₂CH₂C(O)—NH—(CH₂)ⱼN(CH₃)(CH₂)ⱼCOOH, (CH₂CH₂O)ₜCH₂CH₂C(O)—NH—(CH₂)ⱼN⁺(CH₃)₂(CH₂)ⱼCOOH, (CH₂CH₂O)ₜCH₂CH₂C(O)—NH—(CH₂)ⱼN⁺(CH₂—CH₃)₂(CH₂)ⱼCOOH, (CH₂CH₂O)ₜCH₂CH₂C(O)—NH—[CH₂CH(CH₃)O]₅PO₃H₂, (CH₂CH₂O)ₜCH₂CH₂C(O)—NH—C(CH₃)₂CH₂SO₃H, (CH₂CH₂O)ₜCH₂CH₂C(O)—NH—C₆H₄B(OH)₂, (CH₂)ₜ—NH—C(O)—NH—(CH₂)ⱼNH₂, (CH₂)ₜ—NH—C(O)—(CH₂)ⱼ-imidazole, (CH₂)ₜ—NH—C(O)—(CH₂)ⱼ-pyridine amine, (CH₂)ₜ—NH—C(O)—(CH₂)ⱼ-(quinoline-amine), (CH₂)ₜ—NH—C(O)—(CH₂)ⱼ-pyridine amine, (CH₂)ₜ—NH—C(O)—(CH₂)ⱼ-naphthalene amine, (CH₂)ₜ—NH—C(O)—(CH₂)ⱼCH(NH₂)COOH, (CH₂)ₜ—NH—C(O)—(CH₂)ⱼCOOH, (CH₂)ₜ—NH—C(O)—(CH₂)ⱼCH(CH₃)COOH, (CH₂)ₜ—NH—C(O)—(CH₂)ⱼC(CH₃)₂COOH, (CH₂)ₜ—NH—C(O)—(CH₂)ⱼPO₃H₂, (CH₂)ₜ—NH—C(O)—(CH₂)ⱼOPO₃H₂, (CH₂)ₜ—NH—C(O)—(CH₂)ⱼSO₃H, (CH₂)ₜ—NH—C(O)—(CH₂)ⱼOSO₃H, (CH₂)ₜ—NH—C(O)—(CH₂)ⱼB(OH)₂, (CH₂)ₜ—NH—C(O)—CH₂N(CH₃)₂, (CH₂)ₜ—NH—C(O)—CH₂CH₂N(CH₃)₂, (CH₂)ₜ—NH—C(O)—CH₂CH₂CH₂N(CH₃)₂, (CH₂)ₜ—NH—C(O)—CH₂N(CH₂CH₃)₂, (CH₂)ₜ—NH—C(O)—CH₂CH₂N(CH₂CH₃)₂, (CH₂)ₜ—NH—C(O)—CH₂CH₂CH₂N(CH₂CH₃)₂, (CH₂)ₜ—NH—C(O)—CH₂N(CH(CH₃)₂), (CH₂)ₜ—NH—C(O)—CH₂CH₂N((CH(CH₃)₂), (CH₂)ₜ—NH—C(O)—CH₂CH₂CH₂N(CH(CH₃)₂), (CH₂)ₜ—NH—C(O)—CH[CH₂N(CH₃)₂]₂, (CH₂)ₜ—NH—C(O)—CH(COOH)CH—CH₂COOH, (CH₂)ₜ—NH—C(O)—(CH₂)ₜNH(CH₂)ⱼCOOH, (CH₂)ₜ—NH—C(O)—(CH₂)ⱼN(CH₃)(CH₂)ⱼCOOH, (CH₂)ₜ—NH—C(O)—CH₂)ⱼN⁺(CH₃)₂(CH₂)ⱼCOOH, (CH₂)ₜ—NH—C(O)—(CH₂)ⱼN⁺(CH₂—CH₃)₂(CH₂)ⱼCOOH, (CH₂)ₜ—NH—C(O)—[CH₂CH(CH₃)O]₅PO₃H₂, (CH₂)ₜ—NH—C(O)—C(CH₃)₂CH₂SO₃H, (CH₂)ₜ—NH—C(O)—C₆H₄B(OH)₂, (CH₂CH₂O)ₜCH₂CH₂NH—C(O)—(CH₂)ⱼNH₂, (CH₂CH₂O)ₜCH₂CH₂NH—C(O)—(CH₂)ⱼ-imidazole, (CH₂CH₂O)ₜCH₂CH₂NH—C(O)—(CH₂)ⱼ-pyridine amine, (CH₂CH₂O)ₜCH₂CH₂NH—C(O)(CH₂)ⱼ-(quinoline-amine), (CH₂CH₂O)ₜCH₂CH₂NH—C(O)—(CH₂)ⱼ-pyridine amine, (CH₂CH₂O)ₜCH₂CH₂NH—C(O)—(CH₂)ⱼ-naphthalene amine, (CH₂CH₂O)ₜCH₂CH₂NH—C(O)—(CH₂)ⱼCH(NH₂)COOH, (CH₂CH₂O)ₜCH₂CH₂NH—C(O)—(CH₂)ⱼCOOH, (CH₂CH₂O)ₜCH₂CH₂NH—C(O)—(CH₂)ⱼCH(CH₃)COOH, (CH₂CH₂O)ₜCH₂CH₂NH—C(O)—(CH₂)ⱼC(CH₃)₂COOH, (CH₂CH₂O)ₜCH₂CH₂NH—C(O)—(CH₂)ⱼPO₃H₂, (CH₂CH₂O)ₜCH₂CH₂NH—C(O)—(CH₂)ⱼOPO₃H₂, (CH₂CH₂O)ₜCH₂CH₂NH—C(O)—(CH₂)ⱼSO₃H, (CH₂CH₂O)ₜCH₂CH₂NH—C(O)—(CH₂)ⱼOSO₃H, (CH₂CH₂O)ₜCH₂CH₂NH—C(O)—(CH₂)ⱼB(OH)₂, (CH₂CH₂O)ₜCH₂CH₂NH—C(O)—CH₂N(CH₃)₂, (CH₂CH₂O)ₜCH₂CH₂NH—C(O)—CH₂CH₂N(CH₃)₂, (CH₂CH₂O)ₜCH₂CH₂NH—C(O)—CH₂CH₂CH₂N(CH₃)₂, (CH₂CH₂O)ₜCH₂CH₂NH—C(O)—CH₂N(CH₂CH₃)₂, (CH₂CH₂O)ₜCH₂CH₂NH—C(O)—CH₂CH₂N(CH₂CH₃)₂, (CH₂CH₂O)ₜCH₂CH₂NH—C(O)—CH₂CH₂CH₂N(CH₂CH₃)₂, (CH₂CH₂O)ₜCH₂CH₂NH—C(O)—CH₂N(CH(CH₃)₂), (CH₂CH₂O)ₜCH₂CH₂NH—C(O)—CH₂CH₂N((CH(CH₃)₂), (CH₂CH₂O)ₜCH₂CH₂NH—C(O)—CH₂CH₂CH₂N(CH(CH₃)₂), (CH₂CH₂O)ₜCH₂CH₂NH—C(O)—CH[CH₂N(CH₃)₂]₂, (CH₂CH₂O)ₜCH₂CH₂NH—C(O)—CH(COOH)CH—CH₂COOH, (CH₂CH₂O)ₜCH₂CH₂NH—C(O)—(CH₂)ₜNH(CH₂)ⱼCOOH, (CH₂CH₂O)ₜCH₂CH₂NH—C(O)—(CH₂)ⱼN(CH₃)(CH₂)ⱼCOOH, (CH₂CH₂O)ₜCH₂CH₂NH—C(O)—(CH₂)ⱼN⁺(CH₃)₂(CH₂)ⱼCOOH, (CH₂CH₂O)ₜCH₂CH₂NH—C(O)—(CH₂)ⱼN⁺(CH₂—CH₃)₂(CH₂)ⱼCOOH, (CH₂CH₂O)ₜCH₂CH₂NH—C(O)—[CH₂CH(CH₃)O]₅PO₃H₂, (CH₂CH₂O)ₜCH₂CH₂NH—C(O)—C(CH₃)₂CH₂SO₃H, (CH₂CH₂O)ₜCH₂CH₂NH—C(O)—C₆H₄B(OH)₂, where t and j are each independently an integer number of a repeating units, typically selected from between 1 to 6, such as 1, 2, 3, 4, 5 or 6.

A non-limiting example of a charged monomer of Formula II wherein R₄=—OR₆, R₅=CH₃ and R₆=OH is:

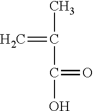

wherein in this example, the monomer would be expected to be deprotonated at physiologic pH (i.e., pH 7.4) and carry a negative charge.

In certain preferred embodiments of star polymers comprising charged monomers, the charged monomer comprises charge groups selected from glycine, beta-alanine, butanoic acid, methyl butanoic acid, dimethylbutanoic acid (DMBA), 3,3′-((2-(6-aminohexanamido)propane-1,3-diyl)bis(oxy))dipropionic acid (referred to as “bis(COOH)”) and 13-(6-aminohexanamido)-6,20-bis((2-carboxyethoxy)methyl)-8,18-dioxo-4,11,15,22-tetraoxa-7,19-diazapentacosanedioic acid (referred to as “tetra(COOH)”).

In some embodiments, polymer arms (A) comprise a monomer, E, that is reactive towards drugs (D). Suitable reactive monomers include but are not limited to any monomer unit bearing a functional group suitable for attachment of drugs (D), including monomers with azide, alkyne, hydrazine, heterocyclic rings, isocyanate, isothiocyanate, aldehyde, ketone, activated carboxylic acid, protected maleimide and amine. Suitable linker chemistries used to link drug molecules (D) to the polymer backbone are discussed throughout the present specification. Note, drugs that act extracellularly may be linked to reactive monomers distributed along the backbone of the polymer arm (A), though, in preferred embodiments drugs that bind to extracellular receptors, particularly targeting molecules are linked to the ends of the polymer arms (A) to maximize solvent exposure. While reactive monomers may comprise functional groups that can impart charge and/or improve water solubility, such as carboxylic acid and hydroxyl groups, respectively, and may also therefore be classified as charged monomer or neutral hydrophilic monomers, the classification of a monomer as a reactive monomer is context-dependent and based on its intended use. For example, monomers comprising carboxylic acids would be considered charged monomers if the carboxylic acid is not used for drug attachment, whereas the same monomers linked to an amine bearing drug molecule, e.g., via an amide bind, would be considered a reactive monomer.

In some embodiments, polymer arms (A) comprise reactive monomers selected from (meth)acrylates and (meth)acrylamides of chemical formula CH₂═CR₈—C(O)—R₇ (“Formula III”). The acryl side group R₇ may be selected from any suitable linker molecule for attachment of drug molecules. Though, in preferred embodiments, R₇ is typically selected from any one or more of the groups consisting of —OH, —NH—NH₂, —NH—NH—C(O)—NH—NH₂, any suitable leaving group (e.g., NHS (cas: 6066-82-6), TT (cas: 202-512-1), etc.), —O(CH₂)ₖ—FG, —O(CH₂)ₖC(O)R₉, —O(CH₂CH₂O)ₖCH₂CH₂—FG, —O(CH₂CH₂O)ₖCH₂CH₂C(O)R₉, —NH(CH₂)ₖ—FG, —NH(CH₂)ₖC(O)R₉, —NH(CH₂CH₂O)ₖCH₂CH₂—FG, —NH(CH₂CH₂O)ₖCH₂CH₂C(O)R₉, —NH(CH₂)ₖNH—C(CO)—(CH₂)ₕ—FG, —NH(CH₂CH₂O)ₖCH₂CH₂NH—C(O)—(CH₂)ₕ—-FG, —NH—CHR₁₀—C(O)—R₉, —NH—CHR₁₀—C(O)—(NH—CHR₁₀—C(O))ₖ—R₉ where k is any integer typically selected from 1 to 6, R₈ can be H or CH₃ and R₉ can be independently selected from, but is not limited to, —OH, —NH—NH₂, —NH—NH—C(O)—NH—NH₂, any suitable leaving group (e.g., NHS, TT, etc.), —O(CH₂)ₕ—FG, —O(CH₂CH₂OₙCH₂CH₂—FG, —NH(CH₂)ₕ—FG, —NH(CH₂CH₂O)CH₂CH₂—FG, —(NH—CHR₁₀—C(O))ₕ—NH—CH₂—FG, —NH—CHR₁₀—C(O)—OH, —NH—CHR₁₀—C(O)—NH—NH₂, —NH—CHR₁₀—C(O)—NH—NH—C(O)—NH—NH₂, —NH—CHR₁₀—C(O)-LG (wherein LG is any suitable leaving group), —(NH—CHR₁₀—C(O))ₕ—NH—(CH₂)_{f}—FG, where h and f are independently any integer typically selected from 1 to 6, R₁₀ is any amino acid side group and FG is any functional group which may be selected from, but not limited to, carboxylic acid, activated carboxylic acids (e.g., carbonylthiazolidine-2-thione, NHS or nitrophenol esters), carboxylic acid anhydrides, amine and protected amines (e.g., tert-butyloxycarbonyl protected amine), OSi(CH₃), CCH, azide, alkyne, stained-alkyne, halogen (e.g., fluoride, chloride), olefins and endo cyclic olefins (e.g., allyl), CN, OH, and epoxy, hydrazines (including hydrazides), carbohydrazides, aldehydes, ketones, carbamates and activated carbamates.

A non-limiting example of a reactive monomer of Formula III wherein R₇ is NH(CH₂)ₖC(O)R₉, R₈ is CH₃, R₉ is NH(CH₂)ₕ—-FG, k is 2, h is 1 and FG is acetylene:

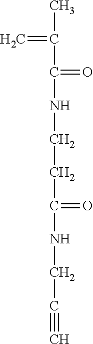

Note: While reactive monomers may comprise multiple functional groups, the functional group specified in the above chemical formulae as “FG” is the functional group that is typically reacted with drug molecules either directly or via a linker to link drug molecules to the reactive monomer. Moreover, the drug molecule linked to the reactive monomer may additionally comprise a linker that is used to indirectly link the drug molecule to the reactive monomer. In certain preferred methods of manufacturing, the drug molecule is typically linked to reactive monomers by post-polymerization modification (i.e., polymer analogous reaction) by reacting drug molecules (optionally linked to linkers) to reactive monomer units distributed along the backbone of polymer arms (as opposed to single monomers), but prior to grafting the polymers arms to the core.

In preferred embodiments of star polymers used for cancer treatment, drug molecules are linked to a self-immolative carbamate that is linked to a peptide that is linked to the reactive monomer, wherein in preferred methods of manufacturing, the drug molecule linked to a self-immolative carbamate that is linked to a peptide with an N-terminal amine is reacted with polymer arms comprising reactive monomers comprising activated esters to yield polymer arms with reactive monomers linked to drug molecules through an amide bond. In other embodiments of star polymers used for cancer treatment, drug molecules are linked to reactive monomers via pH-sensitive linkers, e.g., carbohydrazone, wherein in preferred methods of manufacturing, the drug molecule is linked to a ketone or carbohydrazide that is reacted with polymer arms comprising reactive monomers comprising carbohydrazide or ketone, respectively, to yield polymer arms with reactive monomers linked to drug molecules through a carbohydrazone bond.

It should also be noted that throughout the specification, unless otherwise specified, any general references to the molecular weight of polymers arms (e.g., number average molecular weight, Mn), including preferred ranges of molecular weight of polymer arms, excludes the molecular weight contribution of the reactive monomer beyond the acryl amide or acryl ester, i.e., the molecular weight of any drug molecules and/or linkers linked to the acryl amide or acryl ester of the reactive monomer are not included. In contrast, for experimentally determined values of polymer arm molecular weights, the experimentally determined value is reported, which includes the drug molecules and/or linkers linked to the acryl amide or acryl ester of the reactive monomer.

In some embodiments, the polymer arm (A) comprises a hydrophilic (meth)acrylamide-based homopolymer. A non-limiting example of a homopolymer arm (A) comprising methacrylamide-based monomers is:

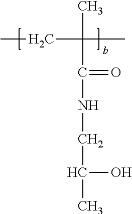

wherein the hydrophilic monomer B is N-(2-hydroxpropyl(methacrylamide)) (HPMA), b is an integer number of monomer units, typically between about 35 to about 420, such as between about 70 to 280 for a target molecular weight between about 10 kDa to about 40 kDa, and wherein the ends of the polymer may be linked to any suitable heterogeneous molecules, such as X1 and Z2 linker precursors, a core (O) and a drug (i.e., D3) or a core (O) and a capping group, respectively.

In some embodiments, the polymer arm (A) comprises a (meth)acrylamide-based copolymer comprising both hydrophilic and charged comonomers. A non-limiting example of a polymer arm (A) comprising a methacrylamide-based copolymer comprising hydrophilic and charged monomers is:

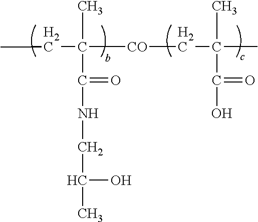

In some embodiments, the polymer arm (A) comprises a (meth)acrylamide-based co-polymer comprising both hydrophilic and reactive comonomers. A non-limiting example of a polymer arm (A) comprising a methacrylamide-based copolymer comprising hydrophilic and reactive monomers is:

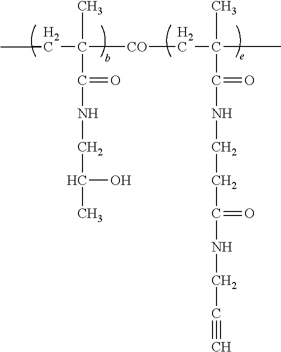

In some embodiments, the polymer arm (A) comprises a (meth)acrylamide-based terpolymer comprising hydrophilic, reactive and charged monomers. A non-limiting example of a polymer arm (A) comprising a methacrylamide-based terpolymer comprising hydrophilic, charged and reactive monomers is:

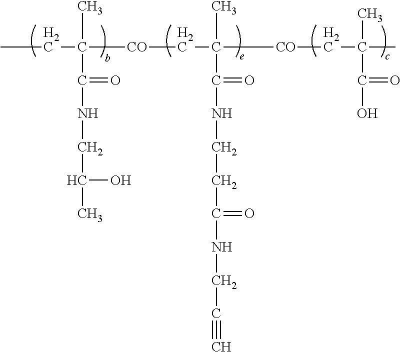

In some embodiments, the polymer arm (A) comprises a (meth)acrylamide-based diblock copolymer. A non-limiting example of a polymer arm (A) comprising a methacrylamide-based diblock copolymer comprising a first block comprising hydrophilic monomers and reactive monomers a second block comprising hydrophilic monomers is shown here for clarity:

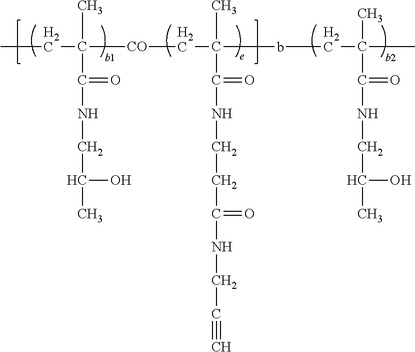

wherein the first block comprises an integer number of repeating units of hydrophilic and reactive monomers denoted by b1 and e; and the other block comprises an integer number of repeating units of a hydrophilic monomer denoted by b2; note that the two blocks in the schematic are separated by brackets [ ], and that “b” delineates the two blocks.

In some embodiments, the polymer arm (A) comprises a (meth)acrylamide-based diblock copolymer, wherein one block comprises reactive monomers and the other block comprises charged monomers. A non-limiting example of a polymer arm (A) comprising a methacrylamide-based diblock copolymer comprising a 1^{st} block with hydrophilic monomers with and reactive monomers and a second block with hydrophilic monomers and reactive monomers is shown here for clarity:

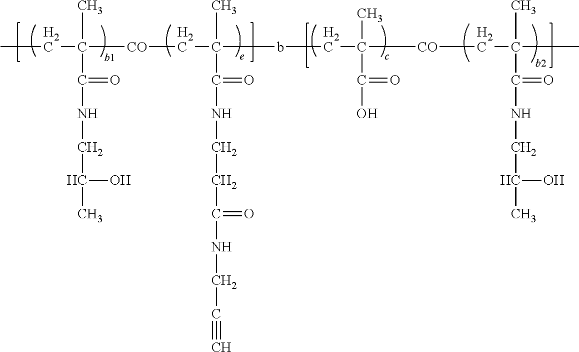

wherein the first block comprises an integer number of repeating units of hydrophilic and reactive monomers denoted by b1 and e; and the second block comprises an integer number of repeating units of charged and hydrophilic monomers denoted by c and b2; note that the two blocks in the schematic are separated by brackets [ ], and that, b, delineates the two blocks.

In the above examples, the reactive monomers may be used to link drug molecules (D). Other examples of reactive monomers are described elsewhere.

In some embodiments, the polymer arm (A) comprises a (meth)acrylamide-based diblock copolymer, wherein one block comprises a terpolymer consisting of reactive monomers, charged monomers and hydrophilic monomers and the other block comprises charged monomers and hydrophilic monomers. A non-limiting example of a polymer arm (A) comprising a methacrylamide-based di-block, wherein the first block comprises hydrophilic monomers, reactive monomers and charged monomers and the second block comprises charged monomers and hydrophilic monomers is shown here for clarity:

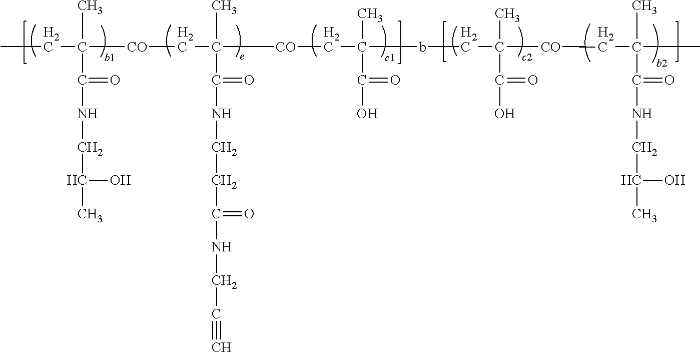

wherein the first block comprises an integer number of repeating units of hydrophilic, reactive and charged monomers denoted by b1, e and c1; and the second block comprises an integer number of repeating units of charged and hydrophilic monomers denoted by c2 and b2, respectively; note that the two blocks in the schematic are separated by brackets [ ], and that “b” delineates the two blocks.

### Polymer Arm (A) Length and Density Considerations

The inventors of the present disclosure observed a direct, linear correlation between polymer arm (A) length (typically expressed as the degree of polymerization or number average molecular weight, Mn) and star polymer radius, and that star polymers with radius between about 5 nm to 30 nm, more preferably between about 7.5 nm and 20 nm, delivered by the intravenous route led to improved biological activity, e.g., for cancer treatment, as compared with star polymers with hydrodynamic size either less than 5 nm radius or greater than 30 nm radius. Based on these findings, the present inventors have identified the optimal polymer arm (A) length, expressed as number average molecular weight (Mn), to achieve the star polymer size, e.g., hydrodynamic radius (Rh), required for certain applications. Preferred polymer arm molecular weights to achieve a given size star polymer needed for different applications are described throughout the specification. Note: Unless otherwise specified, star polymer size refers to hydrodynamic size, e.g., radius or diameter refer to hydrodynamic radius (Rh) or hydrodynamic diameter (Dh), respectively.

The molecular weight of polymer arms (A) of star polymers used for cancer treatment are chosen to ensure that the hydrodynamic size of the star polymer is of sufficient size to prevent renal elimination following intravenous administration but not too large so as to prevent extravasation and entry into the tumor. The optimal polymer arm (A) molecular weight (excluding the molecular weight of any drug molecules and linkers used to link drug molecules to the polymer arms) is between about 5 kDa and 60 kDa, such as 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa and 60 kDa. In preferred embodiments, the polymer arm (A) molecular weight (excluding the molecular weight of any drug molecules and linkers used to link drug molecules to the polymer arms) is between about 5 and 60 kDa, or between about 10 kDa and about 40 kDa, or such as between about 15 kDa and about 55 kDa, such as between about 20 kDa to about 40 kDa, or more preferably between about 25 to about 35 kDa. Note: Sometimes the polymer arm length is expressed as the degree of polymerization. The degree of polymerization, which is the total number of monomer units (equal to the number average molecular weight divided by the average monomer molecular weight), is chosen such that the molecular weight falls within the preferred polymer arm molecular weight ranges provided above, such as between 5 and 60 kDa, such as between about 15 kDa and about 55 kDa, or such as between about 10 kDa and about 40 kDa, or such as between about 20 kDa to about 40 kDa, or more preferably between about 25 to about 35 kDa. Unless otherwise specified, molecular weight of polymer arms and star polymers refers to the number average molecular weight, Mn.

In certain embodiments, wherein the polymer arm is a diblock copolymer, the polymer arm molecular weight is between about 5 and 60 kDa, such as between about 15 kDa and about 55 kDa, such as between about 20 kDa to about 40 kDa, or more preferably between about 25 to about 35 kDa; and, the degree of polymerization block ratio of the first block to the second block is preferably selected between about 2:1 to about 1:5, more preferably about 1:1 to 1:3. Note: Unless otherwise specified, block ratio refers to degree of polymerization block ratio.

In addition to molecular weight, the number of polymer arms (A) attached should also be chosen to meet the demands of the application. For star polymers arraying drug molecules that bind extracellular receptors, the optimal arm number is greater than 3, such as between 3 and 40, preferably between 10 and 30 arms.

For star polymers delivering small molecule drugs to specific tissues other than the liver or spleen, e.g., star polymers delivering amphiphilic or hydrophobic drugs for cancer treatment, an unexpected finding disclosed herein is that therapeutic index improved with increasing arm number. Accordingly, it was found unexpectedly that polymer arm density was inversely proportional to toxicity, with increasing polymer arm density resulting in decreased toxicity. A non-limiting explanation is that increasing the density of polymer arms results in improved shielding thereby preventing uptake by antigen presenting cells e.g., macrophages in the liver in spleen, associated with off-target toxicity. Additionally, it was unknown a priori how arm density would affect release and therefore activity of drug molecules attached to the polymer arms. Unexpectedly, increasing the density of polymer arm from, e.g., 5 polymer arms to about 45 polymer arms, did not affect drug molecule activity (i.e., in vivo efficacy), indicating that the higher polymer arm densities did not impede drug molecule release. While increasing polymer arm density was found to be preferred, an additional unexpected finding was that, for polymer arms between about 10 to about 40 kDa, the efficiency decreases substantially at a density of about 60 polymer arms per star polymer.

Based on these consideration, in some embodiments star polymers with polymers arms have a molecular weight between about 5 and 60 kDa, such as between about 15 kDa and about 55 kDa, such as between about 20 kDa to about 40 kDa, or more preferably between about 25 to about 35 kDa, are at a density of between about 5 to 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59 or 60, with preferred embodiments having between about 15 to 45 arms, or even more preferred between about 25 to about 35 polymer arms.

An additional notable finding was that the optimal number of polymer arms also depends in part on the monomer composition of the polymer arm. Whereas the above ranges apply to polymer arms comprising hydrophilic monomers, reactive monomers (optionally linked to hydrophobic or amphiphilic drug molecules) and/or negatively charged monomers, it was found unexpectedly that lower densities of polymer arms were preferred for certain star polymers comprising polymer arms with positively charged monomers that are pH-responsive, i.e., become positively charged at or below physiologic pH 7.4. Accordingly, for Star polymers comprising polymers arms with positively charged monomers that are pH-responsive at or below physiologic pH, the preferred density was found to be about 5 to about 60 polymers arms, more preferably between about 5 to 35 and more preferably still between about 10 to about 20 polymers arms.

### Linkers

Linkers generally refer to any molecules that join together any two or more different molecules of star polymers, which may additionally perform any one or more of the following functions: (i) increase or decrease water solubility; (ii) increase distance between any two components, i.e., different molecules of the star polymer; (iii) impart rigidity or flexibility; or (iv) control/modulate the rate of degradation/hydrolysis of the link between any two or more different molecules.

Linkers may be used to join any two components of the star polymer, for example, a polymer arm (A) to the core (O) or drug to reactive monomers or ends of the polymer arms by any suitable means. The linker may use covalent or non-covalent means to join any two or more components, i.e., different molecules, for example a polymer arm (A) to the core (O) or a drug molecule (e.g., D2) to reactive monomers. The term “Linker” used in chemical formulae is used to generically refer to any suitable linker molecule. While any suitable linker may be used to join together any two components of the star polymers described herein, preferred linkers that lead to unexpected improvements in activity for certain biological applications are described throughout.

In certain embodiments, a linker may join, i.e., link, any two components of the star polymer through a covalent bond. Covalent bonds are the preferred linkages used to join any two components of the star polymer and ensure that no component is able to immediately disperse from the other components, e.g., drug molecules from the star polymer, following administration to a subject. Moreover, covalent linkages typically provide greater stability over non-covalent linkages and help to ensure that each component of the star polymer is co-delivered to specific tissues and/or cells at or near the proportions of each component that was administered.

In a non-limiting example of a covalent linkage, a click chemistry reaction may result in a triazole that links, i.e., joins together, any two components of the star polymer. In certain embodiments, the click chemistry reaction is a strain-promoted [3+2] azide-alkyne cyclo-addition reaction. An alkyne group and an azide group may be provided on respective molecules comprising the star polymer to be linked by “click chemistry”. In some embodiments, a core (O) comprises a linker precursor X1 bearing an azide functional group that is reactive towards linker precursor X2 bearing an alkyne, for example, an acetylene or a dibenzylcyclooctyne (DBCO).

In some embodiments, a drug with a Z2 linker precursor bearing a thiol functional group is linked to the polymer arms (A) through linker precursor Z1 bearing an appropriate reactive group such as an alkyne, alkene or maleimide, resulting in a thioether bond, or with a pyridyl disulfide, e.g., resulting in a disulfide linkage.

In some embodiments, an amine is provided on one molecule and may be linked to another molecule by reacting the amine with any suitable electrophilic group such as carboxylic acids, acid chlorides, activated esters (for example, NHS ester), which results in an amide bond; the amine may be reacted with alkenes (via Michael addition); the amine make be reacted with aldehydes and ketones (via Schiff base); or the amine may be reacted with activated carbonates or carbamates to yield a carbamate.

There are many suitable linkers that are well known to those of skill in the art and include, but are not limited to, straight or branched-chain carbon linkers, heterocyclic carbon linkers, rigid aromatic linkers, flexible ethylene oxide linkers, peptide linkers, or a combination thereof. In some embodiments, the carbon linker can include a C1-C18 alkane linker, such as a lower alkyl linker, C1-C6 (i.e., from one to six methylene units); the alkane linkers can serve to increase the space between two or more molecules, i.e., different components, comprising the star polymer, while longer chain alkane linkers can be used to impart hydrophobic characteristics. Alternatively, hydrophilic linkers, such as ethylene oxide linkers, may be used in place of alkane linkers to increase the space between any two or more molecules and increase water solubility. In other embodiments, the linker can be an aromatic compound, or poly(aromatic) compound that imparts rigidity. The linker molecule may comprise a hydrophilic or hydrophobic linker. In several embodiments, the linker includes a degradable peptide sequence that is cleavable by an intracellular enzyme (such as a cathepsin or the immuno-proteasome).

In some embodiments, the linker may comprise poly(ethylene oxide) (PEO or PEG). The length of the linker depends on the purpose of the linker. For example, the length of the linker, such as a PEG linker, can be increased to separate components, for example, to reduce steric hindrance, or in the case of a hydrophilic PEG linker can be used to improve water solubility. The linker, such as PEG, may be a short linker that may be at least 2 monomers in length. The linker, such as PEG, may be between about 4 and about 24 monomers in length, such as 4, 5, 6, 7, 8, 9, 10, 11, 12, 13, 14, 15, 16, 17, 18, 19, 20, 21, 22, 23, or 24 monomers in length or more. In some embodiments, drug molecules are linked to the ends of polymer arms (i.e., D3) through PEG linkers.

In some embodiments, polymer arms (A) are linked to the core (O) through a linker X comprising 4 or more ethylene oxide units. Unexpectedly, it was found that X1 linker precursors linked to the core (O) through PEG linkers improved the efficiency of polymer arm (A) coupling to the core (O), particularly for generating star polymers with drug molecules linked to reactive monomer units distributed along the backbone of the polymer arms, e.g., O[D1]-(X-A(D)-[Z]-[D3])n, e.g., O—(X-A(D))n. Specifically, it was observed that the coupling of polymer arms (A) with high densities of drugs molecules (D2) linked to the polymers arms could be improved be using an ethylene oxide linker between the core surface and the functional group (FG) on X1 that reacts with the FG on X2 on the polymer arm to form the linker X. Non-limiting explanations for these findings are that extending the FG present on X1 away from the core into the solvent by using 4 or more ethylene oxide units enables improved coupling by reducing steric hindrance. Thus, in preferred embodiments of star polymers linked to arms with high densities of drug molecules (e.g., >5 mol % or >10 mol % drug molecules), the X1 linker precursor is linked to the core through 4 or more ethylene oxide units, preferably between 4 and 36 ethylene oxide units, such as 4, 5, 6, 7, 8, 9, 10, 11, 12, 13, 14, 15, 16, 17, 18, 19, 20, 21, 22, 23, 24, 25, 26, 27, 28, 29, 30, 21, 31, 33, 34, 35, or 36 ethylene oxide units, though, more preferably between about 12 and 24 ethylene oxide units.

In some embodiments, where the linker comprises a carbon chain, the linker may comprise a chain of between about 1 or 2 and about 18 carbons, such as 1, 2, 3, 4, 5, 6, 7, 8, 9, 10, 11, 12, 13, 14, 15, 16, 17, or 18 carbons in length or more. In some embodiments, where the linker comprises a carbon chain, the linker may comprise a chain of up to about 12 or up to about 20 carbons. In preferred embodiments, drugs (D) are linked to polymer arms through short alkane linkers, typically no more than 6 carbon atoms in length.

In some embodiments, the linker is cleavable under intracellular conditions or within certain tissues (e.g., tumor microenvironment), such that cleavage of the linker results in the release of any component linked to the linker, for example, a small molecule immunostimulant or chemotherapeutic drug (D).

For example, the linker can be cleavable by enzymes localized in intracellular vesicles (for example, within a lysosome or endosome or caveolea) or by enzymes in the cytosol, such as the proteasome or immuno-proteasome. The linker can be, for example, a peptide linker that is cleaved by proteolytic enzymes, including, but not limited to proteases that are localized in intracellular vesicles, such as cathepsins in the lysosomal or endosomal compartment. The peptide linker is typically between 1-6 amino acids, such as 1, 2, 3, 4, 5, or 6. Note: For examples of amino acids and peptides provided in text or chemical structures, the peptides and amino acids are L-amino acids, unless otherwise specified.

Certain peptides, e.g., dipeptides, are known to be hydrolyzed by proteases that include cathepsins, such as cathepsins B and D and plasmin, (see, for example, Dubowchik, G. M. et al. Pharmacology & Therapeutics, 1999, 83 (2), 67-123). For example, a peptide linker that is cleavable by the thiol-dependent protease cathepsin-B, can be used (for example, a Phe-Leu or a Gly-Phe-Leu-Gly (SEQ ID NO: 1) linker). Other examples of such linkers are described, for example, in U.S. Pat. No. 6,214,345, incorporated herein by reference. In a specific embodiment, the peptide linker cleavable by an intracellular protease is a Val-Cit linker or a Phe-Lys linker (see, for example, U.S. Pat. No. 6,214,345, which describes the synthesis of doxorubicin with the Val-Cit linker).

In several embodiments, linkers comprised of peptide sequences of the formula Pn . . . P4-P3-P2-P1 are used to promote recognition by cathepsins, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine; P2 is selected from glycine, serine, leucine, valine or isoleucine; P3 is selected rom acetyl lysine, boc-protected lysine, norleucine (nLeu), glutamine, 6-hydroxy norleucine (abbreviated hnLeu), glycine, serine, alanine, proline, or leucine; and P4 is selected from glycine, serine, arginine, lysine, acetyl lysine (i.e., the epsilon amine is acetylated), Boc protected lysine, aspartic acid, glutamic acid or beta-alanine. In a non-limiting example, a tetrapeptide linker of the formula P4-P3-P2-P1 linked through an amide bond to another molecule and has the sequence Lys-Pro-Leu-Arg (SEQ ID NO: 2). For clarity, the amino acid residues (Pn) are numbered from proximal to distal from the site of cleavage, which is C-terminal to the P1 residue, for example, the amide bond between P1-P1′ is hydrolyzed. Suitable peptide sequences that promote cleavage by endosomal and lysosomal proteases, such as cathepsin, are well described in the literature (see: Choe, Y. et al. J. Biol. Chem. 2006, 281 (18), 12824-12832).

In preferred embodiments of star polymers used for cancer treatment, drug molecules are linked to reactive monomers via enzyme degradable linkers selected from:
a) Single amino acids, —P1-X-D, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine, or most preferably norleucine or alpha-aminobutyric acid;
b) Dipeptides, —P2-P1-X-D, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine, P2 is selected from glycine, serine, leucine, valine or isoleucine;
c) Tripeptides, —P3-P2-P1-X-D, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine, P2 is selected from glycine, serine, leucine, valine or isoleucine; P3 is selected rom acetyle lysine, boc-protected lysine, norleucine, glutamine, 6-hydroxy norleucine, glycine, serine, alanine, proline, or leucine;
d) Tetrapeptides, —P4-P3-P2-P1-X-D, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine, P2 is selected from glycine, serine, leucine, valine or isoleucine; P3 is selected rom acetyle lysine, boc-protected lysine, norleucine, glutamine, 6-hydroxy norleucine, glycine, serine, alanine, proline, or leucine; and P4 is selected from glycine, serine, arginine, lysine, acetyl lysine (i.e., the epsilon amine is acetylated), Boc protected lysine, aspartic acid, glutamic acid or beta-alanine; and,
wherein the linker is linked to the star polymer at either the core, along the polymer backbone or at or near the end of the polymer arms; D is any suitable drug molecule; X is any suitable linker molecule, optionally comprising a self-immolative linker, e.g., PAB.

As disclosed herein, certain enzyme-degradable inker compositions were found to provide unexpected improvements in physicochemical behavior and/or biological activity. Based on these findings, in preferred embodiments of enzyme degradable linkers, P1 is selected from arginine, citrulline, alpha-aminobutyric acid or norleucine, P2 (if present) is selected from valine or serine, P3 (if present); P3 (if present) is selected rom acetyle lysine, boc-protected lysine, norleucine, glutamine, 6-hydroxy norleucine or proline; and P4 (if present) is selected from glycine, beta-alanine or serine. Non-limiting examples of preferred tetra-peptide enzyme degradable linkers include Ser-Pro-Val-aBut, Ser-Pro-Val-Cit, Ser-Lys(Ac)-Val-nLeu, Ser-Lys(Ac)-Val-aBut, Ser-Lys(Ac)-Val-Cit, Ser-nLeu-Val-aBut, Ser-nLeu-Val-Cit, Ser-nLeu-Val-nLeu, Ser-hnLeu-Val-aBut, Ser-hnLeu-Val-Cit, and Ser-hnLeu-Val-nLeu.

In several embodiments, linkers comprised of peptide sequences are selected to promote recognition by the proteasome or immuno-proteasome. Peptide sequences of the formula Pn . . . P4-P3-P2-P1 are selected to promote recognition by proteasome or immuno-proteasome, wherein P1 is selected from basic residues and hydrophobic, branched residues, such as arginine, lysine, leucine, isoleucine and valine; P2, P3 and P4 are optionally selected from leucine, isoleucine, valine, lysine and tyrosine. In a non-limiting example, a cleavable linker of the formula P4-P3-P2-P1 that is recognized by the proteasome is linked through an amide bond at P1 to another molecule and has the sequence Tyr-Leu-Leu-Leu (SEQ ID NO:5). Sequences that promote degradation by the proteasome or immuno-proteasome may be used alone or in combination with cathepsin cleavable linkers. In some embodiments, amino acids that promote immuno-proteasome processing are linked to linkers that promote processing by endosomal proteases. A number of suitable sequences to promote cleavage by the immuno-proteasome are well described in the literature (see: Kloetzel, P.-M. et al. Nat. Rev. Mol. Cell Biol., 2001, 2, 179-187; Huber, E. M. et al. Cell, 2012, 148 (4), 727-738, and Harris, J. L. et al. Chem. Biol., 2001, 8 (12) 1131-1141).

In certain preferred embodiments of star polymers for cancer treatment, drug molecules are linked to linkers comprising an enzyme degradable peptide and may be represented by the formula:

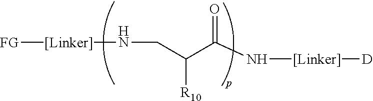

wherein D is a drug molecule; “Linker” is any suitable linker molecule; p denotes an integer number of repeating units of amino acids, though, p is typically 1 to 6 amino acids, such as 1, 2, 3, 4, 5 or 6 amino acids, R₁₀ is any amino acid side group and FG is any suitable functional group for attachment to the star polymer and brackets “[ ]” denote that the group is optional.

In certain preferred embodiments of drug molecules linked to linkers comprising an enzyme degradable peptide, where particularly amphiphilic or hydrophobic drug molecules are linked to reactive monomer units distributed alone the backbone of polymer arms, the drug molecule is linked directly to a peptide that is linked to a Linker that is linked to a functional group, which is shown here for clarity:

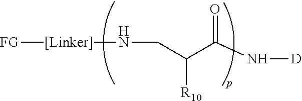

wherein D is any drug molecule; “Linker” is any suitable linker molecule, though, in preferred embodiments the Linker is typically present and selected from short alkyl (e.g., C2 through C6) or PEG (e.g., PEG1 to PEG4) spacers; p denotes an integer number of repeating units of amino acids, though, p is typically 1 to 6 amino acids, such as 1, 2, 3, 4, 5 or 6 amino acids, more preferably 2, 3 or 4 amino acids; R₁₀ is any amino acid side group and FG is any suitable functional group for linking the linker linked to the drug molecule to reactive monomers, though, FG is typically selected from amine, reactive esters, azide, alkyne, hydrazine or ketone functional groups, though, in preferred embodiments the FG is an amine; and, brackets “[ ]” denote that the group is optional.

In the above example, wherein the FG is amine, and the Linker is beta alanine the structure is:

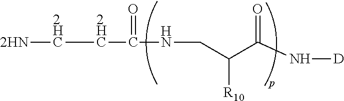

In some preferred embodiments, the drug is linked to the peptide via a self-immolative carbamate linker. A non-limiting example is shown here:

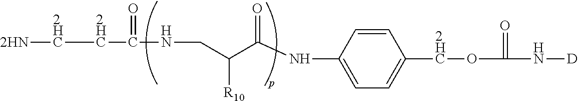

In the above example, wherein p is 4 and the amino acids are Serine-Lysine(Ac)-Valine-Norleucine, the structure is:

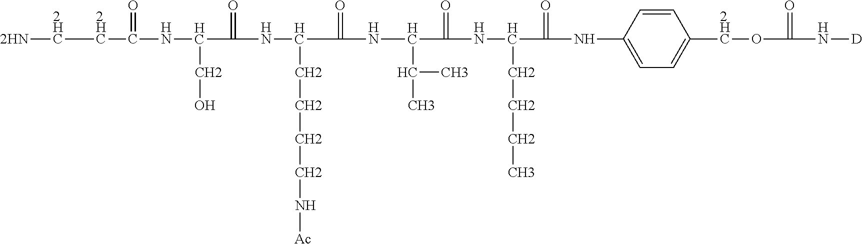

In some embodiments, the drug molecule is linked to a sulfatase degradable linker, wherein hydrolysis of a sulfate by sulfatase enzyme results in release of the drug molecule from the linker. A number of arylsulfatase and alkysulfatase degradable linkers have recently been described (e.g., see: Bargh, J. D. et al. Chem. Sci., 2020, 11, 2375-2380). In some embodiments of the present disclosure, drug molecules are linked to star polymers through sulfatase degradable linkers. Non-limiting examples are shown here for clarity:

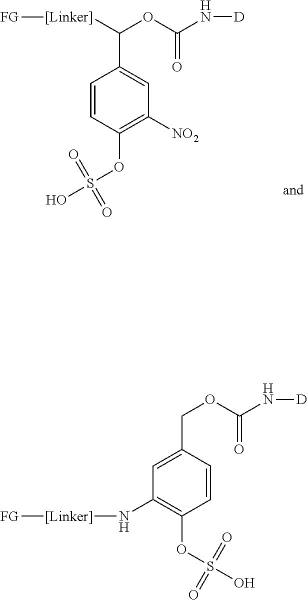

wherein D is any drug molecule; “Linker” is any suitable linker molecule; FG is any suitable functional group for linking the linker linked to the drug molecule to reactive monomers, though, FG is typically selected from amine, reactive esters, azide, alkyne, hydrazine or ketone functional groups, though, in preferred embodiments FG is an amine; and, brackets “[ ]” denote that the group is optional.

Non-limiting examples above the example, wherein the Linker is present and selected from short alkyl linkers, and FG is an amine, is shown here for clarity:

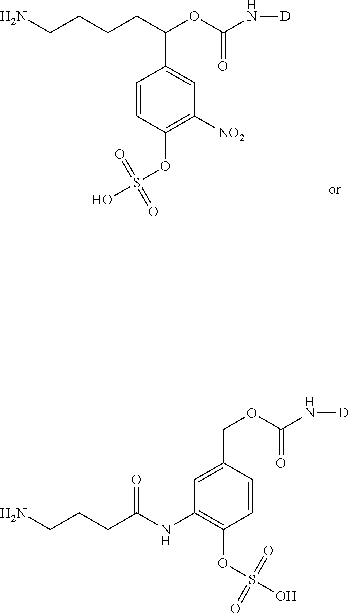

In other embodiments, any two or more components of the star polymer may be joined together through a pH-sensitive linker that is sensitive to hydrolysis under acidic conditions. A number of pH-sensitive linkages are familiar to those skilled in the art and include for example, a hydrazone, carbohydrazone, semicarbazone, thiosemicarbazone, cis-aconitic amide, orthoester, acetal, ketal, silylether or the like (see, for example, U.S. Pat. Nos. 5,122,368; 5,824,805; 5,622,929; Dubowchik, G. M. et al. Pharmacology & Therapeutics, 1999, 83 (2), 67-123; Neville D. M. et al. Biol. Chem., 1989, 264, 14653-14661).

In certain embodiments, different components of the star polymer are linked together through pH-sensitive linkages that are stable at blood pH, e.g., at a pH of about 7.4, but undergo increased rate of hydrolysis at endosome/lysosomal pH, ˜pH 5-6.5. In certain, preferred embodiments of star polymers used for cancer treatment, drug molecules are linked to polymer arms through reactive monomers via a pH-sensitive bonds, such as hydrazone bonds that result from the reaction between a ketone and a hydrazine. Note: The functional group hydrazine linked to a carbonyl is sometimes referred to as hydrazide or carbohydrazide, though, hydrazine is meant to broadly refer to —NH—NH₂ groups, including when linked to carbonyl, e.g., C(O)—NH—NH₂. In certain embodiments of star polymers use for cancer treatment that comprise a first polymer arm comprising drug molecules and a second polymer arm, the second polymer arm is linked to the core through pH-sensitive bonds, such as hydrazone bonds that result from the reaction between a ketone and a hydrazide (or carbohydrazide). pH-sensitive linkages, such as a hydrazone, provide the advantage that the bond is stable at physiologic pH, at about pH 7.4, but is hydrolyzed at lower pH values, such as the pH of intracellular vesicles.

In certain preferred embodiments of star polymers for cancer treatment, drug molecules are linked to linkers comprising a ketone and may be represented by the formula:

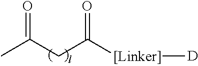

wherein D is any drug molecule; “Linker” is any suitable linker molecule; I denotes an integer number of repeating units, though, I is typically 2 to 5, such as 2, 3, 4 or 5 methylene units, preferably 4; brackets “[ ]” denote that the group is optional; and, wherein the ketone in the above example is used to link the linker linked drug molecule to a reactive monomer through a hydrazone bond.

In the above example, wherein I is 4 and the drug molecule is linked directly (i.e., the “Linker” is absent) to the linker via an amide bond, the structure is:

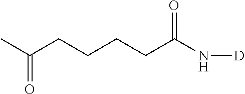

In preferred embodiments, drug molecules linked to ketones are linked to reactive monomers of Formula III through hydrazone or carbohydrazone bonds. Non-limiting examples of drug molecules linker to reactive monomers through hydrazone and carbohydrazone linkers are shown here:

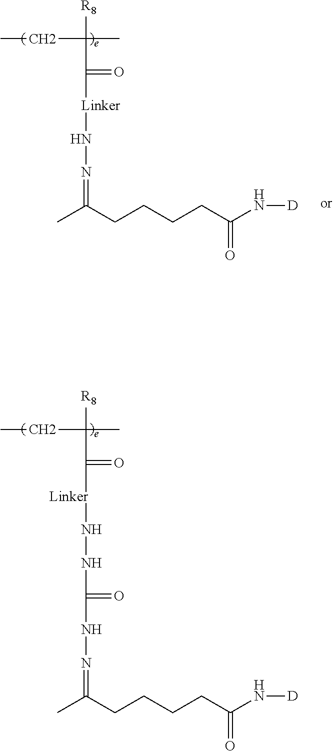

wherein D is any drug molecule, the Linker is any suitable linker molecule, e denotes an integer number of repeating units of the reactive monomer along the polymer arm and R₈ is methyl or H.

Non-limiting examples, wherein in the above examples the Linker is beta-alanine, i.e., R₇ is —NH(CH₂)ₖ—FG, wherein k is 2, and FG is either hydrazide or carbohydrazide, are shown here for clarity:

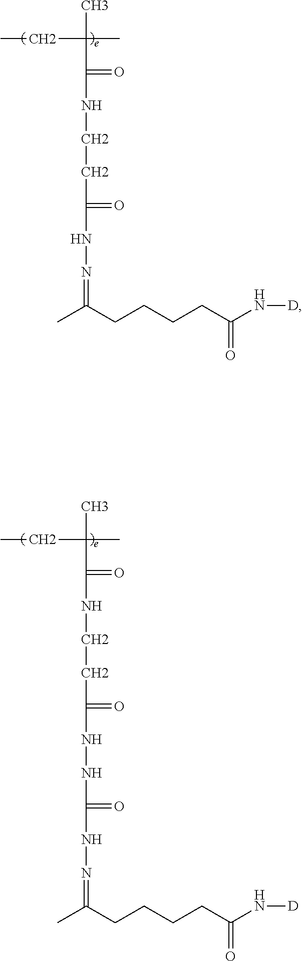

In other embodiments, the linker comprises a linkage that is cleavable under reducing conditions, such as a reducible disulfide bond. Many different linkers used to introduce disulfide linkages are known in the art (see, for example, Thorpe, P. E. et al. Cancer Res., 1987, 47, 5924-5931; Wawrzynczak et al., In Immunoconjugates: Antibody Conjugates in Radioimagery and Therapy of Cancer (C. W. Vogel ed., Oxford U. Press, 1987); Phillips, G. D. L. et al., Cancer Res., 2008, 68 (22), 9280-9290. See also U.S. Pat. No. 4,880,935.).

In yet additional embodiments the linkage between any two components of the star polymer can be formed by an enzymatic reaction, such as expressed protein ligation or by sortase (see: Fierer, J. O. et al. Proc. Natl. Acad. Sci., 2014, 111 (13), 1176-1181, and Theile, C. S. et al. Nat. Protoc., 2013, 8 (9), 1800-1807) chemo-enzymatic reactions (Smith, E. L. et al. Bioconjug. Chem., 2014, 25 (4), 788-795) or non-covalent high affinity interactions, such as, for example, biotin-avidin and coiled-coil interactions (Pechar, M. et al. Biotechnol. Adv., 2013, 31 (1), 90-96) or any suitable means that are known to those skilled in the art (see Chalker, J. M. et al. Acc. Chem. Res., 2011, 44 (9), 730-741, and Dumas, A. et al. Angew Chem. Int. Ed. Engl., 2013, 52 (14), 3916-3921).

### Linkers X and Z

A subset of linkers that perform the specific function of site-selective coupling, i.e., joining or linking together the core (O) with the polymer arm (A), or polymer arm (A) with a drug molecule at the end of the polymer arm (designated “D3” in chemical formulae of star polymers) are referred to as linkers, X and Z, respectively. The linker X forms as a result of the reaction between a linker precursor X1 and a linker precursor X2. For instance, a linker precursor X1 that is linked to the core (O) may react with a linker precursor X2 attached to the polymer arm (A) to form the linker X that joins the polymer arm (A) to the core (O). The linker Z forms as a result of the reaction between a linker precursor Z1 and a linker precursor Z2. For instance, a linker precursor Z1 that is linked to the polymer arm (A) may react with a linker precursor Z2 attached to a ligand D3 to form the linker Z that joins the polymer arm (A) to D3. The linkers X and Z may be formed by any suitable means. In preferred embodiments, the linker precursors used to form X and Z are selected for site-selectivity, i.e., a reaction only takes place between X1 and X2 and/or Z1 and Z2, and between no other groups.

In some embodiments, the linkers X and/or Z are formed as a result of a bio-orthogonal “click chemistry” reaction between the linker precursors, X1/X2 and Z1/Z2, respectively. In some embodiments, the click chemistry reaction is a catalyst free click chemistry reaction, such as a strain-promoted azide-alkyne cycloaddition reaction that does not require the use of copper or any catalyst. Non-limiting examples of linker precursors that permit bio-orthogonal reactions include molecules comprising functional groups selected from azides, alkynes (including strained-alkynes), tetrazines and transcyclooctenes. In some embodiments, a linker precursor Z1 comprising an azide reacts with a linker precursor Z2 to form a triazole linker Z. In other embodiments, a linker precursor X2 comprising a tetrazine reacts with a linker precursor X1 comprising a transcyclooctene (TCO) to form a linker X comprising the inverse demand Diels-Alder ligation product. In some embodiments, a linker precursor X2 comprising an azide reacts with a linker precursor X1 comprising an alkyne to form a linker X comprising a triazole.

### Linker Molecule (Z) Between D3 and the Polymer Arm

Linker molecule (Z) (if present) between the polymer arm D3 at the ends of the polymer arms (A) are formed by the reaction of linker precursors Z1 and Z2 where Z1 is a linker precursor comprising a first reactive functional group and Z2 is a linker precursor comprising a second reactive functional group. A non-limiting example is as follows:

O—[X]-A[(D)]-Z1+Z2-D3→O—([X]-A[(D)]-Z-D3)n

or

[X2]-A[(D)]-Z1+Z2-D3→[X2]-A[(D)]-Z-D3,

### Linker Molecule (X) Between the Core and the Polymer Arm

Linker molecule (X) is formed by the reaction of linker precursors X1 and X2 where X1 is a linker precursor comprising a first reactive functional group and X2 is a linker precursor comprising a second reactive functional group. A non-limiting example is as follows:

O[D]-X1+X2-A[(D)]-[Z]-[D3]→O[D1]-(X-A[(D)]-[Z]-[D3])n,

wherein at least 1 of D1, D2 or D3 are present.

Linker precursors X1 and X2 allow for coupling of the polymer arm (A) with the core (O). For example, a linker precursor X1 that is linked directly or indirectly (e.g., via a linker) to the core (O) may react with a linker precursor X2 that is linked directly or indirectly (via a linker) to the polymer arm (A) to form the linker molecule (X) between the core (O) and the polymer arm (A).

Suitable linker precursors X1 are those that react selectively with linker precursors X2 attached to the polymer arm (A) without linkages occurring at any other site of the polymer arm (A), the linker (Z) (if present) and/or drug molecules (if present). This selectivity is important for ensuring a linkage can be formed between the polymer arm (A) and the core (O) without modification to other components of the star polymer.

In certain embodiments, X1 is a nucleophilic species present on the surface of the core (O). The nucleophilic species may be selected from one or more of the group consisting of —OR₁₇, —NR₁₇R₁₈ and —SR₁₇ where R₁₇ is selected from H and R₁₈ is selected from H, NHR₁₉ or C₁-C₆-alkyl and R₁₉ is selected from H or C₁-C₆-alkyl. In these embodiments, the linker precursor X1 can be reacted with a carboxyl moiety (e.g., activated carboxylic acid) on X2 to form a linker comprising an ester, amide or thioester. In certain embodiments, X1 is NR₁R₂. R₁ and R₂ are each independently selected from the group consisting of H and C₁-C₆-alkyl. In certain specific embodiments, R₁ and R₂ are both H, i.e., X1 on the core is an amine and can be linked to X2 comprising a carboxyl moiety to form an amide bond.

In certain embodiments, the acylation reaction between X1 and X2 can be carried out using a suitable coupling agent. Suitable coupling agents include but are not limited to BOP reagent, DEPBT, N,N′-dicyclohexylcarbodiimide, N,N′-diisopropylcarbodiimide, DMTMM, HATU, HBTU, HCTU, 1-hydroxy-7-azabenzotriazole, hydroxybenzotriazole, PyAOP reagent, PyBOP, thiocarbonyldiimidazole and the like.

In certain other embodiments, the acylation can be carried out by reacting the nucleophilic X1 group with an activated carbonyl moiety. In these embodiments, X2 is an activated carbonyl group of formula —C(O)W where W is a leaving group. Suitable leaving groups include halogen, thiazolidine-2-thione (TT), NHS, nitrophenol, etc. In certain specific embodiments, W is a thiazolidine-2-thione moiety, e.g., X2 comprises thiazolidine-2-thione (TT) and is reacted with X1 comprising an amine to form an amide bond. Note: In some chemical formulae, the leaving group “W” is referred to as “LG.”

In certain embodiments, the linker molecule (X) comprises an optionally substituted alkyl or optionally substituted heteroalkyl group. In certain embodiments, the linker molecule (X) comprises the core structure of a CTA used in a RAFT polymerization to form the polymer arm (A). For example, when the chain transfer agent is 4,4′-azobis(4-cyanovaleric acid) initiator (ACVA) the linker molecule (X) will be a 4-cyanovaleric acid derivative (or 4-cyanopentanoic acid derivative) having the formula —C(O)(CH₂)₂C(CN)(CH₃)—.

In some embodiments, the linker precursor X1 and linker precursor X2 are each covalently attached to both the moieties being coupled. In some embodiments, linker precursor X1 and linker precursor X2 are bifunctional, meaning the linkers include a functional group at two sites, wherein the functional groups are used to couple the linker to the two moieties. The two functional groups may be the same (which would be considered a homobifunctional linker) or different (which would be considered a heterobifunctional linker).

In preferred embodiments of star polymers that comprise a high density of drug molecules (e.g., >5 mol % or >10 mol %) linked to reactive monomers distribute along the polymer arms, the polymer arms are linked to the core through a linker X comprising a triazole formed by the reaction of a linker precursor X1 comprising a strained alkyne reacted with a linker precursor X2 comprising an azide.

### Amplifying Linkers

Some applications of star polymers require high drug molecule density on the surface of the star polymers as well as high molecular weight polymer arms (A). However, the inventors of the present disclosure found that polymer arm molecular weight is directly proportional to hydrodynamic size but inversely related to arm loading (i.e., density on the surface of the star polymer). Therefore, to address this challenge and achieve sufficient densities of D3 on star polymers with sufficient molecular weight polymer arms to achieve a sufficient hydrodynamic size, the present inventors developed novel compositions of star polymers with amplifying linkers that enable the attachment of two or more D3, which may be the same or different, on the ends of each of the polymer arms (A) radiating from the core (O), thereby allowing for an increase in D3 density without further increasing the number of polymer arms.

Suitable amplifying linkers include any bifunctional linker molecule that can join two or more D3 to a single polymer arm (A). Amplifying linkers may be expressed by the formula, (FG1)-T-(FG2)m, wherein FG1 and FG2 are any functional group, T is any suitable linker and m represents the number of FG2 linked to the amplifying linkers and is any integer greater than 1, typically between 2 to 16; wherein the amplifying linker, T, is a dendritic amplifying linker, wherein each monomer of the dendron has an integer number of branches, β, and the dendron can be any generation represented by an integer number, γ. Thus, the multiple by which dendritic amplifying linkers increase functionality (FG1->FG2) can be expressed as g=β^{γ}. In a non-limiting example, for a 4^{th} generation dendron comprised of monomers with 2 branch points, g is equal to 16.

A non-limiting example of a second-generation lysine-based dendron, wherein g=4, is:

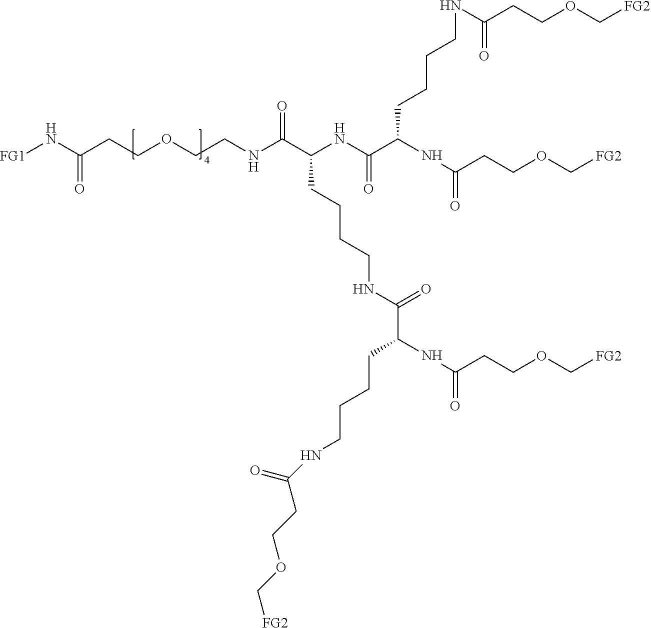

In some embodiments, the amplifying linker has the formula (sulfo-DBCO)-T-(Maleimide)m and is used to install multiple maleimide functional groups onto a polymer arm (A) terminated with an azide functional group. A non-limiting example of a (sulfo-DBCO)-T-(Maleimide)m amplifying linker is:

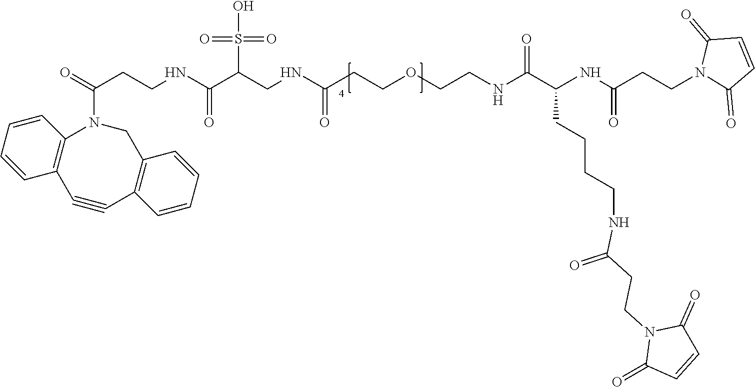

In other embodiments, the amplifying linker has the formula (sulfo-DBCO)-T-(alkyne)m and is used to install multiple alkyne functional groups onto the end of a polymer arm (A) terminated with an azide functional group. A non-limiting example of a (sulfo-DBCO)-T-(alkyne)m amplifying linker is:

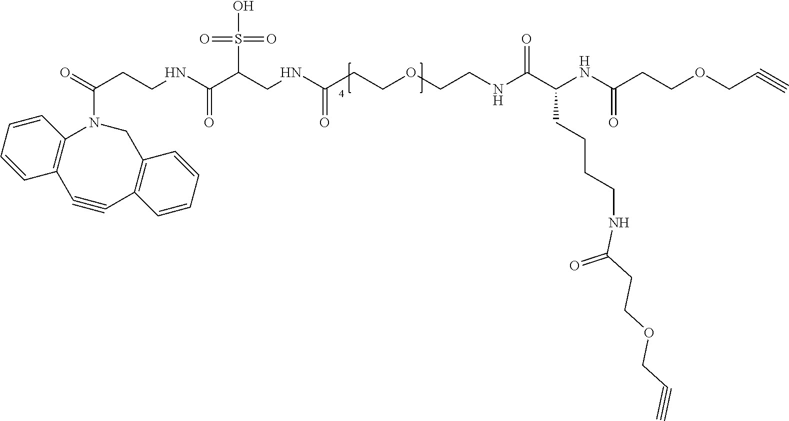

### Selection of X and Z to Meet the Specific Demands of the Application

The linkers, X and Z, may be selected to meet the specific demands of the application. For example, the composition of the linkers X and Z, are selected to achieve high polymer arm (A) and drug loading (i.e., D2 and/or D3) loading and to ensure that coupling of the polymer arm (A) and drug (D2 and/or D3) is regioselective.

A non-limiting example of a route for producing star polymers of the present disclosure, referred to as Route 1, is to link drug molecules D2 and/or D3 to a polymer arm (A), and then attach the D2 and/or D3 functionalized polymer arms to the core (O), for example:

[X2]-A[(D2)]-Z1+Z2-[D3]→[X2]-A[(D2)]-Z-[D3]

O—[X1]+[X2]-A[(D2)]-[Z]-[D3]→O([X]-A[(D2)]-[Z]-[D3])n

where O, A, X1, X2, X, Z1, Z2, D2, D3, n and [ ] are as previously defined herein, and at least one of D2 or D3 is present.

In preferred methods of manufacturing star polymers comprising D2 using Route 1, one or more drug molecules are attached to reactive monomers distributed along a polymer arm that comprises linker precursor X2 and optionally comprises Z1, D3 or a capping group, yielding a polymer arm of formula X2-A(D2)-[Z1, cap or D3], which is then linked to a core (O) comprising linker precursor X1 to generate a star polymer of formula O—(X-A(D)-[Z1, cap or D3])n. In some methods of manufacturing star polymers comprising D2 using Route 1, drug molecules (D2) are linked to reactive through a covalent bond, e.g., an amide bond, either directly or via a linker and the linker X is formed as a result of a click chemistry reaction.

Another non-limiting example of a method of manufacturing star polymers, referred to as Route 2, is to link polymer arms (A) to the core (O) and then attach D2 and/or D3 to the polymer arms (A) radiating therefrom. For example:

O—[X1]+[X2]-A-[Z1]→O([X]-A-[Z1])n

O([X]-A-[Z1])n+D2 and/or +[Z2]-D3→O([X]-A[(D2)]-[Z]-[D3])n

where O, A, X1, X2, X, Z1, Z2, D2, D3, n and [ ] are as previously defined herein and at least one of D2 or D3 are present.

In certain methods of preparing a star polymer using the Route 1 synthetic scheme, the linker precursors Z1 and Z2 are selected to achieve regioselectivity for attachment of the polymer arm (A) to D3. In certain embodiments, the Z2 linker precursor comprises a clickable functional group, e.g., azides, alkynes, tetrazines, transcyclooctynes or other any such suitable molecule, and the Z1 linker precursor is selected to specifically react with the Z2 linker, such as azide/alkyne or tetrazine/transcyclooctyne. In other embodiments, the linker precursor Z2 comprises a thiol or amine, such as a cysteine or lysine that permits regioselective linkage, e.g., to a linker precursor Z2 that comprises a maleimide or activated carbonyl. In certain other embodiments, wherein D3 comprises a peptide, an amino acid on D3, e.g., a cysteine, lysine or alpha-amine of the N-terminal amino acid, is converted to a clickable functional group using a hetero-bifunctional cross-linker. Non-limiting examples include a hetero-bifunctional cross-linker comprising a maleimide linked to an azide; a maleimide linked to an alkyne; a maleimide linked to a tetrazine; a maleimide linked to a transcyclooctyne; an activated carbonyl, e.g., reactive ester linked to an azide; a reactive ester linked to an alkyne; a reactive ester linked to a tetrazine; or a reactive ester linked to a transcyclooctyne, wherein the functional groups of the heterofunctional linker may be linked through any suitable means.

In some embodiments, the star polymer is prepared in either aqueous or organic solvents using the Route 1 synthetic scheme. In certain preparations of a star polymer using the Route 1 synthetic scheme in organic or aqueous solvents, a polymer arm (A) bearing a thiol-reactive functional group, e.g., maleimide, is reacted with a linker precursor Z2 bearing a thiol to form a linker, Z, comprising a thioether bond; then a linker precursor X1 bearing an azide or transcyclooctyne is reacted with a linker precursor X2 bearing an alkyne or tetrazine to form a Linker, X, thereby resulting in a fully assembled star polymer. In other preparations of a star polymer using the Route 1 synthetic scheme in organic or aqueous solvents, a thiol group present on D3 is converted to a clickable group, such as an azide or tetrazine, and the azide or tetrazine Z2 group is reacted with a polymer arm (A) bearing either an alkyne or transcyclooctyne linker precursor Z1 to form a linker, Z; then, the resulting polymer arm (linked to D3) is reacted to a core, (O), using X1/X2 linker precursor pairs selected from either tetrazine/transcyclooctyne or alkyne/azide, respectively.

In other preparations of a star polymer using the Route 1 synthetic scheme in organic or aqueous solvents, a polymer arm (A) bearing an amine-reactive functional group, e.g., activated-ester, is reacted with a linker precursor Z2 bearing an amine to form a linker, Z, comprising an amide bond; then a linker precursor X1 bearing an azide or transcyclooctyne is reacted with a linker precursor X2 bearing an alkyne or tetrazine to form a linker, Z, thereby resulting in a fully assembled star polymer. In other preparations of a star polymer using the Route 1 synthetic scheme in organic or aqueous solvents, an amine group present on D3 is converted to a clickable group, such as an azide or tetrazine, and the azide or tetrazine Z2 group is reactive with a polymer arm (A) bearing either an alkyne or transcyclooctyne linker precursor Z1 to form a linker, Z; then, the resulting polymer arm (A) and D3 conjugate is reacted to a core, (O), using X1/X2 linker precursor pairs selected from either tetrazine/transcyclooctyne or alkyne/azide, respectively.

In still other preparations of a star polymer using the Route 1 synthetic scheme in organic or aqueous solvents, Z2 comprising a clickable reactive group, such as an azide or tetrazine, is introduced to D3, and the azide or tetrazine Z2 group is reacted with a polymer arm (A) bearing either an alkyne or transcyclooctyne linker precursor Z1 to form a linker, Z; then, the resulting polymer arm (A) and D3 conjugate is reacted to a core, (O), using X1/X2 linker precursor pairs selected from either tetrazine/transcyclooctyne or alkyne/azide, respectively. In some embodiments, the Z1 linker precursor comprises 1 or more amino acids that are recognized by an enzyme that catalyzes the linkage of Z1 to Z2 to form the linker Z.

In some embodiments, the star polymer is prepared in organic solvents using the Route 2 synthetic scheme. In certain preparations of a star polymer using the Route 2 synthetic scheme and an organic solvent, a linker precursor X1 bearing an amine functional group is reacted with a linker precursor X2 bearing an activated ester to form a linker, X, comprising an amide bond, and then a linker precursor Z1 bearing an azide is reacted with a linker precursor Z2 bearing an alkyne to form a Linker, Z, comprising a triazole. In other preparations of a star polymer using the Route 2 synthetic scheme and an organic solvent, a linker precursor X1 bearing an amine functional group is reacted with a linker precursor X2 bearing an activated ester to form a linker, X, comprising an amide bond, and then a linker precursor Z1 bearing a tetrazine is reacted with a linker precursor Z2 bearing an TCO to form a Linker, Z. In additional preparations of a star polymer using the Route 2 synthetic scheme and an organic solvent, a linker precursor X1 bearing an amine functional group is reacted with a linker precursor X2 bearing an activated ester to form a linker, X, comprising an amide bond and any unreacted amines are reacted (“capped”), e.g., with acetyl groups by reaction with acetyl chloride or acetic anhydride; then a thiol-reactive Z1 group, e.g., maleimide, is installed on the polymer arms (A), which are reacted with a linker precursor Z2 bearing a thiol group to form a Linker, Z, comprising a thioether linkage. In still other preparations of a star polymer using the Route 2 synthetic scheme and an organic solvent, a linker precursor X1 bearing a TCO group is reacted with a linker precursor X2 bearing a tetrazine to form a linker, X, and then a linker precursor Z1 bearing an activated ester is reacted with a linker precursor Z2 bearing an amine to form a Linker, Z, comprising an amide bond.

In some embodiments, the star polymer, is prepared using the Route 2 synthetic scheme, wherein in the first step either an organic solvent or aqueous solution is used but in the second step an aqueous solution is used, such as may be required due to incompatibility of D3 with organic solvents. A non-limiting example includes the preparation of a star polymer, wherein in the first step in either an organic solvent or aqueous solution, a linker precursor X1 bearing an amine functional group is reacted with a linker precursor X2 bearing an activated ester to form a linker, X, comprising an amide bond, and then in the second step in an aqueous solution a linker precursor Z1 bearing an azide is reacted with a linker precursor Z2 bearing an alkyne to form a linker, Z, comprising a triazole. An additional non-limiting example includes the preparation of a star polymer using the Route 2 synthetic scheme, wherein in the first step in either an organic solvent or aqueous solution, a linker precursor X1 bearing an amine functional group is reacted with a linker precursor X2 bearing an activated ester to form a linker, X, comprising an amide bond and any unreacted amines are reacted (“capped”) prior to installing a thiol-reactive Z1 group, e.g., maleimide, on the polymer arms (A); then in the second step in an aqueous solution, Z1 is reacted with a linker precursor Z2 bearing a thiol group to form a Linker, Z, comprising a thioether linkage. Another non-limiting example includes the preparation of a star polymer using the Route 2 synthetic scheme, wherein in the first step in an organic solvent or aqueous solution, a linker precursor X1 bearing a TCO group is reacted with a linker precursor X2 bearing a tetrazine to form a linker, X, and then in the second step in an aqueous solution a linker precursor Z1 bearing an activated ester is reacted with a linker precursor Z2 bearing an amine to form a Linker, Z, comprising an amide bond.

The synthetic route as well as the choice of linkers used to prepare star polymer ligand display system depends, in part, on the composition of the drug molecules, D2 and/or D3.

For instance, it was observed unexpectedly that the density of relatively high molecular weight, e.g., greater than 10,000 Da, drug molecules (i.e., “D3”) that can be displayed on the star polymer depends, in part, on the synthetic route. Accordingly, the loading of certain D3 with relatively high molecular weight, e.g., greater than 10,000 Da, was higher when the Route 1 synthetic scheme was used as compared with the route 2 scheme. Therefore, in preferred methods of manufacturing star polymer comprising relatively high molecular weight D3, e.g., greater than 10,000 Da, the Route 1 synthetic scheme is used wherein the D3 is linked to the polymer arm (A) and then the resulting polymer arm-D3 conjugate ([X1]-A[(D2)]-[Z]-D3) is linked to a core (O) to form a star polymer.

Similarly, it was observed unexpectedly that the density (mol %) of D2 on the polymer arms of star polymers that can be achieved depends, in part, on the synthetic route. Accordingly, it was generally observed that Route 1 synthetic scheme led to higher densities (mol %) of D2 on polymer arms, as compared with Route 2 synthetic scheme. Therefore, in preferred methods of manufacturing star polymers comprising D2, the Route 1 synthetic scheme is used wherein D2 is linked to the polymer arm (A), and then the resulting polymer arm-D2 conjugate ([X1]-A(D2)-[Z-D3, Z1 or cap]) is linked to a core (O) to form a star polymer.

In some embodiments, the star polymer comprises D3 based on a recombinant protein or glycoprotein that is not suitable for use in organic solvents. In some embodiments, the recombinant protein or glycoprotein is greater than 10,000 Da in molecular weight and not suitable for use in organic solvent, the Route 1 synthetic scheme using aqueous solutions is preferred.

D3 that are relatively low molecular weight, e.g., less than 10,000 Da, produced by synthetic means and suitable for use in organic solvents are least restrictive in terms of options for linker chemistries available for forming the Linkers, X and Z and may be produced by either Route 1 or 2 in organic or aqueous conditions. Unexpectedly, it was observed that the highest densities of D3 on star polymers could be achieved using synthetic Route 2 and organic solvents for the assembly of star polymers displaying D3 with relatively low molecular weight.

Particular linker precursors (X1 and X2, and Z1 and Z2) and resulting linkers (X and Z) presented in this disclosure provide unexpected improvements in manufacturability and improvements in biological activity. Many such linker precursors (X1 and X2, and Z1 and Z2) and linkers (X and Z) may be suitable for the practice of the invention and are described in greater detail throughout.

### Transposition

Those skilled in the art recognize that suitable pairs of functional groups, or complementary molecules, selected to join any two components may be transposable; e.g., functional groups used to join a drug (D) to a reactive monomer may be transposable between the drug and the reactive monomer; linker precursors X1 and X2 may be transposable between X1 and X2; linker precursors for Z1 and Z2 may be transposable between Z1 and Z2; and, linker precursors for X1 and X2 may be transposable between Z2 and Z2. For example, a linker (X) comprised of a triazole may be formed from linker precursors X1 and X2 comprising an azide and alkyne, respectively, or from linker precursors X1 and X2 comprising an alkyne and azide, respectively. Thus, unless stated otherwise herein, any suitable functional group pair resulting in a linker (X or Z, or, e.g., a linker between a pharmaceutically active compound, such as a drug (D) and a reactive monomer, may be placed on either X1 or X2 and Z1 or Z2 or the drug and the reactive monomer.

As disclosed herein, certain linker precursor combinations were found to lead to improved manufacturability. For instance, in the preparation of star polymers with D3 using the Route 1 synthetic scheme in aqueous conditions, the combination of a linker precursor X1 comprising an azide and the linker precursor X2 comprising an alkyne was found to lead to improved arm loading (density) as compared with the linker precursor X1 comprising an alkyne and the linker precursor X2 comprising an azide. A non-binding explanation is that the azide is more accessible than the alkyne for coupling the core (O) to the polymer arm (A) in aqueous conditions.

In other embodiments, wherein the linker X is formed as a result of a reaction between a tetrazine and transcyclooctyne, the combination of a linker precursor X1 comprising a TCO and the linker precursor X2 comprising a tetrazine was found to lead to improved arm loading (density) as compared with the linker precursor X1 comprising a tetrazine and the linker precursor X2 comprising a TCO. A non-binding explanation is that tetrazine functional group was unexpectedly found to be unstable on certain cores (O) comprising multiple amine functional groups. Therefore, in preferred embodiments, wherein the dendrimer core comprises primary amines, the Z2 comprising TCO is used.

Incorporation of X2 and Z1 onto the Polymer Arms (a)

The linker precursors X2 and Z1 may be introduced onto the polymer through any suitable means.

For polymer arms (A) produced by RAFT polymerization, the linker precursors X2 and Z1 may be selectively introduced at the ends of the polymer arms during the initiation of polymerization and capping steps.

Introduction of X2 and Z1 onto the polymer arms (A) using RAFT polymerization can be achieved using specialized CTAs and initiators. In a non-limiting example, the CTA is selected from dithiobenzoates and has the generic structure,

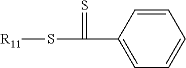

wherein R₁₁ is X2 (or Z1); and, the initiator is selected from the azo class of initiators and has the generic structure, R₂—N═N—R₁₂, wherein, R12 in this example is equivalent to R₁₁ and is X2 (or Z1).

In a non-limiting example, X2 (or Z1) is introduced to the polymer arm during polymerization using a functionalized azo-initiator and a functionalized dithiobenzoate-based CTA:

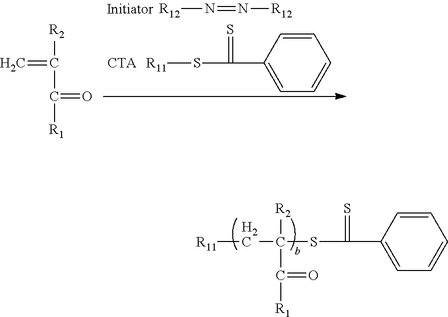

wherein R₁ is —OR₃, —NHR₃ or —N(CH₃)R₃, where R₂ can be H or CH₃, and R₃ is independently selected from any hydrophilic substituent; R₁₁ on. The dithiobenzoate-based CTA and R₁₂ on the initiator are the same and are both X2 (or Z1); and, the resulting polymer comprises an integer number, b, of repeating units of hydrophilic monomers. In this example, in the second step, the dithiobenzoate group on the end of the polymer chain is removed and capped with Z1 (or X2) using a functionalized azo-initiator as shown here:

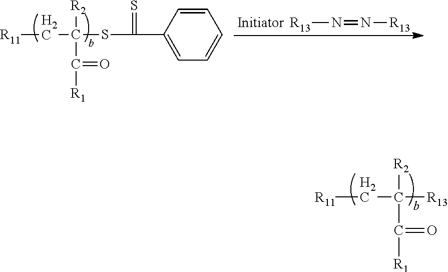

wherein R₁ is —OR₃, —NHR₃ or —N(CH₃)R₃, where R₂ can be H or CH₃, and R₃ is independently selected from any hydrophilic substituent; R₁₁ is X2 (or Z1); b is an integer number of repeating units of hydrophilic monomers and R₁₃ is Z1 (or X2).

In some embodiments, the CTA is based on dithiobenzoate and comprises an activated carbonyl, such as an activated ester, and has the structure

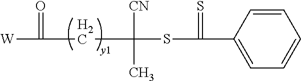

wherein y1 denotes an integer number of methylene units, typically between 1 to 6, and W is a leaving group. A non-limiting example of a dithiobenzoate-based CTA comprising an activated carbonyl is:

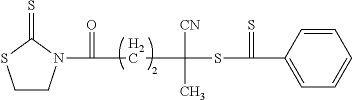

In some embodiments, the CTA is based on dithiobenzoate and comprises a functional group (FG) linked to the CTA through an amide bond and has the structure:

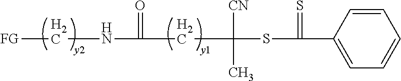

wherein y1 and y2 denote an integer number of repeating methylene units, typically between 1 to 6, and FG is any functional group, such as an azide, alkyne, tert-butyloxycarbonyl protected amine (NH₂—Boc), tert-butyloxycarbonyl protected hydrazide (NHNH-Boc). In a non-limiting example of a dithiobenzoate-based CTA linked to a functional group through an amide bond, the FG is an alkyne, y1=2 and y2=1 and the structure is:

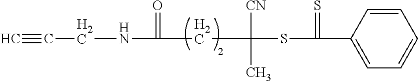

In some embodiments, the azo-initiator comprises an activated carbonyl and has the structure

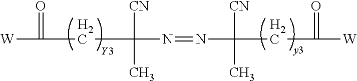

wherein y3 denotes an integer number of methylene units, typically between 1 to 6, and W is a leaving group. A non-limiting example of an azo-initiator comprising an activated carbonyl wherein y3=2 and W is thiazoline-2-thione (“TT group”) is:

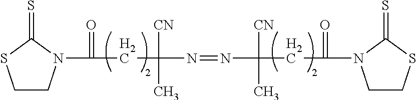

In some embodiments, the azo-initiator comprises a functional group (FG) linked to the initiator through an amide bond, and has the structure:

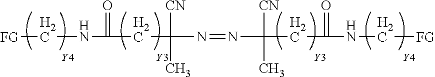

wherein y3 and y4 denote an integer number of methylene units, typically between 1 to 6, and the FG is any functional group, e.g., azide, alkyne, tert-butyloxycarbonyl protected amine (NH₂—Boc), tert-butyloxycarbonyl protected hydrazide (NHNH-Boc), dibenzocyclooctyne (DBCO), bicyclononyne (BCN), methyltetrazine (mTz). In some embodiments, the linker joining the FG to the amide bond may include an ethylene oxide spacer alone or in combination with an aliphatic linker. A non-limiting example of an azo-initiator, wherein in FG is an alkyne, y3=2 and y4=1 is:

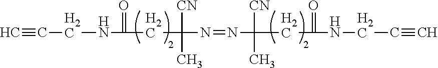

Functionalized initiators and CTAs can be used to incorporate the suitable X2 and Z1 linker precursors onto the polymer during polymerization. In certain embodiments, polymer arms with X2 comprising an activated carbonyl and Z1 comprising an azide are produced in a two-step reaction. In a non-limiting example for the preparation of a polymer arm (A) comprising an activated carbonyl for X2 and an azide for Z1, acrylamide-based monomers are polymerized in the presence of CTA and initiator containing an activated carbonyl as shown here:

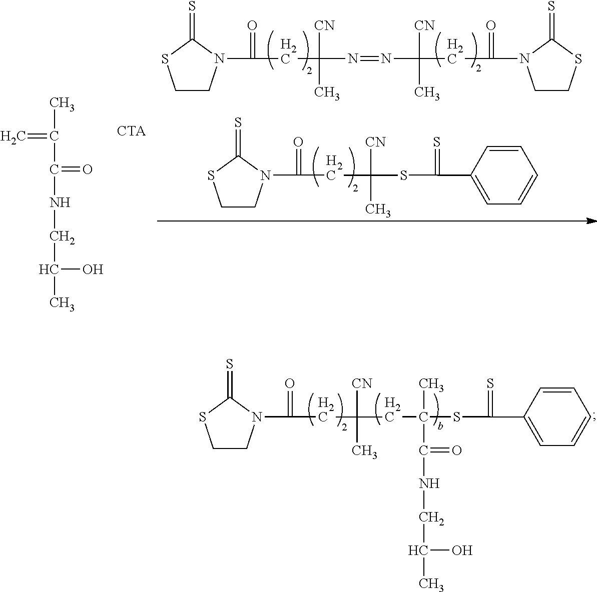

in the second step, the dithiobenzoate group of the polymer arm is replaced with Z1 by reacting (“capping”) the polymer with an initiator containing an azide functional group, as shown here:

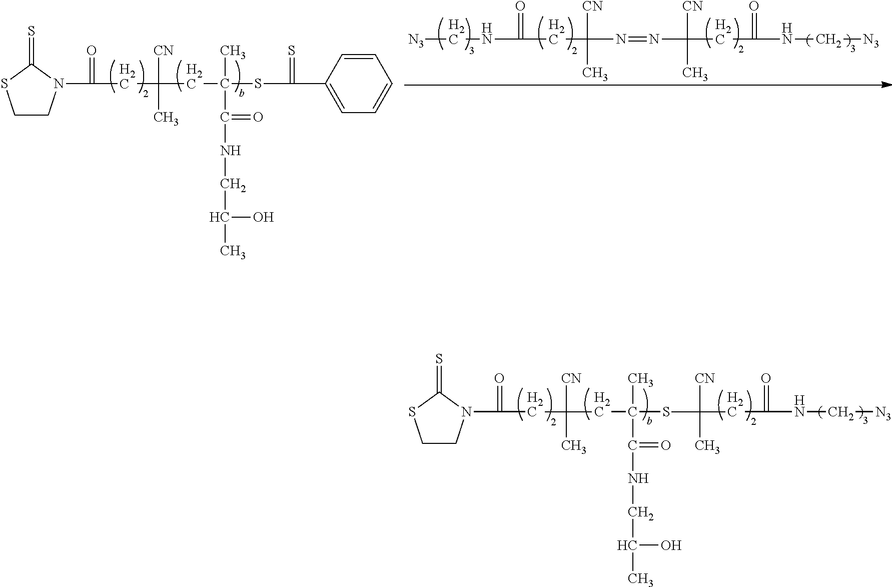

In an alternative non-limiting example for the preparation of a polymer arm (A) comprising an activated carbonyl for X2 and an azide for Z1, acrylamide-based monomers are polymerized in the presence of CTA and initiator containing an azide as shown here:

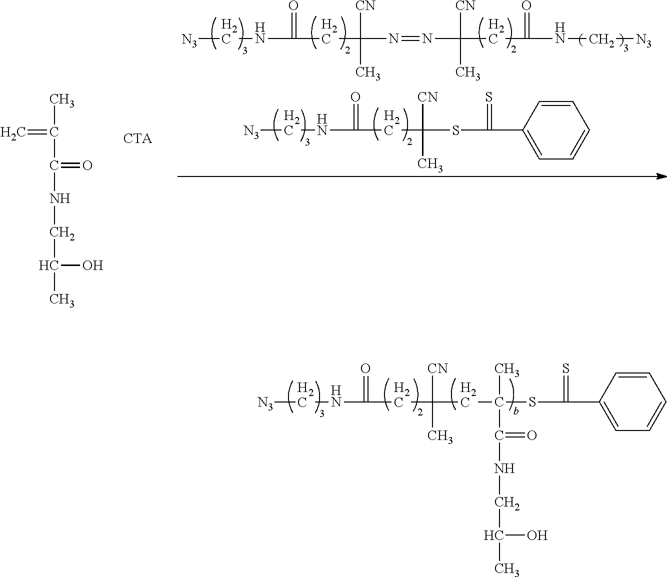

in the second step, the dithiobenzoate group of the polymer arm is replaced with X1 by reacting (“capping”) the polymer with an initiator containing an activated carbonyl group, as shown here:

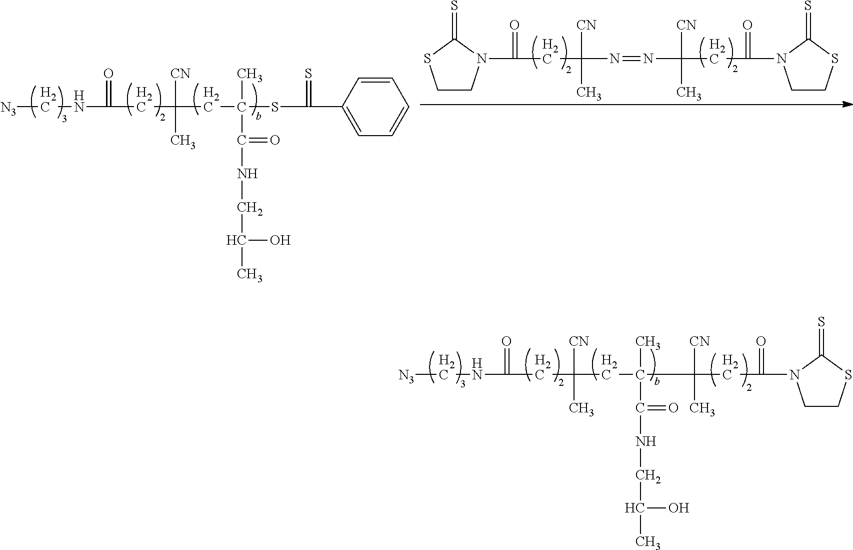

Unexpectedly, it was found that the addition of the Z1 precursor to the polymer arm (A) in the first step, i.e., polymerization of monomers in the presence of Z1-functionalized CTA and Z1-functionalized initiator, followed by the addition of the X2 precursor to the polymer (A) in the second step (i.e., by capping the polymer arm with excess X2 functionalized initiator) led to polymer arms (A) that were less prone to cross-linking cores than polymers arms (A) wherein the X2 is added in the first step. A non-limiting explanation is that the linker precursor X2 or Z1 introduced onto the polymer arm in the first step (polymerization) has the propensity to form a homo-bifunctional polymer arm, X2-A-X2 or Z1-A-Z1, respectively, in the second step (capping). Since X2-A-X2 can cross-link cores, e.g., O—X1+X2-A-X2+X1-o to form O—X-A-X—O, but Z1-A-Z1 cannot, it was determined herein that the route that does not lead to cross-linking, i.e., adding X2 during or after capping, is preferred. Therefore, in preferred embodiments of star polymers, the Z1 linker precursor is optionally added to the polymer arm (A) during polymerization in a first step, and the linker precursor X2 is added to the polymer arm (A) in a second step (capping) by reacting the polymer arm with excess initiator functionalized with X2. This process led to unexpected improvements in manufacturing of star polymers.

Methods for preparing polymer arms with different X2 and Z1 linker precursors groups are described throughout the specification.

Note: While X2, Z1 and D3 may be introduced during the “capping step,” the term cap is used herein to generically refer to an inert group placed at the ends of the polymer arms.

### Process for Attaching Drug Molecules, D2, to the Polymer Arms

For star polymers comprising drug molecules linked to the polymer arms, there exist several synthetic routes for introducing the drug molecule. In preferred methods of manufacturing star polymers with drug molecules (D2) linked to the polymers arms (A), the drug molecule is first attached to the polymer arm (A) to generate a polymer arm comprising one or more drug molecules (D2). Then the polymer arm comprising one or more drug molecules (D2) is grafted to a core (O) to yield a star polymer. This process was found to provide advantages over the attachment of drug molecules to polymer arms (A) already linked to a core (O).

### Selection of Drug Molecules for Surface Array (03)

In certain embodiments, the star polymer comprises arms linked at the ends to drug molecules (“D3”). D3 can be any molecule that acts extracellularly, such as by binding to or associating with soluble or cell surface bound receptors, such as extracellular receptors. The extracellular receptors to which D3 binds may be free, or membrane or cell associated. Non-limiting examples of D3 include synthetic or naturally occurring compounds. Non-limiting examples include protein, peptide, polysaccharide, glycopeptide, glycoprotein, lipid, or lipopeptide-based D3. Examples of proteins include naturally occurring protein ligands, as well as antibodies or antibody fragments that are agonists or antagonists of extracellular receptors. The antibody may be engineered or naturally occurring, i.e., derived from an organism, or a combination thereof, e.g., a partially engineered antibody or antibody fragment. Other examples include synthetic, low-molecular-weight molecules, such as non-naturally occurring heterocycles that bind to extracellular receptors.

The present inventors have unexpectedly found that arrays of D3 on star polymers of formula O—([X]-A[(D)]-[Z]-D3)n show improved receptor binding as well as enhanced biological activity as compared with that observed with D3 arrayed on linear copolymers, or delivered on conventional particle delivery systems based on liposomes.

Advantageously, star polymers of the present disclosure can be modulated to optimize the pharmacokinetics and pharmacodynamics of a range of different D3.

The star polymers of the present disclosure can be used to display D3 and modulate the pharmacokinetics of D3. Alternatively, or in addition, the star polymers of the present disclosure can be used for the delivery of D3 to certain tissues or cell types.

The D3 may be a peptide and the linker precursor (Z2) may be attached to the N-terminal amino acid of the peptide, the C-terminal amino acid of the peptide, or to a side chain of any one or more amino acid residues present in the peptide.

In certain embodiments, the D3 a molecular weight of from about 250 to about 10,000 Da. D3 with relatively low molecular weight, e.g., less than about 10,000 Da, can typically be accessed synthetically and are often suitable for use in organic solvents.

In certain embodiments, the D3 is a peptide that binds to checkpoint molecules, such as PD1, PD-L1 and CTLA-4, such as antagonists of checkpoint molecules. In some embodiments the peptide binds to VEGF receptors, such as peptide-based antagonists of VEGF receptors.

In certain embodiments, D3 is a peptide ligand that binds to B cell receptors and encompasses an epitope(s) derived from an immunogen(s) isolated from infectious organisms or cancer cells. In other embodiments, D3 is a peptide that binds to T cell receptors and encompasses an epitope(s) derived from immunogen(s) isolated from infectious organisms or cancer cells. In still other embodiments, D3 is a peptide that binds to T cell receptors and encompasses an epitope(s) derived from a self-protein. The peptide-based D3 comprising an epitope(s) from infectious organisms may be from any infectious organism, such as a protein or glycoprotein derived from a fungus, bacterium, protozoan or virus. Alternatively, the peptide-based D3 comprises an epitope from a tumor-associated antigen including self-antigens or tumor-specific neoantigens; the peptide-based D3 may also comprise epitopes from self-proteins that are not tumor-associated.

The peptide antigen used as D3 may be any antigen that is useful for inducing an immune response in a subject. The peptide antigen may be used to induce either a pro-inflammatory or tolerogenic immune response depending on the nature of the immune response required for the application. In some embodiments, the peptide antigen is a tumor-associated antigen, such as a self-antigen, neoantigen or tumor-associated viral antigen (e.g., HPV E6/E7). In other embodiments, the peptide antigen is an infectious disease antigen, such as a peptide derived from a protein isolated from a virus, bacteria, fungi, or a protozoan microbial pathogen. In still other embodiments, the peptide antigen is a peptide derived from an allergen or an autoantigen, which is known or suspected to cause allergies or autoimmunity.

The peptide antigen is comprised of a sequence of amino acids or a peptide mimetic that can induce an immune response, such as a T cell or B cell response in a subject. In some embodiments, the peptide antigen comprises an amino acid or amino acids with a post-translational modification, non-natural amino acids or peptide-mimetics. The peptide antigen may be any sequence of natural, non-natural or post-translationally modified amino acids, peptide-mimetics, or any combination thereof, that have an antigen or predicted antigen, i.e., an antigen with a T cell or B cell epitope.

Immunogenic compositions of star polymers displaying peptide-based immunogens may comprise a single antigen, or the star polymer may comprise two or more different peptide antigens each having a unique antigen composition. In some embodiments, the star polymer includes only a single antigen. In some embodiments, the single peptide antigen comprises both B cell and T cell epitopes. In other embodiments, the star polymer comprises two different antigens. In some embodiments, wherein the star polymer comprises two different antigens, one of the antigens comprises a B cell epitope and the other antigen comprises a T cell epitope. In still other embodiments, the star polymer comprises up to 50 different peptide antigens each having a unique antigen composition. In some embodiments, the immunogenic compositions comprise star polymers that each comprise 20 different peptide antigens. In other embodiments, the immunogenic compositions comprise star polymers that comprise 5 different peptide antigens. In some embodiments, the immunogenic compositions comprise a mixture of up to 50 different star polymers each containing a unique peptide antigen. In other embodiments, the immunogenic compositions comprise up to 20 different star polymers each containing a unique peptide antigen. In still other embodiments, the immunogenic compositions comprise a single star polymer containing a single peptide antigen.

The length of the peptide antigen depends on the specific application and the route for producing the peptide antigen (A). The peptide antigen should minimally comprise at least a single T cell or B cell epitope. Therefore, wherein the T cell and/or B cell epitopes of an immunogen are known or can be predicted, a peptide antigen that comprises only the minimal epitopes of the immunogen (sometimes referred to as a minimal immunogen) can be produced by synthetic means and used to induce or modulate immune responses against those specific B cell and/or T cell epitopes that are known or predicted. Such synthetic peptide antigens comprising T cell and/or B cell epitopes typically comprise between about 5 to about 50 amino acids. In preferred embodiments, the peptide antigen produced by synthetic means is between about 7 to 35 amino acids, e.g., 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids. In some embodiments, D3 is a whole protein antigen.

Those skilled in the art recognize that any peptide, protein or post-translationally modified protein (e.g., glycoprotein) that leads to an immune response and is useful in the prevention or treatment of a disease can be selected for use as a peptide antigen for use in the immunogenic compositions of the present invention.

In certain embodiments, the D3 is a saccharide that binds to lectin receptors, such as CD22. In other embodiments, D3 is a synthetic or naturally occurring agonist of extracellular pattern recognition receptors (PRRs) and has immunostimulatory properties, particularly agonists of C-type lectin receptors.

In some embodiments, the D3 binds to C-type lectin receptors (CLRs) and is used to promote uptake by certain antigen presenting cells (APCs). In several embodiments, the ligand that binds to CLRs is a modified mannose and has the structure:

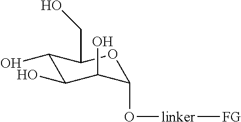

wherein the “linker” is any suitable linker molecule and FG is any suitable functional group that can be used to attach the linker modified mannose to a polymer arm (A). In some embodiments, the linker is PEG and FG is an azide.

In other embodiments, the ligand that binds to CLRs is a tetrasaccharide that binds to DC-SIGN and has the structure:

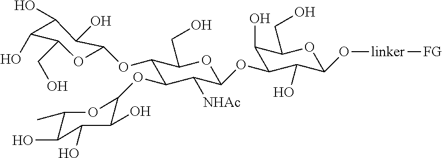

wherein the “linker” is any suitable linker molecule and FG is any suitable functional group that can be used to attach the linker modified mannose to a polymer arm (A). In some embodiments, the linker is PEG and FG is an azide.

In some embodiments, D3 is selected from targeting molecules that bind to specific tissues or specific cells within tissues. In some embodiments, D3 is selected from glucose that binds to glucose transporters upregulated by tumors and tumor vasculature.

Other suitable D3 include therapeutic antibodies or antibody fragments useful for the treatment of a disease. Therapeutic antibody molecules include antibodies directed against pathogens, cancer cells, soluble host proteins, toxins, as well as extracellular receptors and ion channels that may be blocked or stimulated to modulate signalling within the cell.

Suitable antibodies for use as D3 include antibodies directed against tumor antigens. Non-limiting examples of antibodies directed against tumor antigens include antibodies directed against CD19, CD20, CD22, CD30, CD33, CD38, CD51, EGFR, PDGF-R, VEGFR, SLAMF7, integrin αvβ33, carbonic anhydrase 9, HER2, GD2 ganglioside, mesothelin, TAG-72. Suitable antibodies include antibodies against immune checkpoint molecules that can be used to reverse or modulate immune suppression. Non-limiting examples include PD1, PD-L1, OX-40, CTLA-4, 41 BB. Suitable antibodies include agonists of the immune response, including but not limited to antibodies directed against CD40. Suitable antibodies include those that can modify disease, including the prevention, mitigation, or reversal of disease, such as antibodies directed against beta-amyloid, sclerostin, IL-6, TNF-alpha, VEGF, VEGFR, IL-5, IL-12, IL-23, Kallikrein, PCSK9, BAFF, CD125 or similar such targets of antibodies.

In some embodiments, the D3 is a peptide-MHC complex, e.g., a complex of a CD8 or CD4 T cell epitope with an MHC-I or MHC-II epitope, which may be used for inducing tolerance, when not provided with an additional immune stimulus, or may be used for activating and/or expanding T cells when used in combination with an immunostimulatory molecule.

In certain embodiments, D3 has a molecular weight of greater than about 10,000 Da. D3 with relatively high molecular weight, e.g., greater than about 10,000 Da, are typically manufactured using an expression system and are often not suitable for use in organic solvents during the manufacturing of the star polymer.

### Density of D3

The present inventors have unexpectedly found that the density of D3 has a profound impact on biological activity for certain applications described herein. For example, the present inventors have identified that start polymers displaying >5 D3 ligands are optimal for inducing downstream cellular signaling cascades across applications. Specifically, when D3 is a peptide-based B cell immunogen, greater than 5, typically 15 or more ligands, were required to induce B cell activation and the induction of antibodies in vivo. For larger D3, including antibodies, 5 or more ligand molecules per star polymer were found to be suitable for activity.

### Selection of D1 and D2 for Cancer Treatment.

In preferred embodiments of star polymers for cancer treatment, D1 and/or D2 are selected from immunostimulants and/or chemotherapeutics. Note that drug molecules selected for D2 (attachment to polymer arms) are generally useful as D1 (linked to the core of the star polymers). Therefore, unless otherwise specified, examples of D2 disclosed herein should generally be considered suitable examples of D1, and examples of D1 should be considered suitable examples of D2.

Suitable immunostimulants include various agonists of pattern recognition receptors (PRRs). While any class of PRR agonist molecule could potentially be used as an immunostimulants for inducing anticancer immunity (for cancer treatment), it was found that certain classes of immunostimulants lead to unexpectedly enhanced tumor clearance as compared with other classes of immunostimulants. Herein, it is disclosed that preferred immunostimulants are those that induce the production of specific cytokines, i.e., interferons (IFNs) and/or IL-12. Thus, in preferred embodiments of star polymers for cancer treatment, the star polymer includes D2 and/or D1 selected from immunostimulants selected from agonists of Stimulator of Interferon Genes (STING), TLR-3, TLR-4, TLR-7, TLR-8, TLR-7/8, and TLR-9.

Non-limiting examples of TLR-3 agonists include dsRNA, such as Polyl:C and nucleotide base analogs; TLR-4 agonists include lipopolysaccharide (LPS) derivatives, for example, monophosphoryl lipid A (MPL) small molecules such as pyrimidoindole; TLR-7 & -8 agonists include ssRNA and nucleotide base analogs, including derivatives of imidazoquinolines, hydroxy-adenine, benzonaphthyridine and loxoribine; TLR-9 agonists include unmethylated CpG and small molecules that bind to TLR-9; STING agonists include cyclic dinucleotides, and synthetic small molecules, such as alpha-mangostin and its derivatives as well as linked amidobenzimidazole (“diABZI”) and related molecules (see: Ramanjulu, J. M. et al. Nature, 2018, 564, 439-443). Of note, different agonists of TLRs and STING may be described as hydrophilic, amphiphilic, or hydrophobic. Exemplary hydrophilic drug molecules that are agonists of TLRs and/or STING includes nucleic acids. Exemplary amphiphilic and/or hydrophobic drug molecules that bind to TLRs or STING include heterocyclic compounds based on pyrimidoindoles, imidazoquinolines, hydroxy-adenine, benzonaphthyridines, loxoribine, alpha-mangostin and diABZI.

In several embodiments, the star polymer for cancer treatment comprises small molecule drugs (D) with immunostimulant properties selected from imidazoquinoline-based agonists of TLR-7, TLR-8 and/or TLR-7 & -8. Numerous such agonists are known, including many different imidazoquinoline compounds.

Imidazoquinolines are of use as small molecule immunostimulatory drugs (D) used in star polymers found in immunogenic compositions used for vaccination, or for treating cancer or infectious diseases in the absence of a co-administered antigen. Imidazoquinolines are synthetic immunomodulatory compounds that act by binding Toll-like receptors 7 and/or 8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265; and 4,689,338. In some non-limiting embodiments, the imidazoquinoline compound is not imiquimod and/or is not resiquimod.

In some embodiments, the drugs (D) with immunostimulatory properties can be a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines. In general, TLR-7, TLR-8 and TLR-7/8 agonists are described herein as hydrophobic or amphiphilic drug molecules.

In some embodiments, the drug (D) with immunostimulatory properties is an imidazoquinoline with the formula:

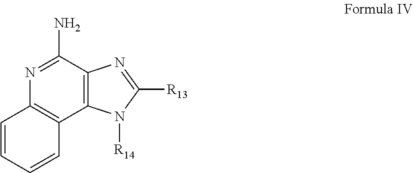

In Formula IV, R₁₃ is selected from one of hydrogen, optionally-substituted lower alkyl, or optionally-substituted lower ether; and R₁₄ is selected from one of optionally substituted arylalkylamine, or optionally substituted lower alkylamine, wherein the amine provides a reactive handle for attachment to a polymer either directly or via a linker. R₁₃ may be optionally substituted to a linker that links to a polymer.

In some embodiments, the R₁₃ included in Formula IV can be selected from hydrogen,

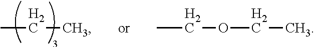

In some embodiments, R₁₄ can be selected from,

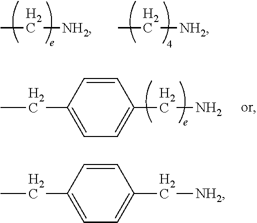

wherein e denotes the number of methylene unites is an integer from 1 to 4.

In some embodiments, R₁₄ can be

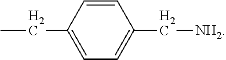

In some embodiments, R₁₄ can be

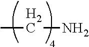

In some embodiments, R₁₃ can be

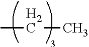

### and R14 can be

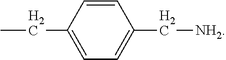

In some embodiments, D2 is selected from agonists of STING. In some embodiments, the agonist of STING is selected from amidobenzimidazole based molecules. A non-limiting example is shown here for clarity, wherein the piperazine ring is used as a reactive handle for linkage either directly or via a linker to reactive monomers:

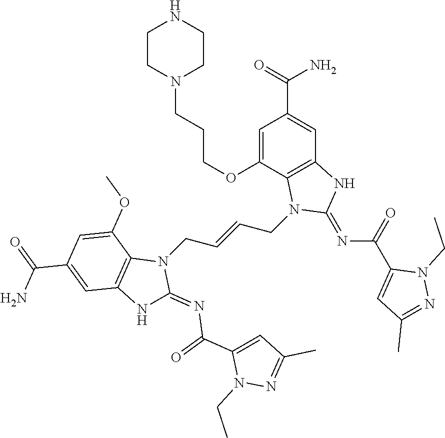 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

In some embodiments, agonist of STING is selected from cyclic dinucleotide-based molecules, which are generally considered hydrophilic drug molecules owing to their negative charge at physiologic pH, pH 7.4. Non-limiting examples of di-AMP based cyclic dinucleotides with either 3,5 linkages, mixed 2,5 and 3,5 linkages, or 2,5 linkages, are shown here for clarity:

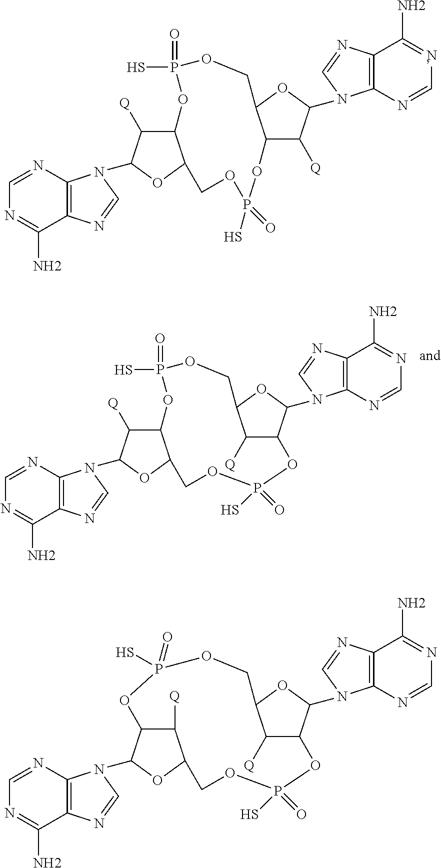

wherein Q is selected from H, OH or halogen atoms (e.g., fluorine) and SH is optionally replaced with OH.

In the above example, wherein Q is equal to OH, the structure is:

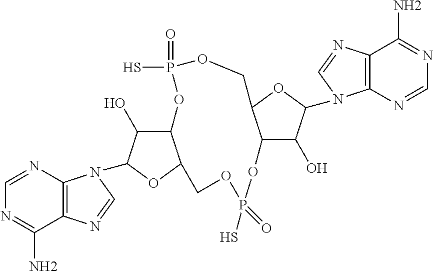 [SMILES: Nc1ncnc2c1ncn2C1OC2COP(=O)(S)OC3C(COP(=O)(S)OC2C1O)OC(n1cnc2c(N)ncnc21)C3O]

In certain embodiments, D2 is selected from chemotherapeutics. Of note, many chemotherapeutic drugs, particularly those based on aromatic heterocycles have hydrophobic or amphiphilic properties and may be described as hydrophobic or amphiphilic drug molecules.

In some embodiments, D2 is selected from alkylating agents (cisplatin, cyclophosphamide & temozolomide as an example), mitotic inhibitors (taxanes and Vinca alkaloids) or antimetabolites (5-fluorouracil, capecitabine & methotrexate as an example).

In other embodiments, D2 is selected from topoisomerase inhibitors (Topoisomerase I inhibitors and Topoisomerase II inhibitors), which are examples of amphiphilic or hydrophobic drug molecules. A non-limiting example is shown here for clarity, wherein the tertiary amine of topotecan is modified to enable conjugation to reactive monomers either directly or via a linker.

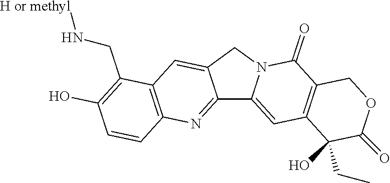

In other embodiments, D2 is selected from tyrosine kinase inhibitors. A non-limiting example is shown here for clarity, wherein the morpholine group of gefitinib, which is an example of an amphiphilic or hydrophobic drug molecule, has been replaced with a piperazine group to enable conjugation to reactive monomers either directly or via a linker.

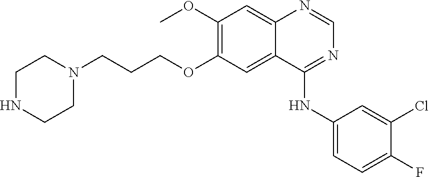 [SMILES: COc1cc2ncnc(Nc3ccc(F)c(Cl)c3)c2cc1OCCCN1CCNCC1]

In other embodiments, D2 is selected from angiogenesis (e.g., anti-VEGF receptor) inhibitors. A non-limiting example is shown here for clarity, wherein the tertiary amine of sunitinib, which is an example of amphiphilic or hydrophobic drug molecule, has been modified to enable conjugation to reactive monomers either directly or via a linker

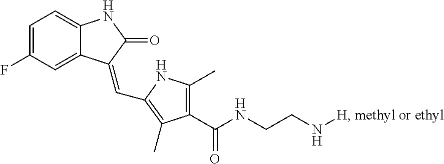

In other embodiments, D2 is selected from tumor antibiotics (anthracycline family, actinomycin-D and bleomycin as an example). In a non-limiting example, the anthracycline is doxorubicin and has the structure:

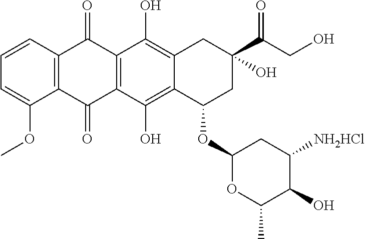

wherein the doxorubicin molecule may be linked to the star polymer arms (A) through the amine or ketone position via an amide or hydrazone bond, respectively. Note that anthracyclines have generally low water solubility and are considered amphiphilic or hydrophobic drug molecules.

While any class of chemotherapeutic could be used, it was found, unexpectedly, that certain classes of chemotherapeutics used in combination with immunostimulants lead to unexpectedly enhanced tumor clearance. Herein, it is disclosed that preferred chemotherapeutics are those that induce either or both reversal of immune-suppression and/or the induction of immunogenic cell death. Thus, in certain embodiments, star polymers of the present disclosure for cancer treatment include immunostimulants and/or chemotherapeutics, wherein the chemotherapeutics are selected from anthracyclines, taxanes, platinum compounds, 5-fluorouracil, cytaribine, and other such molecules that are useful for eliminating or altering the phenotype of suppressor cells in the tumor microenvironment.

Star polymer comprising immunostimulants and/or chemotherapeutics may be used to treat any cancer. Non-limiting examples include hematological tumors, such as leukemias, including acute leukemias (such as 11q23-positive acute leukemia, acute lymphocytic leukemia, acute myelocytic leukemia, acute myelogenous leukemia, myeloblastic leukemia, promyelocytic leukemia, myelomonocytic leukemia, monocytic leukemia, and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia; solid tumors, such as sarcomas and carcinomas, including fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers (including adenocarcinoma, a bronchiolaveolar carcinoma, a large cell carcinoma, or a small cell carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma); skin cancer, such as a basal cell carcinoma, a squamous cell carcinoma, a Kaposi's sarcoma, or a melanoma; and, premalignant conditions, such as variants of carcinoma in situ, or vulvar intraepithelial neoplasia, cervical intraepithelial neoplasia, or vaginal intraepithelial neoplasia.

### Optimization of Star Polymer Compositions for Intravenous Drug Delivery, Particularly for Cancer Treatment

Herein, we report unexpected findings related to how specific parameters of star polymers of the present disclosure can be optimized to improve the therapeutic index of drug molecules dosed by the intravenous route, particularly for cancer treatment. Notably, optimal star polymer properties were found to be applicable to various synthetic and naturally occurring drug molecules with diverse mechanisms of action.

One consideration is the attachment site of drug molecules to star polymers. Drugs may be attached to any suitable functional group on the star polymers of the present disclosure through any suitable means. Functional groups that can be used for attachment of drugs (D) may be located on the core (O), at the ends of the polymer arms (A) and/or in a pendant array along the backbones of the polymer arms (A). While the attachment to the end of the polymer arms (A) was found to be a preferred attachment site for certain drug molecules, e.g., drug molecules that bind extracellular receptors such as antigens that bind to B cell receptors, attachment of drug molecules along the backbones of the polymer arms (A), i.e., through attachment to reactive monomers, was found to the be the preferred attachment site for certain other drug molecule molecules, particularly small molecule drugs and/or amphiphilic or hydrophobic drugs. Indeed, the inventors' results show that high loading of drugs onto star polymers is fundamental to achieving high levels of efficacy and that maximal drug (D) loading for certain drug molecules, particularly amphiphilic or hydrophobic drug molecules, is achieved when such drug molecules are arrayed (as D2) along the backbone of the polymer arms (A).

Based on the unexpected finding disclosed herein that increasing loading of drug molecules results in improved efficacy, preferred embodiments of star polymers include greater than 10 mass percent of drugs, such as between 10 to 80 mass percent. For small molecule drugs (i.e., drugs with low molecular weight), high mass percent loading is only readily achieved by attaching such small molecule drugs at high mol % densities along the backbones of the polymer arms (A). Since the molecular weight of the star polymer without drugs (D) is principally driven by the mass of each polymer arm, the mol % density of drugs (D) attached to the star polymer (i.e., the percentage of monomers of the polymer arms linked to drug molecules) can be modulated to achieve a given mass percent of drug molecules. Accordingly, the mass percent of drug can be approximated using the following equation:

Mass percent drug=((MW D/(MWavg+(MW D*mol % D)))*mol % D)*100;

wherein MW D is the molecular weight of the small molecule drug (D); MWavg is the average MW of the monomers comprising the polymer arm (A), excluding the mass of the drug molecule linked to monomer the reactive monomer (E), and mol % D is the percentage of monomer units (E) that are linked to drug. Note: A polymer with 1 mol % drug (D) means that 1 out of 100 monomer units, specifically reactive monomers, comprising the polymer arms (A) of the star polymer are linked to drug (D). 10 mol % drug (D) means that 10 out of 100 monomer units comprising the polymer arms of the star polymer are linked to drug (D).

In a non-limiting example of a star polymer comprising small molecule drugs (D) with a molecular weight of 300 Da that are attached in a pendant array along the backbone of linear HPMA-based co-polymer arms, comprised of 143 Da HPMA monomers, at a density of about 5 mol %, the mass percent of the small molecule drug is about 9.5 mass %. In certain embodiments of star polymers used for cancer treatment, small molecule drugs between about 200-1,000 Da are arrayed along the polymer arms (A) at a density of between about 4.0 to about 50 mol % to achieve a mass percent of about 10 to about 80 mass %. In other embodiments of star polymers used for cancer treatment, small molecule drugs (D) with about 250-350 Da molecular weight are arrayed along the polymer arms at a density of between about 6 to about 40 mol % to achieve a mass percent of about 10 to about 50 mass %. In still other embodiments of star polymers used for cancer treatment, small molecule drugs (D) with about 350-450 Da molecular weight are arrayed along the polymer arms at a density of between about 5.0 to about 30 mol % to achieve a mass percent of about 10 to about 50 mass %.

While increasing densities of drug molecules on the star polymer are generally preferred, it was observed that increasing the density of amphiphilic or hydrophobic drug molecules to statistical random copolymer arms (A) comprised entirely of hydrophilic monomers (B) and reactive monomers (E), wherein the drug molecules are linked to the reactive monomers, led to an increased propensity of the star polymers to form aggregates in aqueous conditions. While aggregation of star polymers can present challenges to manufacturing, increased propensity of the star polymers to aggregate was also associated with decreased efficacy following intravenous administration. A non-limiting explanation is that star polymers prone to aggregation are cleared from the blood more rapidly by reticuloendothelial cells, which may be preferred for spleen and/or liver target but resulted in reduced amounts of drug reaching tissues other than spleen or liver.

To address the need for attaching high densities of amphiphilic or hydrophobic small molecule drugs to star polymers, two novel compositions of star polymers, referred to as star random copolymers and star diblock copolymers, were developed and first disclosed herein that led to high loading of amphiphilic or hydrophobic small molecule drugs without aggregation.

Preferred embodiments of star random copolymers have the formula O[D1]-([X]-A(D2)-[Z]-[D3])n, wherein O is a core; A is a polymer arm attached to the core, wherein the polymer arm is a random copolymer or terpolymer that comprises hydrophilic monomers and reactive monomers and optionally comprises charged monomers; X is a linker molecule between the core and the polymer arm; Z is a linker molecule between the end of the polymer arm and D3 or a capping group; D1 is a drug molecule linked to the core; D2 is a drug molecule linked to reactive monomers distributed along the backbone of the polymer arm; and, D3 is a drug molecule linked to the ends of the polymer arms; n is an integer number; [ ] denotes that the group is optional; and, D2 is selected from amphiphilic or hydrophobic small molecule drugs linked to the reactive monomers distributed along the backbone of the polymer arm at a density of between 1 mol % and 40 mol %, which may be represented schematically:

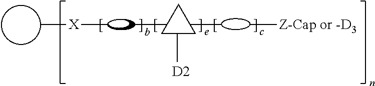

To ensure high loading of amphiphilic or hydrophobic drug molecules onto star random copolymers without aggregation, the composition of the polymer arms comprising star random copolymers must be carefully selected to adequately solubilize the amphiphilic or hydrophobic drug molecules. Accordingly, it was found that for star random copolymers comprising hydrophilic polymer arms that are neutral at physiologic pH, amphiphilic or hydrophobic drug molecules could be linked to polymer arms at densities of between about 1 mol % to 8 mol %, such as 1, 2, 3, 4, 5, 6, 7 or 8 mol %, without causing aggregation, whereas higher densities, i.e., densities generally higher than 8 mol % typically led to aggregation. In contrast, it was found that for star random copolymers comprising hydrophilic polymer arms that comprise charged comonomers and carry net negative or positive charge at physiologic pH, amphiphilic or hydrophobic drug molecules could be linked to such polymer arms at densities of between about 1 mol % to 40 mol %, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mol %, preferably between 5 mol % and 20 mol %, or more preferably between about 7.5 mol % and 15 mol %, without causing aggregation, provided that the density of charged monomers (with a single charged functional group) was at least a factor of 0.5 to 2 times, more preferably between about 0.75 to 1.5 times the density of the amphiphilic or hydrophobic drug molecule. Though, wherein the charged monomer has two charged functional groups, e.g., bis(acid), the density of charged monomer required was found to be about 0.25 to 1 times, more preferably between about 0.375 to 0.75 times the density of the amphiphilic or hydrophobic drug molecule. Further still, wherein the charged monomer has three or four functional groups, e.g., tri(acid) or tetra(acid), the density of charged monomer required was found to be about 0.125 to 0.5 times, more preferably between about 0.2 to 0.375 times the density of the amphiphilic or hydrophobic drug molecule.

In preferred embodiments of star polymers comprising reactive monomers linked to amphiphilic or hydrophobic drug molecules and charged comonomers, the density of charged monomers with a single charged functional group are selected based on the density of attached drug molecule according to Table 1 provided here:

| Hydrophobic or | Monofunctional charged monomer mol % | | | |
|---|---|---|---|---|
| amphiphilic drug | Preferred | Preferred | Most preferred | Most preferred |
| molecule mol % | low | high | low | high |
| 1 | 1 | 2 | 1 | 2 |
| 2 | 1 | 4 | 2 | 3 |
| 3 | 2 | 6 | 2 | 5 |
| 4 | 2 | 8 | 3 | 6 |
| 5 | 3 | 10 | 4 | 8 |
| 6 | 3 | 12 | 5 | 9 |
| 7 | 4 | 14 | 5 | 11 |
| 8 | 4 | 16 | 6 | 12 |
| 9 | 5 | 18 | 7 | 14 |
| 10 | 5 | 20 | 8 | 15 |
| 11 | 6 | 22 | 8 | 17 |
| 12 | 6 | 24 | 9 | 18 |
| 13 | 7 | 26 | 10 | 20 |
| 14 | 7 | 28 | 11 | 21 |
| 15 | 8 | 30 | 11 | 23 |
| 16 | 8 | 32 | 12 | 24 |
| 17 | 9 | 34 | 13 | 26 |
| 18 | 9 | 36 | 14 | 27 |
| 19 | 10 | 38 | 14 | 29 |
| 20 | 10 | 40 | 15 | 30 |
| 21 | 11 | 42 | 16 | 32 |
| 22 | 11 | 44 | 17 | 33 |
| 23 | 12 | 46 | 17 | 35 |
| 24 | 12 | 48 | 18 | 36 |
| 25 | 13 | 50 | 19 | 38 |
| 26 | 13 | 52 | 20 | 39 |
| 27 | 14 | 54 | 20 | 41 |
| 28 | 14 | 56 | 21 | 42 |
| 29 | 15 | 58 | 22 | 44 |
| 30 | 15 | 60 | 23 | 45 |
| 31 | 16 | 62 | 23 | 47 |
| 32 | 16 | 64 | 24 | 48 |
| 33 | 17 | 66 | 25 | 50 |
| 34 | 17 | 68 | 26 | 51 |
| 35 | 18 | 70 | 26 | 53 |
| 36 | 18 | 72 | 27 | 54 |
| 37 | 19 | 74 | 28 | 56 |
| 38 | 19 | 76 | 29 | 57 |
| 39 | 20 | 78 | 29 | 59 |
| 40 | 20 | 80 | 30 | 60 |

wherein the remaining monomer units typically comprise neutral hydrophilic monomers. Note: the bold-faced, italicized numbers represent the most preferred range of densities of drug molecules and charged monomers. For clarity, as depicted in the above table, the most preferred density of amphiphilic or hydrophobic drug molecules (linked to reactive monomers) is about 7 mol % to about 15 mol % and the most preferred range of charged monomers is about 5 mol % to about 23 mol %. In a non-limiting example of a preferred composition of a star polymer comprising amphiphilic or hydrophobic drug molecules and charged monomers, the amphiphilic or hydrophobic drug molecules are attached to the polymer arms at a density of 10 mol % and the charged monomer is attached a density of about 5 mol % to about 20 mol % or most preferably between 8 mol % to about 15 mol %.

In preferred embodiments of star polymers comprising reactive monomers linked to amphiphilic or hydrophobic drug molecules and charged comonomers, the density of charged monomers with two charged functional groups (or “bifunctional charged monomers”), e.g., bis(acid), are selected based on the density of attached drug molecule according to Table 2 provided here:

| Hydrophobic or | | | | |
|---|---|---|---|---|
| amphiphilic | bifunctional charged monomer mol % | | | |
| drug molecule | Preferred | Preferred | Most preferred | Most preferred |
| mol % | low | high | low | high |
| 1 | 0 | 1 | 0 | 1 |
| 2 | 1 | 2 | 1 | 2 |
| 3 | 1 | 3 | 1 | 2 |
| 4 | 1 | 4 | 2 | 3 |
| 5 | 1 | 5 | 2 | 4 |
| 6 | 2 | 6 | 2 | 5 |
| 7 | 2 | 7 | 3 | 5 |
| 8 | 2 | 8 | 3 | 6 |
| 9 | 2 | 9 | 3 | 7 |
| 10 | 3 | 10 | 4 | 8 |
| 11 | 3 | 11 | 4 | 8 |
| 12 | 3 | 12 | 5 | 9 |
| 13 | 3 | 13 | 5 | 10 |
| 14 | 4 | 14 | 5 | 11 |
| 15 | 4 | 15 | 6 | 11 |
| 16 | 4 | 16 | 6 | 12 |
| 17 | 4 | 17 | 6 | 13 |
| 18 | 5 | 18 | 7 | 14 |
| 19 | 5 | 19 | 7 | 14 |
| 20 | 5 | 20 | 8 | 15 |
| 21 | 5 | 21 | 8 | 16 |
| 22 | 6 | 22 | 8 | 17 |
| 23 | 6 | 23 | 9 | 17 |
| 24 | 6 | 24 | 9 | 18 |
| 25 | 6 | 25 | 9 | 19 |
| 26 | 7 | 26 | 10 | 20 |
| 27 | 7 | 27 | 10 | 20 |
| 28 | 7 | 28 | 11 | 21 |
| 29 | 7 | 29 | 11 | 22 |
| 30 | 8 | 30 | 11 | 23 |
| 31 | 8 | 31 | 12 | 23 |
| 32 | 8 | 32 | 12 | 24 |
| 33 | 8 | 33 | 12 | 25 |
| 34 | 9 | 34 | 13 | 26 |
| 35 | 9 | 35 | 13 | 26 |
| 36 | 9 | 36 | 14 | 27 |
| 37 | 9 | 37 | 14 | 28 |
| 38 | 10 | 38 | 14 | 29 |
| 39 | 10 | 39 | 15 | 29 |
| 40 | 10 | 40 | 15 | 30 |

wherein the remaining monomer units typically comprise neutral hydrophilic monomers. Note: the bold-faced, italicized numbers represent the most preferred range of densities of drug molecules and charged monomers. For clarity, as depicted in the above table, the most preferred density of amphiphilic or hydrophobic drug molecules (linked to reactive monomers) is about 7 mol % to about 15 mol % and the most preferred range of bifunctional charged monomers is about 3 mol % to about 11 mol %. In a non-limiting example of a preferred composition of a star polymer comprising amphiphilic or hydrophobic drug molecules and bifunctional charged monomers (e.g., bis(acid), the amphiphilic or hydrophobic drug molecules are attached to the polymer arms at a density of 10 mol % and the charged monomer is attached a density of about 3 mol % to about 10 mol % or most preferably between 4 mol % to about 8 mol %.

In preferred embodiments of star polymers comprising reactive monomers linked to amphiphilic or hydrophobic drug molecules and charged comonomers, the density of charged monomers with three or four charged functional groups (or “trifunctional or tetrafunctional charged monomers”), e.g., tri(acid) or tetra(acid), are selected based on the density of attached drug molecule according to Table 3 provided here:

| Hydrophobic or | tri- or tetrafunctional charged monomer mol % | | | |
|---|---|---|---|---|
| amphiphilic drug | Preferred | Preferred | Most preferred | Most preferred |
| molecule mol % | low | high | low | high |
| 1 | 0 | 1 | 0 | 0 |
| 2 | 0 | 1 | 0 | 1 |
| 3 | 0 | 2 | 1 | 1 |
| 4 | 1 | 2 | 1 | 2 |
| 5 | 1 | 3 | 1 | 2 |
| 6 | 1 | 3 | 1 | 2 |
| 7 | 1 | 4 | 1 | 3 |
| 8 | 1 | 4 | 2 | 3 |
| 9 | 1 | 5 | 2 | 3 |
| 10 | 1 | 5 | 2 | 4 |
| 11 | 1 | 6 | 2 | 4 |
| 12 | 2 | 6 | 2 | 5 |
| 13 | 2 | 7 | 3 | 5 |
| 14 | 2 | 7 | 3 | 5 |
| 15 | 2 | 8 | 3 | 6 |
| 16 | 2 | 8 | 3 | 6 |
| 17 | 2 | 9 | 3 | 6 |
| 18 | 2 | 9 | 4 | 7 |
| 19 | 2 | 10 | 4 | 7 |
| 20 | 3 | 10 | 4 | 8 |
| 21 | 3 | 11 | 4 | 8 |
| 22 | 3 | 11 | 4 | 8 |
| 23 | 3 | 12 | 5 | 9 |
| 24 | 3 | 12 | 5 | 9 |
| 25 | 3 | 13 | 5 | 9 |
| 26 | 3 | 13 | 5 | 10 |
| 27 | 3 | 14 | 5 | 10 |
| 28 | 4 | 14 | 6 | 11 |
| 29 | 4 | 15 | 6 | 11 |
| 30 | 4 | 15 | 6 | 11 |
| 31 | 4 | 16 | 6 | 12 |
| 32 | 4 | 16 | 6 | 12 |
| 33 | 4 | 17 | 7 | 12 |
| 34 | 4 | 17 | 7 | 13 |
| 35 | 4 | 18 | 7 | 13 |
| 36 | 5 | 18 | 7 | 14 |
| 37 | 5 | 19 | 7 | 14 |
| 38 | 5 | 19 | 8 | 14 |
| 39 | 5 | 20 | 8 | 15 |
| 40 | 5 | 20 | 8 | 15 |

wherein the bold-faced the remaining monomer units typically comprise neutral hydrophilic monomers. Note: the italicized numbers represent the most preferred range of densities of drug molecules and charged monomers. For clarity, as depicted in the above table, the most preferred density of amphiphilic or hydrophobic drug molecules (linked to reactive monomers) is about 7 mol % to about 15 mol % and the most preferred range of trifunctional or tetrafunctional charged monomers is about 1 mol % to about 6 mol %. In a non-limiting example of a preferred composition of a star polymer comprising amphiphilic or hydrophobic drug molecules and trifunctional or tetrafunctional charged monomers, the amphiphilic or hydrophobic drug molecules are attached to the polymer arms at a density of 10 mol % and the charged monomer is attached at a density of about 1 mol % to about 5 mol % or most preferably between 2 mol % to about 4 mol %.

For clarity, the above tables (Tables 1-3) and examples apply to star random copolymers comprising D2 selected from amphiphilic or hydrophobic drug molecules and charged monomers that carry net positive or net negative charge at pH 7.4, including negatively charged monomers that become neutral at pH less than pH 7.4.

In contrast, for star random copolymers comprising D2 selected from amphiphilic or hydrophobic drug molecules and pH-responsive positively charged monomers, i.e., monomers that are neutral at pH 7.4, but become positively charged at reduced pH, e.g., tumor pH, the preferred density of pH-responsive positively charged monomers is generally between 3 mol % and 30 mol % or more preferably between 5 mol % and 20 mol %. For star random copolymers comprising D2 comprising hydrophilic drug molecules and pH-responsive positively charged monomers, negatively charged monomers and/or positively charged monomers, the preferred density of pH-responsive positively charged monomers, negatively charged monomers and/or positively charged monomers is generally between 3 mol % and 30 mol % or more preferably between 5 mol % and 20 mol %. Finally, for star diblock copolymers comprising D2 selected from amphiphilic or hydrophobic drug molecules linked to the first block and pH-responsive positively charged monomers linked to the second block, the preferred density of pH-responsive positively charged monomers linked to the second block is generally between 3 mol % and 30 mol % or more preferably between 5 mol % and 20 mol %.

A non-limiting example of a star random copolymer is a star polymer of Formula V comprising polymer arms that comprise hydrophilic monomers (B) of Formula I, reactive monomers (E) of Formula III linked to drug molecules (D2), and optional charged monomers (C) of Formula II, which is shown here for clarity:

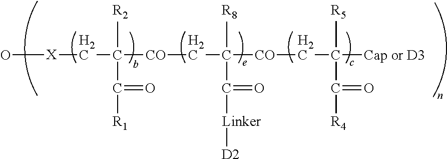

wherein in preferred embodiments of star polymers of Formula V, the hydrophilic monomer (B) is selected from hydrophilic meth(arcylamides) or meth(acrlyates), such as HPMA, HEMA or HEMAM; the linker, X, if present, links the polymer arm to the core through any suitable means, though, preferably through an amide bond; the end of each polymer arm distal to the core is capped, preferably with isobutyronitrile, or is linked to D3 preferably selected from targeting molecules; the core is an amide- or ester-based dendrimer, such as PAMAM- or bis(MPA)-based dendrimers, with generation between 1 to 6, such as 1, 2, 3, 4, 5 or 6 PAMAM dendrimer, preferably generation 3, 4 or 5; the symbols b, e and c are any integer denoting the number of monomers B, E and C, wherein the total number of monomer units is typically between about 50 to about 450 monomer units; co indicates that the monomers are randomly distributed along the backbone of the copolymer; the molecular weight of the polymer arm is between 5,000 and 60,000 Daltons (excluding the mass of the drug molecules), more preferably between 15,000 and 50,000 Daltons, or 20,000 and 40,000 Daltons, most preferably between about 20,00 to about 35,000 Daltons; n is an integer typically selected between 5 and 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 preferably between 10 and 45 polymer arms or more preferably between 20 and 35 polymer arms; the drug molecules (D2) are linked to the reactive monomers through any suitable linker molecule typically selected from enzyme degradable peptide-based linkers, carbamates, such as a self-immolative carbamate linker, e.g., PAB, acid-labile silyl ether, ketal or hydrazone linkers, or combinations thereof, at a density between 1 mol % and 40 mol %; the charged monomer, when present, is typically selected from pH-responsive positively charged monomers or negatively charged monomers that are pH responsive between pH of between about pH 4.5 to about 7.0, more preferably charged monomers with charge groups selected from glycine, beta-alanine, butanoic acid, methyl butanoic acid, dimethylbutanoic acid, 3,3′-((2-(6-aminohexanamido)propane-1,3-diyl)bis(oxy))dipropionic acid (referred to as “bis(COOH)”), 13-(6-aminohexanamido)-6,20-bis((2-carboxyethoxy)methyl)-8,18-dioxo-4,11,15,22-tetraoxa-7,19-diazapentacosanedioic acid (referred to as “tetra(COOH)”), most preferably DMBA, bis(COOH) and tetra(COOH); and, the hydrodynamic radius of the star polymer is between 5 and 30 nm, preferably between 7.5 and 20 nm.

A non-limiting example of a star polymer of Formula V that is neutral at physiologic pH, wherein the polymer arms comprise hydrophilic monomers selected from HPMA is shown here:

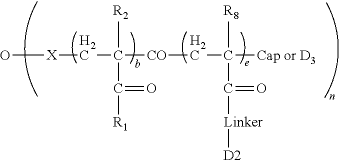

wherein in preferred embodiments of star polymers of Formula V wherein the drug molecules (D2) are selected from amphiphilic or hydrophobic small molecule drugs and the star random copolymer is neutral at physiologic pH, the drug molecules are preferably linked to the polymer arms at a density of between 1 mol % and 8 mol %, more preferably between about 3 mol % and 7 mol %; and, the hydrophilic monomer is preferably distributed along the polymer arm at a density of between about 92 mol % and 99 mol %.

The inventors of the present disclosure found unexpectedly that star polymers that are partially positive to neutral in the blood at physiologic pH but become more highly positively charged at reduced pH are preferred for certain applications, e.g., for cancer treatment. A non-limiting example of a star polymer of Formula V that comprises a star random copolymer comprising polymer arms with pH-responsive positively charged monomers that is partially positive to neutral at physiologic pH but becomes positively charged at lower pH (e.g., tumor pH), wherein the hydrophilic monomer is selected from HPMA and the pH-responsive charged monomers comprise tertiary amines is shown here:

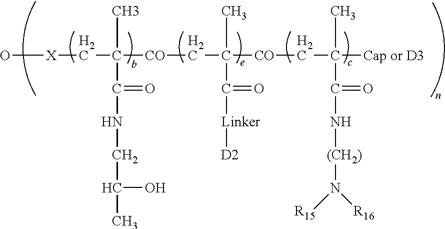

wherein in preferred embodiments the drug molecules are preferably linked (via reactive monomers) to the polymer arms at a density of between 1 mol % and 8 mol %, more preferably between about 2 mol % and 7 mol %; the pH-responsive positively charged monomer is distributed along the polymer arms at a density of 3 mol % to about 30 mol %, or more preferably, between about, 5 mol % to 20 mol %; j is an integer number of repeating units of methylene groups, typically 1 to 6 methylene units, and R₁₅ and R₁₆ are independently selected from hydrogen, methyl, ethyl or isopropyl groups.

The inventors of the present disclosure observed that highly positively charged star polymers were cleared more rapidly from the blood than star polymers with lower magnitude positive charge or neutral charge. Therefore, in certain preferred embodiments of star polymers for cancer treatment, the star polymer comprises polymer arms further comprising charged monomers with amine functional groups that are predominantly (50%) neutral at blood pH, i.e., pH 7.4, but are predominantly positively charged at reduced pH, e.g., pH 6.5. Embodiments of star polymers for cancer treatment that meet these criteria include star polymers comprising polymer arms that further comprise charged monomers with nitrogen heterocycles and/or aromatic amines that have pKa less than 8, more preferably less than pH 7.4. Non-limiting examples of suitable nitrogen heterocycles and/or aromatic amines include imidazole, pyridine, amino pyridine, quinoline, amino quinoline, aniline, naphthalene amine or the like and any derivatives thereof.

A non-limiting example of a star polymer of Formula V that comprises a star random copolymer comprising polymer arms that comprise charged monomers that are predominantly neutral at pH 7.4, but are predominantly charged at pH less than pH 7.4, wherein the hydrophilic monomer is selected from HPMA and the pH-responsive positively charged monomers comprise imidazole is shown here for clarity:

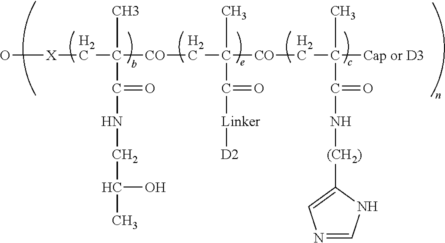

wherein in preferred embodiments the drug molecules are preferably linked (via reactive monomers) to the polymer arms at a density of between 1 mol % and 8 mol %, more preferably between about 3 mol % and 7 mol %; the pH-responsive positively charged monomer is distributed along the polymer arms at a density of 3 mol % to 30 mol %, or more preferably 5 mol % to 20 mol %; and j is an integer number of repeating units of methylene groups, typically 1 to 6 methylene units.

In some embodiments, the charged group is linked to the charged monomer indirectly through a linker. For example, wherein the linker is beta-alanine, the above structure becomes:

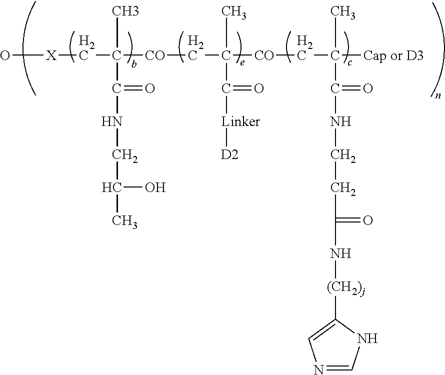

A potential limitation o the use o star polymers of Formula V that are neutral at physiologic pH is that they can aggregate if high densities of amphiphilic or hydrophobic drug molecules are attached. To address this limitation, the inventors of the present disclosure found that star polymers of Formula V that are charged at physiologic pH can be used to incorporate relatively high densities of amphiphilic or hydrophobic drug molecules without the star random copolymers aggregating.

A non-limiting example of a star polymer of Formula V that comprises charged monomers at physiologic pH 7.4, wherein the hydrophilic monomer is selected from HPMA and the charged monomer is a methacrlyamide based monomer is shown here for clarity:

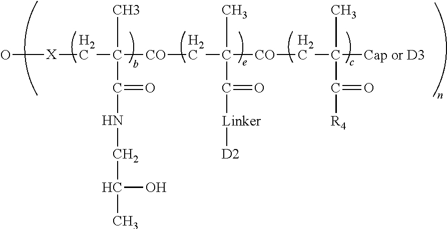

wherein in preferred embodiments the drug molecules (D2) are preferably selected from amphiphilic or hydrophobic drug molecules typically selected from small molecule chemotherapeutics and immunostimulants that are linked to the polymer arms at a density of between 1 mol % and 40 mol %, more preferably between about 5 mol % and 20 mol %, or most preferably between about 7.5 to 15 mol %; the charged monomer is typically selected from negatively charged monomers that are pH responsive between pH of about 4.5 to about 7.0, more preferably charged monomers with charged groups selected from glycine, beta-alanine, butanoic acid, methyl butanoic acid, dimethylbutanoic acid, 3,3′-((2-(6-aminohexanamido)propane-1,3-diyl)bis(oxy))dipropionic acid (referred to as “bis(COOH)”), 13-(6-aminohexanamido)-6,20-bis((2-carboxyethoxy)methyl)-8,18-dioxo-4,11,15,22-tetraoxa-7,19-diazapentacosanedioic acid (referred to as “tetra(COOH)”), most preferably DMBA, bis(COOH) and tetra(COOH) that are distributed along the polymer arms at the preferred densities provided in Table 1 (for monofunctional charged monomers, e.g., a charged monomer comprising DMBA), Table 2 (for bifunctional charged monomers, e.g., a charged monomer comprising bis(acid)) and Table 3 (for tri- or tetra-functional charged monomers, e.g., a charged monomer comprising tetra(acid)); and, the hydrophilic comprises the remaining monomer units.

While both positive and negatively charged monomers were found to be suitable for reducing the propensity of star random copolymers to aggregate when carrying high densities of amphiphilic or hydrophobic drugs, the inventors of the present disclosure observed that star polymers of Formula V comprising negatively charged star random copolymers had high uptake in certain tissues, e.g., tumors, as compared with star random copolymers with positive charge at physiologic pH 7.4, which had high uptake by the liver and spleen. Of note, star random copolymers with positive charge at pH 7.4 are distinct from those pH-responsive positively charged star polymers as the latter are partially positive to neutral at physiologic pH but only become positively charged at lower (e.g., tumor pH), thereby providing improved tumor targeting as compared with the former, which are positively charged in the blood at pH 7.4 and therefore more susceptible to clearance by the liver and spleen.

Therefore, for drug delivery applications other than targeting the liver and/or spleen, preferred embodiments of star polymers of Formula V comprise star random copolymers that are negatively charged at physiologic pH thereby avoiding ant potential liabilities of having positive charge. A non-limiting example of a star polymer of Formula V that comprises a star random copolymer that is negatively charged at physiologic pH 7.4, wherein the hydrophilic monomer is selected from HPMA and the charged monomer comprises a carboxylic acid is shown here for clarity:

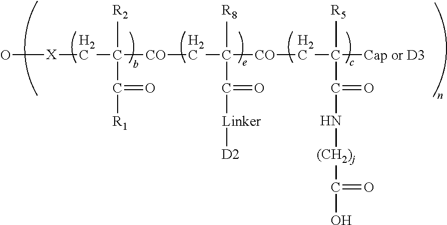

wherein in preferred embodiments the drug molecules (D2) are preferably selected from amphiphilic or hydrophobic drug molecules typically selected from small molecule chemotherapeutics and immunostimulants that are linked to the polymer arms at a density of between 1 mol % and 40 mol %, more preferably between about 5 mol % and 20 mol %, or most preferably between about 7.5 to 15 mol %; the charged monomer is distributed along the polymer arms at the preferred densities provided in Table 1; the hydrophilic monomer comprises the remaining monomer units.

Though any negatively charged monomer that exists as the conjugate base of an acid at physiologic pH may be suitable for use as negatively charged monomers, the inventors of the present disclosure found that certain carboxylic acids are preferred for use as charged comonomers of star polymers of Formula V used for delivering amphiphilic or hydrophobic drugs to tumors. For instance, it was observed that star polymers of Formula V comprising amphiphilic or hydrophobic drug molecules linked to reactive monomers and charged monomers further comprising carboxylic acids that have pKa between about 2.5 to 5.5 led to improved tumor uptake and enhanced efficacy as compared with star polymers of Formula V comprising amphiphilic or hydrophobic drug molecules and charged monomers further comprising carboxylic acids that have pKa either less than 2.0 or above 5.5. A non-limiting explanation is that because conjugate bases as poly(anions) have higher pKa than the single molecules, star random copolymers comprising carboxylic acids with pKa above 5.5 may not be adequately deprotonated at physiologic pH 7.5, whereas star random copolymers comprising carboxylic acids with pKa less than 2.5 may remain deprotonated and negatively charged, even after reaching tumors, thereby preventing cellular uptake. Therefore, star random copolymers comprising carboxylic acids with pKa between about 2.5 to 5.5 (as the single molecule) may be best suited for drug delivery to tumors because the pKa is sufficiently low that, even as a poly(anion), the carboxylic acid may remain deprotonated at physiologic pH and thus aid solubility in the blood but is sufficiently high such that the conjugate base of the carboxylic acid becomes protonated within the tumor, resulting in decreased solubility and/or increased cellular interactions within the acidic tumor microenvironment. Thus, in preferred embodiments of star polymers of Formula V used for cancer treatment, the star random copolymer is negatively charged at physiologic pH and comprises charged monomers that comprise carboxylic acids that have pKa (as the single molecule) between about 2.5 to 5.5, more preferably between about 3.0 to 5.0. Note: Unless otherwise specified, pKa values used herein refer to the pKa of functional groups of single molecules. Nota also that the pKa of a monomer increases by about 1 to 2, or more, units when present at a high density on a polymer, and thus the pKa of a monomer that is about 5.0, would be expected to have a pKa of between about 6.0 to 7.0, or more, when present on a polymer.

Negatively charged monomers that meet the aforementioned criteria and as poly(anions) on star polymers are pH responsive between about 4.5 to about 7.0, include charged monomers with charged groups selected from glycine, beta-alanine, butanoic acid, methyl butanoic acid, dimethylbutanoic acid, 3,3′-((2-(6-aminohexanamido)propane-1,3-diyl)bis(oxy))dipropionic acid (referred to as “bis(COOH)”), 13-(6-aminohexanamido)-6,20-bis((2-carboxyethoxy)methyl)-8,18-dioxo-4,11,15,22-tetraoxa-7,19-diazapentacosanedioic acid (referred to as “tetra(COOH)”), most preferably DMBA, bis(COOH) and tetra(COOH)

A non-limiting example of a star polymer of Formula V comprising a star random copolymer that is negatively charged at physiologic pH and comprises charged monomers further comprising DMBA is shown here for clarity:

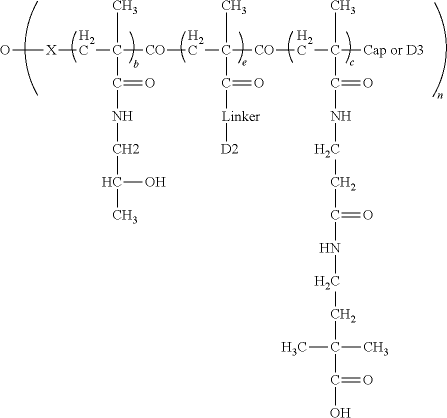

Charged groups may be linked directly or indirectly through a linker molecule. In the above example, wherein the charged monomer is replaced with a methacrylamide comprising 4-amino-2,2-dimethylbutanoic acid hydrochloride (DMBA) (CAS no. 153039-15-7) linked through a beta-alanine linker.

A non-limiting example of the above example, wherein the charged monomer comprising a bis(acid) is shown for clarity:

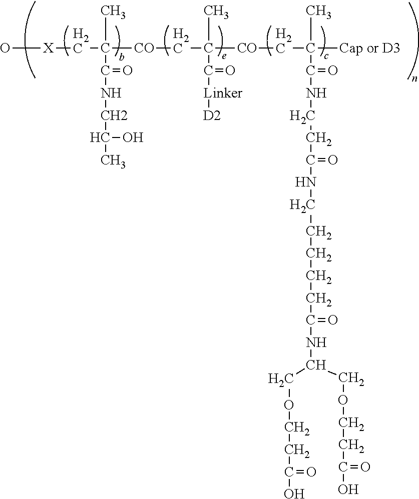

A non-limiting example of the above example, wherein the charged monomer comprising a bis(acid) is shown for clarity:

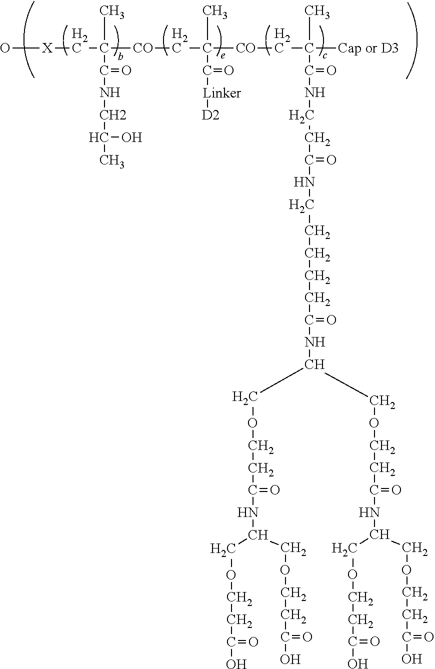

In certain preferred embodiments of star polymers of Formula V used for cancer treatment, drug molecules are attached to reactive monomers through a pH sensitive carbohydrazone bond. A non-limiting example is provided here for clarity:

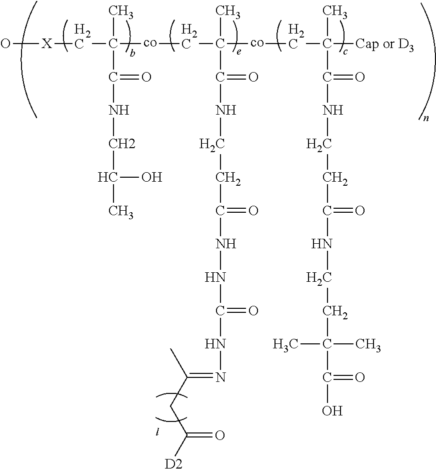

wherein in preferred embodiments the drug molecules (D2) are preferably selected from amphiphilic or hydrophobic drug molecules typically selected from small molecule chemotherapeutics and immunostimulants that are linked to the polymer arms at a density of between 1 mol % and 40 mol %, more preferably between about 5 mol % and 20 mol %, or most preferably between about 7.5 to 15 mol %; the charged monomer is distributed along the polymer arms at the preferred densities provided in Table 1; the hydrophilic monomer comprises the remaining monomer units; and I is an integer typically between 2 to 6, such as 2, 3, 4, 5 or 6, though, preferably I is 4.

In the above example, wherein the core is a PAMAM dendrimer, the linker X comprises a triazole bond, I is equal to 4 and the polymer is capped with isobutyronitrile, the structure is:

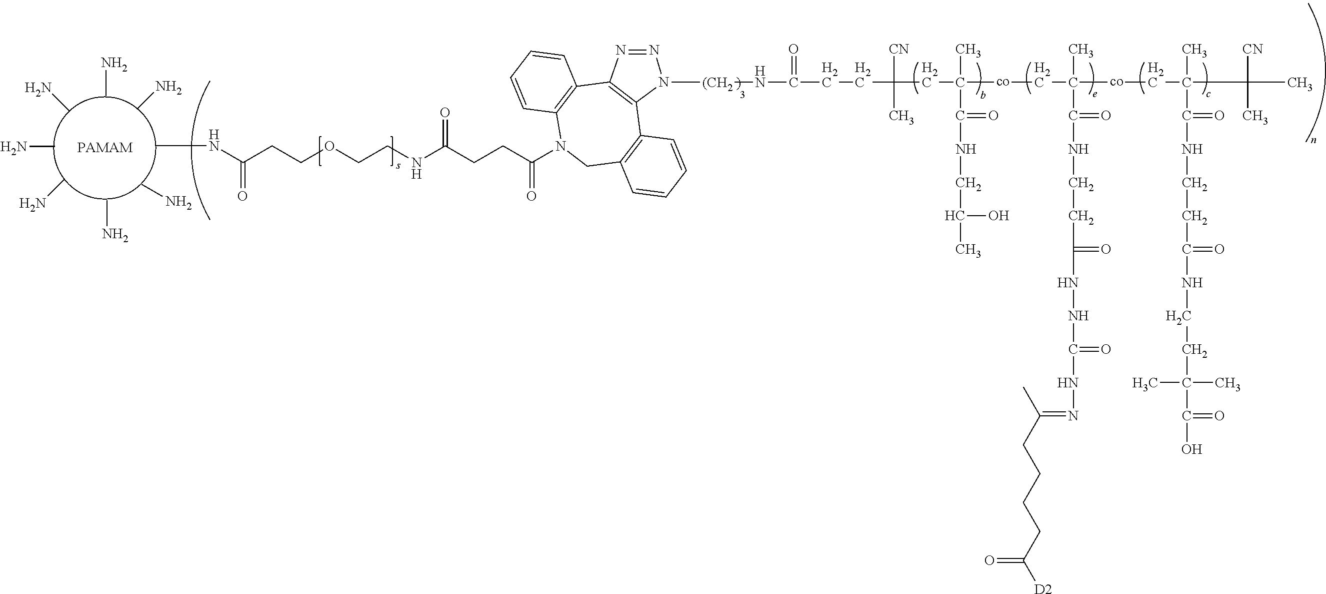

wherein s is an integer typically between 4 and 24, such as 4, 5, 6, 7, 8, 9, 10, 11, 12, 13, 14, 15, 16, 17, 18, 19, 20, 21, 22, 23 or 24.

In certain preferred embodiments of star polymers of Formula V used for cancer treatment, drug molecules are attached to reactive monomers through a peptide linker or optionally via a self-immolative carbamate linked to a peptide linker. A non-limiting example is provided here for clarity:

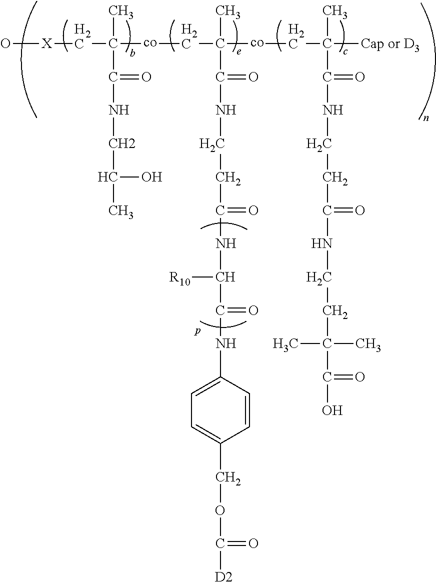

wherein in preferred embodiments, the molecular weight of the polymer arm is between 5,000 and 60,000 Daltons (excluding the mass of the drug molecules), more preferably between 15,000 and 50,000 Daltons or 20,000 and 40,000 Daltons, or most preferably between about 20,00 to about 35,000 Daltons; n is an integer typically selected between 5 and 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 preferably between 10 and 45 polymers arms or more preferably between 20 and 35 polymer arms; the drug molecules (D2) are preferably selected from amphiphilic or hydrophobic drug molecules typically selected from small molecule chemotherapeutics (e.g., anthracyclines) and immunostimulants (e.g., agonists of TLR-7/8 or STING) that are linked to the polymer arms at a density of between 1 mol % and 40 mol %, more preferably between about 5 mol % and 20 mol %, or most preferably between about 7.5 to 15 mol %; the charged monomer is distributed along the polymer arms at the preferred densities provided in Table 1; the hydrophilic monomer comprises the remaining monomer units; p is an integer of amino acids typically between 2 to 6, such as 2, 3, 4, 5 or 6, though, preferably p is 2, 3 or 4, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine; P2 is selected from glycine, serine, leucine, valine or isoleucine; P3 is selected rom acetyl lysine, boc-protected lysine, norleucine (nLeu), glutamine, 6-hydroxy norleucine (abbreviated hnLeu), glycine, serine, alanine, proline, or leucine; and P4 is selected from glycine, serine, arginine, lysine, acetyl lysine (i.e., the epsilon amine is acetylated), Boc protected lysine, aspartic acid, glutamic acid or beta-alanine; the carbamate linker is optional and may be present or absent.

In the above example, wherein the core is a PAMAM dendrimer, the linker X comprises a triazole bond, polymer is capped with isobutyronitrile, and the drug molecule is selected from an imidazoquinoline of Formula IV, the structure is:

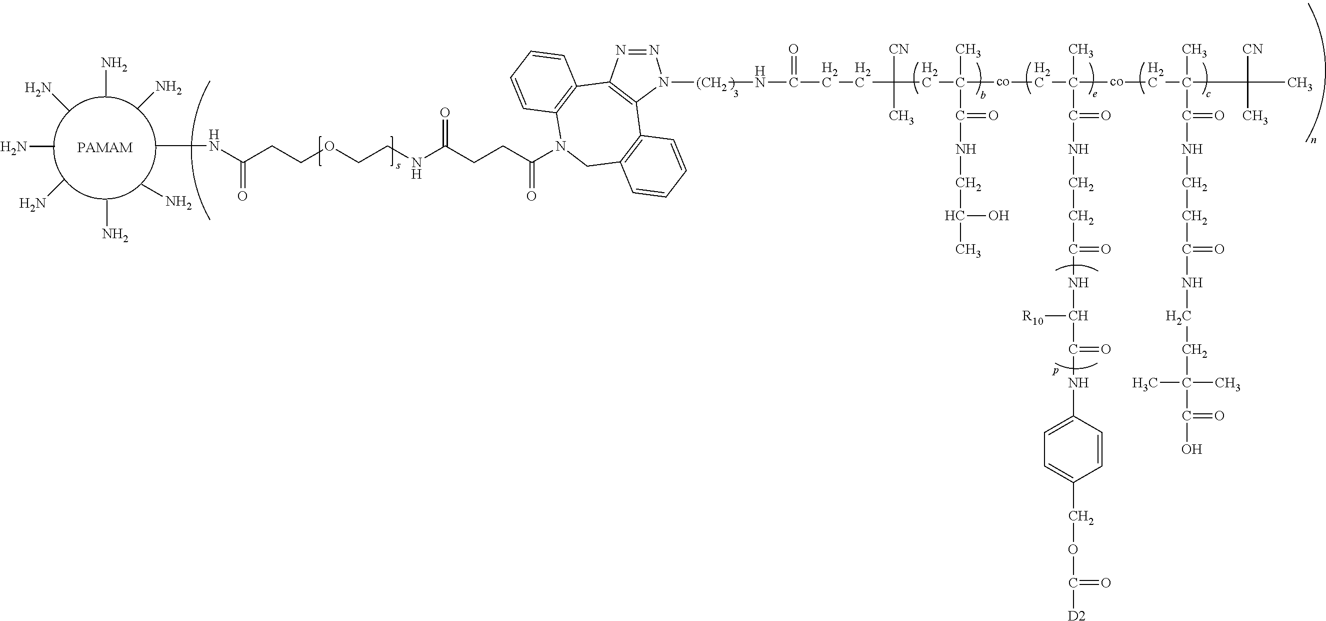

Note: in the above examples, DMBA may be optionally substituted with glycine, beta-alanine, methyl butanoic acid or a bis(acid), tri(acid) or tetra(acid) molecule.

The use of charged monomers in the polymers arms of star random copolymers is, in part, meant to solubilize and/or shield amphiphilic or hydrophobic drug molecules in the blood during circulation. The inventors of the present disclosure also identified that the use of a second polymer arm that is hydrophilic and/or pH-responsive is an alternative means of shielding and/or solubilizing amphiphilic or hydrophobic drug molecules. Accordingly, the inventors found that for star random copolymers comprising a first polymer arm comprising amphiphilic or hydrophobic drug molecules, the addition of a second polymer arm comprising neutral hydrophilic monomers and/or charged monomers reduced the propensity of such star polymers to aggregate. An additional unexpected finding was that the bond linking the second polymer arm to the core had a significant impact on the efficacy of such star polymers used for cancer treatment. For example, for star random copolymers comprising a first polymer arm comprising amphiphilic or hydrophobic drug molecules and a second polymer arm comprising neutral hydrophilic monomers and/or charged monomers, wherein the first polymer arm is linked to the core through an amide bond, linkage of the second polymer arm to the core through pH-sensitive (e.g., hydrazone, ketal, silyl ether, etc.) or reducible linkers (e.g., disulfide) led to improved efficacy as compared with compositions wherein the second polymer arm was linked to the core through an amide bond. Non-limiting explanations are that more rapid shedding of the second arm, as compared with the first arm, leads to improved rate of release of the drug molecule in the tumor microenvironment.

In some embodiments of star random copolymers, e.g., a star polymer of Formula V, used for cancer treatment, the star random copolymer comprises a first polymer arm and a second polymer arm. In a non-limiting example of a star random polymer comprising a first polymer arm and a second polymer arm, the star polymer comprises a first polymer arm that is a random copolymer architecture comprising hydrophilic monomers and reactive monomers linked to drug molecules and a second polymer arm comprising hydrophilic monomers and optionally comprising reactive monomers and charged monomers; additionally wherein the hydrophilic monomers are preferably selected from monomers of Formula I (e.g., HPMA), the reactive monomers are selected from monomers of Formula III, the charged monomers are selected from monomers of Formula II. A non-limiting example is shown here for clarity:

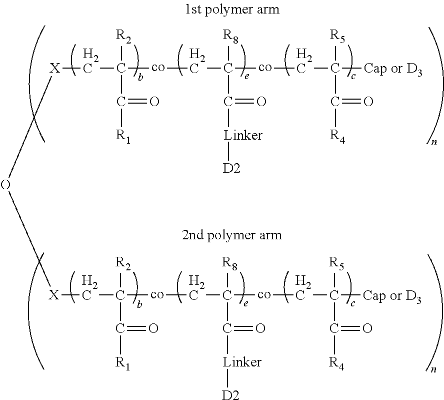

wherein in preferred embodiments, the hydrophilic monomer (B) of the first and second polymer arms is selected from hydrophilic meth(arcylamides) or meth(acrlyates), such as HPMA, HEMA or HEMAM; the linker, X, if present, links the first and second polymer arms to the core through any suitable means, though, preferably, the first polymer arm is linked to the core through a stable amide bond and the second polymer arm is linked to the core through a pH-sensitive hydrazone, silyl ether or ketal bond; the end of each polymer arm distal to the core is capped or linked to D3 comprising a targeting molecule; the core is an amide- or ester-based dendrimer, such as PAMAM- or bis(MPA)-based dendrimers, with generation between 1 to 6, such as 1, 2, 3, 4, 5 or 6 PAMAM dendrimer, preferably generation 3, 4 or 5; the symbols b, e and c are any integers denoting the number of monomers B, E and C, wherein the total number of monomer units is typically between about 50 to about 450 monomer units; co indicates that the monomers are randomly distributed along the backbone of the copolymer arms; the molecular weight of the polymer arms is between 5,000 and 60,000 Daltons (excluding the mass of the drug molecules), preferably between 10,000 and 40,000 Daltons; n is an integer number of polymers arms, wherein the total number of first and second polymers arms is typically between 3 and 40, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, preferably between 5 and 35 total polymers arms or more preferably between 10 and 30 total polymer arms; drug molecules (D2), which are typically selected from amphiphilic or hydrophobic drug molecules are linked to the reactive monomers through any suitable linker molecule, though preferably through an amide, carbamate or acid-labile silyl ether, ketal or hydrazone bound at a density between 1 mol % and 40 mol %, though, preferably between 5 mol % and 20 mol % or between about 7.5 mol % and 15 mol %; and, the hydrodynamic radius of the star polymer is between 5 and 40 nm, preferably between 7.5 and 20 nm.

For star random copolymers comprising two or more different arms, the inventors of the present disclosure identified the optimal number and composition of polymer arms that lead to unexpected improvements in biological activity. For instance, the inventors of the present disclosure identified that for star polymers comprising a first polymer arm and a second polymer arm, wherein the first polymer arm comprises amphiphilic or hydrophobic drug molecules (D2) linked to reactive monomers and the second polymer arm comprises hydrophilic monomers and optionally includes charged monomers and/or reactive monomers linked to hydrophilic drug molecules, which may additionally comprises targeting molecules, the optimal number, composition and length (molecular weight) of the second polymer arm depends on the length (molecular weight) and density of the amphiphilic or hydrophobic drug molecule attached to the first polymer arm. Non-limiting exemplary combinations include:
Star polymers wherein the first polymer arm comprises amphiphilic or hydrophobic drug molecules attached at a density of between 20 mol % and 80 mol % and the second polymer arm comprises neutral hydrophilic monomers; first polymer arm has a molecular weight (excluding the molecular weight of the drug molecules) between about 5 kDa and 60 kDa, and the second polymer arm has a molecular weight of between about 5 kDa and 60 kDa, and the total number of polymer arms attached to the core is between about 10 and 40 polymer arms, wherein 20% or more of the polymer arms are selected from the second polymer arm; and,
Star polymers wherein the first polymer arm comprises amphiphilic or hydrophobic drug molecules attached at a density of between 20 mol % and 80 mol % and the second polymer arm comprises neutral hydrophilic monomers; first polymer arm has a molecular weight (excluding the molecular weight of the drug molecules) between about 10 kDa and 40 kDa, more preferably between about 10 kDa and 30 kDa, and the second polymer arm has a molecular weight of between about 20 kDa and 60 kDa, more preferably between about 30 kDa and 50 kDa and the total number of polymer arms attached to the core is between about 10 and 40 polymer arms, wherein 20% or more of the polymer arms are selected from the second polymer arm, though, more preferably 25% to 50% of the polymer arms are selected from the second polymer arm.

An additional unexpected finding was the rate of hydrolysis of the linkage between the polymer arms and the core can also be used to modulate biological activity. For instance, the inventors of the present disclosure observed that for star polymers used for cancer treatment, wherein the first polymer arm comprises amphiphilic or hydrophobic drug molecules and the second polymer arm comprises neutral hydrophilic monomers, use of amide linkers between the first polymer arm and the core resulted in improved efficacy as compared with use of more hydrolytically labile linkers, whereas linking the second polymer arm to the core through linkers with moderate hydrolytic stability, e.g., carbohydrazones, led to improved efficacy as compared with the use more stable amide bonds, or less stable hydrazones.

Therefore, in preferred embodiments of star polymers that comprise a first polymer arm that comprises amphiphilic or hydrophobic drug molecules and a second polymer arm that comprises neutral hydrophilic monomers, the first polymer arm is linked through an amide bond and the second polymer arm is linked through a pH-sensitive hydrazone (or carbohydrazone), silyl ether or ketal bond. A

A non-limiting example of a star polymer that comprises a first polymer arm that comprises hydrophilic monomers, reactive monomers linked to amphiphilic or hydrophobic drug molecules and optionally includes charged monomers, and a second polymer arm that comprises neutral hydrophilic monomers and optionally includes charged monomers, wherein the first polymer arm is linked to the core through a stable amide bond and the second polymer arm is linked to the core through a pH-sensitive carbohydrazone; additionally, wherein the hydrophilic monomers are selected from monomers of Formula I (e.g., HPMA), the charged monomers are selected from charged monomer of Formula II, and the reactive monomers are selected from reactive monomers of Formula III, is shown here for clarity:

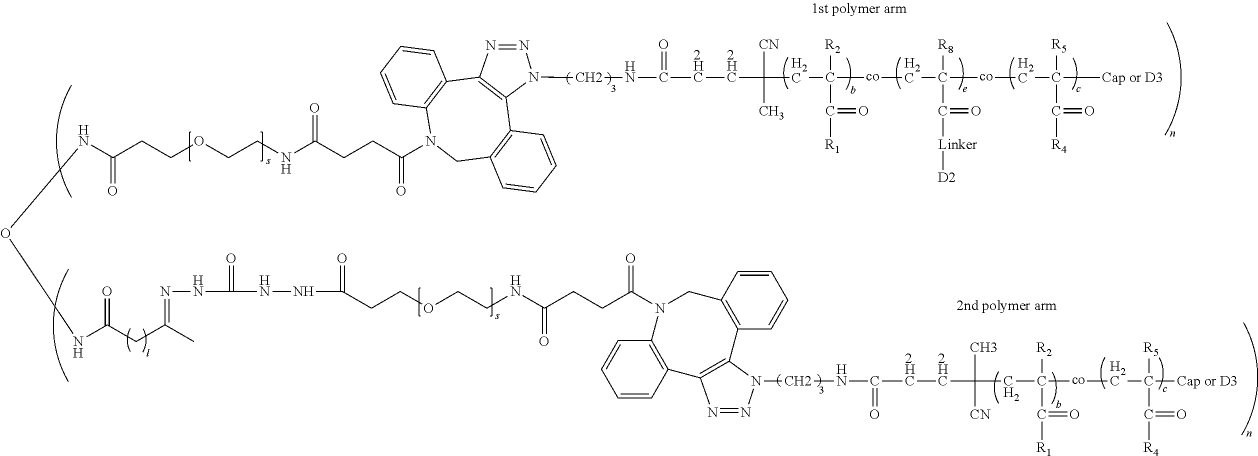

As an alternative to the use of star random copolymers comprising charged monomers, the inventors of the present disclosure also found that certain compositions of star diblock copolymers could incorporate high densities of amphiphilic or hydrophobic small molecule drugs without aggregation. More specifically, while the inventors of the present disclosure found that attaching high densities, e.g., greater than 5 or 10 mol %, of amphiphilic or hydrophobic small molecules drugs along the arms (via reactive monomers) of star random copolymers required the use of charged monomers to solubilize the arms and prevent aggregation, the inventors also found that star polymers comprising polymer arms with diblock architecture could be used to for incorporating high densities of amphiphilic or hydrophobic drug molecules without causing aggregation. Accordingly, the inventors found that for star diblock copolymers comprising polymers arms (A) consisting of diblock copolymers comprising a first block and a second block wherein the first block is linked to the core and the second block is distal to the core and linked to a capping group or D3 either directly or via the linker Z, attachment of high densities of amphiphilic or hydrophobic drug molecules to the first block was well tolerated and did not require inclusion of charged monomers on either block of the polymers to ensure that the star polymers were stable, provided that the block ratio, that is the degree of polymerization block ratio of the first block to the second block was sufficient for the second block to provide sufficient surface coverage (shielding) of the first block. Accordingly, the inventors found that star diblock copolymers could accommodate between 1 and 80 mol % drug molecules on the first block, though, preferably between 5 and 40 mol %, or most preferably between 10-30 mol % drug molecules (i.e., D2) on the first block, provided that the degree of polymerization block ratio was between 2:1 and 1:5, though, preferably between about 1:1 to 1:2, or between about 1:1 to 1:3. Note, density of a drug molecule (D2) on a first block of a diblock polymer refers to the density of the drug molecule (D2) on that block, i.e., the first block.

Based on the above observations, preferred embodiments of star diblock copolymers (sometimes referred to as star diblock polymers, or SDB) have the general formula O[D1]-([X]-A(D2)-[Z]-[D3])n, wherein O is a core; A is a polymer arm attached to the core, wherein the polymer arm is a diblock copolymer that comprises a first block and a second block that is proximal and distal to the core, respectively; additionally wherein the first block comprises hydrophilic monomers and reactive monomers linked to drug molecules and the second block comprises hydrophilic monomers and optionally includes charged monomers; X is a linker molecule between the core and the polymer arm; Z is a linker molecule between the end of the polymer arm and D3 or a capping group; D1 is a drug molecule linked to the core; D2 is a drug molecule linked to reactive monomers distributed along the backbone of the polymer arm; and, D3 is a drug molecule linked to the ends of the polymer arms; n is an integer number; [ ] denotes that the group is optional; and, D2 is selected from amphiphilic or hydrophobic small molecule drugs linked to the reactive monomers distributed along the backbone of the first block of the polymer arm at a density of between 1 mol % and 80 mol %; and the first to second block ratio is between about 2:1 and 1:3, or between about 2:1 and 1:5, which may be represented schematically:

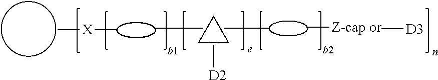

Or, wherein the second block comprises charged monomers the star diblock copolymer may be represented schematically:

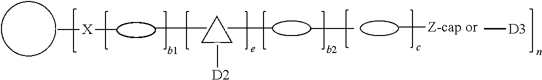

wherein, an integer number, n, of polymer arms with diblock architecture, i.e., —(B)b-co-(E(D))e-b-(B)b2- or —(B)b-co-(E(D))e-b-(B)b2-co-(C)c-, are linked to a core, O, through a linker, X; wherein the polymer arm comprises an integer number, b1, of hydrophilic monomers (B) and an integer number, e, of reactive monomers (E) linked to drug molecules (D) on the first block of the polymer arm (A) that is proximal to the core of the star polymer, and an integer number, b2, of hydrophilic monomers and (if present) an integer number of charged monomers, c, on the second block of the polymer arm (A); additionally wherein the distal ends of each of the polymer arms are either capped with a capping group or linked to a drug molecule (D3).

A non-limiting example of a star diblock copolymer is a star polymer of Formula VI comprising polymer arms with diblock architecture with both hydrophilic monomers (B) of Formula I and reactive monomers (E) of Formula III linked to drug molecules (D) on a first block of the polymer arm (A) that is proximal to the core, wherein the second block distal to the core comprises hydrophilic monomers of Formula I and optionally includes charged monomers of Formula II.

A non-limiting example of a star polymer of Formula VI is shown here for clarity

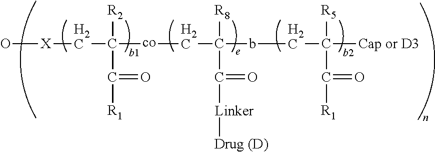

wherein in preferred embodiments of star polymers of Formula VI, the hydrophilic monomer (B) is selected from hydrophilic meth(arcylamides) or meth(acrlyates), such as HPMA, HEMA or HEMAM; the linker, X, is present, links the polymer arm to the core through any suitable means, though, preferably through an amide bond; the end of each polymer arm distal to the core is capped, preferably with isobutyronitrile, or linked to D3 preferably selected from targeting molecules; the core is an amide- or ester-based dendrimer, such as PAMAM- or bis(MPA)-based dendrimers, with generation between 1 to 6, such as 1, 2, 3, 4, 5 or 6 PAMAM dendrimers, preferably generation 3, 4 or 5; the symbols b, e and c are any integer denoting the number of monomers B, E and C, where the numbers 1 and 2 following the symbol b denote first block and second block, respectively; the total number of monomer units is typically between about 50 to about 450 monomer units; italicized b separates the first block from the second block and co indicates that the monomers are randomly distributed along that block of the copolymer; the total molecular weight of the polymer arm is between 5,000 and 60,000 Daltons (excluding the mass of the drug molecules), more preferably between 15,000 and 50,000 Daltons or 20,000 and 40,000 Daltons, or most preferably between about 20,00 to about 35,000 Daltons; the first to second block ratio is about 2:1 to 1:5, or about 2:1 to 1:3, more preferably between about 1:1 to 1:3, or about 1:1 to 1:2; n is an integer typically selected between 5 and 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 preferably between 10 and 45 polymers arms, or more preferably between 20 and 35 polymer arms; the drug molecules (D) are linked to the reactive monomers through any suitable linker molecule typically selected from enzyme degradable peptide-based linkers, carbamates, such as a self-immolative carbamate linker, e.g., PAB, acid-labile silyl ether, ketal or hydrazone linkers, or combinations thereof, at a density between 1 mol % and 80 mol %, more preferably between about 5 mol % and 40 mol % or most preferably between about 10 mol % and 30 mol %; and, the hydrodynamic radius of the star polymer is between 5 and 30 nm, preferably between 7.5 and 20 nm.

While certain compositions of star polymers of Formula VI enabled high loading of amphiphilic or hydrophobic drugs without requiring the use of charged monomers to prevent aggregation, for certain applications of star polymers of Formula VI, it was found to be beneficial to include charged monomers. Accordingly, the inventors of the present disclosure found that star polymers of Formula VI used for cancer treatment that included pH-responsive monomers that become positively charged at pH less than pH 7.4 (e.g., tumor pH) led to improved efficacy as compared with star polymers of Formula VI that are neutral at pH less than pH 7.4. A non-limiting explanation is that such star polymers are neutral in the blood and avoid capture by reticuloendothelial cells but become positively charged in the tumor thereby increasing their interactions with cells in the tumor microenvironment. Therefore, in preferred embodiments of star polymers of Formula VI used for cancer treatment, the star polymer comprises diblock copolymer arms, wherein the second block of the diblock copolymer arms comprise a charged monomer that is neutral at physiologic pH but becomes protonated and is positively charged at pH less than pH 7.4, e.g., at about pH 6.5.

A non-limiting example of a star polymer of Formula VI that comprises a star diblock copolymer comprising polymer arms with pH-responsive positively charged monomers but is neutral at physiologic pH, wherein the pH-responsive charged monomers comprise tertiary amines is shown here:

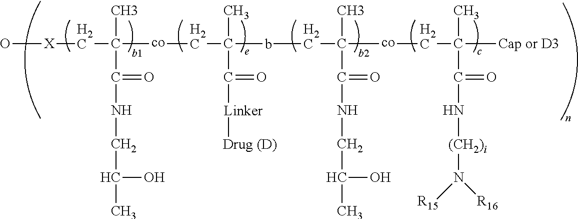

wherein the charged monomer is distributed alone the second block at a density of between 3 to 60 mol %, or 3 to 40 mol %, though, preferably between about 5 to 20 mol %; i is an integer number of repeating units of methylene groups, typically 2 to 6 methylene units, and R₁₅ and R₁₆ are independently selected from hydrogen, methyl, ethyl or isopropyl groups, though, preferably, R₁₅ and R₁₆ are both methyl groups.

The inventors of the present disclosure found that the aforementioned star polymers had utility for delivering a broad variety of different synthetic and naturally occurring molecules for myriad biomedical applications. The following sections describe specific examples of star polymers that have particular utility for certain applications.

### Optimization of Star Polymer Carriers of Sting Agonists

Certain preferred embodiments of star polymers for cancer treatment comprise STING agonists (STINGa). In addition to the aforementioned of star polymer compositions leading to unexpected improvements in biological activity of drug molecules used for cancer treatment, the inventors of the present disclosure identified that linker composition and architecture were key parameters impacting efficacy of STINGa linked to star polymers used for cancer treatment. Accordingly, the inventors of the present disclosure found that, while amphiphilic or hydrophobic STINGa (e.g., pip-diABZI) linked directly to star polymers through an amide bond were inactive in vivo, the same molecules linked to star polymers through enzyme (cathepsin) degradable peptides or acid labile hydrazone, silyl ether or ketal bonds were highly active in vivo. Thus, in preferred embodiments of amphiphilic or hydrophobic STINGa linked to star polymers, the STINGa is linked to the star polymers through enzyme (cathepsin) degradable peptides (either directly or via a carbamate) or acid labile hydrazone, silyl ether or ketal bonds. An additional notable finding was that the rate of release of the STINGa from the star polymer also impacted the therapeutic index as well as the capacity of the STING to prime anticancer T cell immunity. Notably, slowing the rate of release of the STINGa from the star polymer by using enzyme degradable peptides that require two steps (e.g., histone deacetylase and cathepsin recognition) or more stable acid-labile bonds, e.g., carbohydrazone (from carbohydrazide) versus hydrazone, led to improved therapeutic index and anticancer T cell priming. Architecture was also found to impact the efficacy of star polymer carriers of STINGa. Notably, star random copolymers of STINGa were more effective for promoting tumor clearance than star polymers based on star diblocks with the STINGa linked to one block or star polymers with STINGa linked to the ends of the star polymer (i.e., D3).

Therefore, in preferred embodiments, STINGa are linked to reactive monomers distributed along the backbone of the polymer arms of star random copolymers through enzyme-degradable amide linkages or acid-labile bonds. Based on these criteria, preferred compositions of star polymers delivering STINGa were identified and are described below.

In certain preferred embodiments of star polymers delivering STINGa for cancer treatment, the star polymer is a star polymer of Formula V comprising polymer arms that comprise hydrophilic monomers (B) of Formula I, reactive monomers (E) of Formula III linked to STINGa and optionally includes charged monomers (C) of Formula II, which is shown here for clarity:

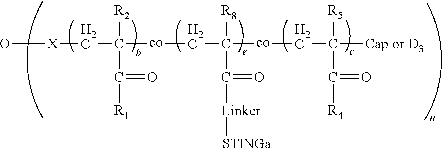

wherein the hydrophilic monomer (B) is typically selected from hydrophilic meth(arcylamides) or meth(acrlyates), such as HPMA, HEMA or HEMAM; the linker, X, if present, links the polymer arm to the core through any suitable means, though, preferably through an amide bond; the end of each polymer arm distal to the core is capped, preferably with isobutyronitrile; the core is an amide- or ester-based dendrimer, such as PAMAM- or bis(MPA)-based dendrimers, with generation between 1 to 6, such as 1, 2, 3, 4, 5 or 6 PAMAM dendrimer, preferably generation 3, 4 or 5; the symbols b, e and c are any integer denoting the number of monomers B, E and C, wherein the total number of monomer units is typically between about 50 to about 450 monomer units; co indicates that the monomers are randomly distributed along the backbone of the copolymer; the molecular weight of the polymer arm is between 5,000 and 60,000 Daltons (excluding the mass of the drug molecules), more preferably between 15,000 and 50,000 Daltons or 20,000 and 40,000 Daltons, most preferably between about 20,00 to about 35,000 Daltons; n is an integer typically selected between 5 and 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 preferably between 10 and 45 polymers arms or more preferably between 20 and 35 polymer arms; the drug molecules (D) are linked to the reactive monomers through any suitable linker molecule typically selected from enzyme degradable peptide-based linkers, carbamates, such as a self-immolative carbamate linker, e.g., PAB, acid-labile silyl ether, ketal or hydrazone linkers, or combinations thereof, at a density between 1 mol % and 40 mol %, or more preferably 5 mol % and 20 mol % or most preferably between 7.5 mol % and 15 mol %; the charged monomer, when present, is typically selected from pH-responsive positively charged monomers or negatively charged monomers that are pH responsive between pH of between about pH 4.5 to about 7.0, more preferably charged monomers with charge groups selected from glycine, beta-alanine, butanoic acid, methyl butanoic acid, dimethylbutanoic acid, 3,3′-((2-(6-aminohexanamido)propane-1,3-diyl)bis(oxy))dipropionic acid (referred to as “bis(COOH)”), 13-(6-aminohexanamido)-6,20-bis((2-carboxyethoxy)methyl)-8,18-dioxo-4,11,15,22-tetraoxa-7,19-diazapentacosanedioic acid (referred to as “tetra(COOH)”), most preferably DMBA, bis(COOH) and tetra(COOH); and, the hydrodynamic radius of the star polymer is between 5 and 30 nm, preferably between 7.5 and 20 nm.

In the above example, wherein the STINGa is hydrophobic or amphiphilic (e.g., diABZI based STINGa) and the charged monomer is selected from pH-responsive charged monomers that are neutral at physiologic pH 7.4 but are positively charged at pH less than pH 7.4, e.g., at pH 6.5 or less, a non-limiting example is:

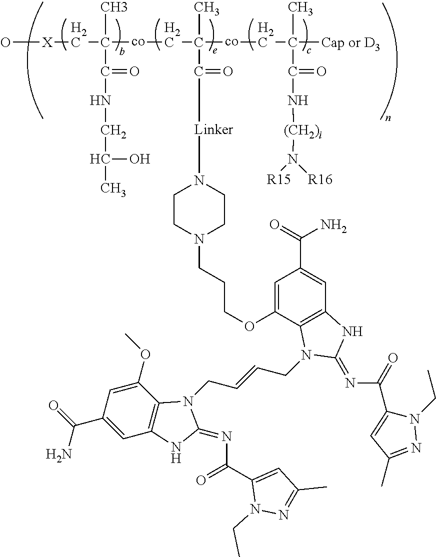

wherein the diABZI-based STINGa is preferably linked at a density between 1 mol % and 8 mol %, though, more preferably between 3 mol % and 7 mol %; i is an integer number of repeating units of methylene groups, typically 2 to 6 methylene units; R₁₅ and R₁₆ are independently selected from hydrogen, methyl, ethyl or isopropyl groups; and, the hydrodynamic radius of the star polymer is between 5 and 30 nm, preferably between 7.5 and 20 nm.

In certain preferred embodiments of star polymers delivering STINGa for cancer treatment, wherein the STINGa is hydrophobic or amphiphilic (e.g., diABZI based STINGa), the star polymer is a star polymer of Formula V comprising polymer arms that comprise hydrophilic monomers (B) of Formula I (e.g., HPMA), reactive monomers (E) of Formula III linked to STINGa and pH-responsive charged monomers of Formula II comprising carboxylic acids that are negative (i.e., deprotonated) at physiologic pH 7.4 but are neutral at pH less than pH 7.4, e.g., at pH 6.5 or less. A non-limiting example is shown here for clarity:

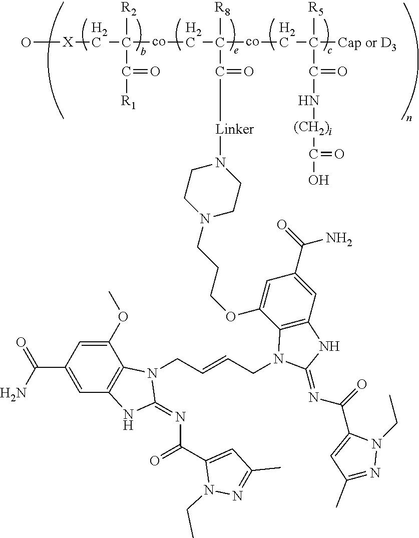

wherein the linker, X, if present, links the polymer arm to the core through any suitable means, though, preferably through an amide bond; the end of each polymer arm distal to the core is capped, preferably with isobutyronitrile; the core is an amide- or ester-based dendrimer, such as PAMAM- or bis(MPA)-based dendrimers, with generation between 1 to 6, such as 1, 2, 3, 4, 5 or 6 PAMAM dendrimer, preferably generation 3, 4 or 5; the symbols b, e and c are any integer denoting the number of monomers B, E and C, wherein the total number of monomer units is typically between about 50 to about 450 monomer units; co indicates that the monomers are randomly distributed along the backbone of the copolymer; the molecular weight of the polymer arm is between 5,000 and 60,000 Daltons (excluding the mass of the drug molecules), more preferably between 15,000 and 50,000 Daltons or 20,000 and 40,000 Daltons, most preferably between about 20,00 to about 35,000 Daltons; n is an integer typically selected between 5 and 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 preferably between 10 and 45 polymers arms or more preferably between 20 and 35 polymer arms; the drug molecules (D) are linked to the reactive monomers through any suitable linker molecule typically selected from enzyme degradable peptide-based linkers, carbamates, such as a self-immolative carbamate linker, e.g., PAB, acid-labile silyl ether, ketal or hydrazone linkers, or combinations thereof, at a density between 1 mol % and 40 mol %, though, more preferably between 5 mol % and 20 mol % or most preferably between 7.5 mol % and 15 mol %; the charged monomer is distributed along the polymer arms at the preferred density summarized in Table 1 (e.g., where D2 is attached at a density of 10 mol %, the charged monomer is preferably attached at a density of about 5 mol % to about 20 mol % or most preferably between 8 mol % to about 15 mol %); i is an integer number of repeating units of methylene groups, typically 1 to 4 methylene units, though, preferably 2 methylene units; and, the hydrodynamic radius of the star polymer is between 5 and 30 nm, preferably between 7.5 and 20 nm. In the above example, the charged monomer may optionally comprise glycine, beta-alanine, butanoic acid, methyl butanoic acid, DMBA, bis(COOH), tris(COOH) or tetra(COOH), provided that for bis(COOH) and tris(COOH)/tetra(COOH) the preferred densities for the charged monomer correspond to Table 2 and 3, respectively.

In the above example, wherein the polymer arms are linked to the core through an amide bond, e.g., via a cynanovaleroyl linker, the hydrophilic monomer (B) is HPMA, the charged monomer is a methacrylic acid substituted with DMBA via a beta-alanine linker, the reactive monomer is methacrylamide based, and the polymer is capped with isobutyronitrile, the structure is:

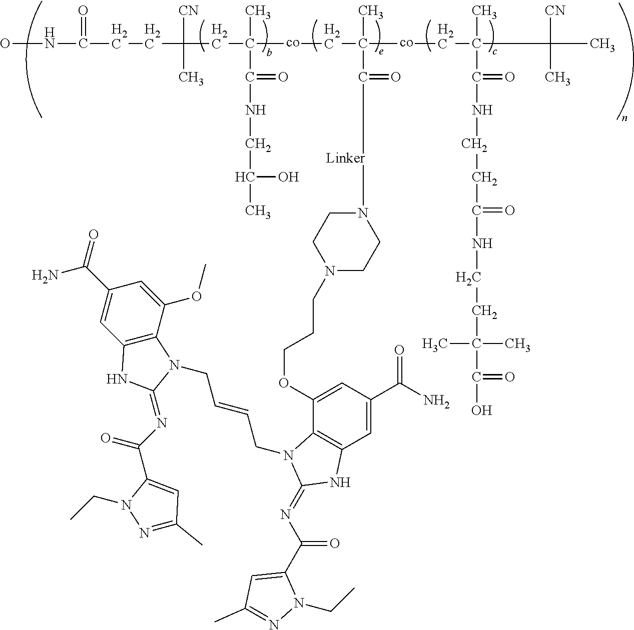

In the above example, wherein the amphiphilic or hydrophobic STINGa (e.g., diABZI) is linked either directly or via a carbamate linker to a peptide-based linker, the structure is:

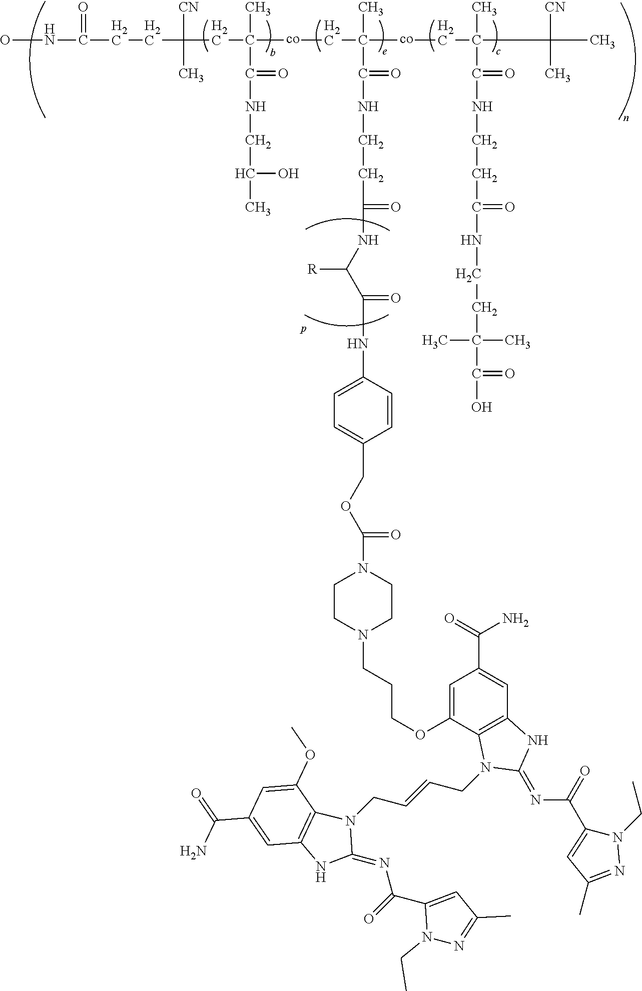

wherein p is an integer, preferably 2 to 6, denoting the number of amino acids and R is any suitable group, typically selected from naturally occurring amino acid side groups and modified side groups, e.g., acetylated or other suitably modified variants thereof.

In preferred embodiments, the amphiphilic or hydrophobic STINGa (e.g., diABZI) is linked to PAB, which is linked to a dipeptide, tripeptide or tetrapeptide, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine; P2 is selected from glycine, serine, leucine, valine or isoleucine; P3 is selected rom acetyl lysine, boc-protected lysine, norleucine (nLeu), glutamine, 6-hydroxy norleucine (abbreviated hnLeu), glycine, serine, alanine, proline, or leucine; and P4 is selected from glycine, serine, arginine, lysine, acetyl lysine (i.e., the epsilon amine is acetylated), Boc protected lysine, aspartic acid, glutamic acid or beta-alanine; the carbamate linker is optional and may be present or absent. A non-limiting example wherein the amphiphilic or hydrophobic STINGa (e.g., diABZI) is linked to PAB, which is linked to a dipeptide, Val-Cit, is shown here for clarity:

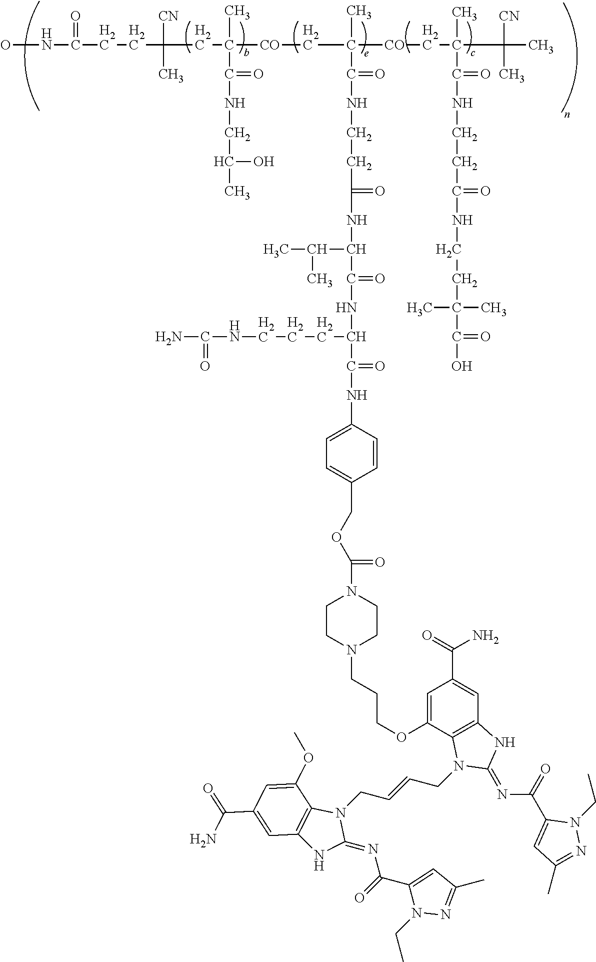

In the above example, the linker linking the amphipilic or hydrophobic a (e.g., diABZI) to the reactive monomer may alternatively comprise a hydrazone bond. A non-limiting example is shown here for clarity:

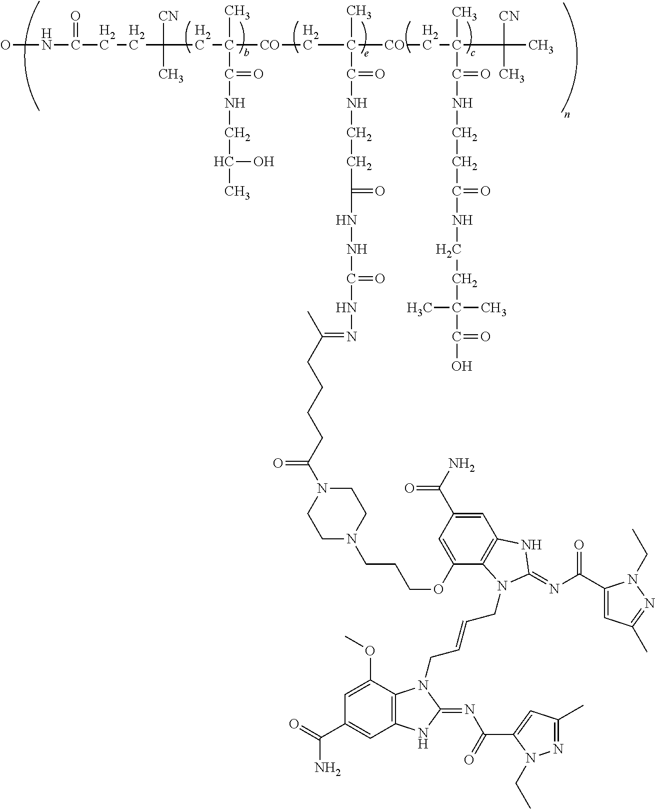

As CDN-based STINGa are negatively charged at physiologic pH, high densities of CDN-based STINGa can be attached to the polymer arms of star random copolymers without aggregation occurring. In certain preferred embodiments of star polymers delivering CDN-based STINGa for cancer treatment, the star polymer is a star polymer of Formula V comprising polymer arms that comprise hydrophilic monomers (B) of Formula I, reactive monomers (E) of Formula III linked to CDN-based STINGa and optionally includes charged monomers (C) of Formula II, which is shown here for clarity:

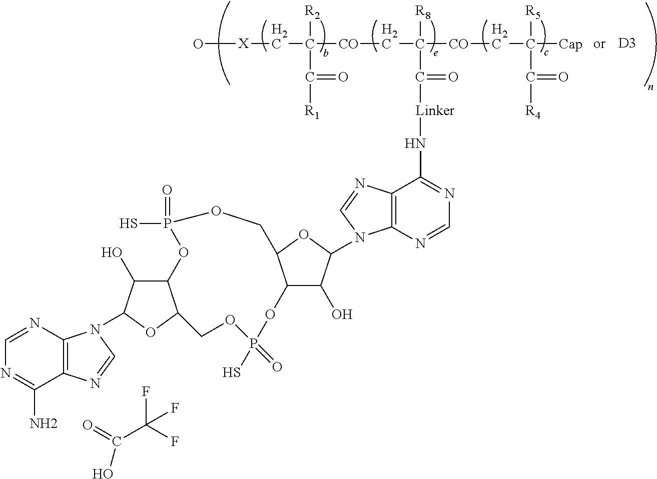

wherein the hydrophilic monomer (B) is typically selected from hydrophilic meth(arcylamides) or meth(acrlyates), such as HPMA, HEMA or HEMAM; the linker, X, if present, links the polymer arm to the core through any suitable means, though, preferably through an amide bond; the end of each polymer arm distal to the core is capped, preferably with isobutyronitrile; the core is an amide- or ester-based dendrimer, such as PAMAM- or bis(MPA)-based dendrimers, with generation between 1 to 6, such as 1, 2, 3, 4, 5 or 6 PAMAM dendrimer, preferably generation 3, 4 or 5; the symbols b, e and c are any integer denoting the number of monomers B, E and C, wherein the total number of monomer units is typically between about 50 to about 450 monomer units; co indicates that the monomers are randomly distributed along the backbone of the copolymer; the molecular weight of the polymer arm is 5,000 and 60,000 Daltons (excluding the mass of the drug molecules), more preferably between 15,000 and 50,000 Daltons or 20,000 and 40,000 Daltons, most preferably between about 20,00 to about 35,000 Daltons; n is an integer typically selected between 5 and 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 preferably between 10 and 45 polymers arms or more preferably between 20 and 35 polymer arms; the CDN-based STINGa are linked to the reactive monomers through any suitable linker molecule, though preferably through an enzyme degradable linker, more preferably via a cathepsin degradable peptide or sulfatase cleavable linker, optionally further comprising a self-immolative carbamate (e.g., PAB) at a density between 1 mol % and 40 mol %, though, preferably between about 5 mol % to 40 mol % or most preferably 10 mol % and 30 mol %; and, the hydrodynamic radius of the star polymer is between 5 and 30 nm, preferably between 7.5 and 20 nm.

In the above example, wherein the polymer arms are linked to the core through an amide bond, e.g., via a cynanovaleroyl linker, the hydrophilic monomer (B) is HPMA, the CDN-based STINGa is linked to methacrylamide-bases reactive monomers through a peptide, charged monomers (C) are absent, and the polymer arm is capped with isobutyronitrile, the structure is:

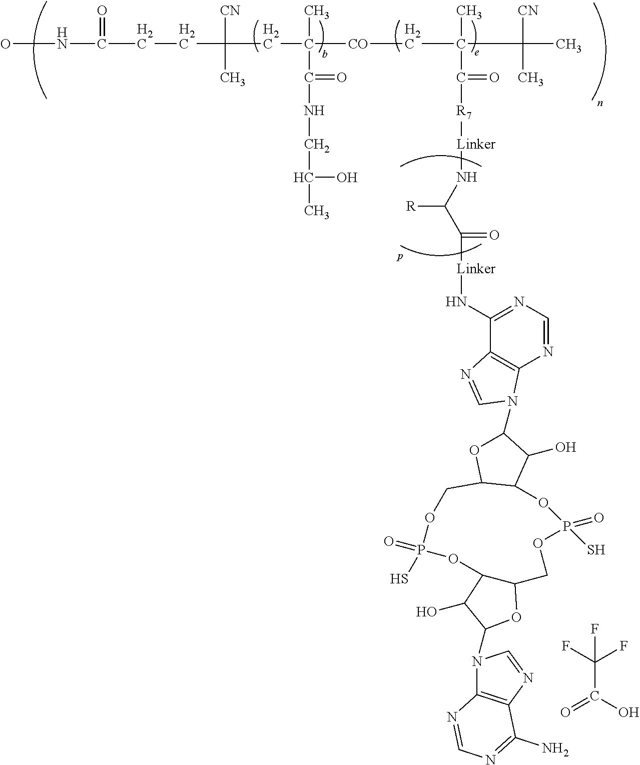

wherein p is an integer, preferably 2 to 6, denoting the number of amino acids in the peptide linker; R is any suitable group, typically selected from naturally occurring amino acid side groups and modified side groups, e.g., acetylated or other suitably modified variants thereof; and “Linkers” are any suitable linker molecules, wherein the linker between the CDN and the peptide is typically a self-immolative carbamate linker (e.g., PAB).

In preferred embodiments, the amphiphilic CDN-based STINGa is linked to a self-immolative carbamate linker (e.g., PAB), which is linked to a dipeptide, tripeptide or tetrapeptide, wherein P1 is selected from arginine, lysine, acetyl lysine (i.e., the epsilon amine is acetylated), Boc protected lysine (i.e., the epsilon amine is Boc protected), citrulline, glutamine, threonine, leucine, norleucine, alpha-aminobutyric acid (abbreviated as “a-But” herein) or methionine; P2 is selected from glycine, serine, leucine, valine or isoleucine; P3 is selected rom acetyl lysine, boc-protected lysine, norleucine (nLeu), glutamine, 6-hydroxy norleucine (abbreviated hnLeu), glycine, serine, alanine, proline, or leucine; and P4 is selected from glycine, serine, arginine, lysine, acetyl lysine (i.e., the epsilon amine is acetylated), Boc protected lysine, aspartic acid, glutamic acid or beta-alanine, which is linked either directly or via a linker (e.g., beta-alanine) to the reactive monomer.

### Optimization of Star Polymer Carriers of TLR-7/8A for Cancer Treatment

Small molecule TLR-7/8a can stimulate the innate and adaptive immune system to promote tumor killing but must be formulated in macromolecular or particle carriers to avoid systemic toxicity and localize activity within the tumor microenvironment and tumor draining lymph nodes.

General compositions of star polymers suitable for delivery of amphiphilic or hydrophobic drug molecules, including, e.g., amphiphilic or hydrophobic TLR-7/8a (e.g., imidazoquinolines, benzonapthyridines, thiazoquinolines, etc.) for cancer treatment were described in the preceding sections. Though, the inventors of the present disclosure identified specific compositions of star polymers of Formula V and Formula VI linked to amphiphilic or hydrophobic TLR-7/8a that led to unexpected improvements in activity.

For instance, star random copolymer carriers of TLR-7/8a led to higher magnitude innate immune cell activation as compared with star diblock copolymers. Though, for both star random copolymer and star diblock copolymer architectures, linking TLR-7/8a through enzyme degradable or pH labile linkers led to significantly higher activity than TLR-7/8a linked directly to polymers through amide bonds not (known) to be recognized by proteases. This was unexpected as TLR-7/8a linked to polymers through stable amide bonds have been shown to be effective when used as adjuvants for vaccines. Among different pH labile groups evaluated, TLR-7/8a linked to star random copolymers through pH-sensitive hydrazones were shown to provide significantly higher activity as compared with TLR-7/8a linked to star random copolymers through stable amide bonds. An additional unexpected finding, however, was that the length of the ketone linker precursor attached to TLR-7/8a and used to form a hydrazone bond had a major impact on stability and therefore loading of the TLR-7/8a. Accordingly, while TLR-7/8a linked to oxopentanoic acid (sometimes referred to as levulinic acid) and 5-oxohexanoic acid had the tendency to cyclize, TLR-7/8a linked (via amide bond) to a 6-oxohepatnaoic acid based ketone linker led to higher star polymer loading and improved biological activity. The rate of release of the TLR-7/8a from the star polymers, including star random copolymers, was found to have a major impact on biological activity, with linkers providing moderates rates of release leading to the highest efficacy for tumor regression. Finally, to achieve high loading (>10 mol %) of amphiphilic or hydrophobic TLR-7/8a on star random copolymers associated with significantly higher activity than those with lower loading, charged comonomers were required to prevent aggregation.

Based on the above observations, preferred embodiments of star polymer carriers of TLR-7/8a are those of Formula V or VI, wherein the TLR-7/8a is linked to the polymer backbone at densities greater than 10 mol % to reactive monomers through an enzyme degradable or pH labile, e.g., hydrazone, more preferably a carbohydrazone.

In certain preferred embodiments of star polymers delivering TLR-7/8a for cancer treatment, wherein the TLR-7/8a is hydrophobic or amphiphilic (e.g., imidazoquinolines, benzonapthyridines, thiazoquinolines, etc.), the star polymer is a star polymer of Formula V comprising polymer arms that comprise hydrophilic monomers (B) of Formula I (e.g., HPMA), reactive monomers (E) of Formula III linked to TLR-7/8a and pH-responsive charged monomers of Formula II comprising carboxylic acids that are negative (i.e., deprotonated) at physiologic pH 7.4 but are neutral at pH less than pH 7.4, e.g., at pH 6.5 or less. A non-limiting example is shown here for clarity:

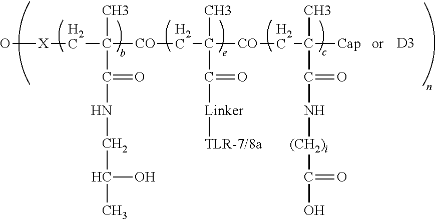

wherein the linker, X, if present, links the polymer arm to the core through any suitable means, though, preferably through an amide bond; the end of each polymer arm distal to the core is capped, preferably with isobutyronitrile; the core is an amide- or ester-based dendrimer, such as PAMAM- or bis(MPA)-based dendrimers, with generation between 1 to 6, such as 1, 2, 3, 4, 5 or 6 PAMAM dendrimer, preferably generation 3, 4 or 5; the symbols b, e and c are any integer denoting the number of monomers B, E and C, wherein the total number of monomer units is typically between about 50 to about 450 monomer units; co indicates that the monomers are randomly distributed along the backbone of the copolymer; the molecular weight of the polymer arm is between 5,000 and 60,000 Daltons (excluding the mass of the drug molecules), more preferably between 15,000 and 50,000 Daltons or 20,000 and 40,000 Daltons, most preferably between about 20,00 to about 35,000 Daltons; n is an integer typically selected between 5 and 60, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 preferably between 10 and 45 polymers arms or more preferably between 20 and 35 polymer arms; the drug molecules (D) are linked to the reactive monomers through any suitable linker molecule typically selected from enzyme degradable peptide-based linkers, carbamates, such as a self-immolative carbamate linker, e.g., PAB, acid-labile silyl ether, ketal or hydrazone linkers, or combinations thereof, at a density between 1 mol % and 40 mol %, though, more preferably between 5 mol % and 20 mol % or most preferably between 7.5 mol % and 15 mol %; the charged monomer is distributed along the polymer arms at the preferred density summarized in Table 1 (e.g., where D2 is attached at a density of 10 mol %, the charged monomer is preferably attached at a density of about 5 mol % to about 20 mol % or most preferably between 8 mol % to about 15 mol %); i is an integer number of repeating units of methylene groups, typically 1 to 4 methylene units, though, preferably 2 methylene units; and, the hydrodynamic radius of the star polymer is between 5 and 30 nm, preferably between 7.5 and 20 nm. In the above example, the charged monomer may optionally comprise glycine, beta-alanine, butanoic acid, methyl butanoic acid, DMBA, bis(COOH), tris(COOH) or tetra(COOH), provided that for bis(COOH) and tris(COOH)/tetra(COOH) the preferred densities for the charged monomer correspond to Table 2 and 3, respectively.

In the above example, wherein the TLR-7/8a is selected from an imdazoquinoline-based TLR-7/8a of Formula IV, the polymer arms are linked to the core through an amide bond, e.g., via a cynanovaleroyl linker, the hydrophilic monomer (B) is HPMA, the charged monomer is a methacrylic acid substituted with beta-alanine linked to DMBA, the polymer is capped with isobutyronitrile, and the TLR-7/8a is linked to the polymer via a carbohydrazone, the structure is:

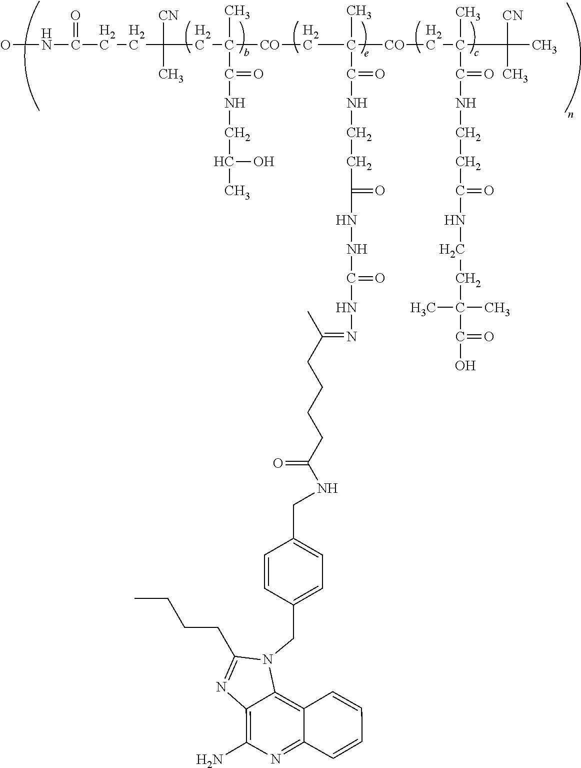

In the above example, wherein the TLR-7/8a is selected from an imdazoquinoline-based TLR-7/8a of Formula IV linked to the polymer backbone via a terapeptide (R₁₀ is any suitable amino acid side chain) and an optional self-immolative carbamate linker, the structure becomes:

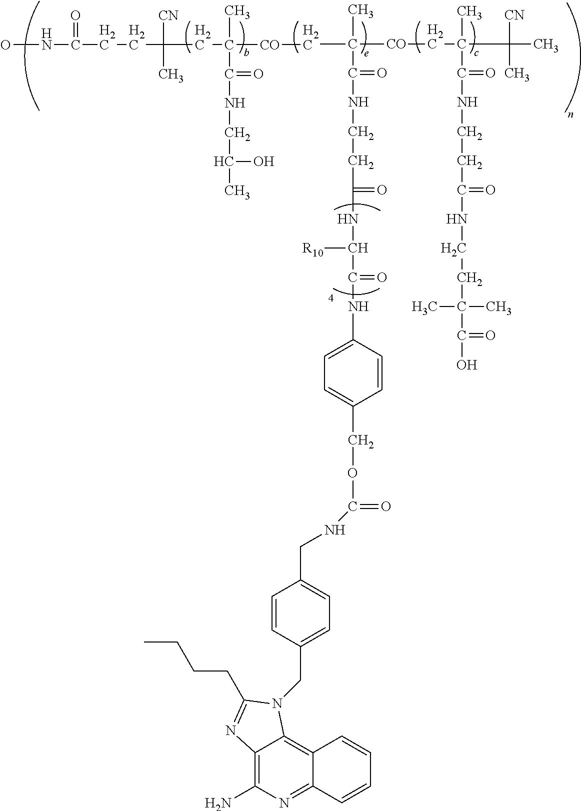

### Optimization of Star Polymer Carriers of Chemotherapeutic Drugs

General compositions of star polymers suitable for delivery of chemotherapeutic drugs for cancer treatment were described in the preceding sections. Though, specific examples are provided below in this subsection for clarity.

In certain preferred embodiments of star polymers delivering chemotherapeutics for cancer treatment, the chemotherapeutic is selected from anthracyclines (e.g., doxorubicin). A non-limiting example is shown here for clarity:

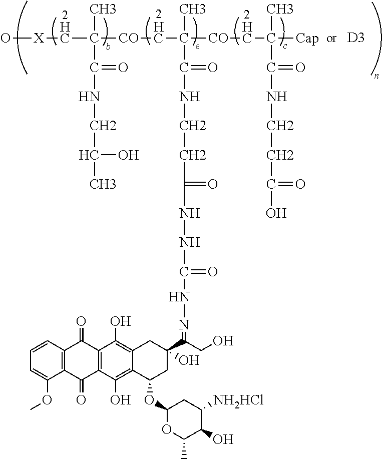

In certain preferred embodiments of star polymers delivering chemotherapeutics for cancer treatment, the chemotherapeutic is selected from topoisomerase inhibitors, including camptothecin and its analogs (e.g., topotecan). A non-limiting example is shown here for clarity, wherein topotecan is modified to enable conjugation to a self-immolative carbamate linker (i.e., PAB) that is linked to a peptide that is linked to the reactive monomer via beta alanine, wherein p is an integer number, typically 2, 3 or 4 amino acids and R₁₀ is any suitable amino acid side chain:

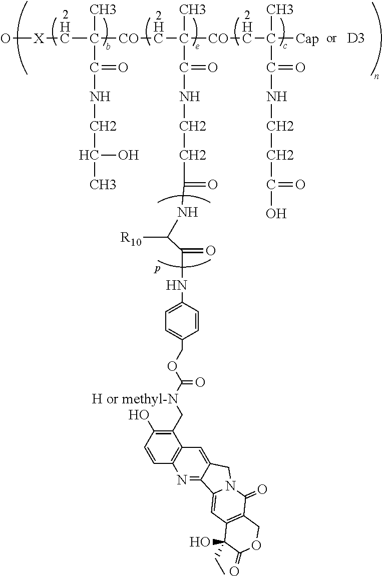

In certain preferred embodiments of star polymers delivering chemotherapeutics for cancer treatment, the chemotherapeutic is selected from nucleotide analogs. A non-limiting example is shown here for clarity, wherein the nucleotide analog cytarabine is linked to a self-immolative carbamate linker (i.e., PAB) that is linked to a peptide that is linked to the reactive monomer via beta alanine, wherein p is an integer number, typically 2, 3 or 4 amino acids and R₁₀ is any suitable amino acid side chain:

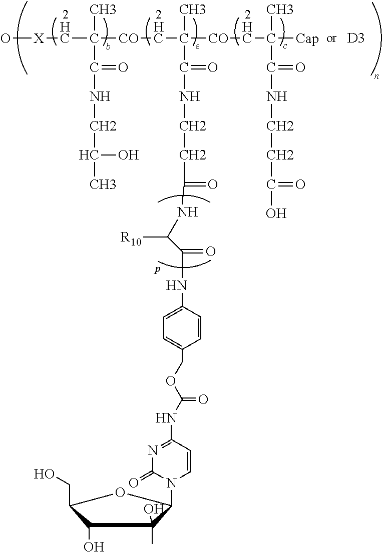

In certain preferred embodiments of star polymers delivering chemotherapeutics for cancer treatment, the chemotherapeutic is selected from retinoid receptor agonists. A non-limiting example is shown here for clarity, wherein bexarotene is linked to a peptide that is linked to the reactive monomer via ethylene diamine linked to methacrylic acid, wherein p is an integer number, typically 2, 3 or 4 amino acids and R₁₀ is any suitable amino acid side chain:

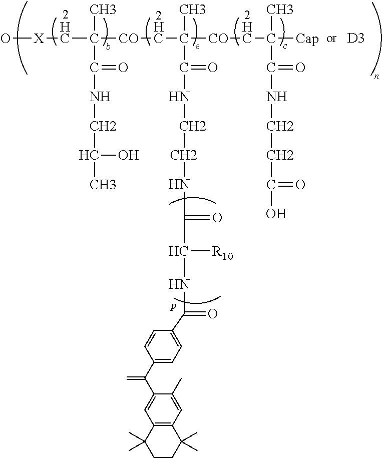

In certain preferred embodiments of star polymers delivering chemotherapeutics for cancer treatment, the chemotherapeutic is selected from antimetabolites (e.g., methotrexate). A non-limiting example is shown here for clarity, wherein methotrexate is linked to a self-immolative carbamate linker (i.e., PAB) that is linked to a peptide that is linked to the reactive monomer via beta alanine, wherein p is an integer number, typically 2, 3 or 4 amino acids and R₁₀ is any suitable amino acid side chain:

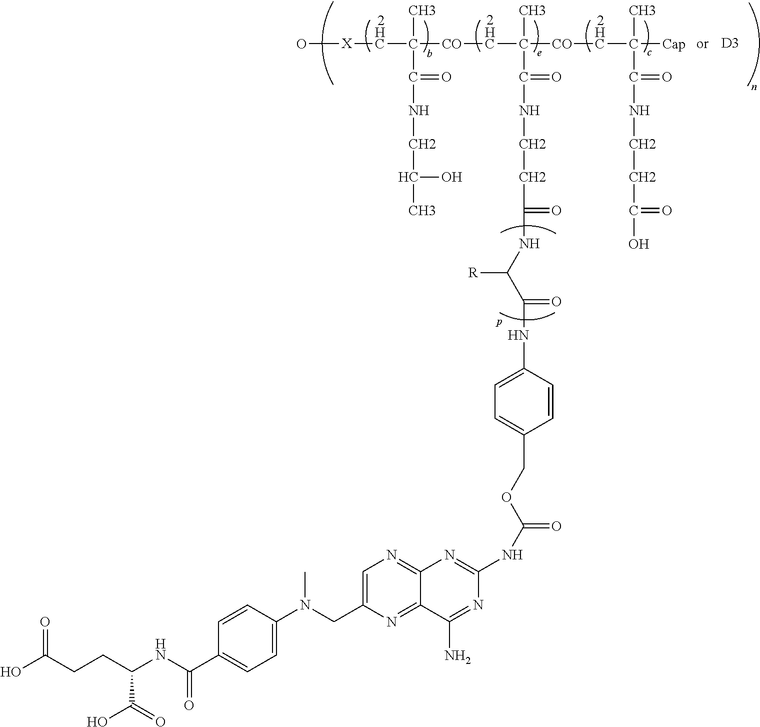

In certain preferred embodiments of star polymers delivering chemotherapeutics for cancer treatment, the chemotherapeutic is selected from kinase inhibitors (e.g., gefitinib). A non-limiting example is shown here for clarity, wherein modified (i.e., morpholine has been replaced with piperazine) gefitinib is linked to a self-immolative carbamate linker (i.e., PAB) that is linked to a peptide that is linked to the reactive monomer via beta alanine, wherein p is an integer number, typically 2, 3 or 4 amino acids and R₁₀ is any suitable amino acid side chain:

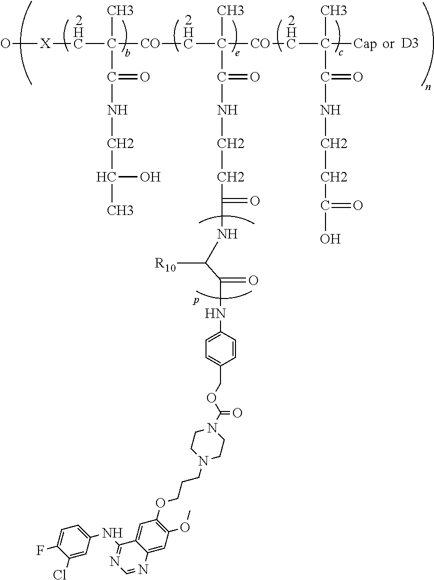

In certain preferred embodiments of star polymers delivering chemotherapeutics for cancer treatment, the chemotherapeutic is selected from VEGF receptor antagonists (e.g., sunitinib). A non-limiting example is shown here for clarity, wherein modified (i.e., amine is modified to enable conjugation) sunitinib is linked to a self-immolative carbamate linker (i.e., PAB) that is linked to a peptide that is linked to the reactive monomer via beta alanine, wherein p is an integer number, typically 2, 3 or 4 amino acids and R₁₀ is any suitable amino acid side chain:

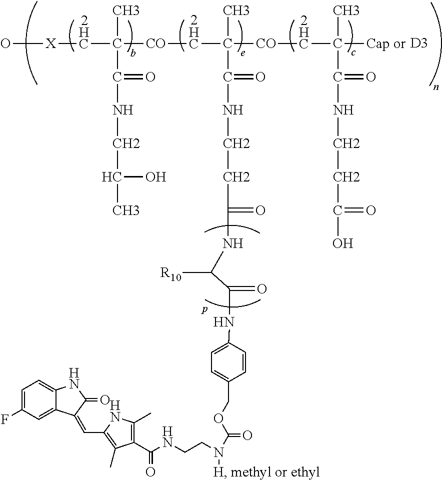

### EXAMPLES

The following preparations of compounds and intermediates are given to enable those skilled in the art to more clearly understand and to practice the present disclosure. They should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., or from about 0° C. to about 125° C. or at about room (or ambient) temperature, e.g., about 20° C.

Compounds of Formula (I) and subformulae and species described herein, including those where the substituent groups as defined herein, can be prepared as illustrated and described below.

In therapeutic applications described herein, the compounds can be formulated using techniques and formulations generally may be found in Remington, The Science and Practice of Pharmacy, (20th ed. 2000). For injection, the compounds may be formulated and diluted in aqueous solutions, such as in physiologically compatible buffers such as Hank's solution, Ringer's solution, or physiological saline buffer.

The following abbreviations are used in the text:

| | |
|---|---|
| AIBN | azobisisobutyronitrile |
| APCI | atmospheric pressure chemical ionization |
| AUC | area under curve |
| Boc | tert-butyloxycarbonyl |
| BOP | benzotriazol-1-yloxytris(dimethylamino)phosphonium |
| | hexafluorophosphate |
| CPI | cysteinylprolyl imide |
| CTA | chain transfer agent |
| CV | column volume |
| DCM | dichloromethane |
| DEPBT | 3-(diethoxyphosphoryloxy)-1,2,3-benzotriazin-4(3H)-one |
| DI | deionized |
| DIC | N,N′-diisopropylcarbodiimide |
| DIEA | N,N-diisopropylethylamine |
| DLS | dynamic light scattering |
| DLS | dynamic light scattering |
| DMAC | dimethylacetamide |
| DMAc | dimethylacetamide |
| DMAP | 4-dimethylaminopyridine |
| DMF | dimethylformamide |
| DMSO | dimethyl sulfoxide |
| DMTMM | 4-(4,6-dimethoxy-1,3,5-triazin-2-yl)-4-methyl-morpholinium |
| | chloride |
| EDC | 1-ethyl-3-(-3-dimethylaminopropyl) carbodiimide |
| | hydrochloride |
| ESI-MS | electrospray ionization mass spectrometry |
| Et₂O | diethyl ether |
| Et₃N | triethylamine |
| EtOAc | ethyl acetate |
| Fmoc | fluorenylmethoxycarbonyl |
| GPC-MALS | gel permeation chromatography multi-angle light scattering |
| h | hour |
| HATU | 1-[bis(dimethylamino)methylene]-1H-1,2,3-triazolo[4,5-b] |
| | pyridinium 3-oxide hexafluorophosphate |
| HBTU | 3-[bis(dimethylamino)methyliumyl]-3H-benzotriazol-1- |
| | oxide hexafluorophosphate |
| HCTU | O-(1H-6-chlorobenzotriazole-1-yl)-1,1,3,3- |
| | tetramethyluronium hexafluorophosphate |
| HPLC | high-pressure liquid chromatography |
| | liter |
| M | molar |
| MeOH | methanol |
| min | minute |
| mL | milliliter |
| Mn | number average molecular weight |
| MW | molecular weight |
| Mw | weight average molecular weight |
| MWCO | molecular weight cut off |
| NHS | N-hydroxysuccinimide |
| PDI | polydispersity |
| PyAOP | (7-azabenzotriazol-1-yloxy)tripyrrolidinophosphonium |
| | hexafluorophosphate |
| PyBOP | benzotriazol-1-yloxytripyrrolidinophosphonium |
| | hexafluorophosphate |
| r.t. | room temperature |
| RAFT | reversible addition-fragmentation chain transfer |
| Rg | radius of gyration |
| Rh | hydrodynamic radius |
| sat′d | saturated |
| tBuOH | tertiary butyl alcohol |
| TCO | trans-cyclooctene |
| TFA | trifluoroacetic acid |
| THF | tetrahydrofuran |
| THPTA | tris-hydroxypropyltriazolylmethylamine |
| wt | weight |

### Example 1—Synthesis of Drug Molecules (D) for Attachment to Star Polymers

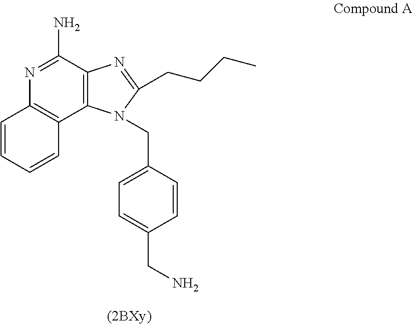 [SMILES: CCCCc1nc2c(N)nc3ccccc3c2n1Cc1ccc(CN)cc1]

Compound A, 1-(4-(aminomethyl)benzyl)-2-butyl-1H-imidazo[4,5-c]quinolin-4-amine, referred to as 2BXy, is a TLR-7/8 agonist that was synthesized as previously described (see Lynn, G. M. et al. Nat. Biotechnol., 2015, 33 (11), 1201-1210, and Shukla, N. M. et al. Bioorg. Med. Chem. Lett., 2010, 20 (22), 6384-6386). Note: The primary amine on xylene at the N1 position provided a reactive handle for attachment to star polymers either directly or through a linker. ¹H NMR (400 MHz, DMSO-d₆) δ 7.77 (dd, J=8.4, 1.4 Hz, 1H), 7.55 (dd, J=8.4, 1.2 Hz, 1H), 7.35-7.28 (m, 1H), 7.25 (d, J=7.9 Hz, 2H), 7.06-6.98 (m, 1H), 6.94 (d, J=7.9 Hz, 2H), 6.50 (s, 2H), 5.81 (s, 2H), 3.64 (s, 2H), 2.92-2.84 (m, 2H), 2.15 (s, 2H), 1.71 (q, J=7.5 Hz, 2H), 1.36 (q, J=7.4 Hz, 2H), 0.85 (t, J=7.4 Hz, 3H). MS (APCI) calculated for C₂₂H₂₅N₅, m/z 359.2, found 360.3.

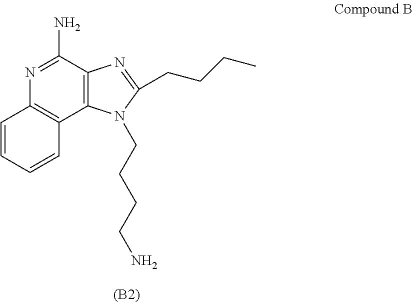 [SMILES: CCCCc1nc2c(N)nc3ccccc3c2n1CCCCN ; "(B2)"]

Compound B, sometimes referred to as “2B,” 1-(4-aminobutyl)-2-butyl-1H-imidazo[4,5-c]quinolin-4-amine, is a TLR-7/8 agonist that was synthesized as previously described (Lynn, G. M. et al. Nat. Biotechnol., 2020, 38, 320-332). Note: The butyl amine group at the N1 position provided a reactive handle for attachment to star polymers either directly or through a linker. ¹H NMR (400 MHz, DMSO-d₆) δ 8.03 (d, J=8.1 Hz, 1H), 7.59 (d, J=8.1 Hz, 1H), 7.41 (t, J=7.41 Hz, 1H), 7.25 (t, J=7.4 Hz, 1H), 6.47 (s, 2H), 4.49 (t, J=7.4 Hz, 2H), 2.91 (t, J=7.78 Hz, 2H), 2.57 (t, J=6.64 Hz, 1H), 1.80 (m, 4H), 1.46 (sep, J=7.75 Hz, 4H), 0.96 (t, J=7.4 Hz, 3H). MS (ESI) calculated for C₁₈H₂₅N₅, m/z 311.21, found 312.3.

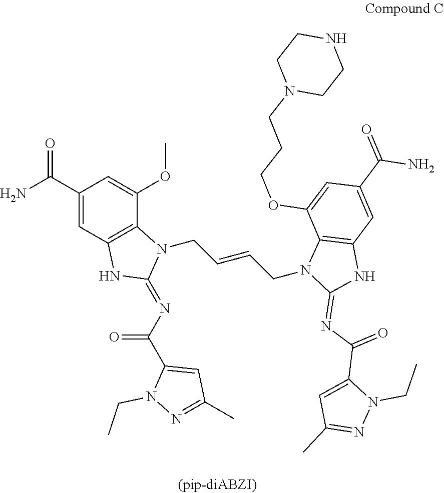 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

Compound C, sometimes referred to as “pip-diABZI” is a piperarzine modified linked amidobenzimidazole-based STING agonist that was synthesized in a similar manner as was described for a morpholine derivative (“Compound 3” in the reference Ramanjulu, J. M. et al. Nature, 2018, 564, 439-443), as summarized here:

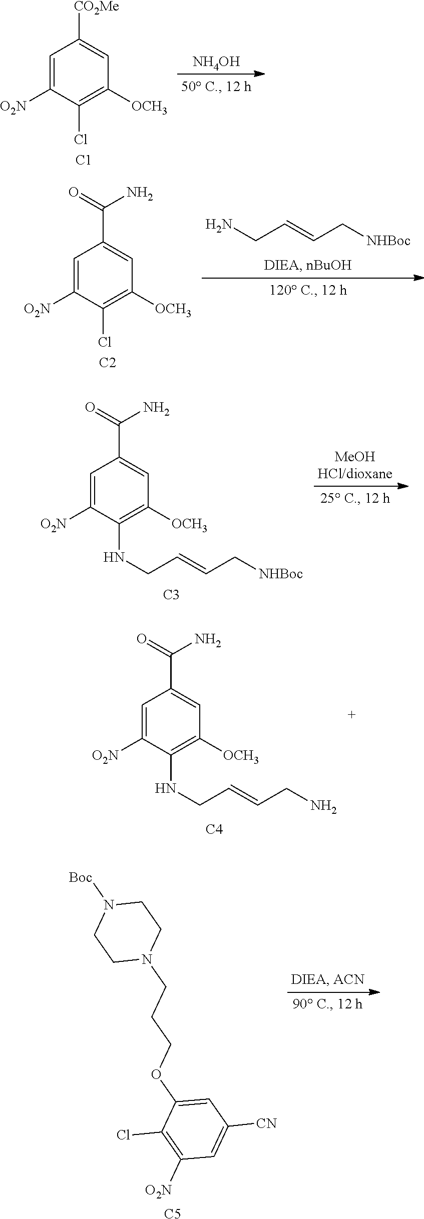

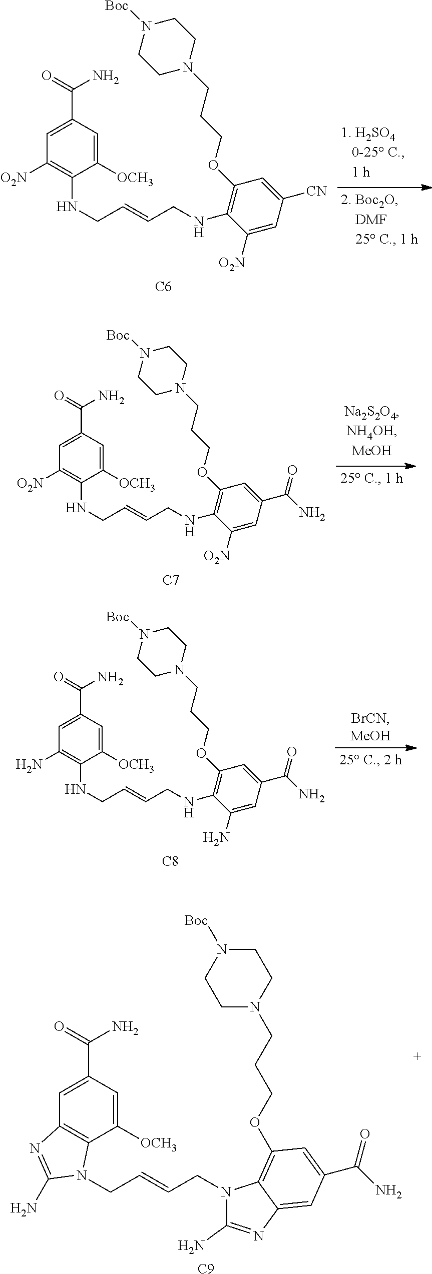

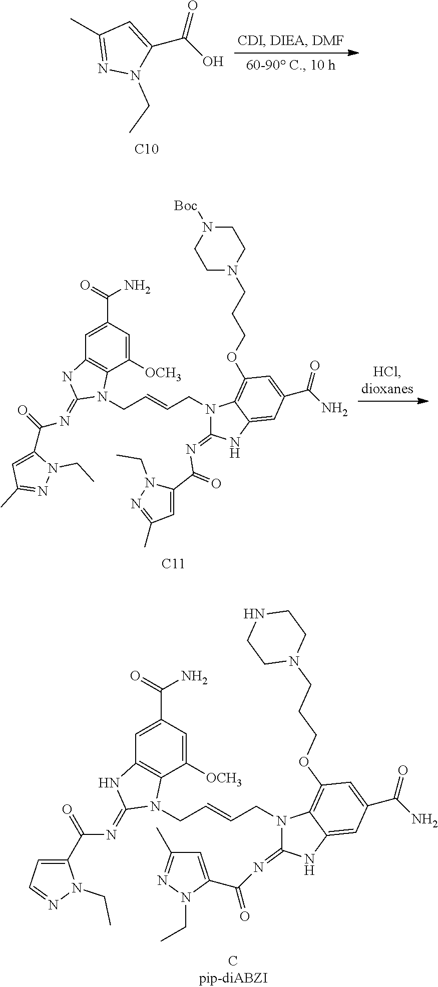 [SMILES: CCn1nc(C)cc1C(=O)O ; "C10" ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OC(C)(C)C)CC3)c21 ; "C11" ; CCn1nccc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

Amidation of methyl 4-chloro-3-methoxy-5-nitrobenzoate C1 with ammonium hydroxide afforded C2. Installation of the Boc-protected (E)-but-2-ene-1,4-diamine by nucleophilic aromatic substitution at the activated chloride, C2, afforded intermediate C3; acid-catalyzed deprotection of the Boc-protected amine afforded C4. The nucleophilic aromatic substitution of the primary alkyl amine of C4 at the 2-chloro position of C5 afforded intermediate C6. Hydrolysis of the nitrile to the amide was achieved by careful treatment with sulfuric acid which concomitantly cleaved the Boc-protected group of the piperazine; reinstallation of the Boc-group afforded C7. The reduction of the aryl nitro groups of C7 was affected under basic conditions with sodium dithionite to provide C8. Treatment with cyanogen bromide facilitated construction of the di-1H-benzo[d]imidazol-2-amine ring systems, C9. Activation of pyrazole-5-carboxylate C10 with carbonyl diimidazole and subsequent displacement by C9 provided penultimate intermediate, C11. Final protecting group removal with HCl in dioxanes provided C, pip-diABZI, as the hydrochloride salt. Note: The piperazine was introduced to provide a reactive-handle for attachment to star polymers either directly or through a linker. Sometimes pip-diABZI is referred to generically as “diABZI,” when linked to polymers, including star polymers. ¹H NMR (400 MHz, DMSO-d₆) conforms to structure. HPLC purity at 220 nm, 99.8% AUC. MS (ESI) calculated for C₄₂H₅₂N₁₄O₆, m/z 848.42, found 849.5.

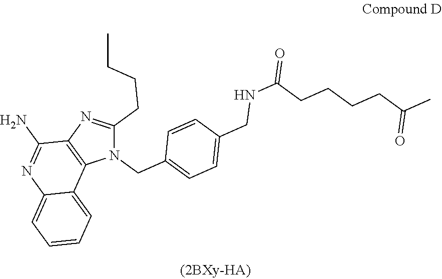 [SMILES: CCCCc1nc2c(N)nc3ccccc3c2n1Cc1ccc(CNC(=O)CCCCC(C)=O)cc1]

Compound D, N-(4-((4-amino-2-butyl-1H-imidazo[4,5-c]quinolin-1-yl)methyl)benzyl)-6-oxoheptanamide, referred to as 2BXy-HA is a TLR-7/8 agonist that was modified with a ketone, 6-oxohepantanoic acid (HA), to enable linkage to star polymers through a pH-sensitive hydrazone bond. To a solution of 6-oxoheptanoic acid (36 mg, 0.25 mmol) in DCM (5.0 mL) was added EDC (48 mg, 0.25 mmol). Sequentially, 1-(4-(aminomethyl)benzyl)-2-butyl-1H-imidazo[4,5-c]quinolin-4-amine (50 mg, 0.14 mmol), Et₃N (21 mg, 0.15 mmol) and DMAP (3.0 mg, 0.025 mmol) were added and stirred for 16 h at room temperature. The solution was partitioned between DCM (30 mL) and water (15 mL). The organic layer was washed with sat'd NH₄Cl (15 mL), sat'd NaHCO₃ (2×15 mL), dried over Na₂SO₄, filtered and concentrated. Upon drying, the product was isolated as a light yellow/brown foamy solid. HPLC purity at 220 nm, >95.0% AUC. MS (ESI) calculated for C₂₉H₃₅N₅O₂, m/z 485.3, found 486.2.

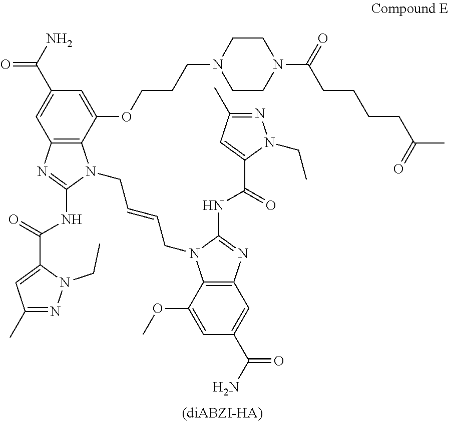 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCCCC(C)=O)CC3)c21]

Compound E, (E)-1-(4-(5-carbamoyl-2-(1-ethyl-3-methyl-1H-pyrazole-5-carboxamido)-7-(3-(4-(6-oxoheptanoyl)piperazin-1-yl)propoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide, referred to as pip-diABZI-HA (or sometimes herein as “diABZI”). Note: A ketone, 6-oxohepantanoic acid (HA), was introduced to enable linkage to star polymers through a pH-sensitive hydrazone bond. To 6-oxoheptanoic acid (0.80 mg, 0.056 mmol) in DMF (0.5 mL) was added (E)-1-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (5 mg, 0.0059 mmol). DIEA (3.0 mg, 0.023 mmol) was added followed by HATU (2.0 mg, 0.0056 mmol). The solution was stirred for 2 hours. The DMF was removed, the sample was dried under vacuum, and used in the subsequent step without further purification or characterization. HPLC purity at 220 nm, >95.0% AUC. MS (ESI) calculated for C₄₉H₆₂N₄O₈, m/z 974.5, found 488 (m/2).

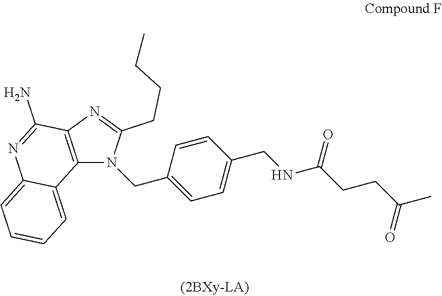 [SMILES: CCCCc1nc2c(N)nc3ccccc3c2n1Cc1ccc(CNC(=O)CCC(C)=O)cc1]

Compound F, N-(4-((4-amino-2-butyl-1H-imidazo[4,5-c]quinolin-1-yl)methyl)benzyl)-4-oxopentanamide, referred to as 2BXy-levulonic acid or “2BXy-LA” is a TLR-7/8a agonist that was modified with a ketone, levulinic acid (LA), to enable linkage to star polymers through a pH-sensitive hydrazone bond. Compound F was prepared in a manner similar to that which was described for Compound D except levulinic acid was used in place of 6-oxoheptanoic acid. Upon purification on silica gel however, cyclization of the levulinic acid was observed and 1-(4-((4-amino-2-butyl-1H-imidazo[4,5-c]quinolin-1-yl)methyl)benzyl)-5-methyl-1,3-dihydro-2H-pyrrol-2-one had formed.

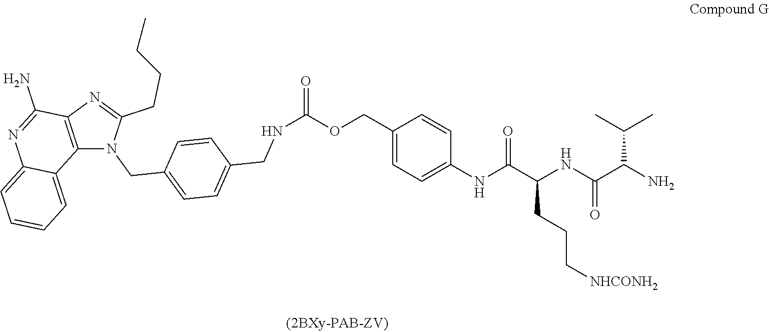

Compound G, 4-((S)-2-((R)-2-amino-3-methylbutanamido)-5-ureidopentanamido)benzyl (4-((4-amino-2-butyl-1H-imidazo[4,5-c]quinolin-1-yl)methyl)benzyl)carbamate referred to as 2BXy-PAB-ZV is a TLR-7/8 comprising a carbamate linker that is linked to an enzyme (cathepsin) degradable peptide linker, wherein the N-terminal amine is used as reactive handle for attachment to polymers, including the star polymers described herein. To 1-(4-(aminomethyl)benzyl)-2-butyl-1H-imidazo[4,5-c]quinolin-4-amine or 2BXy (25 mg, 0.069 mmol) in DMF (1.0 mL) was added (9H-fluoren-9-yl)methyl ((R)-3-methyl-1-(((S)-1-((4-((((4-nitrophenoxy)-carbonyl)oxy)methyl)phenyl)amino)-1-oxo-5-ureidopentan-2-yl)amino)-1-oxobutan-2-yl)carbamate (56 mg, 0.073 mmol) and potassium carbonate (24 mg, 0.17 mmol). The mixture was heated at 60° C. for 7 h. The DMF was removed and the material was purified by reversed-phase chromatography (5% acetonitrile/95% water to 100% acetonitrile; (w/0.05% TFA)). The purified Fmoc-protected intermediate was dried overnight. The material was then dissolved in 20% piperidine in DMF and stirred for 1 h at rt. The DMF was removed in vacuo. The product was isolated cleanly after trituration with diethyl ether.

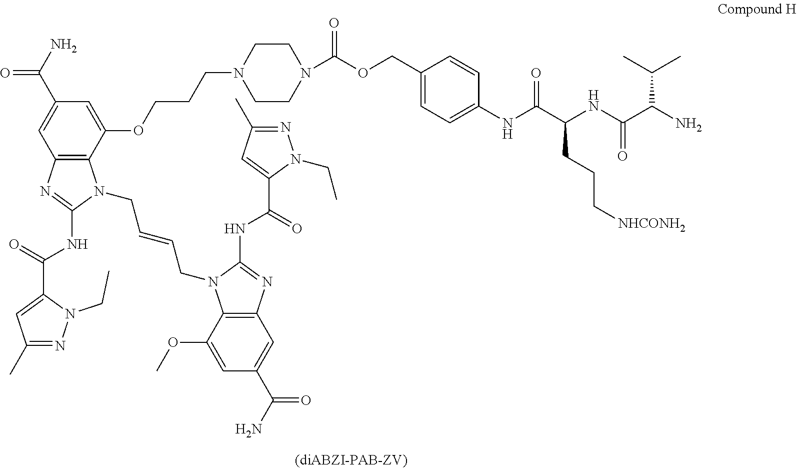

Compound H, 4-((S)-2-((R)-2-amino-3-methylbutanamido)-5-ureidopentanamido)benzyl 4-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperazine-1-carboxylate referred to as diABZI-PAB-ZV; this compound is a STING agonist linked to a carbamate linker that is linked to an enzyme (cathepsin) degradable peptide linker, wherein the N-terminal amine is used as reactive handle for attachment to polymers, including the star polymers described herein. Compound H was prepared in a manner analogous to Compound G. In place of 2BXy, pip-diABZI was used. HPLC purity at 220 nm, >95% AUC. MS (ESI) calculated for C₆₁H₇₉N₁₉NaO₁₁, m/z 1276.1, found 1277.3.

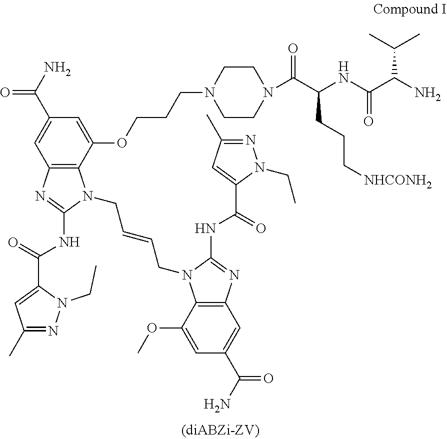

Compound I, 7-(3-(4-((S)-2-((R)-2-amino-3-methylbutanamido)-5-ureidopentanoyl)piperazin-1-yl)propoxy)-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide referred to as diABZI-ZV; this compound is a STING agonist that is linked to an enzyme (cathepsin) degradable peptide linker, wherein the N-terminal amine is used as reactive handle for attachment to polymers, including the star polymers described herein. To a solution of pip-diABZI hydrochloride salt (25 mg, 0.028 mmol) and (S)-2-((R)-2-((((9H-fluoren-9-yl)methoxy)carbonyl)amino)-3-methylbutanamido)-5-ureidopentanoic acid (14 mg, 0.028 mmol) in DMF (1 mL) was added DIEA (21 mg, 0.17 mmol). The solution was cooled to 0° C., HATU (11 mg, 0.030 mmol) was added and then allowed to warm to r.t. overnight. The DMF was removed and the material was purified by reversed-phase chromatography (5% acetonitrile/95% water to 100% acetonitrile; (w/0.05% TFA)). The purified Fmoc-protected intermediate was dried overnight. The material was dissolved in 20% piperidine (in DMF) and stirred for 1 h at rt. The DMF was removed in vacuo. The product was isolated cleanly after trituration with diethyl ether. HPLC purity at 220 nm, >95% AUC. MS (ESI) calculated for C₅₃H₇₂N₁₈O₉, m/z 1105.2, found 1106.4.

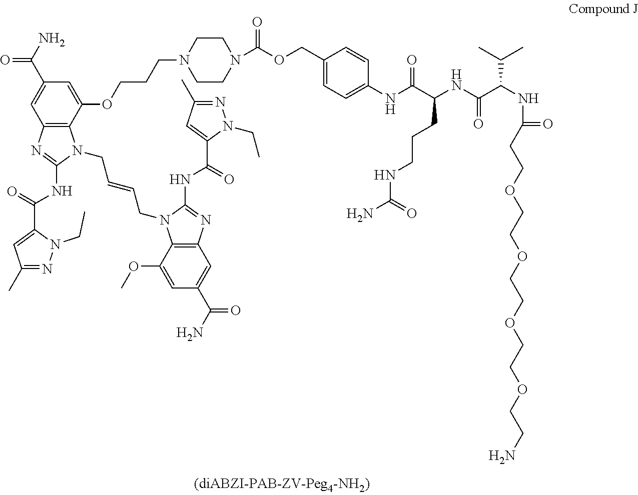 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCN)C(C)C)cc4)CC3)c21]

Compound J, 4-((17R,20S)-1-amino-17-isopropyl-15, 18-dioxo-20-(3-ureidopropyl)-3,6,9,12-tetraoxa-16,19-diazahenicosan-21-amido)benzyl 4-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperazine-1-carboxylate, also referred to as diABZI-PAB-ZV-Peg₄-NH₂; this compound is a STING agonist that is linked to a carbamate linker that is linked to an enzyme (cathepsin) degradable peptide linker that is linked to a PEG linker, wherein the primary amino on the PEG linker is used as reactive handle for attachment to polymers, including the star polymers described herein. To Compound H, diABZI-PAB-ZV (15 mg, 0.0087 mmol) in DMF (600 μL) was added Et₃N (1.3 mg, 0.013 mmol) followed by Fmoc-Peg₄.NHS ester (5.6 mg, 0.095 mmol). The slightly turbid solution was heated at 60° C. for 6 hours. To this solution was added 20% piperidine in DMF (400 μL). The crude material was purified by preparative reversed-phase chromatography using a gradient of 0% acetonitrile/water to 30% acetonitrile/water (w/0.05% TFA). HPLC purity at 220 nm, >95% AUC. MS (ESI) calculated for C₅₃H₇₂N₁₈O₉, m/z 1105.2, found 1106.4.

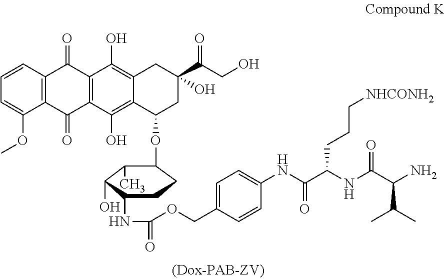

Compound K, 4-((S)-2-((S)-2-amino-3-methylbutanamido)-5-ureidopentanamido)benzyl ((2S,3S,4S,6R)-3-hydroxy-2-methyl-6-(((1 S,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)carbamate, also referred to as Dox-PAB-ZV. This compound is an antracycline based chemotherapeutic that is linked to a carbamate that is linked to an enzyme-degradable (cathepsin) linker, wherein the N-terminal amine is used as reactive handle for attachment to polymers, including the star polymers described herein. Compound K could be prepared in a similar manner as was described for the preparation of Compound G except that doxorubicin is used in place of 2BXy.

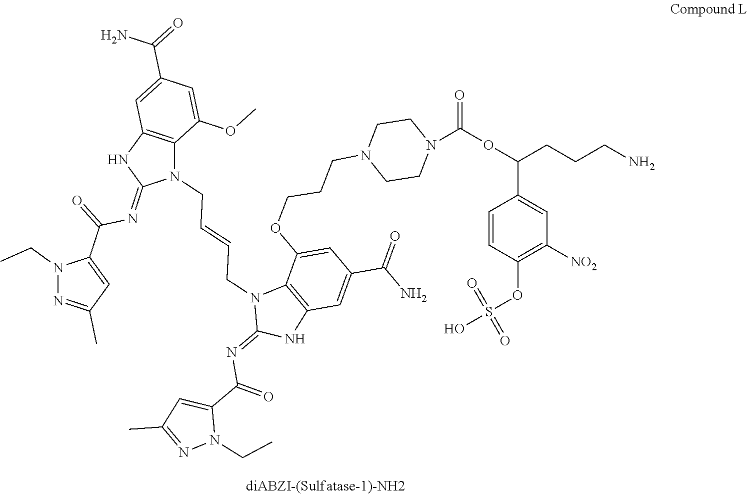 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OC(CCCN)c4ccc(OS(=O)(=O)O)c([N+](=O)[O-])c4)CC3)c21]

Compound L, 4-amino-1-(3-nitro-4-(sulfooxy)phenyl)butyl 4-(3-(((E)-6-carbamoyl-3-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyl)piperazine-1-carboxylate. This compound is a STING agonist linked to an enzyme (sulfatase) degradable linker, reactive handle for attachment to polymers, including the star polymers described herein.

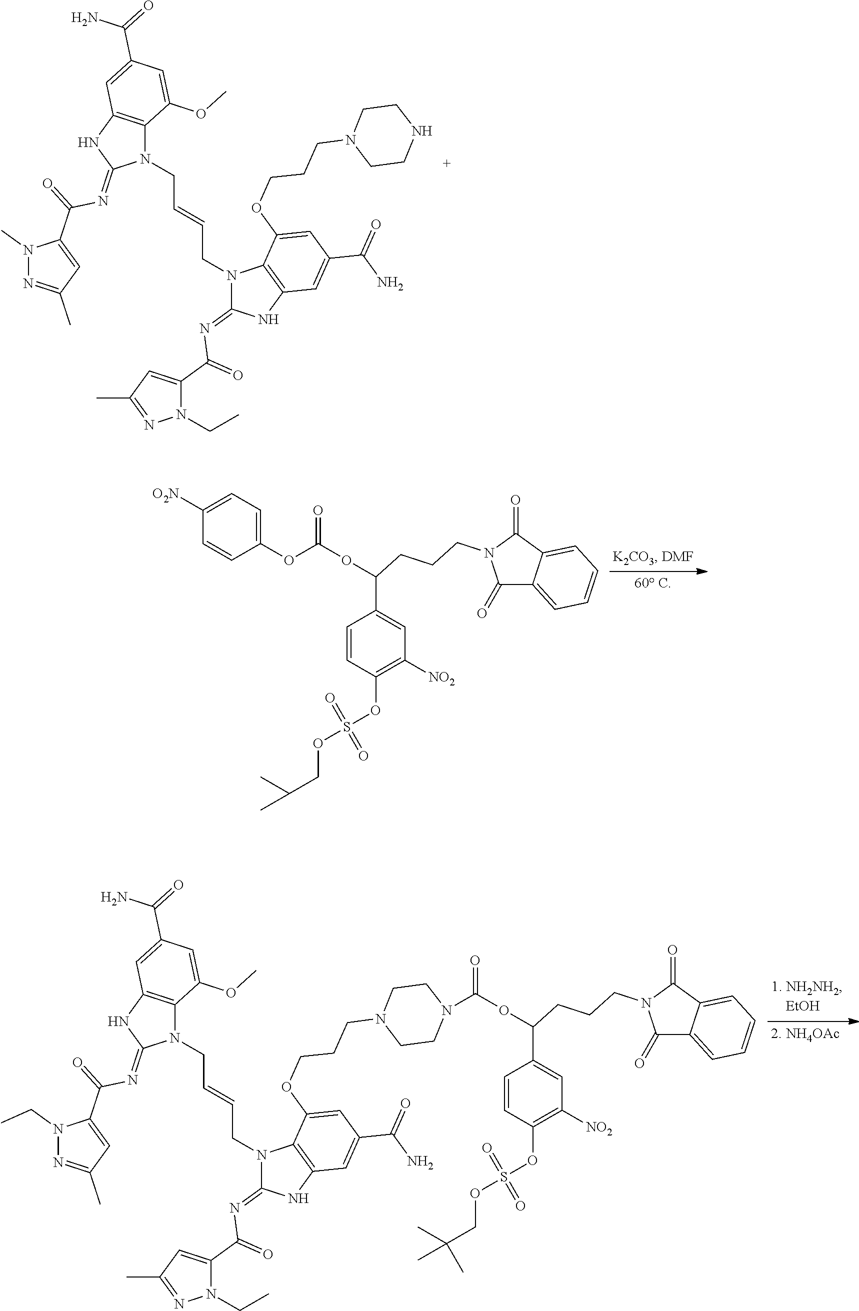 [SMILES: CCn1nc(C)cc1C(=O)/N=c1\[nH]c2cc(C(N)=O)cc(OCCCN3CCNCC3)c2n1C/C=C/Cn1/c(=N/C(=O)c2cc(C)nn2C)[nH]c2cc(C(N)=O)cc(OC)c21 ; CC(C)COS(=O)(=O)Oc1ccc(C(CCCN2C(=O)c3ccccc3C2=O)OC(=O)Oc2ccc([N+](=O)[O-])cc2)cc1[N+](=O)[O-] ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OC(CCCN4C(=O)c5ccccc5C4=O)c4ccc(OS(=O)(=O)OCC(C)(C)C)c([N+](=O)[O-])c4)CC3)c21]

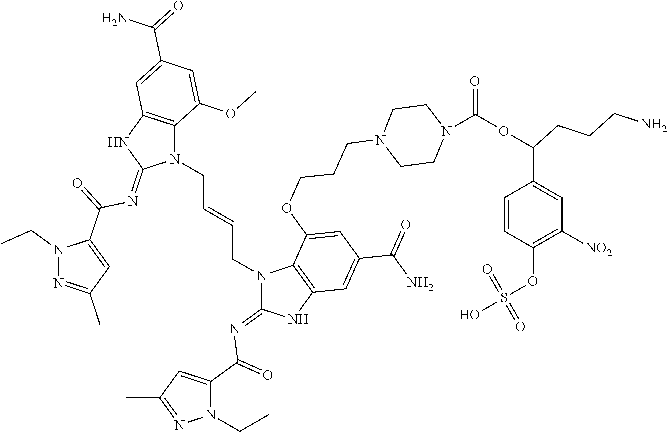 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OC(CCCN)c4ccc(OS(=O)(=O)O)c([N+](=O)[O-])c4)CC3)c21]

Compound L can be prepared in a manner similar to that shown in the scheme above. Reaction of pip-diABZI and PNP activated sulfatase linker-1 in the presence of potassium carbonate will afford the carbamate intermediate. Cleavage of the phthalimide with hydrazine and then cleavage of the neopentyl protecting group of the sulfate with ammonium acetate will afford the desired compound, L.

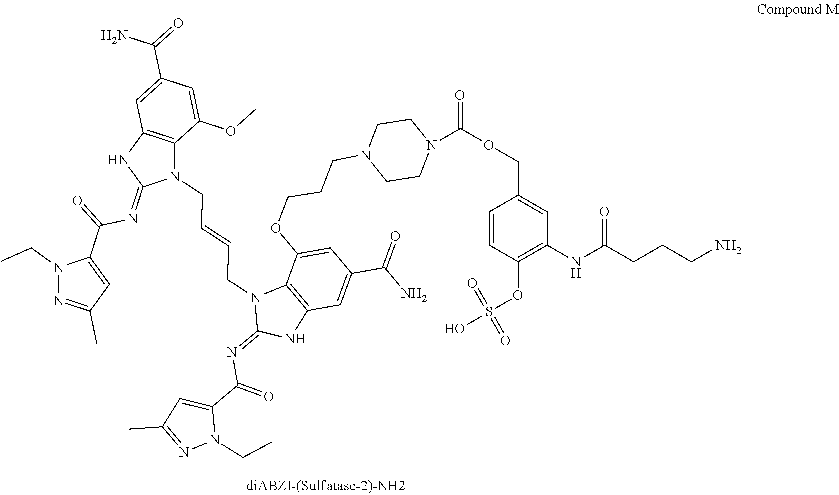 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(OS(=O)(=O)O)c(NC(=O)CCCN)c4)CC3)c21]

Compound M, 4-(4-aminobutanamido)-2-(((4-(3-(((E)-6-carbamoyl-3-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyl)piperazine-1-carbonyl)oxy)methyl)-5-(sulfooxy)benzene-1-ylium. This compound is a STING agonist linked to an enzyme (sulfatase) degradable linker, reactive handle for attachment to polymers, including the star polymers described herein.

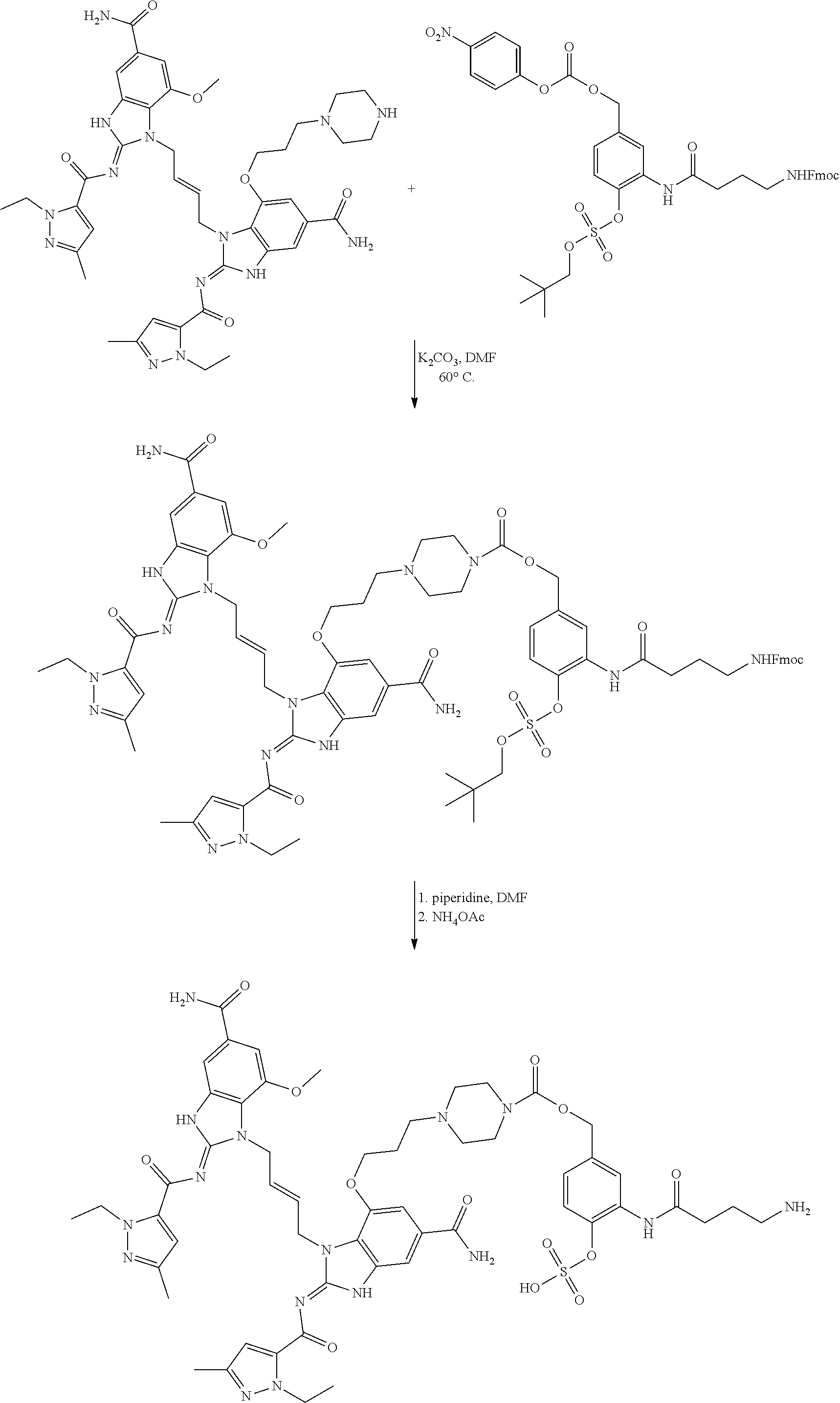

Compound M can be prepared in a manner similar to that shown in the scheme above. Reaction of pip-diABZI and PNP activated sulfatase linker-2 in the presence of potassium carbonate will afford the carbamate intermediate. Removal of the (9H-fluoren-9-yl)methylcarbamate with piperidine in DMF and hydrolysis of the neopentyl sulfate protecting group with ammonium acetate will afford Compound M.

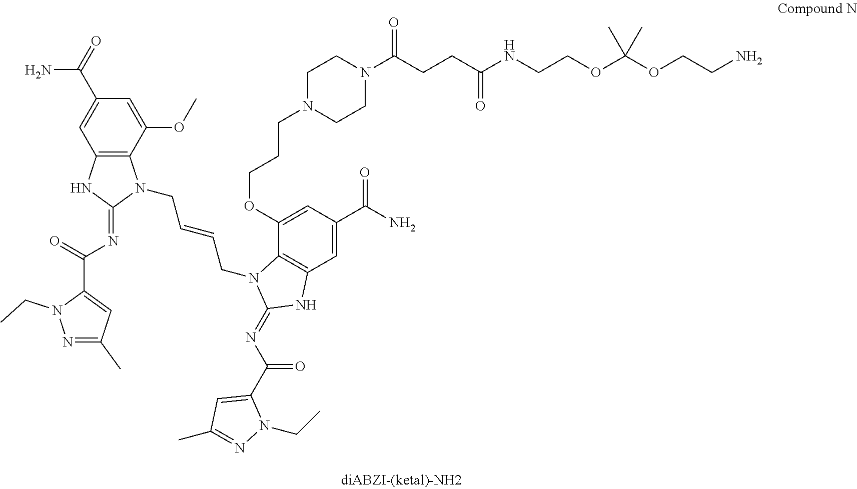 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCC(=O)NCCOC(C)(C)OCCN)CC3)c21]

Compound N, (E)-7-(3-(4-(4-((2-((2-(2-aminoethoxy)propan-2-yl)oxy)ethyl)amino)-4-oxobutanoyl)piperazin-1-yl)propoxy)-1-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazole-5-carboxamide. This compound is a STING agonist linked a pH sensitive ketal, wherein the primary amine provides a reactive handle for attachment to polymers, including the star polymers described herein.

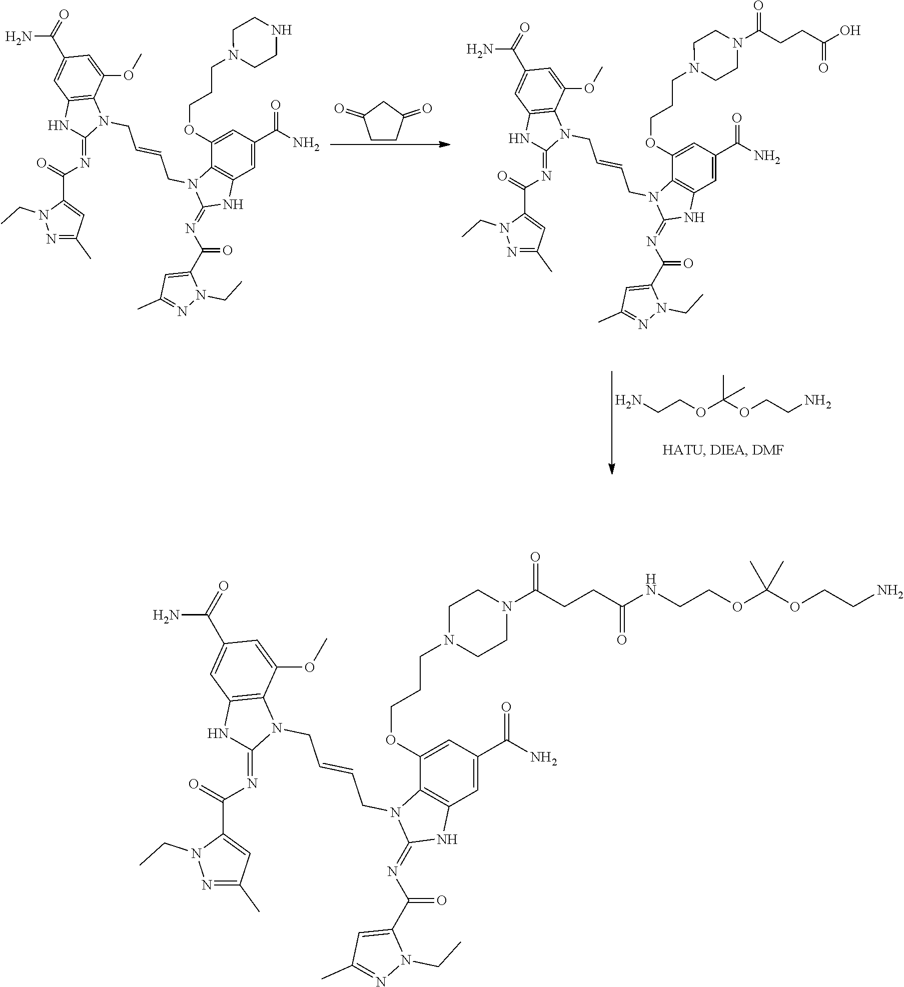 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21 ; O=C1CCC(=O)C1 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCC(=O)O)CC3)c21 ; CC(C)(OCCN)OCCN ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCC(=O)NCCOC(C)(C)OCCN)CC3)c21]

Compound N can be prepared as shown in the scheme above. Condensation of pip-diABZi with succinic anhydride affords the key carboxylic acid intermediate. Subsequent coupling with 2,2′-(propane-2,2-diylbis(oxy))bis(ethan-1-amine) in the presence of HATU and DIEA will afford desired Compound N.

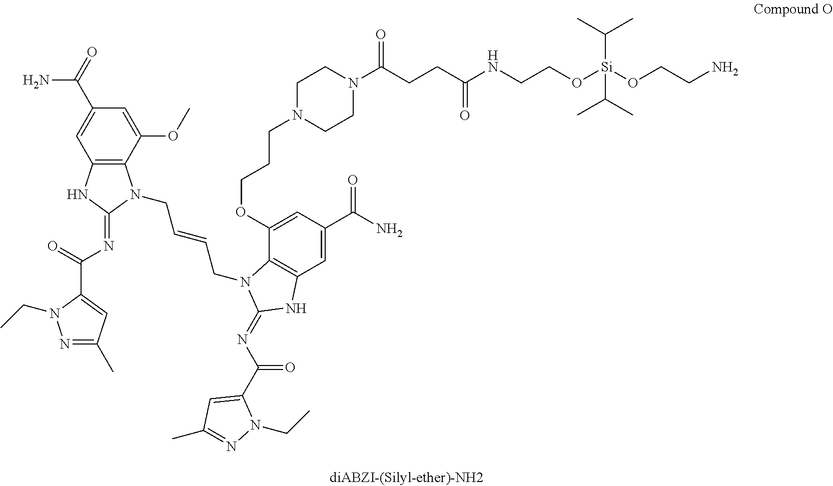 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCC(=O)NCCO[Si](OCCN)(C(C)C)C(C)C)CC3)c21]

Compound O, (E)-7-(3-(4-(1-amino-4,4-diisopropyl-9-oxo-3,5-dioxa-8-aza-4-siladodecan-12-oyl)piperazin-1-yl)propoxy)-1-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazole-5-carboxamide. This compound is a STING agonist linked a pH silyl ether, wherein the primary amine provides a reactive handle for attachment to polymers, including the star polymers described herein.

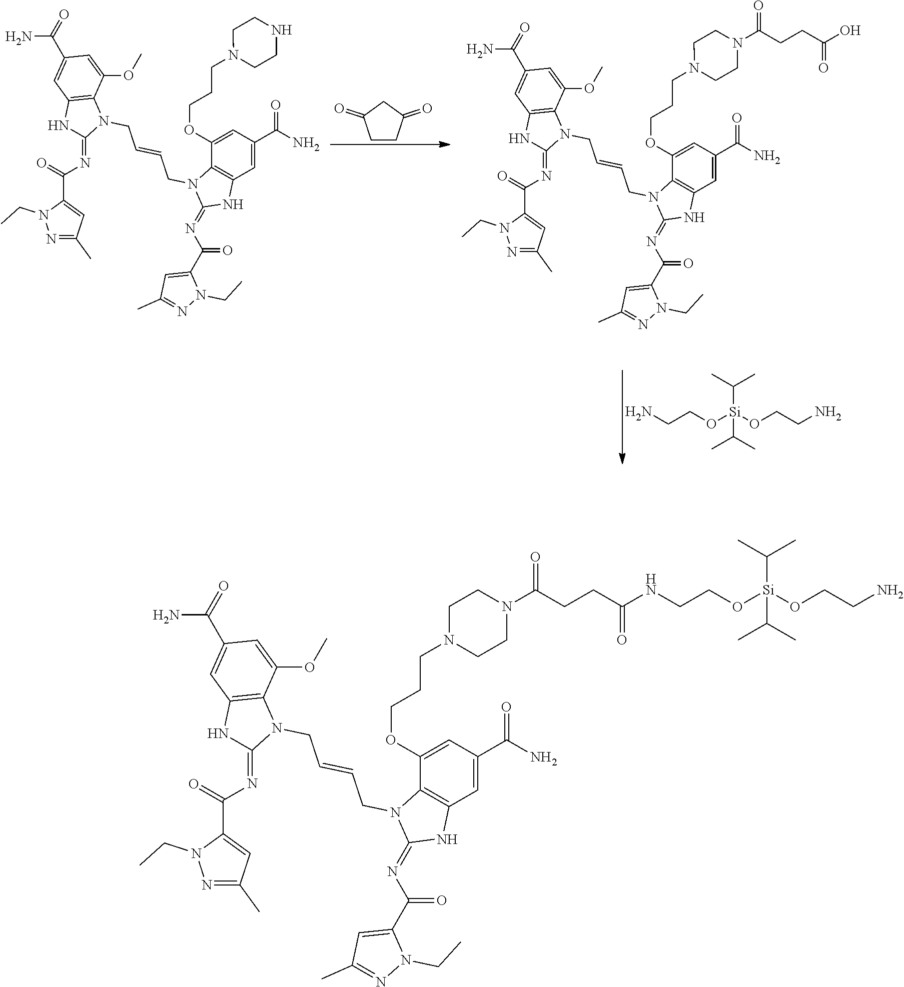 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21 ; O=C1CCC(=O)C1 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCC(=O)O)CC3)c21 ; CC(C)[Si](OCCN)(OCCN)C(C)C ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCC(=O)NCCO[Si](OCCN)(C(C)C)C(C)C)CC3)c21]

Compound O can be prepared in a manner similar to that which was described for Compound N.

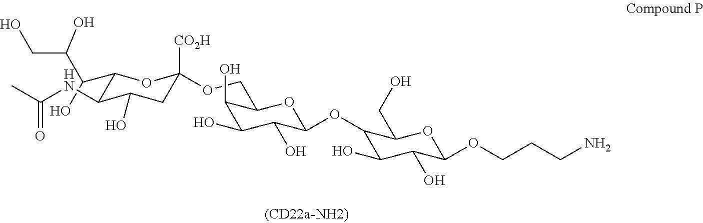 [SMILES: CC(=O)NC1C(O)CC(OCC2OC(OC3C(CO)OC(OCCCN)C(O)C3O)C(O)C(O)C2O)(C(=O)O)OC1C(O)C(O)CO]

Compound P, referred to as CD22a (or CD22 ligand) was synthesized as previously described by WuXi AppTex (Philadelphia, PA) in a similar manner as previously described (Yang, Z.-Q. et al. Carbohydrate Research, 2002, 337 (18), 1605-1613). The primary amine in the structure provides a reactive handle for attachment to polymers, including star polymers described herein. MS (ESI) calculated for C₂₆H₄₆N₂O₁₉, m/z 690.27, found 691.3. ¹H NMR (400 MHz, D₂O) δ 4.51-4.45 (m, 1H), 4.42-4.36 (m, 1H), 4.07-3.91 (m, 3H), 3.90-3.71 (m, 8H), 3.71-3.55 (m, 7H), 3.54-3.45 (m, 2H), 3.35-3.28 (m, 1H), 3.12 (t, J=6.8 Hz, 2H), 2.65 (dd, J=4.5, 12.5 Hz, 1H), 2.04-1.90 (m, 5H), 1.75 (t, J=12.3 Hz, 1H).

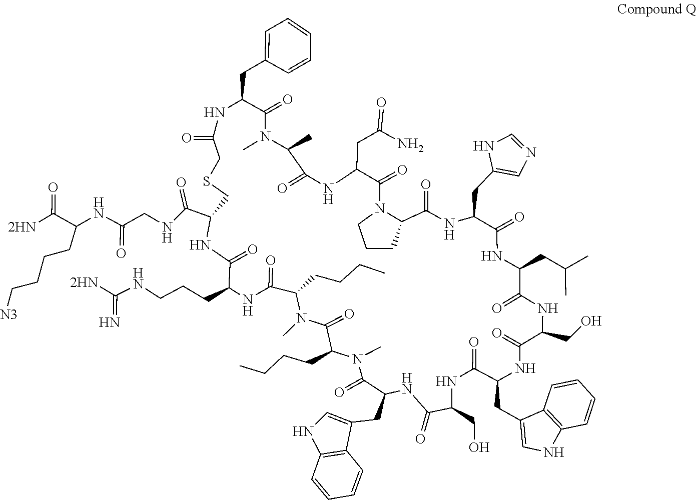

Compound Q. Peptide-57 check-point inhibitor (CPI) with azido-lysine in position 14 was synthesized by Genscript for given amino acid sequence as follows with cyclization at 1(acetic acid) and 15(Cys) locations via thioether linkage: {Aceticacid}F{nme-ALA}NPHLSWSW{NMe-Nle}{NMe-Nle}RCG{Lys(N3)}. Peptide-57 with Gly-NH2 in position 14 was originally reported by Bristol-Myers Squibb Company, US 20140294898 A1, 2014 to act as an inhibitor of human PD-1/PD-L1 interactions. Note: The azide functional group provides a reactive handle for attachment to polymers, including star polymers described herein. MS (ESI) calculated for C₉₅H₁₃₆N₂₈O₂₀S, m/z 2021.02, found 2022.5.

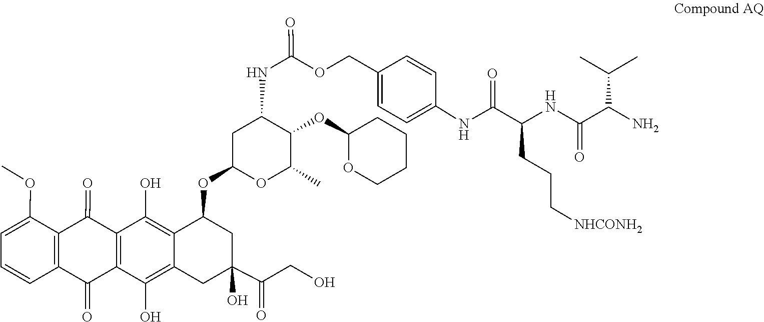

Compound AQ, referred to as Val-Cit-PAB-pirarubicin was synthesized by combining Fmoc-Val-Cit-PAB-PNP (41.5 mg, 0.054 mmol) with pirarubicin (34.0 mg, 0.054 mmol) in DMAC (1.7 mL). The solution was stirred for 16 hours at room temperature and the desired product, Fmoc-Val-Cit-PAB-pirarubicin, was precipitated by the addition of cold diethyl ether (35 mL). The desired product was collected by vacuum filtration (63 mg, 100% yield). This Fmoc-protected intermediate was used in the next synthetic step without additional purification or characterization. The Fmoc-protected intermediate (63 mg, 0.054 mmol) was dissolved in DMF (2.1 mL). Piperidine (210 μL) was added, the reaction was stirred for 2 minutes, and then the product was precipitated by the addition of cold diethyl ether (40 mL). The desired product was collected by vacuum filtration; the solid was washed with additional, cold diethyl ether (10 mL) and dried to afford 37 mg (72% yield) of a pure (95% AUC at 220 nm) solid. MS (EI) calculated for C₅₁H₆₄N₆O₁₇, m/z 1032.43, found, 1033.5 (M+H)+.

Other Peptide Linkers

Additional peptide linkers were synthesized by standard solid-phase peptide synthesis (SPPS) by Genscript (Piscataway, NJ), as summarized in the Table A below. The peptide linker sequence is the peptide that was synthesized by SPPS, cleaved from the resin and purified by HPLC. Drug molecules were coupled to the “peptide linker sequences” using HATU coupling either directly or via a PAB linker, followed by simultaneous Boc and tBu deprotection to yield different “linker-drug conjugates.” Boc=tert-butoxycarbonyl; tBu=tert-butyl; A′=beta-alanine; V=valine; Z=citrulline; S=serine; P=proline; K=lysine; Ac=acetyl; B=amino-butyric acid; nL=norleucine. Note: The N-terminus of beta-alanine is a reactive handle for linking the linker-drug conjugate either directly (or indirectly via a linker) to reactive monomers distributed along the backbone of polymer arms.

TABLE A
Peptide-based linkers.

| Cmpd | | | |
|---|---|---|---|
| # | Peptide linker sequence | MW | E.g., linker-drug conjugate |
| R | Boc-A′VZ | 445.42 | A′VZ-Drug |
| S | Boc-A′S(tBu)PVZ | 685.59 | A′SPVZ-Drug |
| T | Boc-A′S(tBu)K(Ac) VZ | 758.29 | A′SK(Ac)VZ-Drug |
| U | Boc-A′S(tBu)K(Boc) VZ | 816.70 | A′SKVZ-Drug |
| V | Boc-A′VK(Ac) | 485.45 | A′VK(Ac)-Drug |
| W | Boc-A′VK(Boc) | 516.40 | A′VK-Drug |
| X | Boc-A′VB | 373.35 | A′VB-Drug |
| Y | Boc-A′S(tBu)PVB | 613.13 | A′SPVB-Drug |
| Z | Boc-A′S(tBu)K(Ac) VB | 686.18 | A′SK(Ac) VB-Drug |
| AA | Boc-A′S(tBu)K(Boc)VB | 743.71 | A′SKVB-Drug |
| AB | Boc-A′S(tBu)K(Ac)S(tBu)B | 730.11 | A′SK(Ac)SB-Drug |
| AC | Boc-A′S(tBu)K(Boc)S(tBu)B | 788.63 | A′SKSB-Drug |
| AD | Boc-A′VnL | 401.41 | A′VnL-Drug |
| AE | Boc-A′S(tBu) PVnL | 641.63 | A′SPVnL-Drug |
| AF | Boc-A′S(tBu)K(Ac)S(tBu)nL | 758.92 | A′SK(Ac) SnL-Drug |
| AG | Boc-A′S(tBu)K(Boc)S(tBu)nL | 815.80 | A′SKSnL-Drug |

Note:
Drug molecules were linked to peptide-based linkers either directly or via a carbamate (e.g., PAB) linker.

### Example 2—Synthesis of Monomers, Initiators, CTAs and Amplifying Linkers

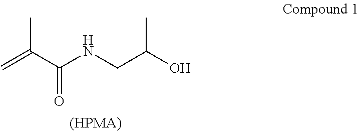

Compound 1. N-(2-Hydroxypropyl)methacrylamide (HPMA) is an example of a hydrophilic monomer (B), specifically methacrylamide-based monomer. HPMA was synthesized by reacting 1-amino-2-propanol with methacryloyl chloride. To a 1 L round-bottom flask equipped with magnetic stir bar, 1-amino-2-propanol (60.0 mL, 0.777 mol), sodium bicarbonate (60.27 g, 0.717 mol), 4-methoxyphenol (1.00 g, 8.1 mmol), and 200 mL of dichloromethane (DCM) were added. The flask was immersed in an acetone-dry ice bath for 15 min with vigorous stirring. Methacryloyl chloride (70.0 mL, 0.723 mol) dissolved in 80 mL of DCM was added dropwise under Ar (g) over 3 h. The reaction was allowed to proceed at r.t. for another 30 min. After removing the salt, crude product was purified via flash chromatography using a silica gel column (Biotage SNAP ultra 100 g) and gradient eluent DCM/MeOH with MeOH increased from 0 to 10% (v/v). The solid thus obtained after solvent removal was then recrystallized from acetone to yield HPMA as white crystal (22.4 g, 21.6%). ESI-MS: m/z=144.1 (M+H)+.

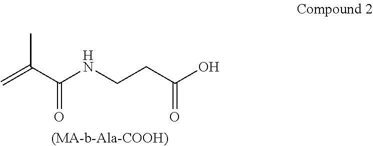

Compound 2. N-methacryloyl-3-aminopropanoic acid (MA-b-Ala-COOH) was synthesized by reacting beta-alanine (15.07 g, 169.1 mmol) to methacrylic anhydride (28.6 g, 185.5 mmol) in the presence of 4-methoxyphenol (0.218 g, 1.76 mmol) in a 100 mL round bottom flask at r.t. over weekend. The mixture was purified by flash chromatography using a silica gel column (Biotage SNAP ultra 100 g) and gradient eluent DCM/MeOH with MeOH increased from 0 to 10% (v/v). After combining fractions and removing solvent, product was recrystallized from EtOAc/Et₂O (1/1 v/v) at −20° C., yielding a white crystal (15.22 g, 57.3% yield). ¹H NMR (DMSO-d₆, ppm): δ12.25 (s, 1H), 7.96 (s, 1H), 5.63 (s, 1H), 5.32 (s, 1H), 3.30 (q, 2H), 2.43 (t, 3H), 1.81 (s, 3H).

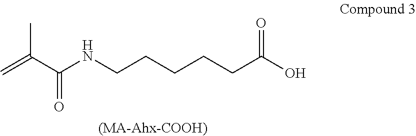

Compound 3. N-methacryloyl-6-aminohexanoic acid (MA-Ahx-COOH) was synthesized by reacting 6-aminohexonic acid (0.252 g, 1.92 mmol) to methacrylic anhydride (0.582 g, 3.78 mmol) in the presence of 4-methoxyphenol (4 mg, 0.03 mmol) in a 20 mL scintillation vial at r.t. overnight. The product was purified by recrystallizing from EtOAc/Et₂O (1/1 v/v) at −20° C., yielding a white crystal. ¹H NMR (D₂O, ppm): δ1.32 (—CH₂CH₂CH₂COOH), 1.52 (—CH₂CH₂COOH), 1.58 (—NHCH₂CH₂—), 1.88 (—CH₃), 2.35 (—CH₂COOH), 3.22 (—NHCH₂—), 5.35 and 5.61 (CH₂═CH).

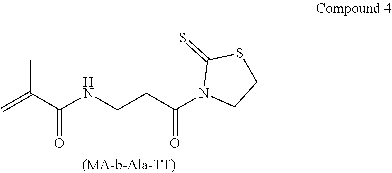

Compound 4. N-Methacryloyl-3-aminopropanoic acid-thiazolidine-2-thione (MA-b-Ala-TT) is an example of a reactive monomer (E). MA-b-Ala-TT was prepared by reacting Compound 2, MA-b-Ala-COOH (5.05 g, 32 mmol), 1,3-thiazolidine-2-thione (4.39 g, 37 mmol), EDC (8.09 g, 42 mmol), DMAP (0.45 g, 4 mmol), and 100 mL DCM were mixed in a 250 mL round bottom flask. It was allowed to react 1 h before the product was washed by 1 M HCl (2×) and DI water (1×). Upon solvent removal, yellow solid product was collected (7.15 g, 86.1% yield). ¹H NMR (DMSO-d₆, ppm): δ7.96 (s, 1H), 5.63 (s, 1H), 5.32 (s, 1H), 4.91 (t, 2H), 3.32 (m, 6H), 1.78 (s, 3H). ESI-MS: m/z=281.0 (M+Na)+.

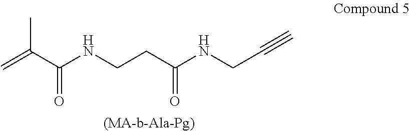

Compound 5. MA-b-Ala-Pg is an example of a reactive monomer (E). MA-b-Ala-Pg was prepared by reacting Compound 4, MA-b-Ala-TT (2.067 g, 8.01 mmol) to propargylamine (0.473 g, 8.588 mmol) in the presence of triethylamine (0.799 g, 7.892 mmol) in a 22 mL DCM for 1.5 h at r.t. The product was purified by recrystallizing from acetone at −20° C. for two times, yielding a white crystal (1.08 g, 69.5% yield). ¹H NMR (DMSO-d₆, ppm): δ8.35 (t, 1H), 7.96 (t, 3H), 5.62 (s, 1H), 5.31 (s, 1H), 3.83 (d, 2H), 3.28 (q, 2H), 3.12 (s, 1H), 2.27 (t, 2H), 1.78 (s, 3H).

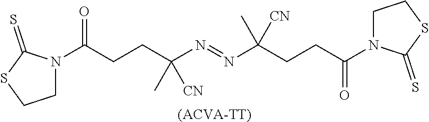

Compound 6. 2-[1-Cyano-1-methyl-4-oxo-4-(2-thioxo-thiazolidin-3-yl)-butylazo]-2-methyl-5-oxo-5-(2-thioxothiazolidin-3-yl)-pentanenitrile, “ACVA-TT,” is a TT-functionalized initiator, which can be used to incorporate TT, activated carbonyl groups, to the ends of the polymer arms (A) during polymerization or capping (i.e., by replacing the CTA of a living polymer). ACVA-TT was synthesized by activating the carboxylic acids in 4,4′-azobis(4-cyanovaleric acid) (ACVA-COOH) with 2-thiazoline-2-thiol via N,N′-diisopropylcarbodiimide (DIC) coupling reaction. To a 20 mL scintillation vial, ACVA-COOH (501.5 mg, 1.79 mmol), 2-thiazoline-2-thiol (411.8 mg, 3.46 mmol), 4-(dimethylamino)pyridine (DMAP, 10.6 mg, 0.087 mmol), and 15 mL of DCM were added. The mixture was stirred vigorously in an ice-bath for 15 min before DIC (497.1 mg, 3.94 mmol) was added. The mixture was allowed to slowly warm up to r.t. and react for another 15 min before it was washed with saturated solution of NaHCO₃ (20 mL×2), DI water (20 mL×1). The organic phase was then dried over MgSO₄ and evaporated to yield dry product, which was purified by recrystallizing from DCM/Et₂O at −20° C. After decanting the solvent, bright yellow powder was obtained (658.3 mg, 76.2%). ESI-MS: m/z=483.1 (M+H)+.

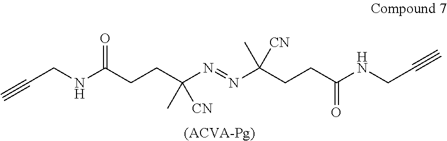

Compound 7. 4-Cyano-4-(1-cyano-3-ethynylcarbamoyl-1-methylpropylazo)-N-ethynyl-4-methylbutyramide, “ACVA-Pg,” is a propargyl functionalized initiator, which can be used to incorporate Pg groups to the ends of polymer arms (A) during polymerization or capping (i.e., by replacing the CTA of a living polymer). ACVA-Pg was synthesized by reacting ACVA-TT with 3-amino-1-propyne. To a 20 mL scintillation vial, ACVA-TT (329.7 mg, 0.684 mmol), 3-amino-1-propyne (99.76 mg, 1.81 mmol), and 10 mL of DCM were added. Triethylamine (253 μL, 1.82 mmol) was then added to the mixture. The reaction was allowed to proceed for another 1 h at r.t. before solvent was removed. The crude product was purified via flash chromatography using a C-18 column (Biotage SNAP Ultra C-18) and a gradient of 0-95% acetonitrile in H₂O (0.05% TFA) over 20 CVs (product eluted at 30-40% acetonitrile). Fractions containing pure product were pool and dried to yield white solid (190.3 mg, 78.5%). ESI-MS: m/z=355.2 (M+H)⁺.

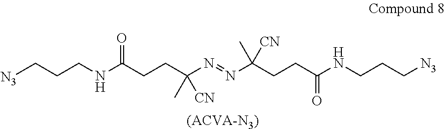 [SMILES: CC(C#N)(CCC(=O)NCCCN=[N+]=[N-])/N=N/C(C)(C#N)CCC(=O)NCCCN=[N+]=[N-] ; "(ACVA-N3)"]

Compound 8. ACVA-N3 is an azide-functionalized initiator, which can be used to incorporate azide groups to the ends of polymer arms (A) during polymerization or capping (i.e., by replacing the CTA of a living polymer). ACVA-N3 was synthesized by reacting ACVA with 1-azido-3-propanamine. To a 20 mL scintillation vial, ACVA (250.0 mg, 0.893 mmol), 1-azido-3-propanamine (187.7 mg, 1.87 mmol), and 5 mL of DCM were added. EDC (375.2, 1.96 mmol) was then added to the mixture over 20 min. The reaction was allowed to proceed for another 1 h at r.t. before solvent was removed. The crude product was recrystallized from EtOAc/Et₂O to yield white solid (130.0 mg, 32.8%). ESI-MS: m/z=445.2 (M+H)⁺.

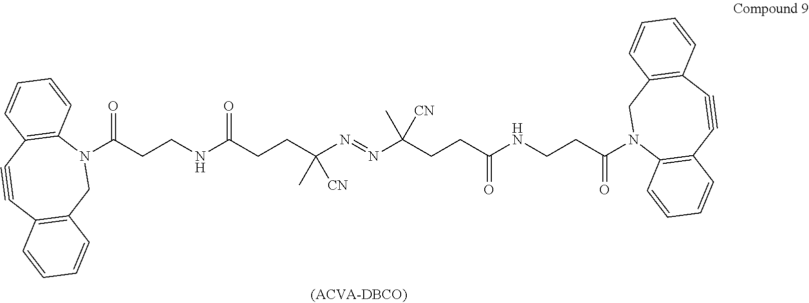 [SMILES: CC(C#N)(CCC(=O)NCCC(=O)N1Cc2ccccc2C#Cc2ccccc21)/N=N/C(C)(C#N)CCC(=O)NCCC(=O)N1Cc2ccccc2C#Cc2ccccc21]

Compound 9. ACVA-DBCO is a DBCO functionalized initiator, which is an example of a strained-alkyne functionalized initiator that can be used to incorporate strained-alkynes to the ends of polymer arms (A) during polymerization or capping (i.e., by replacing the CTA of a living polymer). ACVA-DBCO was synthesized by reacting ACVA-TT with DBCO-amine. To a 20 mL scintillation vial, ACVA-TT (201.4 mg, 0.417 mmol), DBCO-amine (229.2 mg, 0.829 mmol), and 1 mL of DCM were added. The reaction was allowed to proceed for 1 h at r.t. before solvent was removed. The crude product was purified by flash chromatography using a silica gel column and a gradient of 0-5% MeOH in DCM to yield white solid (314.4 mg, 95.1%). ESI-MS: m/z=797.3 (M+H)⁺.

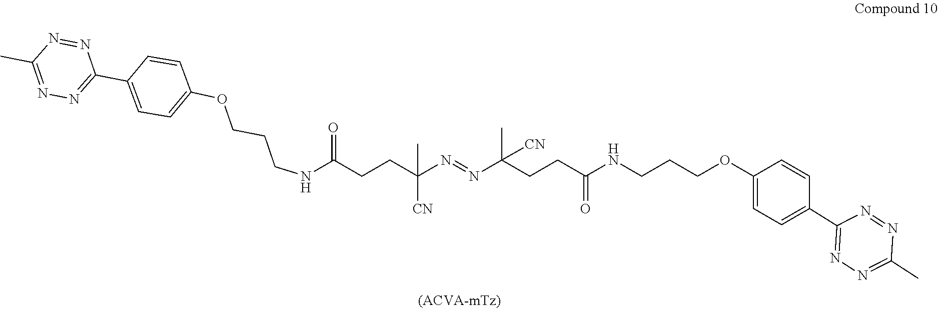

Compound 10. ACVA-mTz is a methyletrazinme functionalized initiator, which is an example of a tetrazine functionalized initiator that can be used to incorporate tetrazines to the ends of polymer arms (A) during polymerization or capping (i.e., by replacing the CTA of a living polymer). ACVA-mTz was synthesized by reacting ACVA-TT with methyltetrazine propylamine (mTz-amine) using triethylamine as the catalyst. To a 20 mL scintillation vial, ACVA-TT (162.2 mg, 0.427 mmol), mTz-amine (120.8 mg, 0.492 mmol), trimethylamine (124.9 μL, 0.896 mmol), and 4 mL of DCM were added. The reaction was allowed to proceed for 1 h at r.t. before solvent was removed. The crude product was purified by flash chromatography using a C-18 column to yield white solid (166.8 mg, 53.2%). ESI-MS: m/z=735.3 (M+H)⁺.

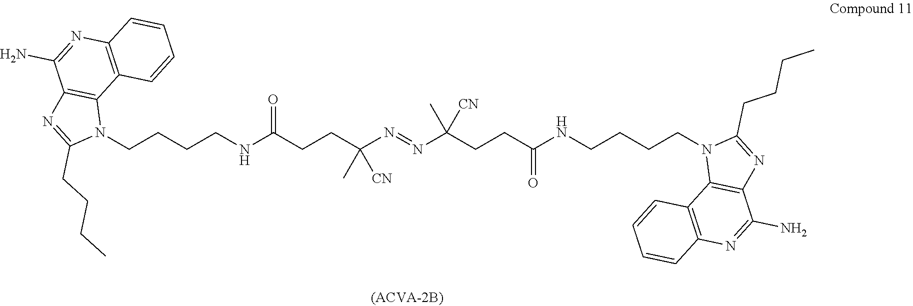 [SMILES: CCCCc1nc2c(N)nc3ccccc3c2n1CCCCNC(=O)CCC(C)(C#N)/N=N/C(C)(C#N)CCC(=O)NCCCCn1c(CCCC)nc2c(N)nc3ccccc3c21]

Compound 11. ACVA-2B is a 2B functionalized initiator, which is an example of a TLR-7/8a (and more broadly drug, (D)) functionalized initiator that can be used to incorporate TLR-7/8a to the ends of polymer arms (A) during polymerization or capping (i.e., by replacing the CTA of a living polymer). ACVA-2B was synthesized by reacting ACVA-TT with 2B. To a 20 mL scintillation vial, ACVA-TT (200.5 mg, 0.415 mmol), 2B, Compound B, (258.7 mg, 0.831 mmol), and 1 mL of DCM were added. The reaction was allowed to proceed for 1 h at r.t. before solvent was removed. The crude product was purified on a preparatory HPLC system using a gradient of 27-47% acetonitrile/H₂O (0.05% TFA) over 12 minutes on an Agilent Prep C-18 column, 50×100 mm, 5 μm. The product fractions were pooled and lyophilized yielding white solid (214.7 mg, 59.5%). ESI-MS: m/z=868.2 (M+H)⁺.

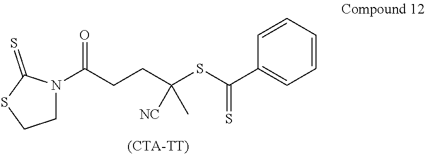

Compound 12. Dithiobenzoic acid 1-cyano-1-methyl-4-oxo-4-(2-thioxothiazolidin-3-yl)butyl ester, “CTA-TT,” is a TT-functionalized chain transfer agent (CTA), which can be used to introduce TT functional groups onto polymer arms (A) during polymerization. CTA-TT was synthesized by activating the carboxylic acid in 4-cyano-4-(phenylcarbonothioylthio)pentanoic acid (CTA-COOH) with 2-thiazoline-2-thiol. To a 20 mL scintillation vial, CTA-COOH (499.8 mg, 1.79 mmol), 2-thiazoline-2-thiol (196.5 mg, 1.65 mmol), DMAP (8 mg, 0.065 mmol), and 10 mL of DCM were added. The mixture was stirred vigorously in an ice-bath for 15 min before EDC (446.2 mg, 2.33 mmol) was added. The mixture was allowed to slowly warm up to r.t. and react for another 15 min before it was washed with saturated solution of NaHCO₃ (10 mL×2) and DI water (10 mL×2). The organic phase was then dried over MgSO₄ and evaporated to yield dry product, which was purified on a preparatory HPLC system using a gradient of 58-78% acetonitrile/H₂O (0.05% TFA) over 12 minutes on an Agilent Prep C-18 column, 30×100 mm, 5 μm. The product eluted at 6.5 minutes and the product fractions were pooled and lyophilized yielding red viscous liquid (400.0 mg, 63.8%). ESI-MS: m/z=381.0 (M+H)⁺.

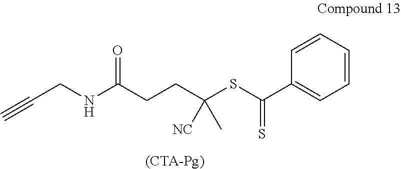 [SMILES: C#CCNC(=O)CCC(C)(C#N)SC(=S)c1ccccc1 ; "(CTA-Pg)"]

Compound 13. Dithiobenzoic acid 1-cyano-1-methyl-3-prop-2-ynylcarbamoylpropyl ester “CTA-Pg,” is a Pg-functionalized CTA, which can be used to introduce Pg functional groups onto polymer arms (A) during polymerization. CTA-Pg was synthesized by reacting CTA-COOH with 3-amino-1-propyne. To a 20 mL scintillation vial, CTA-COOH (100.0 mg, 0.358 mmol), 3-amino-1-propyne (21.69 mg, 0.394 mmol), HATU (272.2 mg, 0.716 mmol), DIEA (185.0 mg, 1.432 mmol), and 4 mL of DMF were added. The mixture was stirred at r.t. for 2 h before it was washed with saturated solution of NaHCO₃ (10 mL×2) and brine (10 mL×1). The organic phase was then dried over MgSO₄ and evaporated to yield dry product, which was purified on a preparatory HPLC system using a gradient of 40-70% acetonitrile/H₂O (0.05% TFA) over 12 minutes on an Agilent Prep C-18 column, 50×100 mm, 5 μm. The product eluted at 8.5 minutes and the product fractions were pooled and lyophilized yielding red viscous liquid (54.0 mg, 47.7%). ESI-MS: m/z=317.1 (M+H)⁺.

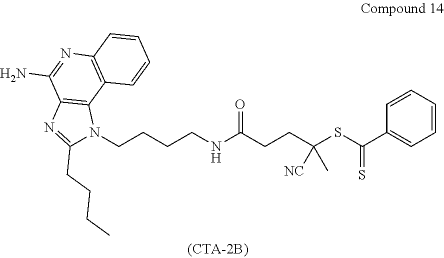 [SMILES: CCCCc1nc2c(N)nc3ccccc3c2n1CCCCNC(=O)CCC(C)(C#N)SC(=S)c1ccccc1]

Compound 14. CTA-2B, is a 2B-functionalized CTA, which is an example of a TLR-7/8a or more broadly (drug) functionalized CTA that can be used to introduce TLR-7/8a functional groups onto polymer arms (A) during polymerization. CTA-2B was synthesized by reacting CTA-NHS with 2B, Compound B. To a 20 mL scintillation vial, CTA-NHS (200.6 mg, 0.533 mmol), 2B (165.6 mg, 0.532 mmol), and 3 mL of DCM were added. The reaction was allowed to proceed for 40 min at r.t. before it was washed with DI water (10 mL×2). The organic phase was then dried over MgSO₄ and evaporated to yield dry product as dark red solid (250 mg, 82.1%). ESI-MS: m/z=573.7 (M+H)⁺.

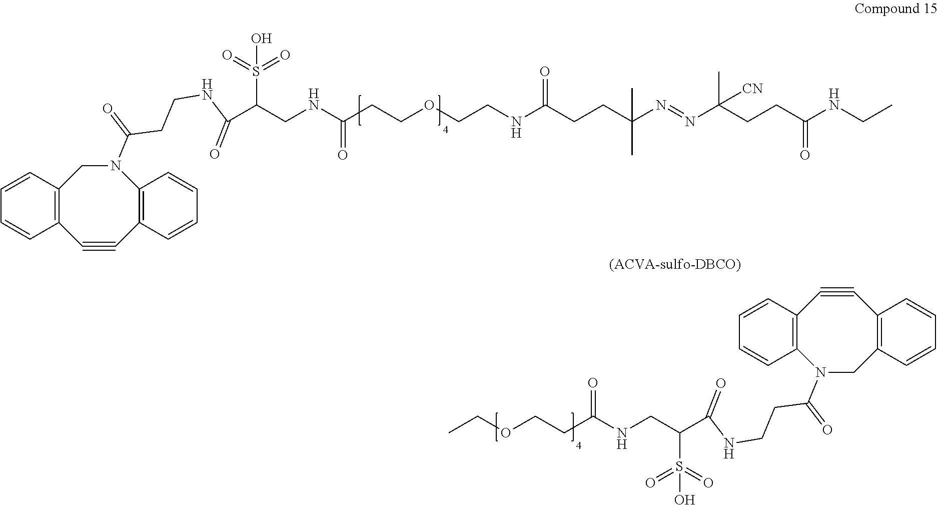

Compound 15. ACVA-sulfo-DBCO, is an example of a water-soluble strained-alkyne functionalized initiator, which can be used to introduce water-soluble strained alkynes onto the ends of polymer arms (A) during polymerization or capping. ACVA-sulfo-DBCO was synthesized by reacting ACVA-TT with sulfo-DBCO-PEG4-amine. ACVA-TT (32.2 mg, 0.067 mmol) and sulfo-DBCO-PEG4-amine (100.0 mg, 0.148 mmol) were dissolved in 2 mL of DCM before triethylamine (30.0 mg, 0.30 mol) was added. The reaction was allowed to proceed for 1 h at r.t. The crude product was purified by flash chromatography using a silica gel column (Biotage SNAP ultra 25 g), and a gradient of 5-20% MeOH in DCM over 20 CVs (product eluted at 18% MeOH). Fractions containing pure product were combined and dried to yield final product (115.2 mg, 84.1%). ESI-MS: m/z=797.4 [(M+2H)]²⁺.

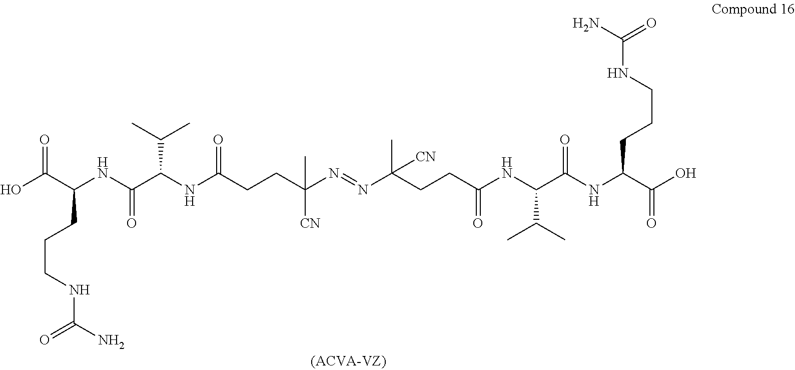 [SMILES: CC(C)[C@H](NC(=O)CCC(C)(C#N)/N=N/C(C)(C#N)CCC(=O)N[C@H](C(=O)N[C@@H](CCCNC(N)=O)C(=O)O)C(C)C)C(=O)N[C@@H](CCCNC(N)=O)C(=O)O]

Compound 16. ACVA-VZ is an example of a degradable peptide-functionalized initiator, which can be used to introduce degradable peptides onto the ends of polymer arms (A) during polymerization or capping. ACVA-VZ was synthesized by reacting ACVA-TT with valine-citrulline (VZ) peptide. ACVA-TT (62.3 mg, 0.13 mmol) and VZ (100.0 mg, 0.36 mmol) were dissolved in 1 mL of DMSO before triethylamine (44.2 mg, 0.44 mmol) was added. The reaction was allowed to proceed for 2 h at r.t. The crude product was purified on a preparatory HPLC system using a gradient of 16-31% acetonitrile/H₂O (0.05% TFA) over 12 minutes on an Agilent Prep C-18 column, 50×100 mm, 5 μm. The product fractions were pooled and lyophilized to yield final product (91.5 mg, 89.1%).

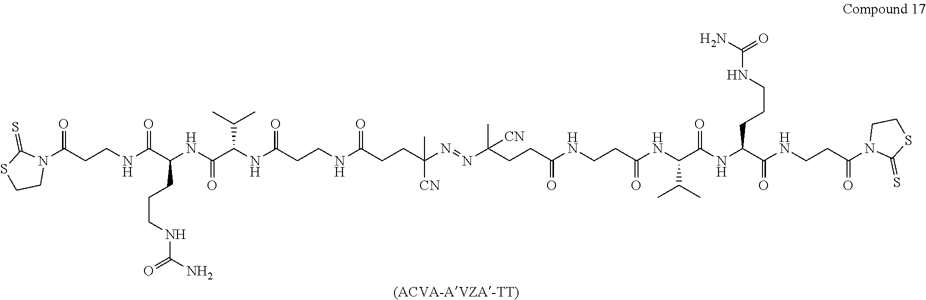 [SMILES: CC(C)[C@H](NC(=O)CCNC(=O)CCC(C)(C#N)/N=N/C(C)(C#N)CCC(=O)NCCC(=O)N[C@H](C(=O)N[C@@H](CCCNC(N)=O)C(=O)NCCC(=O)N1CCSC1=S)C(C)C)C(=O)N[C@@H](CCCNC(N)=O)C(=O)NCCC(=O)N1CCSC1=S]

Compound 17. ACVA-A′VZA′-TT is an example of a TT-activated degradable peptide-functionalized initiator, which can be used to introduce TT-activated degradable peptides onto the ends of polymer arms (A) during polymerization or capping. ACVA-A′VZA′-TT was synthesized by reacting ACVA-TT with p-alanine-valine-citrulline-p-alanine (A′VZA′) peptide to afford ACVA-A′VZA′, followed by activating the carboxylic acids with 2-thiazoline-2-thiol. ACVA-TT (26.0 mg, 0.054 mmol) and A′VZA′ (50.0 mg, 0.12 mmol) were dissolved in 1.5 mL of DMSO before triethylamine (48.6 mg, 0.48 mmol) was added. The reaction was allowed to proceed for 2 h at r.t. The crude product was purified on a preparatory HPLC system using a gradient of 5-40% acetonitrile/H₂O (0.05% TFA) over 12 minutes on an Agilent Prep C-18 column, 30×100 mm, 5 μm. Fractions containing targeted product were pooled and lyophilized to yield ACVA-A′VZA′ (53.0 mg, 91.1%). ACVA-A′VZA′ (10.0 mg, 0.0093 mmol) and 2-thiazoline-2-thiol (2.8 mg, 0.02 mmol) were dissolved in DMF before 1-[bis(dimethylamino)methylene]-1H-1,2,3-triazolo[4,5-b]pyridinium 3-oxide hexafluorophosphate (HATU) (7.1 mg, 0.019 mmol) and triethylamine (3.8 mg, 0.037 mmol) were added. The reaction was allowed to proceed for 2 h at r.t. before the crude product was purified on a preparatory HPLC system to yield final product ACVA-A′VZA′-TT.

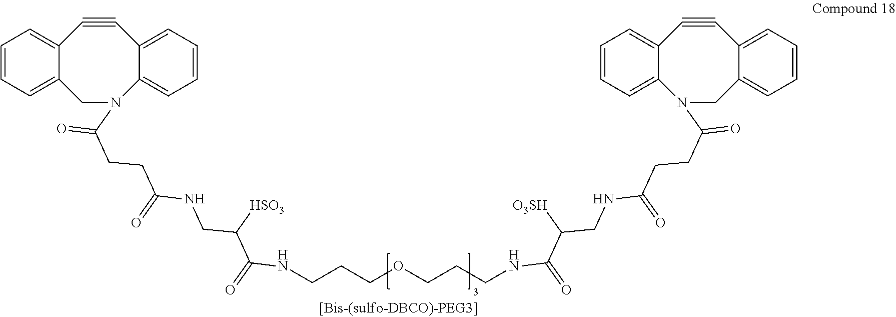

Compound 18. Bis(sulfo-DBCO)-PEG3 is a homo-bifunctional linker that was synthesized by reacting NH2-PEG3-NH2 with sulfo-DBCO-tetrafluorophenyl (TFP) ester. NH2-PEG3-NH2 (8.3 mg, 0.037 mmol) and sulfo-DBCO-TFP ester (50.0 mg, 0.083 mmol) were dissolved in 1 mL of DCM before triethylamine (16.0 mg, 0.16 mmol) was added. The reaction was allowed to proceed for 1 h at r.t. The crude product was purified by flash chromatography using a silica gel column and a gradient of 10-20% MeOH in DCM (product eluted at 10% MeOH). Fractions containing pure product were combined and dried to yield final product (45.2 mg, 109.6%). ESI-MS: m/z=1097 (M+H)⁺.

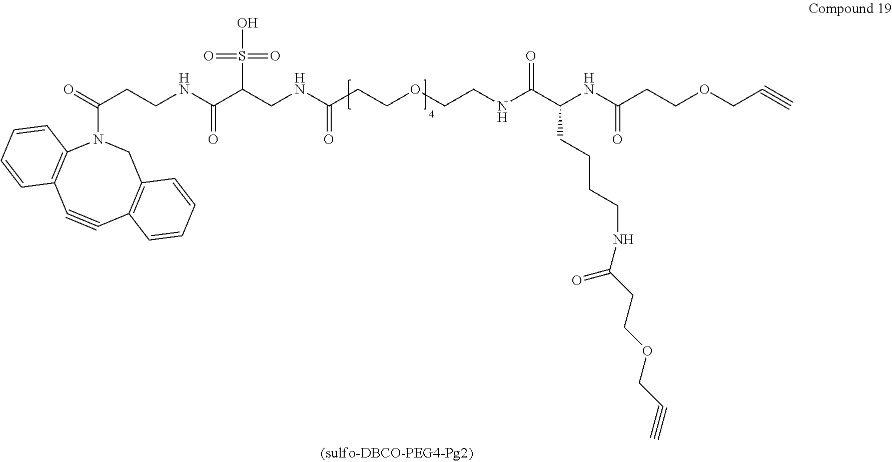

Compound 19. Amplifying linker sulfo-DBCO-PEG4-Pg2 was synthesized in three steps using propargyl NHS ester, amino-PEG4-sulfo-DBCO, and Boc-Lys(Boc)-OH as the starting materials. Boc-Lys(Boc)-OH (1.0 g, 2.89 mmol, 1 eq), TT (378.5 mg, 3.18 mmol, 1.1 eq) and EDC (719.4 mg, 3.75 mmol, 1.3 eq) were dissolved in 10 mL of DCM. DMAP (35.3 mg, 0.29 mmol, 0.1 eq) as a 100 mg/mL stock solution in DCM was added. The solution turned bright yellow and was allowed to react at room temperature for 1 h. DCM was removed under vacuum before the crude product was dissolved in 700 μL of DMSO and precipitated in 50 mL of 0.1 M HCl (twice) and DI water. The intermediate, Boc-Lys(Boc)-TT was provided as a yellow solid.

Boc-Lys(Boc)-TT (238.1 mg, 0.53 mmol, 2.41 eq) and sulfo-DBCO-PEG4-NH2 (150.5 mg, 0.22 mmol, 1 eq) were dissolved in DMSO following the addition of TEA (74.2 μL, 0.53 mmol, 2.41 eq). The reaction was stirred at room temperature for 1 h. The product was purified by flash reverse phase chromatography using a gradient of 0-95% acetonitrile/H₂O (0.05% TFA) over 20 CVs. Pure fractions were combined, frozen at −80° C. and lyophilized to afford the intermediate Boc-Lys(Boc)-PEG4-sulfo-DBCO as an off white solid. Boc-Lys(Boc)-PEG4-sulfo-DBCO (77.9 mg, 0.08 mmol, 1 eq) was dissolved in 700 μL of DCM. Then, 5 μL of DI water, 5 μL of triisopropylsilane (TIPS), and 300 μL of TFA was added to the reaction flask. The Boc deprotection reaction was allowed to proceed for 30 minutes at room temperature. DCM and TFA were removed by blowing air over the reaction mixture before the intermediate, NH2-Lys(NH2)-PEG4-sulfo-DBCO was dried under high vacuum to yield a dark oil.

NH2-Lys(NH2)-PEG4-sulfo-DBCO (37 mg, 0.046 mmol, 1 eq) was dissolved in 1 mL of DMSO before TEA (19.3 μL, 0.14 mmol, 3 eq) was added. After stirring for 5 minutes at room temperature, propargyl NHS ester (22.8 mg, 0.1 mmol, 2.2 eq) was added to the reaction flask. After 1 h the reaction was complete and confirmed by LC-MS. The product, sulfo-DBCO-PEG4-Pg2 was used without further purification. ESI-MS: m/z=1023.4 (M+H)⁺.

### Example 3—Synthesis of Polymer Arms (A)

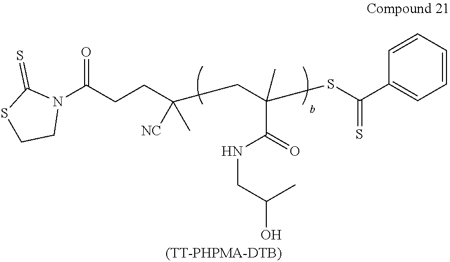

Compound 20 is a polymer arm (A) example of a homopolymer comprised of hydrophilic monomers (B). TT-functionalized poly[N-(2-hydroxypropyl)methacrylamide] (TT-PHPMA-DTB) was synthesized via the RAFT polymerization of HPMA using CTA-TT as a chain transfer agent and ACVA-TT as an initiator in tert-butanol (tBuOH) at 70° C. for 16 h. The initial monomer concentration [HPMA]₀=1 mol/L, the molar ratio [CTA-TT]₀:[ACVA-TT]₀=1:0.5, and [HPMA]₀:[CTA-TT]₀ varied to obtain polymers with different chain lengths. The following procedure was employed for a typical polymerization to produce TT-PHPMA-DTB targeting a molecular weight of 10 kDa: HPMA (572.0 mg, 4.00 mmol) was dissolved in 4 mL of tBuOH. CTA-TT (15.2 mg, 0.040 mmol) and ACVA-TT (9.65 mg, 0.020 mmol) were dissolved in anhydrous DMSO before mixing with the monomer solution. The mixture was transferred to a 5 mL ampule, which was sealed with a rubber septum and sparged with Ar (g) at r.t. for 30 min. The flask was then immersed in a water circulator preheated to 70° C. and polymerized for 16 h. The polymer was purified by precipitating against acetone for 3 times. After drying in vacuum oven overnight, light pink powder was obtained (277.3 mg, 40.1% yield). Number-average (Mn) and weight-average molecular weight (M,) were 10.05 kDa and 10.30 kDa, respectively, and polydispersity (PDI) was 1.02 measured by GPC-MALS. The chain end functionalities measured by UV-Vis spectroscopy [ϵ₃₀₅ (TT)=10300 L/(mol·cm), ϵ₃₀₅ (DTB)=12600 L/(mol·cm)] showed that (TT+DTB) %=95.3%.

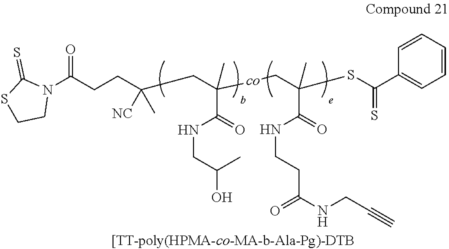

Compound 21 is a polymer arm (A) example of a co-polymer with hydrophilic monomers and reactive monomers (E) with alkyne groups. TT-poly(HPMA-co-MA-b-Ala-Pg)-DTB random copolymer was synthesized via the RAFT polymerization of HPMA and MA-b-Ala-Pg using CTA-TT as a chain transfer agent and ACVA-TT as an initiator in tert-butanol (tBuOH)/N,N-dimethylacetamide (DMAc) at 70° C. for 16 h. The initial monomer concentration [EM]₀=[HPMA+MA-b-Ala-Pg]₀=1 mol/L and the molar ratio [CTA-TT]₀:[ACVA-TT]₀=1:0.5. [ΣM]₀:[CTA-TT]₀ is varied to target polymers with different chain lengths, while the molar percentage of reactive site-containing comonomer MA-b-Ala-Pg controls the maximum number of cargo molecules (e.g., small molecule drugs, peptides) each polymer chain carries. The following procedure was employed for a typical polymerization to produce TT-poly(HPMA-co-MA-b-Ala-Pg)-DTB targeting 5 mol % of comonomer MA-b-Ala-Pg and a molecular weight of 40 kDa: HPMA (340.7 mg, 2.375 mmol) and MA-b-Ala-Pg (24.1 mg, 0.125 mmol) were dissolved in 2.13 mL of tBuOH. CTA-TT (3.2 mg, 0.008 mmol) as a 100 mg/mL stock solution in anhydrous DMAc and ACVA-TT (2.0 mg, 0.004 mmol) as a 50 mg/mL stock solution in anhydrous DMAc were then added to the monomer solution. The mixture was transferred to a 5 mL ampule, which was sealed with a rubber septum and sparged with Ar (g) at r.t. for 30 min. The flask was then immersed in a water circulator preheated to 70° C. and polymerized for 16 h. The resulted polymer was purified by precipitating against acetone for 3 times. After drying in vacuum oven overnight, light pink powder was obtained (208.9 mg, 57.7% yield). Number-average (Mn) and weight-average molecular weight (Mw) were 39.27 kDa and 42.85 kDa, respectively, and polydispersity (PDI) was 1.09 measured by GPC-MALS. The chain end functionalities measured by UV-Vis spectroscopy [ϵ₃₀₅ (TT)=10300 L/(mol·cm), ϵ₃₀₅ (DTB)=12600 L/(mol·cm)] showed that (TT+DTB) %=121.8%.

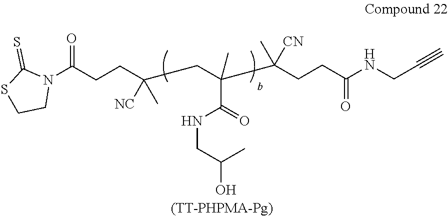

Compound 22 is a polymer arm (A) example of a homopolymer comprised of hydrophilic monomers (B) with two different end group functionalities (heterotelechelic). The propargyl functionality was introduced by reacting TT-PHPMA-DTB with 10-20 molar excess of ACVA-Pg. Example of reaction: Dry polymer TT-PHPMA-DTB (198 mg, 19.7 μmol) and ACVA-Pg (70.3 mg, 198.9 μmol) was dissolved in 3.0 mL of anhydrous DMSO. The solution was transferred to a 5 mL ampule, which was sealed with a rubber septum and sparged with Ar (g) at r.t. for 30 min. The flask was then immersed in a water circulator preheated to 70° C. and reacted for 3 h. The polymer was purified by precipitating against acetone for 3 times. After drying in vacuum oven overnight, off-white powder was obtained. Mn and Mw were 10.80 kDa and 12.10 kDa, respectively, and PDI was 1.12 measured by GPC-MALS. The chain end functionalities measured by UV-Vis spectroscopy [ϵ₃₀₅ (TT)=10300 L/(mol·cm)] showed that (TT) %=100%. Note: In this example, the TT group was added to the polymer during the polymerization step and the Pg functionality was added to the other end during capping.

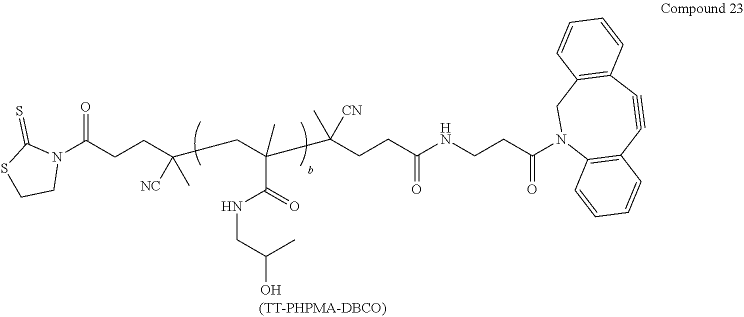

Compound 23 is a polymer arm (A) example of a homopolymer comprised of hydrophilic monomers (B) with two different end group functionalities (heterotelechelic). TT-PHPMA-DBCO was synthesized using the same method as described for as Compound 22, except that ACVA-Pg was replaced by ACVA-DBCO. Note: In this example, the TT group was added to the polymer during the polymerization step and the strained-alkyne functionality was added to the other end during capping.

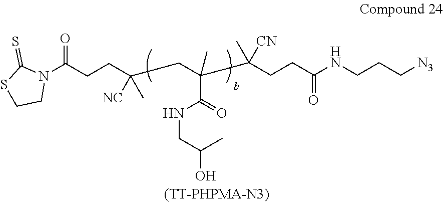

Compound 24 is a polymer arm (A) example of a homopolymer comprised of hydrophilic monomers (B) with two different end group functionalities (heterotelechelic). TT-PHPMA-N3 was synthesized using the same method as described for as Compound 22, except that ACVA-Pg was replaced by ACVA-N3. Note: In this example, the TT group was added to the polymer during the polymerization step and the N3 functionality was added to the other end during capping.

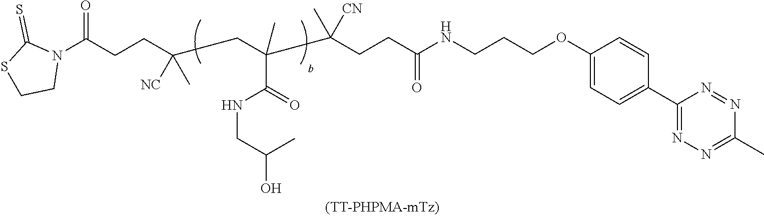

Compound 25 is a polymer arm (A) example of a homopolymer comprised of hydrophilic monomers (B) with two different end group functionalities (heterotelechelic). TT-PHPMA-mTz was synthesized using the same method as described for as Compound 22, except that ACVA-Pg was replaced by ACVA-mTz. Note: In this example, the TT group was added to the polymer during the polymerization step and the methyltetrazine functionality was added to the other end during capping.

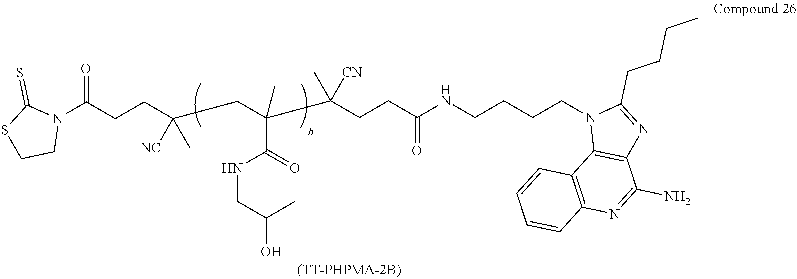

Compound 26 is a polymer arm (A) example of a homopolymer comprised of hydrophilic monomers (B) with two different end group functionalities (heterotelechelic). TT-PHPMA-2B was synthesized using the same method as described for as Compound 22, except that ACVA-Pg was replaced by ACVA-2B. Note: In this example, the TT group was added to the polymer during the polymerization step and the 2B functionality was added to the other end during capping.

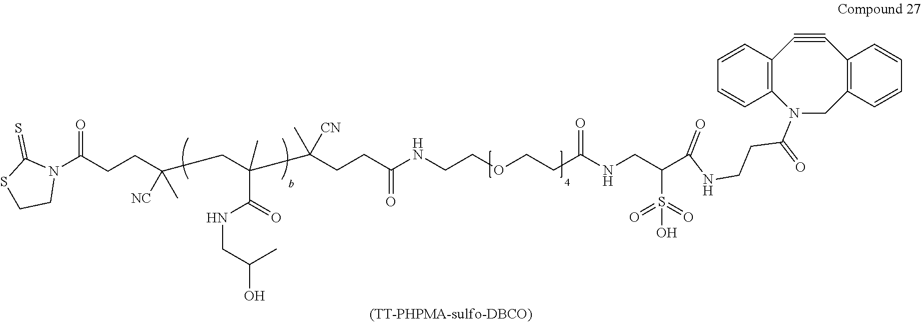

Compound 27 is a polymer arm (A) example of a homopolymer comprised of hydrophilic monomers (B) with two different end group functionalities (heterotelechelic). TT-PHPMA-sulfo-DBCO was synthesized in the same manner as Compound 22, TT-PHPMA-Pg except that ACVA-Pg was replaced with ACVA-sulfo-DBCO. Note: In this example, the TT group was added to the polymer during the polymerization step and the water-soluble strained-alkyne functionality was added to the other end during capping.

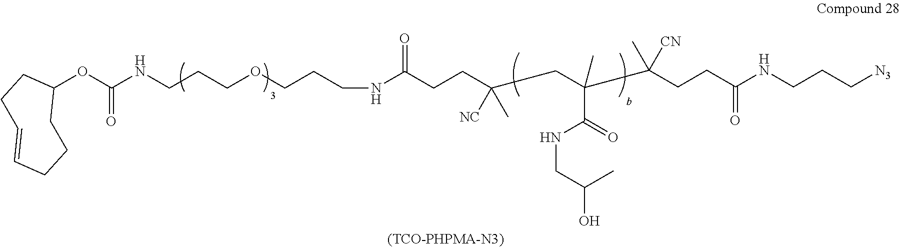

Compound 28. is a polymer arm (A) example of a homopolymer comprised of hydrophilic monomers (B) with two different end group functionalities (heterotelechelic). TCO-PHPMA-N3 was synthesized by reacting the carbonylthiazolidine-2-thione (TT) of Compound 24, TT-PHPMA-N3, with 5-7 molar excess of TCO-PEG3-amine using triethylamine as the catalyst. The following procedure was employed for a typical synthesis procedure for TCO-PHPMA-N3 from TT-PHPMA-N3: TT-PHPMA₄₀ₖ-N3 (62.1 mg, 1.6 μmol) and TCO-PEG3-amine (3.5 mg, 9.6 μmol) were dissolved in 800 μL of anhydrous DMSO. Triethylamine (1.3 mg, 12.7 μmol) was then added to the mixture and the reaction was allowed to proceed for 5 h at r.t. The product was purified by precipitating against acetone (6-8× volume) for three times. After drying in vacuum oven overnight, off-white solid was obtained (57.9 mg, 92.4%).

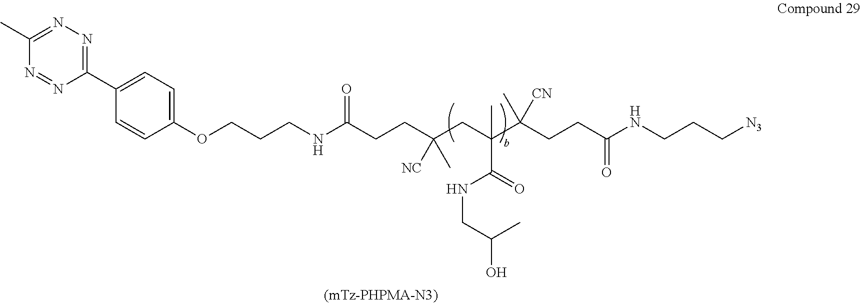

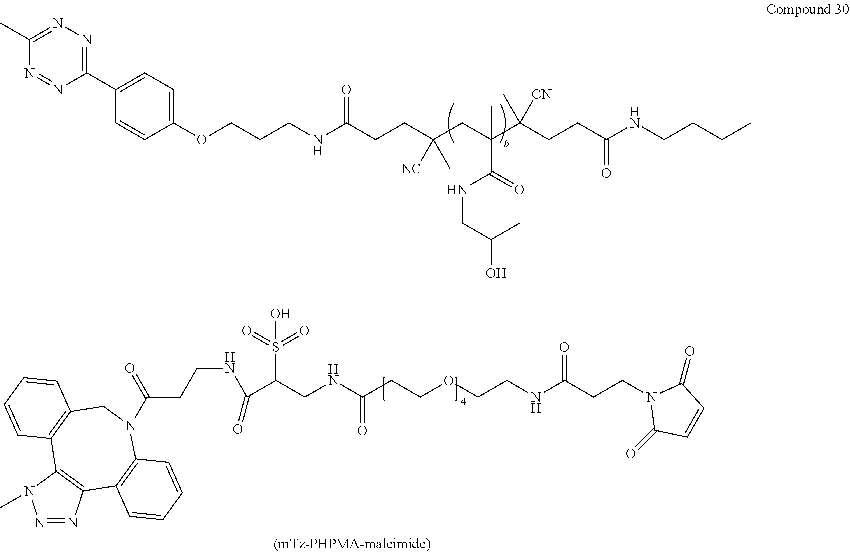

Compound 30 is a polymer arm (A) example of a homopolymer comprised of hydrophilic monomers (B) with two different end group functionalities (heterotelechelic). mTz-PHPMA-maleimide was synthesized by reacting the azide group (N3) of Compound 29, mTz-PHPMA-N3, with 10 molar excess of sulfo-DBCO-PEG4-maleimide. The following procedure was employed for a typical synthesis procedure for mTz-PHPMA-MI from mTz-PHPMA-N3: mTz-PHPMA₅₆ₖ-N3 (11.9 mg, 0.21 μmol) was dissolved in 50 μL of anhydrous DMSO before sulfo-DBCO-PEG4-maleimide (1.8 mg, 100 ma/mL in anhydrous DMSO, 2.1 μmol) was added. The reaction was allowed to proceed for 16 h at r.t. before the product was purified by precipitating against acetone (6-8× volume) for three times. After drying in vacuum oven overnight, light pink solid was obtained (9.2 mg, 76.2%).

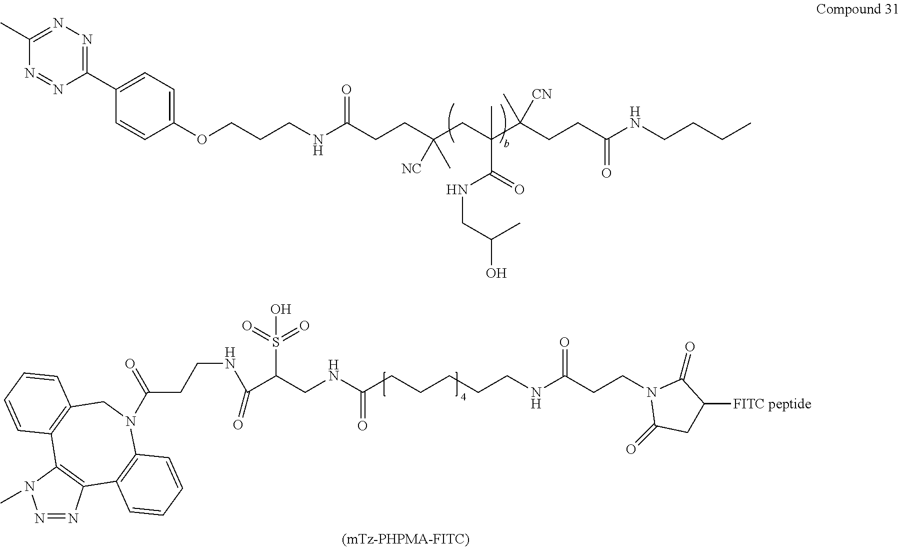

Compound 31 is a polymer arm (A) example of a homopolymer comprised of hydrophilic monomers (B) with two different end group functionalities (heterotelechelic). mTz-PHPMA-FITC peptide was synthesized by conjugating a peptide containing a FITC dye (FITC-Ahx-GSGSGSCG) to Compound 30, mTz-PHPMA-maleimide through maleimide-thiol coupling chemistry. The following procedure was employed for a typical synthesis: mTz-PHPMA₅₆ₖ-maleimide (2.0 mg, 0.036 μmol) was dissolved in 10 μL of anhydrous DMSO before FITC-peptide (2.0 mg, 20 mg/mL in anhydrous DMSO, 0.047 μmol) was added. The reaction was allowed to proceed for 16 h at r.t. before characterized using gel permeation chromatography (GPC). The resulted conjugate showed targeted UV absorbance at 488 nm (FITC absorbance wavelength) where the original polymer has no absorbance.

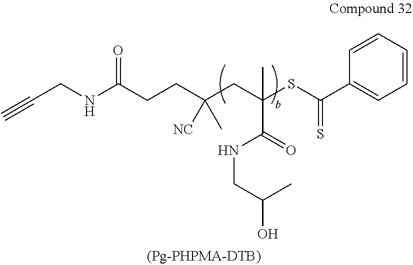

Compound 32 is a polymer arm (A) example of a homopolymer comprised of hydrophilic monomers (B) with two different end group functionalities (heterotelechelic). Note: The dithiobenzoate (DTB) present on the polymer indicates that the polymer is living and can add on additional monomers or can be capped. Pg-PHPMA-DTB was synthesized using the same method as described for as Compound 20, except that ACVA-TT and CTA-TT were replaced by ACVA-Pg and CTA-Pg.

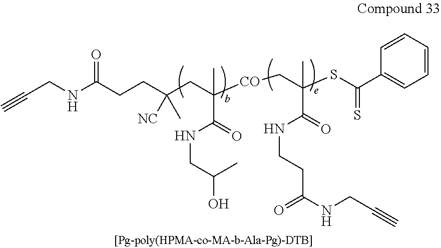

Compound 33 is a polymer arm (A) example of a copolymer comprised of hydrophilic monomers (B) and reactive monomers (E) with two different end group functionalities (i.e., the polymer arm is heterotelechelic). Note: The dithiobenzoate (DTB) present on the polymer indicates that the polymer is living and can add on additional monomers or can be capped. Pg-poly(HPMA-co-MA-b-Ala-Pg)-DTB random copolymer was synthesized following the same synthetic procedure as described for Compound 21, TT-poly(HPMA-co-MA-b-Ala-Pg)-DTB, except using CTA-Pg and ACVA-Pg. Light pink powder was obtained with 48.2% yield. Number-average (Mn) and weight-average molecular weight (Mw) were 36.34 kDa and 40.06 kDa, respectively, and polydispersity (PDI) was 1.10 measured by GPC-MALS. The chain end functionalities measured by UV-Vis spectroscopy [ϵ₃₀₅ (DTB)=12600 L/(mol·cm)] showed that DTB %=112.5%.

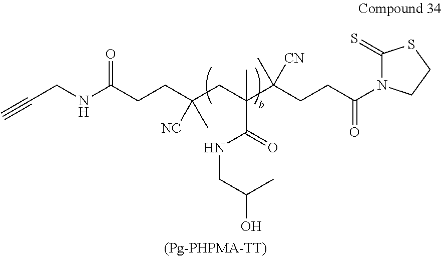

Compound 34, Pg-PHPMA-TT, was synthesized from Compound 32 using the same method as described for as Compound 22 except that ACVA-TT was used instead of ACVA-Pg. Note: In this example, the Pg group was added to the polymer during the polymerization step and the TT functionality was added to the other end during capping.

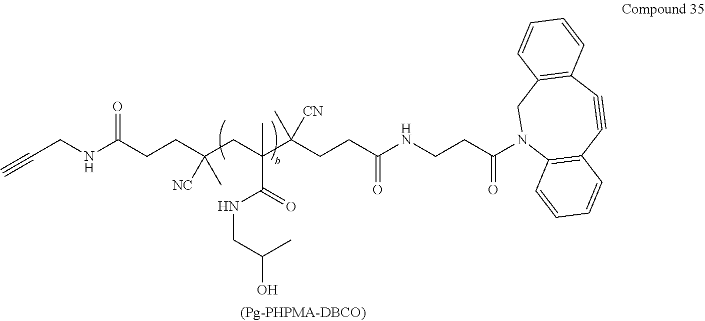

Compound 35. Pg-PHPMA-DBCO was synthesized using the same method as described for as Compound 34 except that ACVA-DBCO was used instead of with ACVA-TT. Note: In this example, the Pg group was added to the polymer during the polymerization step and the strained-alkyne functionality was added to the other end during capping.

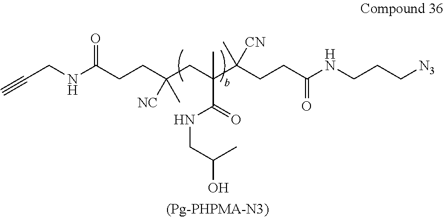

Compound 36. Pg-PHPMA-N3 was synthesized using the same method as described for as Compound 34 but ACVA-N3 was used instead of with ACVA-TT. Note: In this example, the Pg group was added to the polymer during the polymerization step and the azide functionality was added to the other end during capping.

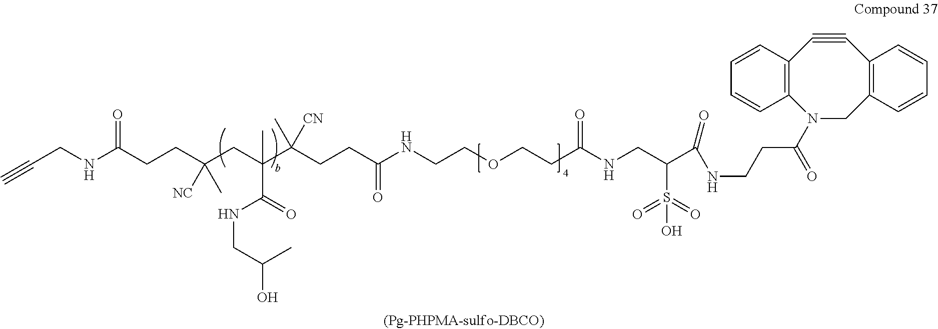

Compound 37. Pg-PHPMA-sulfo-DBCO was synthesized using the same method as described for Compound 34, Pg-PHPMA-TT, except that ACVA-TT was replaced by ACVA-sulfo-DBCO. Note: In this example, the Pg group was added to the polymer during the polymerization step and the water-soluble strained-alkyne functionality was added to the other end during capping.

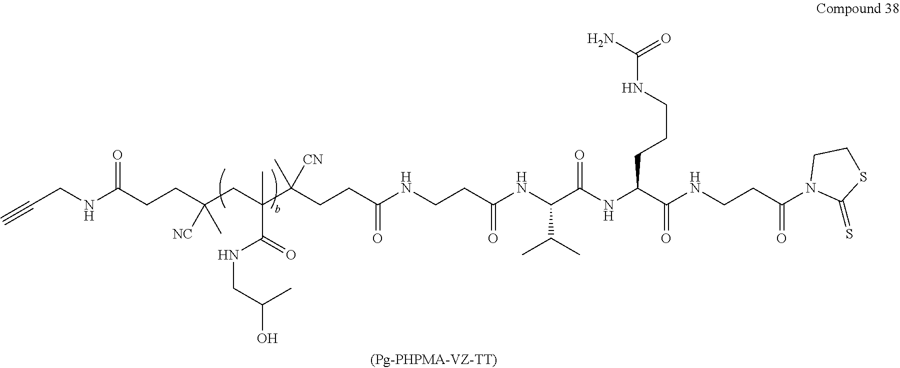

Compound 38. Pg-PHPMA-VZ-TT was synthesized using the same method as described for Compound 34, Pg-PHPMA-TT, except that ACVA-TT were replaced by ACVA-VZ-TT. Note: In this example, the Pg group was added to the polymer during the polymerization step and the TT-activated peptide was added to the other end during capping.

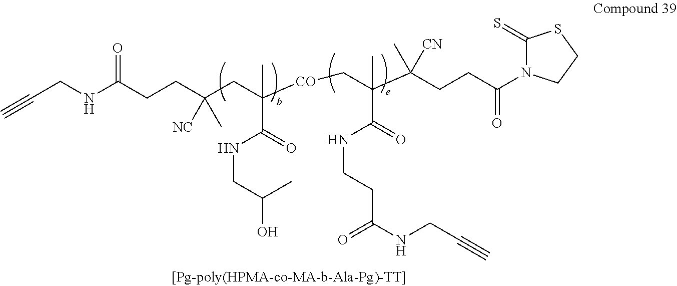

Compound 39. Pg-poly(HPMA-co-MA-b-Ala-Pg)-TT was synthesized by capping Compound 33 Pg-poly(HPMA-co-MA-b-Ala-Pg)-DTB with ACVA-TT using the same method as described for Compound 34, Pg-PHPMA-TT. Note: In this example, the Pg group was added to the polymer during the polymerization step and the TT functionality was added to the other end during capping.

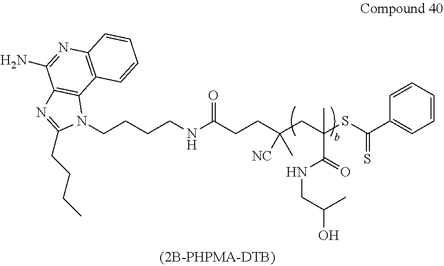

Compound 40. 2B-PHPMA-DTB was synthesized using the same method as described for Compound 20, TT-PHPMA-DTB, except that ACVA-TT and CTA-TT were replaced by ACVA-2B and CTA-2B, and [M]₀:[CTA-2B]₀ is adjusted to target Mn=10 kDa. Light pink powder was obtained with 48.2% yield. Number-average (Mn) and weight-average molecular weight (Mw) were 11.86 kDa and 12.82 kDa, respectively, and polydispersity (PDI) was 1.08 measured by GPC-MALS.

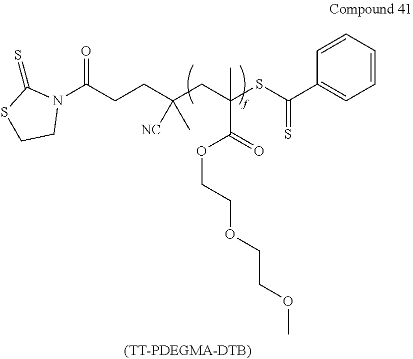

Compound 41. TT-PDEGMA-DTB was synthesized via the RAFT polymerization of DEGMA using CTA-TT as a chain transfer agent and ACVA-TT as an initiator in 1,4-dioxane/DMSO at 70° C. for 3 h. The initial monomer concentration [DEGMA]₀=4.0 mol/L, the molar ratio [CTA-TT]₀:[ACVA-TT]₀=1:0.2, and [DEGMA]₀:[CTA-TT]₀ varied to obtain polymers with different chain lengths. The following procedure was employed for a typical polymerization to produce TT-PDEGMA-DTB targeting a molecular weight of 20 kDa: DEGMA (1003.0 mg, 5.32 mmol) was dissolved in 1.3 mL of 1,4-dioxane. CTA-TT (16.87 mg, 0.044 mmol) as a 100 mg/mL stock solution in anhydrous DMSO and ACVA-TT (4.28 mg, 0.009 mmol) as a 50 mg/mL stock solution in anhydrous DMSO were added to the monomer solution. The mixture was transferred to a 5 mL ampule, which was sealed with a rubber septum and sparged with Ar (g) at r.t. for 30 min. The flask was then immersed in a water circulator preheated to 70° C. and polymerized for 3 h. The polymer was purified by precipitating against diethyl ether for 3 times. After drying in vacuum oven overnight, pink solid was obtained (460.7 mg, 45.2% yield). Number-average (Mn) and weight-average molecular weight (Mw) were 21.53 kDa and 22.09 kDa, respectively, and polydispersity (PDI) was 1.03 measured by GPC-MALS.

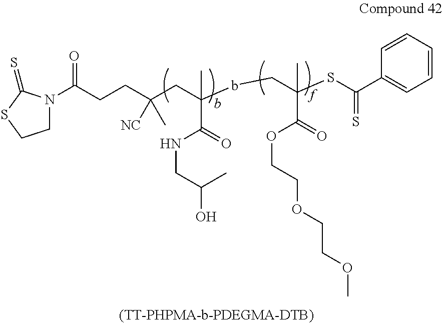

Compound 42. TT-PHPMA-b-PDEGMA-DTB was synthesized via a chain-extension polymerization through the RAFT mechanism of DEGMA using Compound 20, TT-PHPMA-DTB, as the macromolecular chain transfer agent (macro-CTA) and 2,2′-azobis(2-methylpropionitrile) (AIBN) as an initiator in tBuOH/DMAc (5/5, v/v) at 70° C. for 16 h. [DEGMA]₀=0.67 mol/L and [macro-CTA]₀:[AlBN]₀=1:0.2. For example, when TT-PHPMA12.8 k-DTB was used as the macro-CTA, [DEGMA]₀:[macro-CTA]₀ was adjusted to 100 to target Mn (PDEGMA)=20 kDa. TT-PHPMA-DTB (257.0 mg, 20.0 μmol) was dissolved in 1.5 mL of anhydrous DMAc. AIBN (0.66 mg, 4.0 μmol) as a 50 mg/mL stock solution in anhydrous DMAc, DEGMA (376.4 mg, 2.00 mmol) and 1.5 mL of anhydrous tBuOH was then added to the macro-CTA solution. The mixture was transferred to a 5 mL ampule, which was sealed with a rubber septum and sparged with Ar (g) at r.t. for 30 min. The flask was then immersed in a water circulator preheated to 70° C. and polymerized for 18 h. The polymer was purified by precipitating against diethyl ether for 3 times. After drying in vacuum oven overnight, light pink solid was obtained (537.1 mg, 84.8% yield). Number-average (Mn) and weight-average molecular weight (Mw) were 32.27 kDa and 34.33 kDa, respectively, and polydispersity (PDI) was 1.06 measured by GPC-MALS.

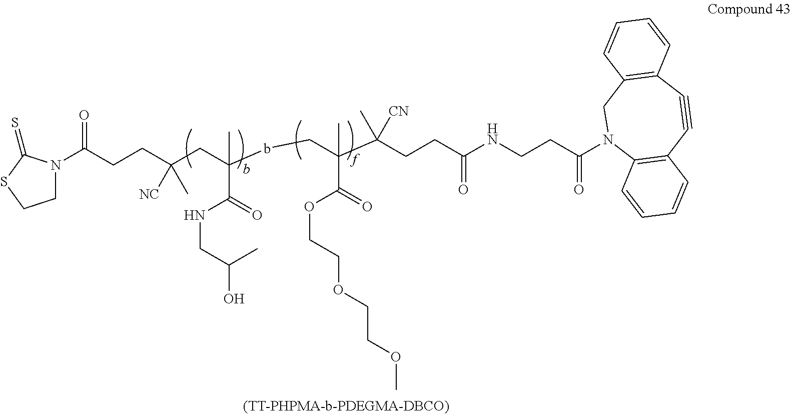

Compound 43. TT-PHPMA-b-PDEGMA-DBCO was synthesized by capping Compound 42, TT-PHPMA-b-PDEGMA-DTB, with ACVA-DBCO using the same method as described for Compound 23, TT-PHPMA-DTB.

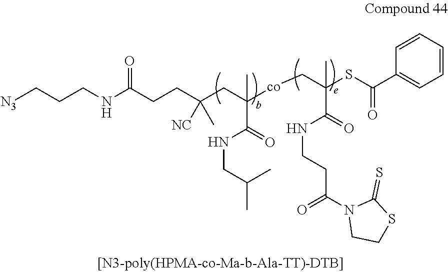

Compound 44. N3-poly(HPMA-co-Ma-b-Ala-TT)-DTB was synthesized via the RAFT polymerization of HPMA and Ma-b-Ala-TT using CTA-N3 as a chain transfer agent and ACVA-N3 as an initiator in 1:1 tert-butanol (tBuOH) and dimethylacetamide (DMAc) at 70° C. for 16 h. The initial monomer concentration [HPMA/Ma-b-Ala-TT]₀=1 mol/L with [HPMA]₀:[Ma-b-Ala-TT]₀=7:3, the molar ratio [CTA-N3]₀:[ACVA-N3]₀=1:0.5, and [HPMA/Ma-b-Ala-TT]₀:[CTA-N3]₀ varied to obtain polymers with different chain lengths. The following procedure was employed for a typical polymerization to produce N3-poly(HPMA-co-Ma-b-Ala-TT)-DTB targeting molecular weight of 40 kDa: HPMA (1503.50 mg, 10.50 mmol) was dissolved in 9.5 mL tBuOH. Ma-b-Ala-TT (1162.60 mg, 4.50 mmol) was dissolved in 9.5 mL anhydrous DMAc and combined with HPMA solution. CTA-N3 (19.70 mg, 0.055 mmol) and ACVA-N3 (12.10 mg, 0.027 mmol) were dissolved in anhydrous DMAc before mixing with monomer solution. The mixture was transferred to a 20 mL ampule, which was sealed with a rubber septum and sparged with Ar (g) at r.t. for 45 min. The flask was then immersed in a water circulator preheated to 70° C. and polymerized for 16 h. The polymer was purified by precipitating against acetone three times. After drying in a vacuum oven overnight, an orange powder was obtained (1498 mg, 55.8% yield). Number-average (Mn) and weight-average molecular weight (Mw) were 36.63 kDa and 37.71 kDa, respectively, and polydispersity (PDI) was 1.03 measured by GPC-MALS. The arrayed functionality was measured by UV-Vis spectroscopy [ϵ₃₀₅ (TT)=10300 L/(mol·cm)] showed 34.2 mol % TT. The reactive monomer in this example comprises a TT leaving group, which promotes nucleophilic attack and displacement of the TT. Any drug molecule, charged molecule or reactive molecule with an amine or linked to a linker with an amine reactive handle can be used to displace the TT group and form an amide bond linking the drug molecule, charged molecule or reactive molecule directly (or indirectly via a linker) to the polymer backbone. In some embodiments, when a charged molecule is linked to the reactive monomer, the reactive monomer may then be classified as a charged monomer, i.e., the route to generating a charged monomer can occur via a reactive monomer.

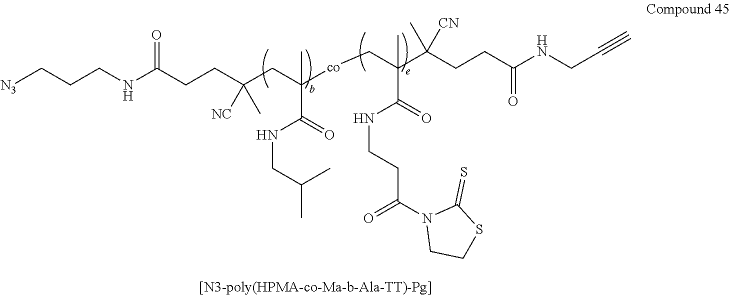

Compound 45 is an example of a polymer arm comprised of a copolymer with hydrophilic monomers (B) and reactive monomer (E). N3-poly(HPMA-co-Ma-b-Ala-TT)-Pg was synthesized by capping Compound 44, N3-poly(HPMA-co-Ma-b-Ala-TT)-DTB with ACVA-Pg following the same synthetic procedure as Compound 22.

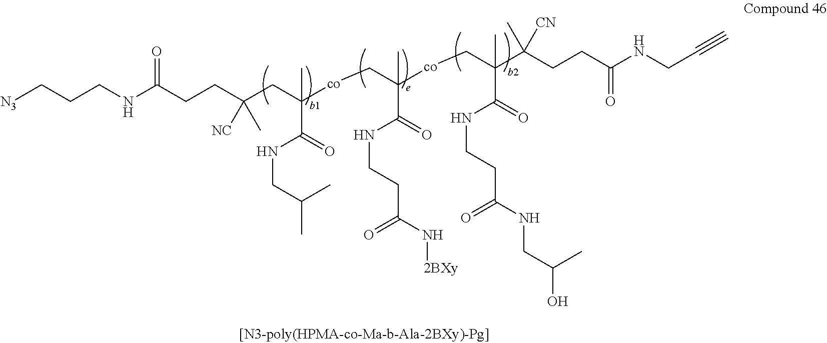

Compound 46 is an example of a polymer arm comprised of a copolymer with hydrophilic monomers (B) and reactive monomer (E), wherein the reactive monomers are linked to a drug (D, specifically “D2”), i.e., the TLR-7/8a, 2BXy, through an amide bond. N3-poly(HPMA-co-Ma-b-Ala-2BXy)-Pg was synthesized by reacting the carbonylthiazolidine-2-thione (TT) groups of Compound 45 with 2BXy (Compound A) and amino-2-propanol in the molar ratio [2BXy]:[amino-2-propanol]=1:2. Specifically, N3-poly(HPMA-co-Ma-b-Ala-TT)-Pg (40.00 mg, 1.05 μmol polymer, 72 μmol TT) and 2 mL of DMSO were added to a 20 mL scintillation vial. The polymer was fully dissolved before the addition of 2BXy (7.80 mg, 21.77 μmol) and triethylamine (15.10 μL, 110 μmol). The reaction was allowed to proceed at r.t. for 2 h before the addition of amino-2-propanol (4.50 mg, 60 μmol) and additional hour afterward. The polymer was then purified by dialysis against methanol for 2 h three times using reconstituted cellulose (RC) membrane with a molecular weight cutoff (MWCO) of 20 kDa. The polymer was collected by precipitating against diethyl ether and dried overnight in a vacuum oven. The product was obtained as a white powder (31.4 mg, 70.6% yield). Mn and Mw were 50.21 kDa and 54.95 kDa, respectively, and PDI was 1.09 measured by GPC-MALS. The 2BXy content measured by UV-Vis spectroscopy [ϵ₃₂₅ (2BXy)=5012 L/(mol·cm) showed 10.28 mol % 2BXy.

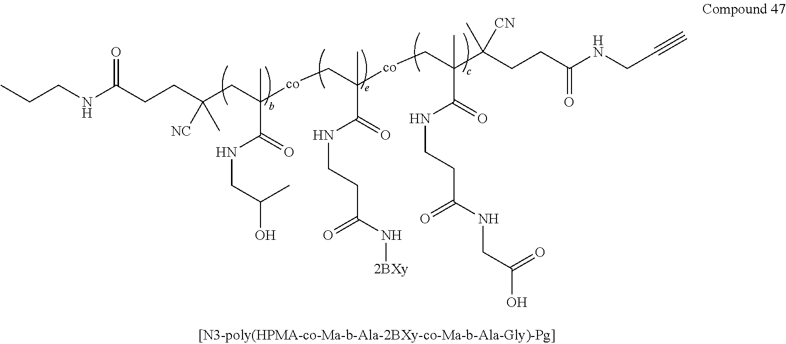

Compound 47 is an example of a polymer arm comprised of a terpolymer with hydrophilic monomers (B), reactive monomers (E) linked to a drug (D2), i.e., the TLR-7/8a, 2BXy, and charged monomers (C) with a carboxylic acid group, which are negatively charged at pH 7.4. Note: Drug is linked to the reactive monomer through an amide bond. N3-poly(HPMA-co-Ma-b-Ala-2BXy-co-Ma-b-Ala-Gly)-Pg was synthesized in the same manner as Compound 46 but glycine was used instead of amino-2-propanol and the ratio of DMSO:PBS(1×)=4:1 was used as the solvent.

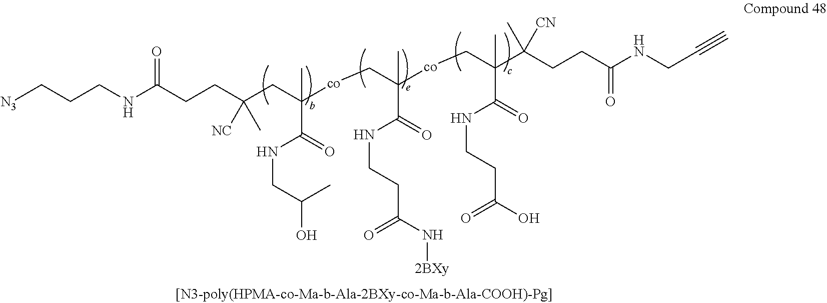

Compound 48 is an example of a polymer arm comprised of a terpolymer with hydrophilic monomers (B), reactive monomers (E) linked to a drug (D2), i.e., the TLR-7/8a, 2BXy, and charged monomers (C) with a carboxylic acid group, which is negatively charged at pH 7.4. Note: The drug is linked to the reactive monomer through an amide bond. N3-poly(HPMA-co-Ma-b-Ala-2BXy-co-Ma-b-Ala-COOH)-Pg was synthesized in the same manner as Compound 46 but amino-2-propanol was not used; instead the remaining TT groups were hydrolyzed with 0.01 M NaOH after addition of 2BXy.

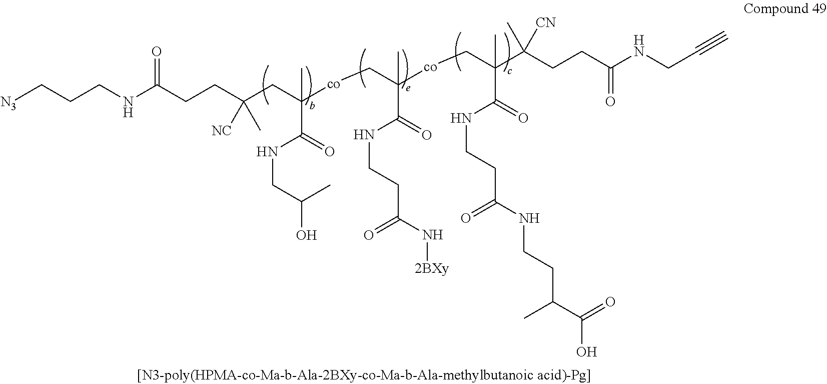

Compound 49 is an example of a polymer arm comprised of a terpolymer with hydrophilic monomers (B), reactive monomers (E) linked to a drug (D2), i.e., the TLR-7/8a, 2BXy, and charged monomers (C) with a carboxylic acid group, which is negatively charged at pH 7.4. Note: The drug is linked to the reactive monomer through an amide bond. N3-poly(HPMA-co-Ma-b-Ala-2BXy-co-Ma-b-Ala-methylbutanoic acid)-Pg was synthesized in the same manner as Compound 46 but 4-amino-2-methylbutanoic acid was used instead of amino-2-propanol.

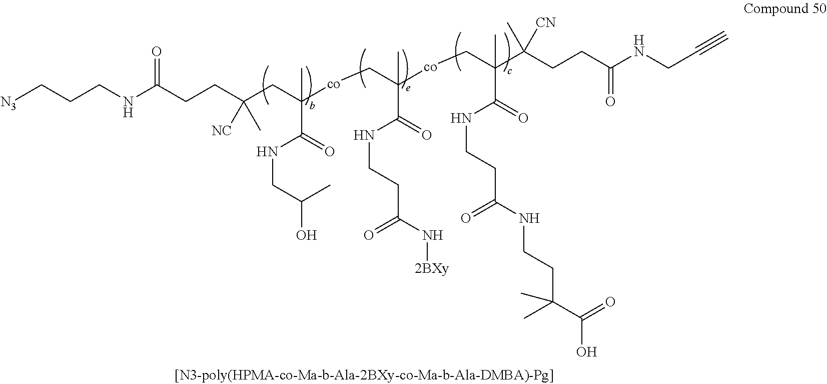

Compound 50 is an example of a polymer arm comprised of a terpolymer with hydrophilic monomers (B), reactive monomers (E) linked to a drug (D2), i.e., the TLR-7/8a, 2BXy, and charged monomers (C) with a carboxylic acid group, i.e., 4-amino-2,2-dimethylbutanoic acid (DMBA), which is negatively charged at pH 7.4. Note: The drug is linked to the reactive monomer through an amide bond. N3-poly(HPMA-co-Ma-b-Ala-2BXy-co-Ma-b-Ala-DMBA)-Pg was synthesized in the same manner as Compound 46 but 4-amino-2,2-dimethylbutanoic acid was used instead of amino-2-propanol.

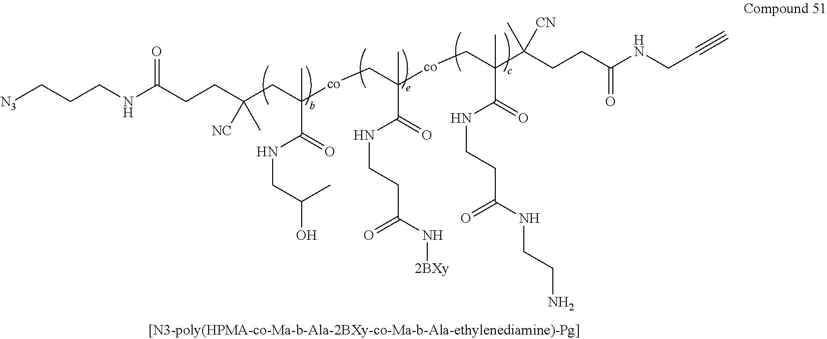

Compound 51 is an example of a polymer arm comprised of a terpolymer with hydrophilic monomers (B), reactive monomers (E) linked to a drug (D2), i.e., the TLR-7/8a, 2BXy, and charged monomers (C) with an amine group, which is positively charged at pH 7.4. Note: The drug is linked to the reactive monomer through an amide bond. N3-poly(HPMA-co-Ma-b-Ala-2BXy-co-Ma-b-Ala-ethylenediamine)-Pg was synthesized in the same manner as Compound 46 but ethylenediamine was used instead of amino-2-propanol.

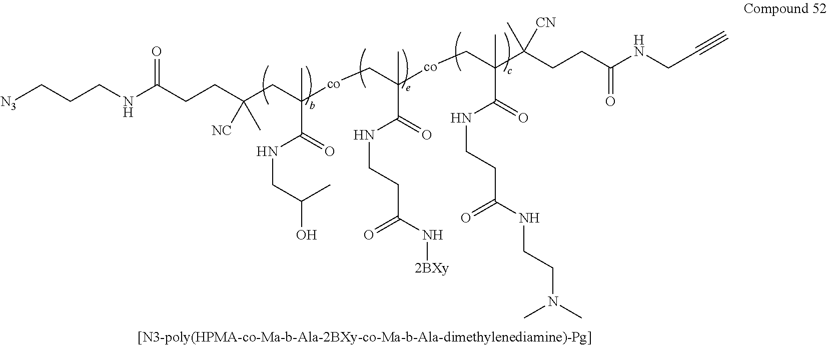

Compound 52 is an example of a polymer arm comprised of a terpolymer with hydrophilic monomers (B), reactive monomers (E) linked to a drug (D2), i.e., the TLR-7/8a, 2BXy, and charged monomers (C) with a tertiary amine group, which is partially positively charged at pH 7.4. Note: The drug is linked to the reactive monomer through an amide bond. N3-poly(HPMA-co-Ma-b-Ala-2BXy-co-Ma-b-Ala-dimethylethylenediamine)-Pg was synthesized in the same manner as Compound 46 but N,N′-dimethylethylenediamine was used instead of amino-2-propanol.

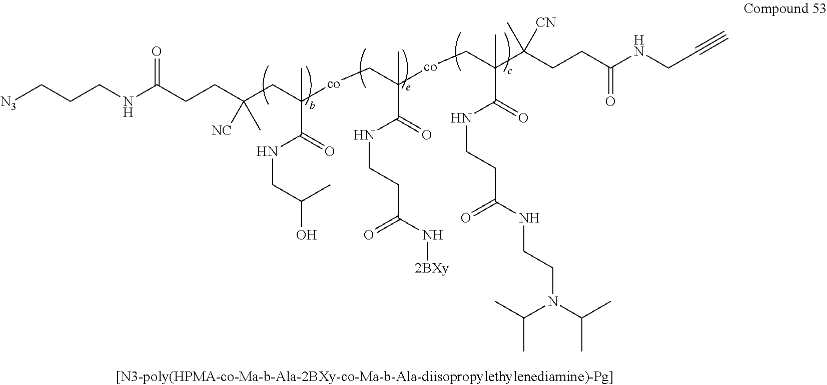

Compound 53 is an example of a polymer arm comprised of a terpolymer with hydrophilic monomers (B), reactive monomers (E) linked to a drug (D2), i.e., the TLR-7/8a, 2BXy, and charged monomers (C) with a tertiary amine group, which is partially positively charged at pH 7.4. Note: The drug is linked to the reactive monomer through an amide bond. N3-poly(HPMA-co-Ma-b-Ala-2BXy-co-Ma-b-Ala-diisopropylethylenediamine)-Pg was synthesized in the same manner as Compound 46 but N,N′-diisopropylethylenediamine was used instead of amino-2-propanol.

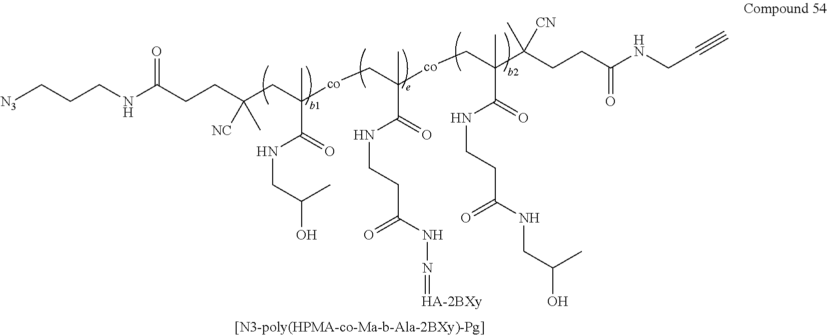

Compound 54 is an example of a polymer arm comprised of hydrophilic monomers (B) and reactive monomers (E) linked to a drug (D2), i.e., the TLR-7/8a, 2BXy, through a hydrazone bond. N3-poly(HPMA-co-Ma-b-Ala-HZ-2BXy)-Pg was synthesized by reacting the TT groups of Compound 44 with hydrazine monohydrate and amino-2-propanol in the molar ratio [hydrazine]:[amino-2-propanol]=1:2 and forming a hydrazone linkage to Compound D, 2BXy-HA, through these polymer-bound hydrazides. Specifically, N3-poly(HPMA-co-Ma-b-Ala-TT)-Pg (10.00 mg, 0.26 μmol) and 100 μL of methanol were added to a 2 mL vial. The polymer was fully dissolved before the addition of hydrazine monohydrate (0.27 mg, 5.43 μmol). The reaction was allowed to proceed at r.t. for 30 minutes before the addition of amino-2-propanol (1.02 mg, 13.61 μmol) and additional hour afterward. The 2BXy-HA (3.17 mg, 6.53 μmol) and 32 μL DMSO were added to the vial just prior to addition of acetic acid (20.61 μL, 360 μmol). The reaction was allowed to proceed at r.t. overnight. The polymer was then purified by dialysis against methanol for 2 h three times using reconstituted cellulose (RC) membrane with a molecular weight cutoff (MWCO) of 25 kDa. The polymer was collected by precipitating against diethyl ether and dried overnight in a vacuum oven. The product was obtained as a white powder. Mn and Mw were 59.61 kDa and 61.09 kDa, respectively, and PDI was 1.02 measured by GPC-MALS. The 2Bxy content measured by UV-Vis spectroscopy [ϵ₃₂₅ (2Bxy)=5012 L/(mol·cm) showed 9.79 mol % 2Bxy.

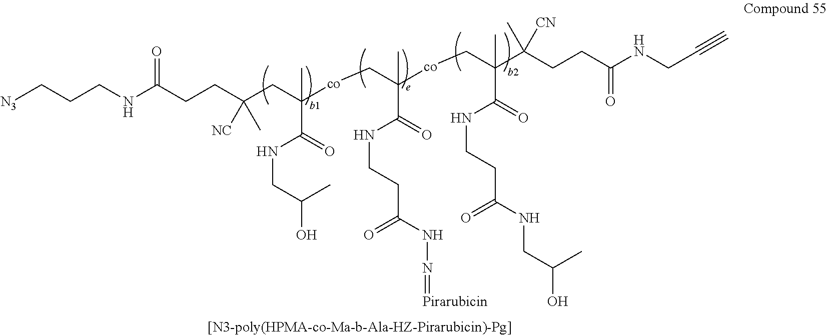

Compound 55 is an example of a polymer arm comprised of hydrophilic monomers (B) and reactive monomers (E) linked to a drug (D2), i.e., the chemotherapeutic anthracycline, Pirarubicin, through a hydrazone bond. N3-poly(HPMA-co-Ma-b-Ala-HZ-Pirarubicin)-Pg was synthesized in the same manner as Compound 54 but pirarubicin, which contains a ketone, was used instead of 2BXy-HA.

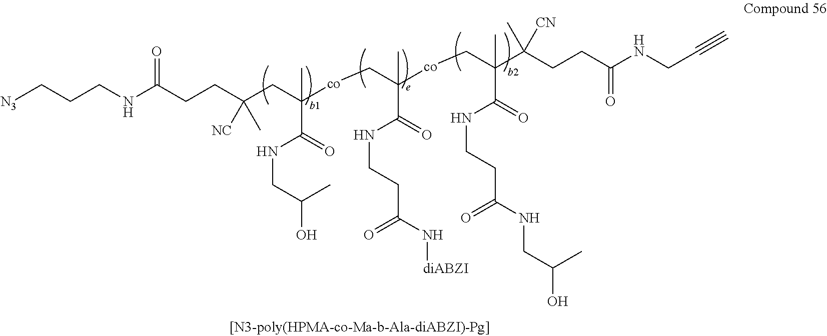

Compound 56 is an example of a polymer arm comprised of hydrophilic monomers (B) and reactive monomers (E) linked to a drug (D2), i.e., the STING agonist pip-diABZI, through an amide bond. N3-poly(HPMA-co-Ma-b-Ala-diABZI)-Pg was synthesized in the same manner as Compound 46 but Compound C, pip-diABZI, was used instead of 2BXy.

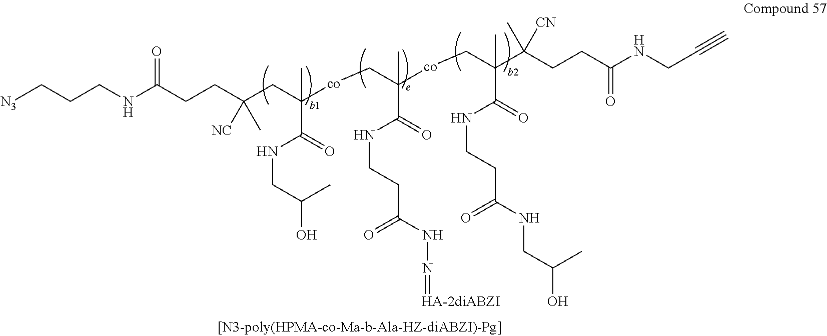

Compound 57 is an example of a polymer arm comprised of hydrophilic monomers (B) and reactive monomers (E) linked to a drug (D2), i.e., the STING agonist pip-diABZI-HA, through a hydrazone bond. N3-poly(HPMA-co-Ma-b-Ala-HZ-diABZI)-Pg was synthesized in the same manner as Compound 54 but Compound E, diABZI-HA, was used instead of 2BXy-HA and DMSO was used as the solvent.

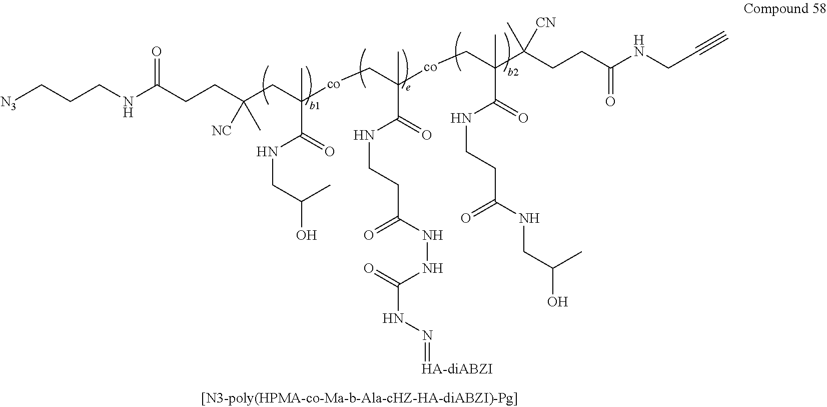

Compound 58. [N3-poly(HPMA-co-MA-b-Ala-cHZ-HA-diABZI)-Pg] is an example of a polymer arm comprised of hydrophilic monomers (B) and reactive monomers (E) linked to a drug (D2), i.e., the STING agonist diABZI, through a pH-sensitive carbohydrozone bond. N3-poly(HPMA-co-Ma-b-Ala-cHZ-diABZI)-Pg was synthesized by reacting the TT groups of Compound 44 with carbohydrazide and amino-2-propanol in the molar ratio [carbohydrazide]:[amino-2-propanol]=1:3 and forming a hydrazone linkage to Compound E, diABZI-HA, through these polymer-bound hydrazides. Specifically, N3-poly(HPMA-co-Ma-b-Ala-TT)-Pg (4.00 mg, 6.9 μmoles of TT) dissolved in 200 μL of anhydrous DMSO was added to a 1.5 mL tube. The polymer was fully dissolved before the addition of amino-2-propanol (0.34 mg, 4.5 μmol in 16.8 μL of DMSO). The reaction was allowed to proceed at r.t. for 2 h before carbohydrazide (0.81 mg, 9.0 μmol in 40.4 μL of DMSO). The reaction was allowed to proceed at r.t. overnight. The polymer was then purified by dialysis against methanol for 2 h three times using reconstituted cellulose (RC) membrane with a molecular weight cutoff (MWCO) of 25 kDa. Into the purified polymer, diABZI-HA (1.75 mg, 1.8 μmol in 87.4 μL of DMSO) was added prior to addition of acetic acid (15.7 μL, 275 μmol). The reaction was allowed to proceed at r.t. overnight. The product was obtained as a white powder. Mn and Mw were 45.7 kDa and 48.5 kDa, respectively, and PDI was 1.060 measured by GPC-MALS. The diABZI content measured by UV-Vis spectroscopy showed 7.5 mol % diABZI.

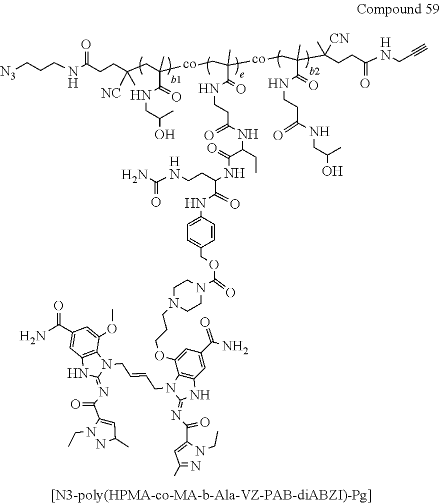

Compound 59. [N3-poly(HPMA-co-MA-b-Ala-VZ-PAB-diABZI)-Pg] is an example of a polymer arm comprised of hydrophilic monomers (B) and reactive monomers (E) linked to a drug (D2), i.e., the STING agonist diABZI, through an enzyme (i.e., cathepsin)-degradable valine-citrulline-PAB. N3-poly(HPMA-co-Ma-b-Ala-VZ-PAB-diABZI)-Pg was synthesized by reacting the carbonylthiazolidine-2-thione (TT) groups of Compound 45 with Compound H, diABZI-PAB-Cit-Val, and amino-2-propanol in the molar ratio [diABZI-PAB-Cit-Val]:[amino-2-propanol]=1:3. Specifically, N3-poly(HPMA-co-Ma-b-Ala-TT)-Pg (3.33 mg, 5.72 μmol TT) and 166 μL of anhydrous DMSO were added to a 1.5 mL tube. The polymer was fully dissolved before the addition of diABZI-PAB-Cit-Val (1.76 mg, 1.40 μmol in 87.8 μL of DMSO) and triethylamine (0.87 mg, 8.57 μmol in 43.4 μL DMSO). The reaction was allowed to proceed at r.t. overnight before the addition of amino-2-propanol (2.15 mg, 28.6 μmol in 107.4 μL DMSO) and additional 2 hours afterward. The polymer was then purified by precipitating against diethyl ether (3 rounds) and dried overnight in a vacuum oven. The product was obtained as a white powder (3.4 mg, 67% yield). Mn and Mw were 61.6 kDa and 68.1 kDa, respectively, and PDI was 1.105 measured by GPC-MALS. The diABZI content measured by UV-Vis spectroscopy [ϵ₃₂₀ (diABZI)=23822 L/(mol·cm) showed 8.31 mol % diABZI.

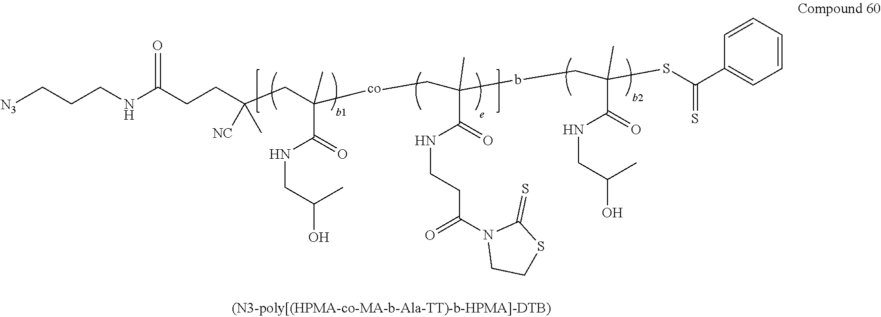

Compound 60. N3-poly[(HPMA-co-Ma-b-Ala-TT)-b-HPMA]-DTB was synthesized via a chain-extension polymerization through the RAFT mechanism of HPMA using Compound 44, N3-poly(HPMA-co-Ma-b-Ala-TT)-DTB, as a macromolecular chain transfer agent (macro-CTA) and 2,2′-azobis(2-methylpropionitrile) (AIBN) as an initiator in tBuOH/DMAc (6/4, v/v) at 70° C. for 18 h. [HPMA]₀:[macro-CTA]₀ was varied to obtain block copolymers with different chain lengths. The initial monomer concentration [HPMA]₀=0.9 mol/L and the molar ratio [macro-CTA]₀:[AlBN]₀=1:0.2. For example, HPMA (258.3 mg, 1.80 mmol) was dissolved in 1.2 mL of anhydrous tBuOH. N3-poly(HPMA-co-Ma-b-Ala-TT)-DTB (208.5 mg, 9.0 μmol) was dissolved in 0.8 mL of anhydrous DMAc before mixing with the monomer solution. AIBN (0.26 mg, 1.67 μmol) as a 50 mg/mL stock solution in anhydrous DMAc was then added to the mixture. The mixture was transferred to a 5 mL ampule, which was sealed with a rubber septum and sparged with Ar (g) at r.t. for 20 min. The flask was then immersed in a water circulator preheated to 70° C. and polymerized for 18 h. The polymer was purified by precipitating against acetone/diethyl ether (3/1, v/v) for 3 times. After drying in vacuum oven overnight, light orange powder was obtained (277.0 mg, 59.3% yield). Number-average (Mn) and weight-average molecular weight (Mw) were 33.07 kDa and 37.06 kDa, respectively, and polydispersity (PDI) was 1.12 measured by GPC-MALS. The TT functionalities measured by UV-Vis spectroscopy [ϵ₃₀₅ (TT)=10300 L/(mol·cm), ϵ₃₀₅ (DTB)=12600 L/(mol·cm)] showed that the number of TT and DTB functionalities per polymer chain is 26 (12.6 mol % TT).

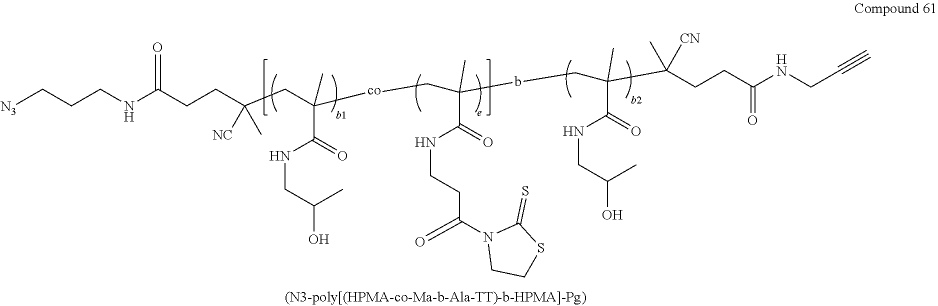

Compound 61 is an example of a polymer arm with di-block architecture comprised of hydrophilic monomers (B) and reactive monomers (E) on one block and only hydrophilic monomers on the other block. Note: In this example the di-block polymer is heterotelechelic with different functionalities on each end of the polymer arm. N3-poly[(HPMA-co-Ma-b-Ala-TT)-b-HPMA]-Pg was synthesized by capping Compound 60 using ACVA-Pg in the same manner as Compound 22.

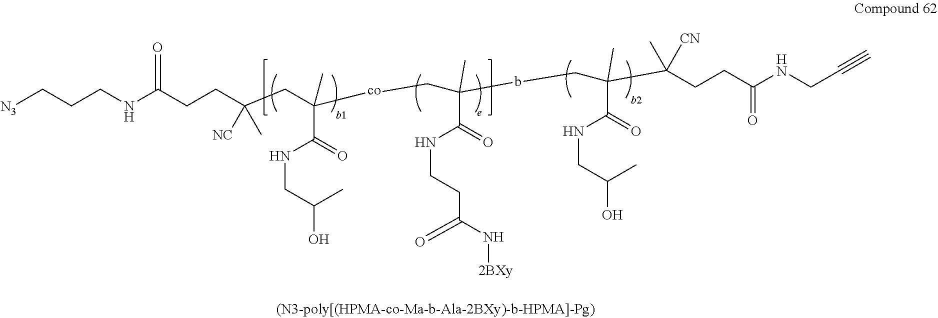

Compound 62 is an example of a polymer arm with di-block architecture comprised of hydrophilic monomers (B) and reactive monomers (E) linked to drug (D2, i.e., the TLR-7/8a, 2BXy) through an amide bond on one block and only hydrophilic monomers on the other block. Note: In this example the di-block polymer is heterotelechelic with different functionalities on each end of the polymer arm. N3-poly[(HPMA-co-Ma-b-Ala-2BXy)-b-HPMA]-Pg was synthesized by reacting the carbonylthiazolidine-2-thione (TT) groups of Compound 61 with excess 2BXy (Compound A). Specifically, N3-poly[(HPMA-co-Ma-b-Ala-TT)-b-HPMA]-Pg (30.0 mg, 0.91 μmol, 22.5 μmol TT groups) and 0.6 mL of anhydrous DMSO were added to a 20 mL scintillation vial. The polymer was fully dissolved before the addition of 2BXy (8.3 mg, 23.1 μmol, dissolved in 900 μL anhydrous DMSO) and triethylamine (3.5 μL, 82.0 μmol). The reaction was allowed to proceed at r.t. for overnight. The product was then purified precipitating against diethyl ether and dried overnight in a vacuum oven. The product was obtained as a white powder (26.8 mg, 70.0% yield). Mn and Mw were 35.8 kDa and 45.8 kDa, respectively, and PDI was 1.28 measured by GPC-MALS. The 2BXy content measured by UV-Vis spectroscopy [ϵ₃₂₅ (2BXy)=5012 L/(mol·cm) showed 11.62 mol % 2BXy.

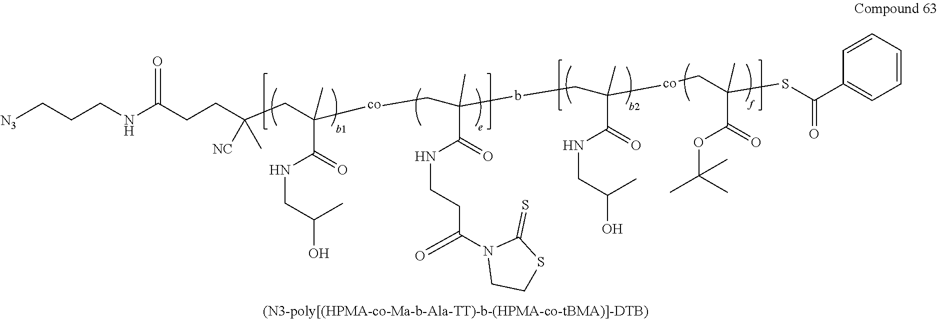

Compound 63. N3-poly[(HPMA-co-Ma-b-Ala-TT)-b-(HPMA-co-tBMA)]-DTB was synthesized in the same manner as Compound 60 by polymerizing tert-butyl methacrylate (tBMA) and HPMA at ratio [HPMA]₀:[tBMA]₀=9:1.

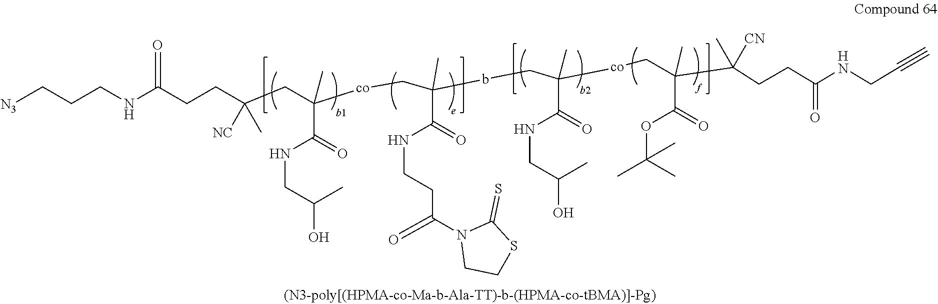

Compound 64. N3-poly[(HPMA-co-Ma-b-Ala-TT)-b-(HPMA-co-tBMA)]-Pg was synthesized in the same manner as Compound 61.

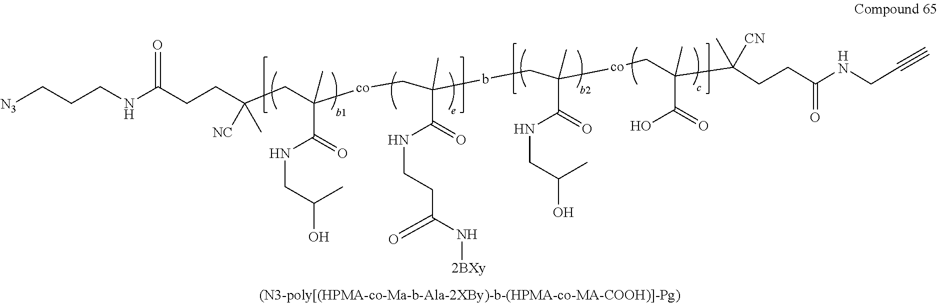

Compound 65 is an example of a polymer arm with di-block architecture comprised of hydrophilic monomers (B) and reactive monomers (E) linked to drug (D2, i.e., the TLR-7/8a, 2BXy) through an amide bond on one block and both hydrophilic monomers (B) and charged monomers (C) with a carboxylic acid functional group on the other block. Note: In this example the di-block polymer is heterotelechelic with different functionalities on each end of the polymer arm. N3-poly[(HPMA-co-Ma-b-Ala-2BXy)-b-(HPMA-co-Ma-COOH]-Pg was synthesized by reacting Compound 64 with 2BXy following the same protocol as Compound 62. Then tBMA was deprotected by dissolving the polymer in 95/2.5/2.5 TFA/TIPS/H₂O at 10 mM and sonicating for 5 minutes. The following procedure was employed for a typical deprotection: N3-poly[(HPMA-co-Ma-b-Ala-2BXy)-b-(HPMA-co-tBMA)]-Pg (45.4 mg, 1.15 μmol) was dissolved in 100 μL 95/2.5/2.5 TFA/TIPS/H₂O and sonicated for 5 minutes. The polymer was then purified by precipitating against diethyl ether three times. After drying in a vacuum oven overnight, a white powder was obtained. Number-average (Mn) and weight-average molecular weight (Mw) were 39.5 kDa and 50.1 kDa, respectively, and polydispersity (PDI) was 1.27 measured by GPC-MALS. The 2BXy content measured by UV-Vis spectroscopy [ϵ₃₂₅ (2BXy)=5012 L/(mol·cm) showed 10.8 mol % 2BXy.

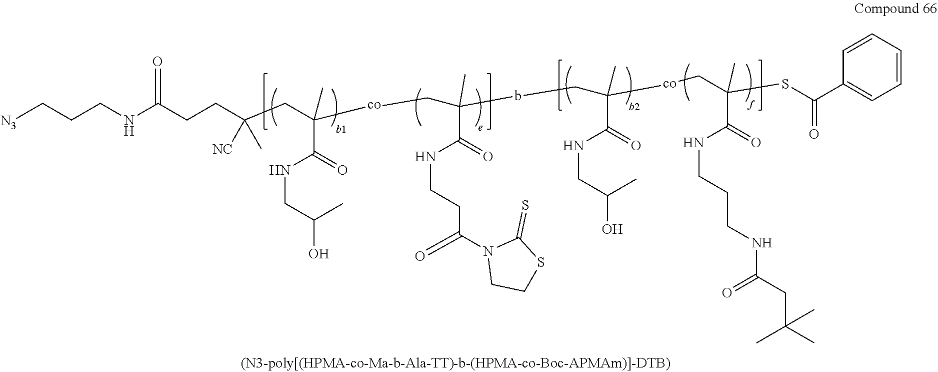

Compound 66. N3-poly[(HPMA-co-Ma-b-Ala-TT)-b-(HPMA-co-Boc-APMAm)]-DTB was synthesized in the same manner as Compound 63 but tBMA was replaced with N-(t-Boc-aminopropyl)methacrylamide (Boc-APMAm).

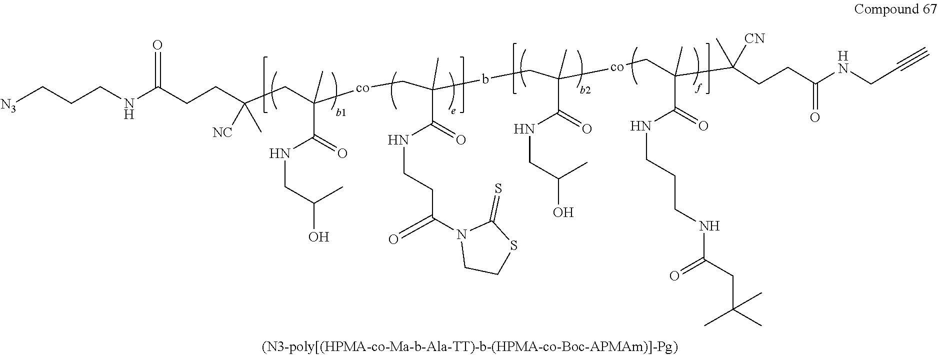

Compound 67. N3-poly[(HPMA-co-Ma-b-Ala-TT)-b-(HPMA-co-Boc-APMAm)]-Pg was synthesized in the same manner as Compound 61.

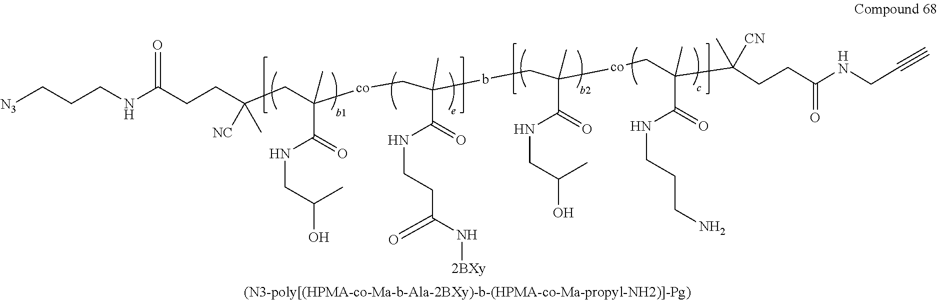

Compound 68 is an example of a polymer arm with di-block architecture comprised of hydrophilic monomers (B) and reactive monomers (E) linked to drug (D2, i.e., the TLR-7/8a, 2BXy) through an amide bond on one block and both hydrophilic monomers (B) and charged monomers (C) with an amide functional group on the other block. Note: In this example the di-block polymer is heterotelechelic with different functionalities on each end of the polymer arm. N3-poly[(HPMA-co-Ma-b-Ala-2BXy)-b-(HPMA-co-Ma-propyl-NH2)]-Pg was synthesized in the same manner as Compound 65.

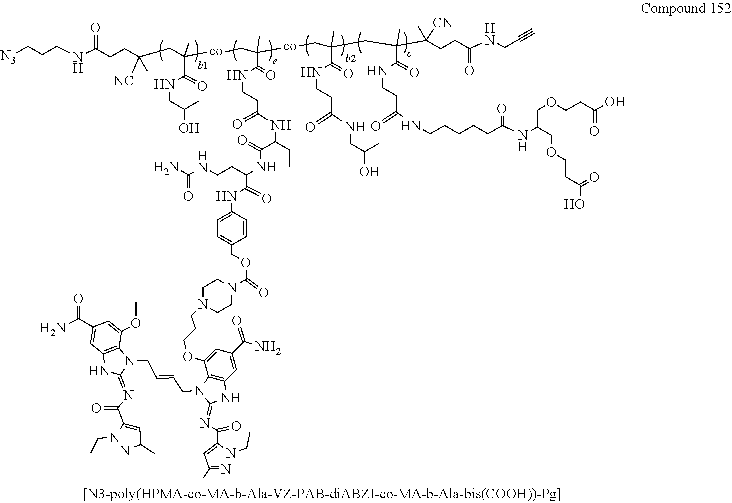

Compound 152. [N3-poly(HPMA-co-MA-b-Ala-VZ-PAB-diABZI-co-MA-b-Ala-bis(COOH))-Pg] is an example of a polymer arm comprised of hydrophilic monomers (B) and reactive monomers (E) attached to a drug (D2) via enzyme degradable linker and negatively charged groups, e.g., bis(COOH). N3-poly(HPMA-co-Ma-b-Ala-VZ-PAB-diABZI-co-MA-b-Ala-bis(COOH))-Pg can be synthesized with varied mol % of diABZI and bis(COOH) charge groups by tuning [diABZI-PAB-Cit-Val]:[bis(COOH)]. For an example, polymer arm with 10 mol % of diABZI and 6 mol % of bis(COOH) was synthesized by reacting the carbonylthiazolidine-2-thione (TT) groups of Compound 45 with Compound H, diABZI-PAB-Cit-Val, and bis(COOH) in the molar ratio=5:3. Specifically, N3-poly(HPMA-co-Ma-b-Ala-TT)-Pg (5.0 mg, 10.5 μmol TT) dissolved in 100 μL of anhydrous DMSO were mixed with diABZI-PAB-Cit-Val (3.3 mg, 2.6 μmol in 66.4 μL of DMSO) and triethylamine (2.7 mg, 26.3 μmol). The reaction was allowed to proceed at r.t. for 3 h before the addition of bis(COOH) (0.6 mg, 1.6 mmol) in 11.1 μL of anhydrous DMSO and triethylamine (2.7 mg, 26.3 μmol). After overnight reaction at r.t., amino-2-propanol (3.2 mg, 42.1 μmol) was added to quench the remaining reactive monomer. The product was then purified by precipitating against diethyl ether (4 rounds) and dried overnight in a vacuum oven. The product was obtained as a white powder. Mn and PDI were 65.2 kDa and 1.3, respectively, measured by GPC-MALS. The diABZI content measured by UV-Vis spectroscopy [ϵ₃₂₀ (diABZI)=23822 L/(mol·cm) showed 9.1 mol % diABZI.

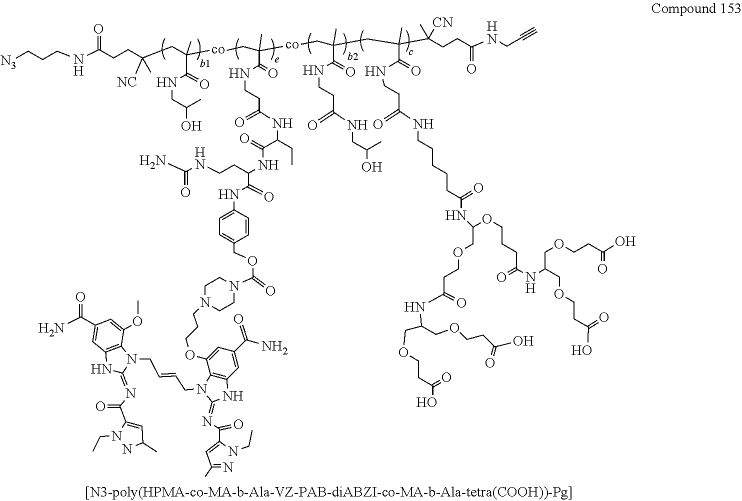

Compound 153. N3-poly(HPMA-co-MA-b-Ala-VZ-PAB-diABZI-co-MA-b-Ala-tetra(COOH))-Pg was synthesized in the same manner as Compound 152 by replacing the charge group with tetra(COOH).

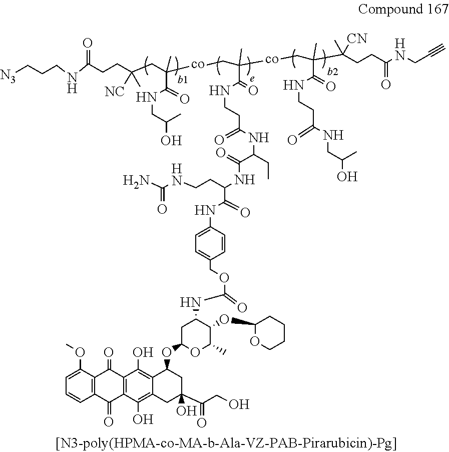

Compound 167. N3-poly(HPMA-co-MA-b-Ala-VZ-PAB-Pirarubicin)-Pg was synthesized in the same manner as Compound 59 by replacing the drug molecule with Compound AQ.

### Example 4—Functionalization of Dendrimer Cores with X1

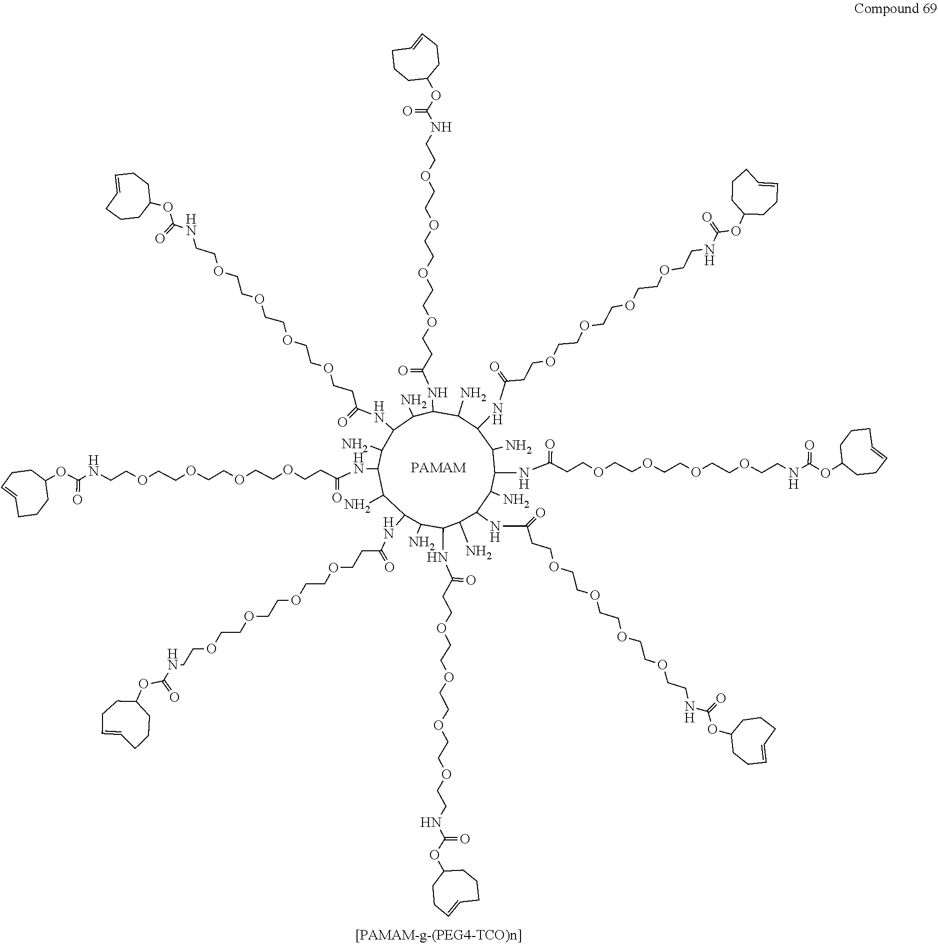

Compound 69 is an example of an X1 linker precursor linked to a core through a PEG linker. Trans-Cyclooctene (TCO)-functionalized G3 PAMAM dendrimer, PAMAM(G3)-g-(PEG4-TCO)n, was synthesized by reacting TCO-PEG4-NHS ester with G3 PAMAM dendrimer cores. The following procedure was employed to produce PAMAM Gen 3.0 dendrimers with 16 TCO functional groups (PAMAM Gen3-16TCO): Into a 20 mL scintillation vial, TCO-PEG4-NHS ester solution (30.9 μL, 100 mg/mL in methanol, 5.79 μmol), PAMAM Gen 3.0 dendrimer solution (14.48 μL, 20 wt % in methanol, 0.36 μmol), and 250 μL of anhydrous DMSO were added. Methanol solvent was then removed by applying vacuum before the addition of triethylamine (1.6 μL, 11.6 μmol). The mixture was allowed to stir overnight at r.t. Triethylamine was removed by applying vacuum and the solution was stored at −20° C. for future use (assuming 100% yield). Note: The TOO group on the X1 linker precursor enables attachment to polymer arms with X2 linker precursor comprising tetrazine.

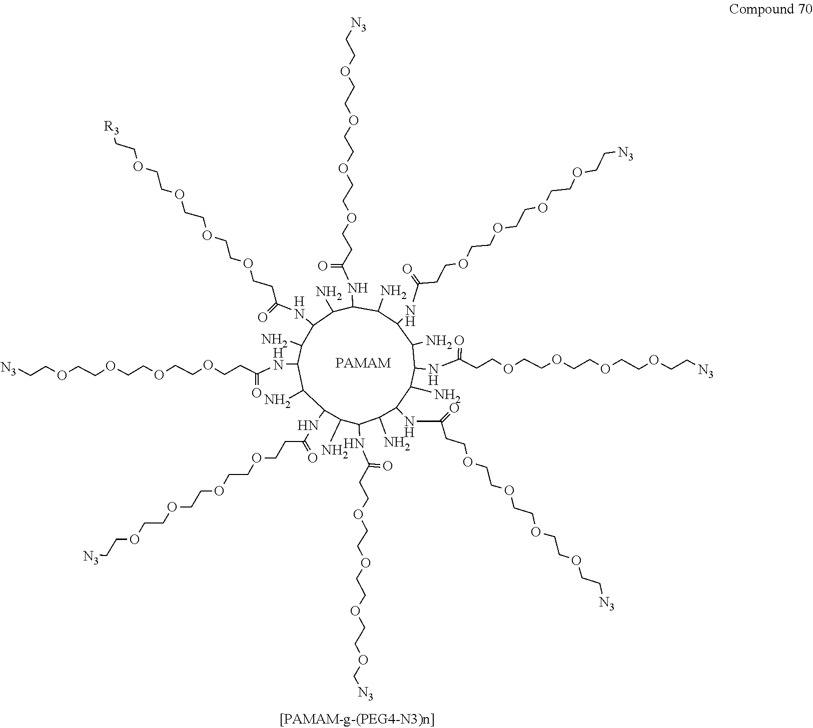

Compound 70 is an example of an X1 linker precursor linked to a core through a PEG linker. Azide-functionalized G5 PAMAM dendrimer, PAMAM(G5)-g-(PEG4-N3)n, was synthesized by reacting N3-PEG4-NHS ester with PAMAM cores. The following procedure was employed to produce PAMAM Gen 5.0 dendrimers with 64 azide functional groups (PAMAM Gen5-64N3): Into a 20 mL scintillation vial, N3-PEG4-NHS ester solution (21.6 μL, 100 mg/mL in methanol, 5.55 μmol), PAMAM Gen 5.0 dendrimer solution (62.7 μL, 5 wt % in methanol, 86.7 nmol), and 125 μL of anhydrous DMSO were added. Methanol solvent was then removed by applying vacuum before the addition of triethylamine (1.54 μL, 11.1 μmol). The mixture was allowed to stir overnight at r.t. Triethylamine was removed by applying vacuum and the solution was stored at −20° C. for future use (assuming 100% yield). Note: The azide group on the X1 linker precursor enables attachment to polymer arms with X2 linker precursor comprising alkynes.

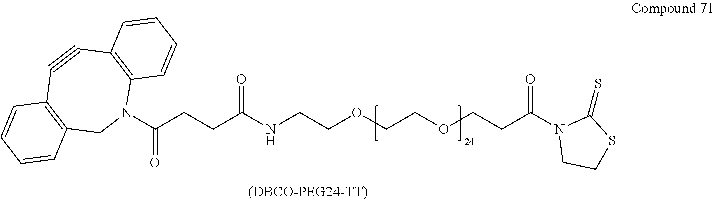

Compound 71. DBCO-PEG24-TT was synthesized via a two-step reaction from the starting compound Amino-PEG24-Acid. Amino-PEG24-acid (400 mg, 1 eq) was dissolved in THF to a concentration of 100 mg/mL. DBCO-NHS ester (154 mg, 1.1 eq) was dissolved in THF to a concentration of 50 mg/mL and added to the solution of Amino-PEG24-acid. Triethylamine (71 mg, 2 eq) was then added to the reaction mixture, which was incubated overnight with stirring at room temperature. reacted overnight at room temperature. The crude product was purified on a preparatory HPLC using a gradient of 25-55% acetonitrile/H₂O (0.05% TFA) over 12 minutes on an Agilent Prep C-18 column, 50×100 mm, 5 μm. The product fractions were pooled and lyophilized yielding light yellow oily solid DBCO-PEG24-acid (271.9 mg, 54.4%). DBCO-PEG24-acid (265.8 mg, 1 eq) was then dissolved in DCM to a concentration of 50 mg/mL. Thiazolidine-2-thione (24.3 mg, 1.1 eq) was likewise dissolved in DCM to a concentration of 100 mg/mL and added to the solution of DBCO-PEG24-acid. 1-Ethyl-3-(3-dimethylaminopropyl)carbodiimide (EDC) (86 mg, 2.4 eq) was dissolved in DCM to a concentration of 100 mg/mL and added to the reaction mixture. The reaction mixture was then cooled on wet ice and 4-Dimethylaminopyridine (DMAP) (1.1 mg, 0.05 eq) was added as a catalyst. The reaction was allowed to warm to room temperature while reacting for two hours, after which the product DBCO-PEG24-TT was purified on a preparatory HPLC using a gradient of 37-67% acetonitrile/H₂O (0.05% TFA) over 12 minutes on an Agilent Prep C-18 column, 50×100 mm, 5 μm. The product fractions were pooled and lyophilized yielding yellow oily solid DBCO-PEG24-TT (206.9 mg, 72.5%).

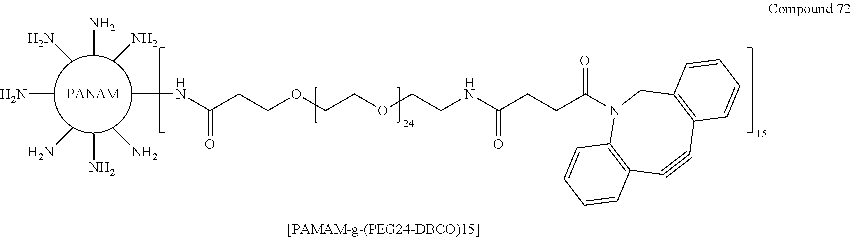

Compound 72 is an example of an X1 linker precursor linked to a core through a PEG linker, wherein the PEG in this example has 24 units of ethylene oxide. PAMAM(G5)-g-(PEG24-DBCO)₁₅ was synthesized by reacting DBCO-PEG24-TT with PAMAM dendrimer to yield a PAMAM dendrimer functionalized with 15 DBCO moieties with an extended 24-PEG linker. DBCO-PEG24-TT (20 mg, 15 eq) was dissolved in 0.6 mL of THF and added to PAMAM generation 5 (G5) (25 mg, 1 eq, 5 wt % in MeOH). The reaction was allowed to proceed for two hours at room temperature and monitored via analytical HPLC. Unreacted DBCO-PEG24-TT or hydrolyzed DBCO-PEG24-acid was then removed via dialysis against 200 mL pure THF using a 25 kDa MWCO RC membrane. Dialyzed product was diluted with 2 mL DMSO, after which THF was removed by vacuum. Product concentration in DMSO was then determined by DBCO UV absorbance from the extinction coefficient. Yield 65.3%. Note: The strained-alkyne (i.e., DBCO) group on the X1 linker precursor enables attachment to polymer arms with X2 linker precursor comprising azides to form the linker X comprising a triazole.

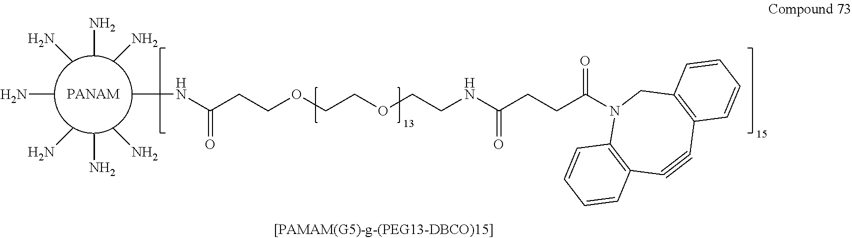

Compound 73 is an example of an X1 linker precursor linked to a core through a PEG linker, wherein the PEG in this example has 13 units of ethylene oxide. PAMAM(G5)-g-(PEG13-DBCO)₁₅ was synthesized by reacting DBCO-PEG13-NHS ester with PAMAM dendrimer in the same manner as Compound 72.

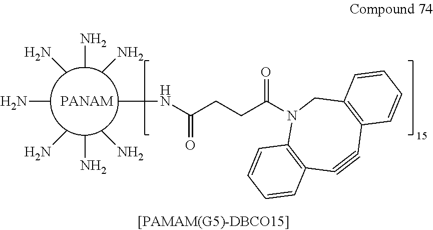

Compound 74 is an example of an X1 linker precursor linked to a core through a short linker. PAMAM(G5)-g-DBCO15 was synthesized by reacting DBCO-amine with PAMAM dendrimer in the same manner as Compound 72.

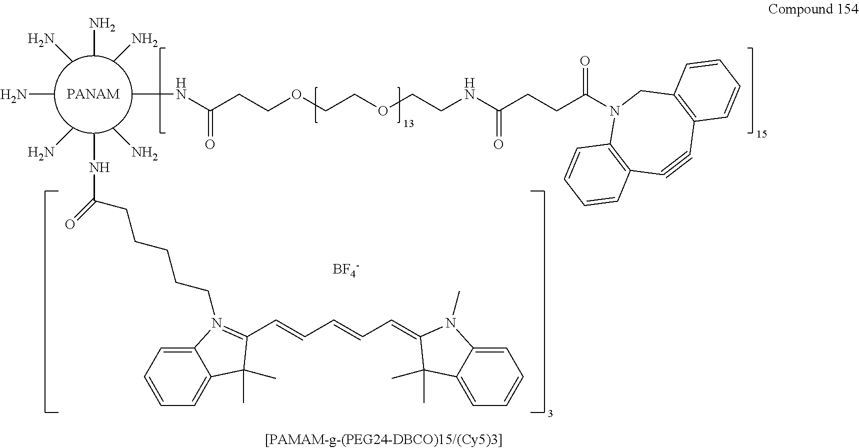

Compound 154. PAMAM-g-(PEG24-DBCO)15/(Cy5)3 is a fluorophore-tagged PAMAM dendrimer core with DBCO functional groups. The type and number of fluorescent dye molecule can be varied for different applications. Herein is an example inserting 3 dye molecules per each dendrimer core. Precursor Compound 72 was dissolved in anhydrous DMSO and mixed with Cyanine5 NHS ester (Cy5-NHS) (Lumiprobe, Cat #53020) pre-disslved in anhydrous DMSO at a ratio of [PAMAM]/[Cy5-NHS]=1/3. The mixture was vortexed and then allowed to react at room temperature overnight. Cy5-NHS ester was 100% converted to product which was confirmed by HPLC, and the mixture was used without further purification.

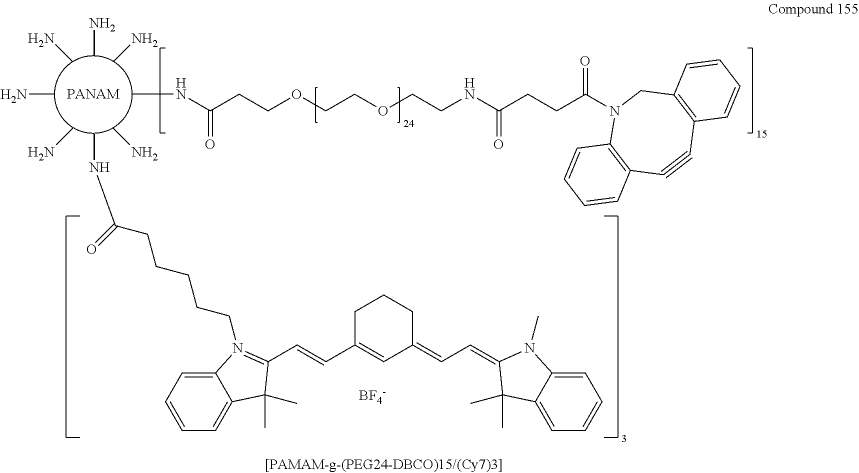

Compound 155. PAMAM-g-(PEG24-DBCO)15/(Cy7)3 was synthesized in the same manner as Compound 154 by replacing the fluorophore with Cyanine7 NHS ester (Lumiprobe, Cat #55020).

### Example 5—Synthesis of Star Polymers by Route 1

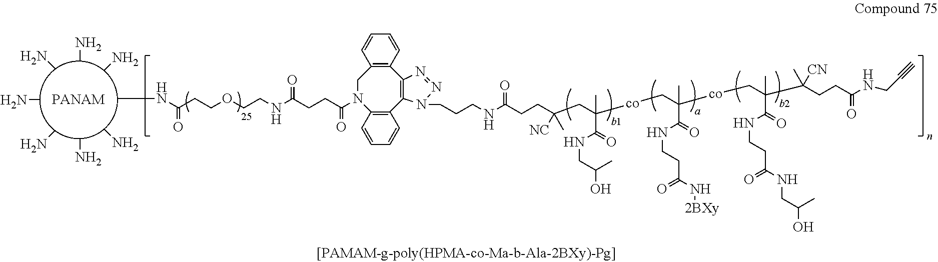

Compound 75 is an example of a star polymer with polymer arms comprised of hydrophilic monomers (B) and reactive monomers (E) linked to drug (D2, i.e., the TLR-7/8a, 2BXy) through an amide bond. PAMAM-g-poly(HPMA-co-Ma-b-Ala-2BXy)-Pg was synthesized by reacting Compound 72 PAMAM(G5)-g-(PEG24-DBCO)₁₅ with Compound 46 to yield a polymer. Example synthesis: N3-poly(HPMA-co-Ma-b-Ala-2Bxy)-Pg (3.55 mg, 75.0 nmol) and PAMAM(G5)-g-(PEG24-DBCO)₁₅ (0.501 mg, 150 nmol) were dissolved in 200 μL DMSO. The reaction was allowed to proceed at r.t. overnight. Reaction solution was precipitated in diethyl ether and dried overnight in vacuum oven to yield white powder. Number-average (Mn) and weight-average molecular weight (Mw) were 818.3 kDa and 998.4 kDa, respectively, and polydispersity (PDI) was 1.22 measured by GPC-MALS. Using Mn it was determined that the star NP was composed of 15.3 arms.

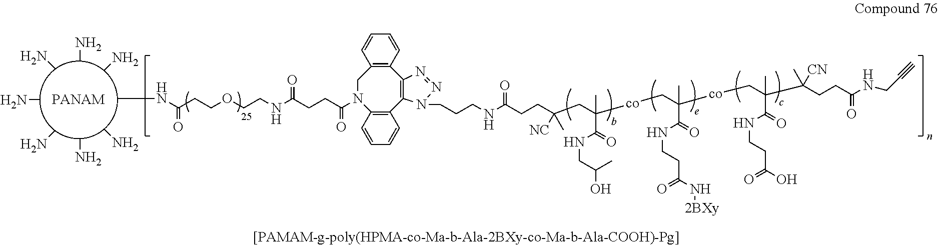

Compound 76 is an example of a star polymer with polymer arms comprised of hydrophilic monomers (B), reactive monomers (E) linked to drug (D2, i.e., the TLR-7/8a 2BXy) through an amide bond, and charged monomers (C) with a carboxylic acid functional group. PAMAM-g-poly(HPMA-co-Ma-b-Ala-2BXy-co-Ma-b-Ala-COOH)-Pg was synthesized using Compound 72 and Compound 48 in the same manner as Compound 75.

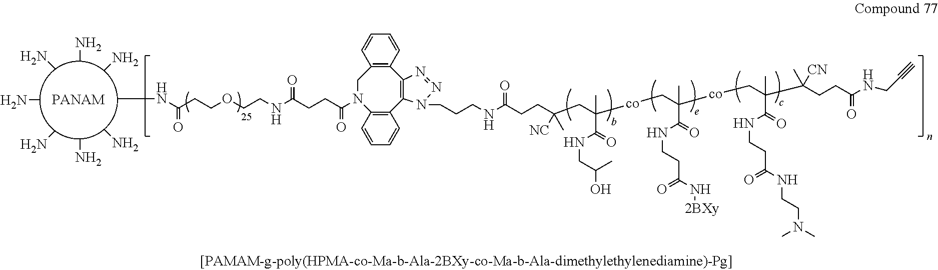

Compound 77 is an example of a star polymer with polymer arms comprised of hydrophilic monomers (B), reactive monomers (E) linked to drug (D2, i.e., the TLR-7/8a, 2BXy) through an amide bond, and charged monomers with a tertiary amine functional group. PAMAM-g-poly(HPMA-co-Ma-b-Ala-2BXy-co-Ma-b-Ala-dimethylethylenediamine)-Pg was synthesized using Compound 72 and Compound 52 in the same manner as Compound 75.

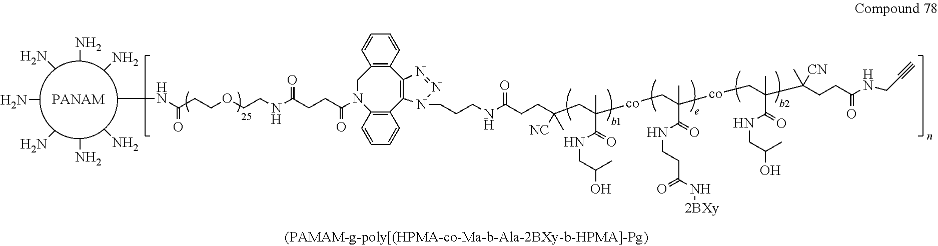

Compound 78 is an example of a star polymer with polymer arms with di-block architecture comprised of hydrophilic monomers (B) and reactive monomers (E) linked to drug (D), i.e., the TLR-7/8a, 2BXy, through an amide bond on one block proximal to the star polymer core and only hydrophilic monomers (B) on the other block distal to the core. PAMAM-g-poly[(HPMA-co-Ma-b-Ala-2BXy)-b-HPMA]-Pg was synthesized using Compound 72 and Compound 62 in the same manner as Compound 75.

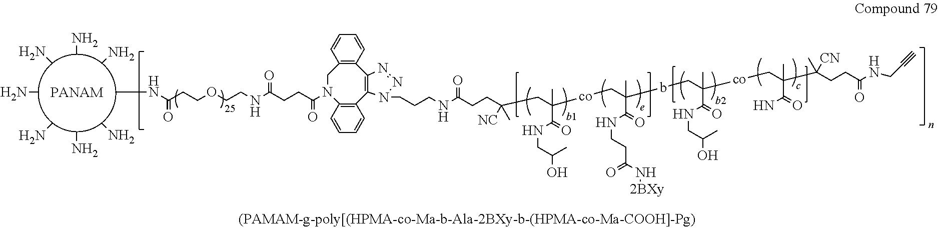

Compound 79 is an example of a star polymer with polymer arms with di-block architecture comprised of hydrophilic monomers (B) and reactive monomers (E) linked to drug (D), i.e., the TLR-7/8a, 2BXy, through an amide bond on one block proximal to the star polymer core, and both hydrophilic monomers (B) and charged monomers (C) with a carboxylic acid functional group on the other block distal to the core. PAMAM-g-poly[(HPMA-co-Ma-b-Ala-2BXy)-b-(HPMA-co-Ma-COOH]-Pg was synthesized using Compound 72 and Compound 65 in the same manner as Compound 75.

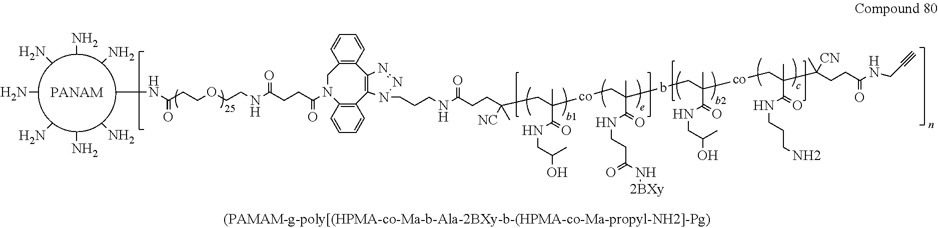

Compound 80 is an example of a star polymer with polymer arms with di-block architecture comprised of hydrophilic monomers (B) and reactive monomers (E) linked to drug (D), i.e., the TLR-7/8a, 2BXy, through an amide bond on one block proximal to the star polymer core, and both hydrophilic monomers (B) and charged monomers (C) with an amine functional group on the other block distal to the core. PAMAM-g-poly[(HPMA-co-Ma-b-Ala-2BXy)-b-(HPMA-co-Ma-propyl-NH2]-Pg was synthesized using Compound 72 and Compound 68 in the same manner as Compound 75.

### Example 6—Synthesis of Star Polymers for Ligand Display by Route 2

For Route 2 synthesis of star polymers, polymer arms are grafted to the core first to generate a star polymer, followed by conjugation of D2 and/or D2 to the star polymer.

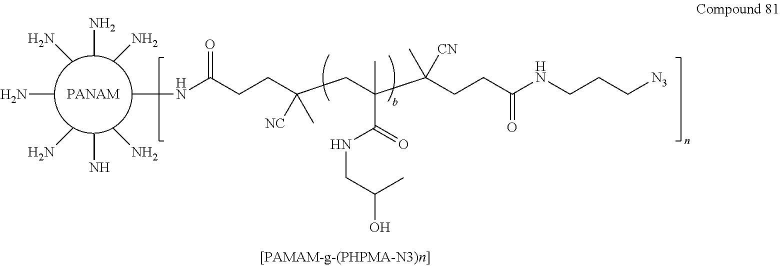

Compound 81 is an example of a star polymer, wherein the polymers arms (A) are linked to the core through a linker X that comprises an amide and are terminated with a Z1 linker precursor that comprises an azide. The following procedure was employed to produce azide-functionalized star NP with TT/NH2 linkages [PAMAM-g-(PHPMA-N3)n] by acylation between TT on PHPMA arm and primary amine on PAMAM core: TT-PHPMA-N3 (376.3 mg, 7.68 μmol) was dissolved in 1.5 mL of anhydrous DMSO in a 15 mL falcon tube. PAMAM dendrimer generation 3.0 solution (19.2 μL of 20 wt % in MeOH solution, 15.36 μmol of —NH2 groups) was added to the tube. The reaction was allowed to proceed at r.t. overnight. The star polymer was purified using spin column (Amicon, 70 mL, MWCO 50 kDa) and lyophilized to yield white solid (300.0 mg, 78.9% yield). Number-average (Mn) and weight-average molecular weight (Mw) were 848.9 kDa and 914.4 kDa, respectively, and polydispersity (PDI) was 1.08 measured by GPC-MALS.

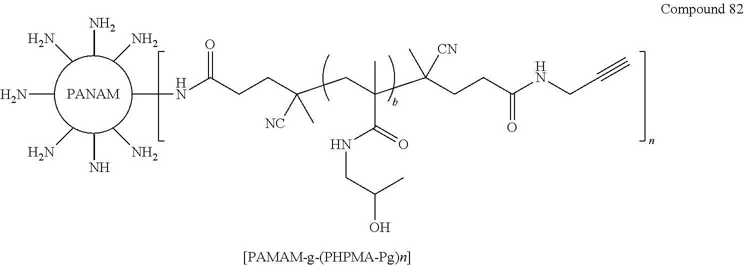

Compound 82 is an example of a star polymer, wherein the polymers arms (A) are linked to the core through a linker X that comprises an amide and are terminated with a Z1 linker precursor that comprises a propargyl (acetylene). Propargyl-functionalized star polymers with TT/NH2 linkages [PAMAM-g-(PHPMA-Pg)n] were prepared by acylation between TT-PHPMA-Pg and primary amine on PAMAM dendrimer using the same method as described for Compound 81.

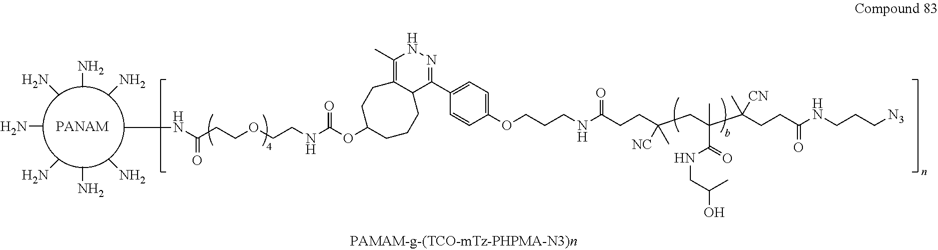

Compound 83 is an example of a star polymer, wherein the polymers arms (A) are linked to the core through a linker X that comprises the product of methyltetrazine and TCO and are terminated with a Z1 linker precursor that comprises an azide. Azide-functionalized star polymers with mTz/TCO linkages [PAMAM-g-(TCO-mTz-PHPMA-N3)n] were prepared using “click” chemistry between the mTz group on Compound 29, mTz-PHPMA-N3 and TCO groups on Compound 69, PAMAM-TCO dendrimer in the same manner as described for as described for Compound 81.

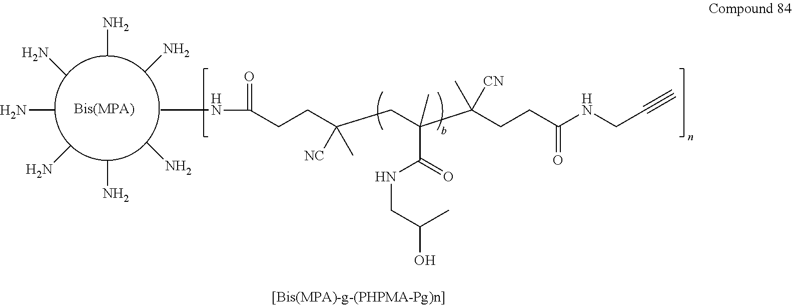

Compound 84 is an example of a star polymer, wherein the polymers arms (A) are linked to the core through a linker X that comprises an amide and are terminated with a Z1 linker precursor that comprises a propargyl. Bis(MPA)-g-(PHPMA-Pg)n was synthesized using the same method as described for Compound 82, PAMAM-g-(PHPMA-Pg)n, except that PAMAM dendrimer was replaced by bis(MPA) and triethylamine (TEA) was added to deprotonate amine groups on bis(MPA) core, with TT/NH2/TEA=0.8/1/1. White solid was obtained with 22.4% yield. Number-average (Mn) and weight-average molecular weight (Mw) were 327.2 kDa and 388.5 kDa, respectively, and polydispersity (PDI) was 1.19 measured by GPC-MALS.

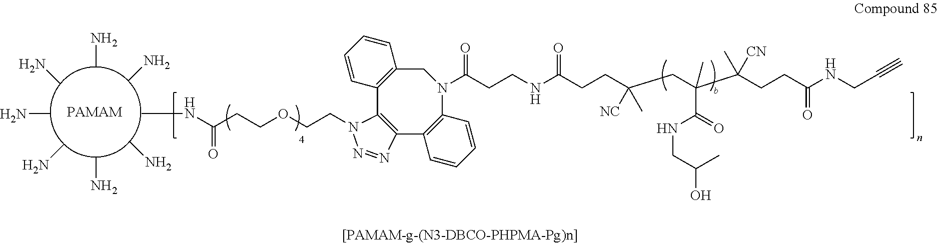

Compound 85 is an example of a star polymer, wherein the polymers arms (A) are linked to the core through a linker X that comprises a triazole and are terminated with a Z1 linker precursor that comprises a propargyl. Propargyl-functionalized star polymers with DBCO/N3 linkages [PAMAM-g-(N3-DBCO-PHPMA-Pg)n] were prepared using “click” chemistry between the DBCO group on Compound 35, Pg-PHPMA-DBCO and azide groups on Compound 70, PAMAM-N3 dendrimer in the same manner as described for as described for Compound 81.

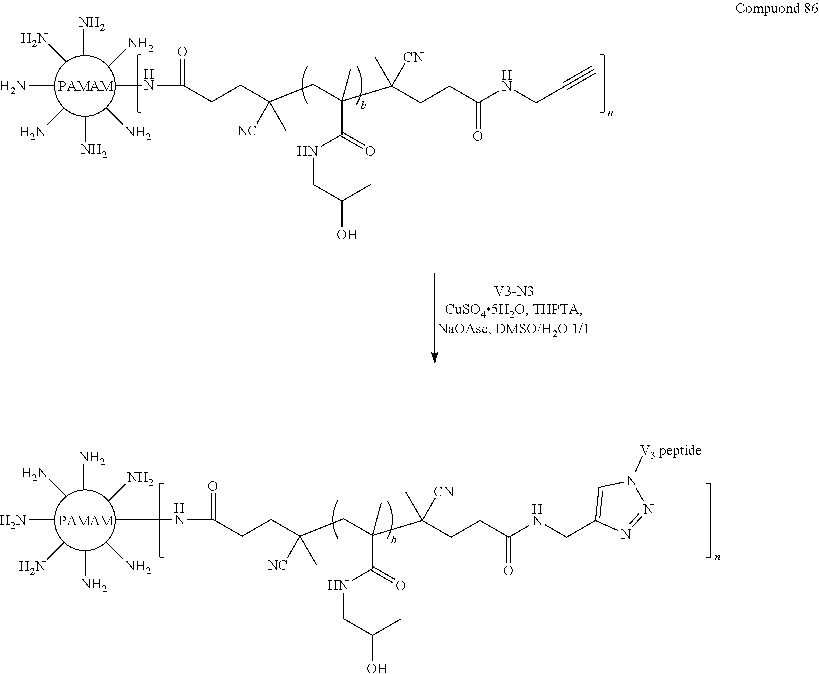

Compound 86. Star polymers displaying multiple B cell immunogens (peptide-N3 or “V3-N3”) on the surface was synthesized via copper-catalyzed alkyne-azide “click” chemistry. [peptide-N3]₀:[Pg]₀ molar ratio is adjusted to vary V3 loading per each star molecule and HPLC was used to ensure quantitative conversion. For example, star polymer PAMAM-g-(PHPMA15 k-Pg)₃₀] (1.5 mg, 100 nmol Pg), V3-N3 (0.27 mg, 78 nmol), CuSO₄·5H₂O (0.40 mg, 1.6 μmol), sodium ascorbate (NaOAsc, 0.32 mg, 1.6 μmol), and THPTA (0.69 μg, 1.6 μmol) were mixed in 87 μL of DMSO/H₂O cosolvent (1/1 v/v). The reaction was allowed to proceed at r.t. overnight. HPLC characterization was performed to confirm quantitative conversion of V3-N3 peptide. The reaction mixture was diluted to 3× the original volume with MeOH/H₂O cosolvent (1/1, v/v). The product was then purified by dialyzing against 2 rounds of MeOH/H₂O (1/1, v/v) with 0.01% ethylenediaminetetraacetic acid (EDTA), MeOH/H₂O cosolvent (1/1, v/v) and 2 rounds of H₂O. The resulting solution was lyophilized to yield off-white solid product (1.2 mg, 67.8% yield).

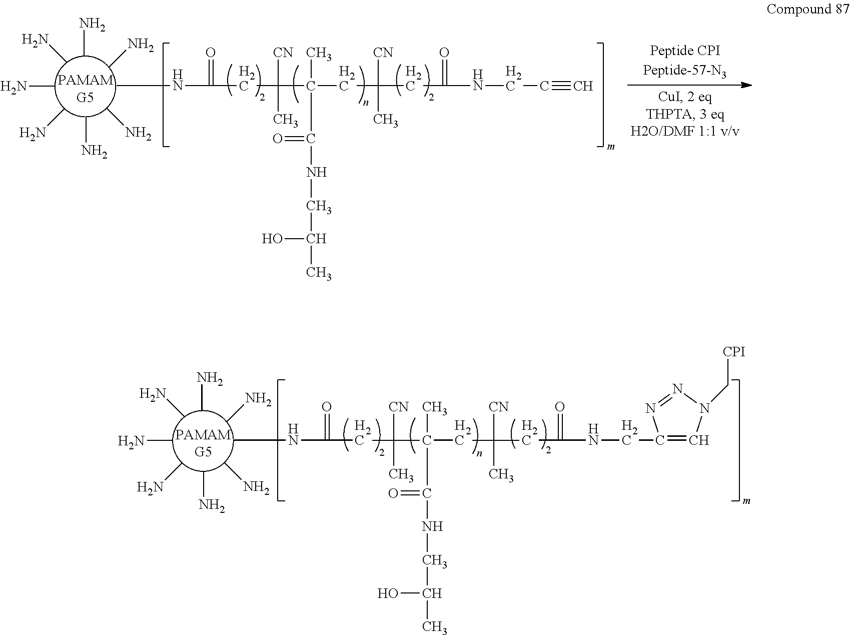

Compound 87. A star polymer displaying D3 (Compound Q, peptide-based macrocyclic checkpoint inhibitor) on the surface was synthesized via copper-catalyzed alkyne-azide (CuAAC) “click” chemistry, as summarized in the scheme, above. [peptide-N3]₀:[Pg]₀ molar ratio is adjusted to vary Compound Q loading per each star polymer molecule and HPLC was used to ensure quantitative conversion. For example, propargyl terminated star polymer (Compound 82) PAMAM-g-(PHPMA30 k-Pg)24] (2.3 mg, 74.2 nmol Pg), Compound Q (0.15 mg, 74.2 nmol), CuI (0.028 mg, 148.4 nmol), and THPTA (0.097 μg, 222.6 nmol) were mixed in 33 μL of DMF/H2O cosolvent (1/1 v/v) pre-sparged with argon gas. The reaction was allowed to proceed at room temperature overnight. HPLC characterization was performed to confirm quantitative conversion of Compound Q. The reaction mixture was diluted to 3× the original volume with MeOH/H₂O cosolvent (1/1, v/v) and purified by dialysis using a 10 kDa MWCO regenerated cellulose membrane. Dialysis was performed first for 16 h against MeOH/H₂O (1/1, v/v) with 25 mM ethylenediaminetetraacetic acid (EDTA), second for 3 hours against MeOH/H₂O cosolvent (1/1, v/v) with 2.5 mM EDTA, third against and MeOH/H₂O cosolvent (1/1, v/v) without EDTA followed by fourth round of dialysis against 100% MeOH. MeOH was then removed by under reduced pressure and the product was dissolved in DMSO for storage at reduce temperature (−20° C.). The peptide concentration of the purified conjugate was determined using an absorbance measurement in MeOH at 280 nm with extinction coefficient 10,018 L/(mol·cm) (930 μg conjugate, 37.9% yield).

Compounds 88-93. Variants of Star-p(HPMA-CPI) were synthesized similarly as described for Compound 87 using CuAAC for conjugation of Compound Q (with Z2 linker precursor comprising an azide) to star-polymers (with Z1 linker precursor comprising an alkyne) with varying dendrimer (i) core generation number (G2 or G5), (ii) number of polymer arms and (iii) polymer arm molecular weight. The resulting star polymers are summarized in Table B, below.

TABLE B
Star polymers displaying D3, wherein D3 = a peptide-based CPI.

| | | | | | Star polymer O(-X-A-Z-D3)n, | | |
|---|---|---|---|---|---|---|---|
| | PAMAM | | Polymer arm | | O(-X-A-Z1 or X-A-Z-D3)n, | | |
| | Dendrimer Core | | (Pg-PHPMA-TT) | TT/NH2/TEA | Star | Polymer | |
| Cmpd | | # of | Mn | molar | polymer Mn | Arm # | D3 |
| # | Generation | NH2 | (kDa) | ratio | (kDa) | (n) | # |
| 87 | G5 | 128 | 32.3 | 0.46/1/1 | 750.3 | 24.2 | 24 |
| 88 | G2 | 16 | 7.1 | 0.76/1/1 | 50.9 | 6.7 | 6 |
| 89 | G5 | 128 | 7.1 | 0.46/1/1 | 266.6 | 33.6 | 6 |
| 90 | G5 | 128 | 7.1 | 0.46/1/1 | 266.6 | 33.6 | 30 |
| 91 | G2 | 16 | 39.9 | 0.76/1/1 | 286.6 | 7.1 | 6 |
| 92 | G5 | 128 | 39.9 | 0.46/1/1 | 1221.2 | 30.6 | 6 |
| 93 | G5 | 128 | 39.9 | 0.46/1/1 | 1221.2 | 30.6 | 30 |

Note:
In the above table, Compound 89 and Compound 92 have approximately 30 polymer arms, but only 20% (i.e., 6) of the polymer arms are linked to D3, the other 80% (~24) polymer arms are terminated/or “capped” with the linker precursor Z1.

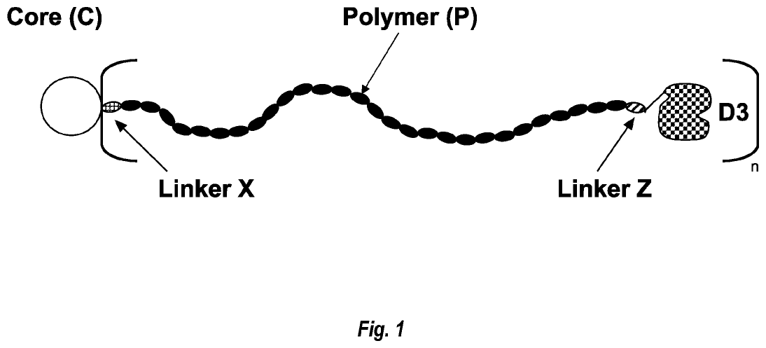

Compound 87 is a star polymer with a drug molecule (D3, i.e., a macrocyclic peptide-based CPI) linked to the ends of the polymer arms, which may be depicted schematically as shown in FIG. 1. Compound Q was modified to include an azido-lysine as a reactive handle (i.e., Z2) for conjugation to polymer arms with a linker precursor Z1 comprising an acetylene group. After conjugation of Compound Q to a star polymer to generate Compound 87, the physicochemical properties and biological activity were evaluated.

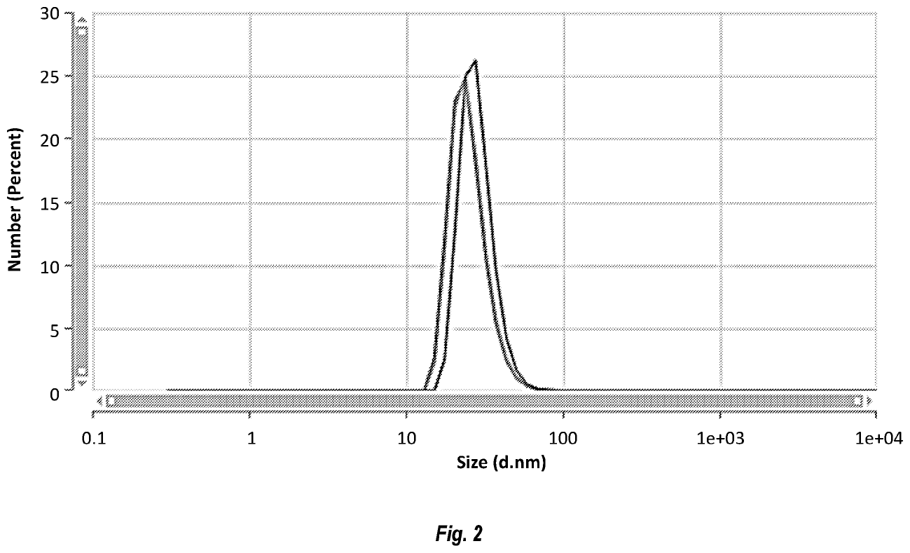

As shown in FIG. 2, conjugation of Compound Q, which is amphiphilic, to the star polymer had minimal impact on hydrodynamic behavior as determined by DLS. To evaluate what impact linking the macrocyclic peptide-based CPI to star polymers had on biological activity, we evaluated the capacity of Compound 87 to inhibit PD-1/PD-L1 interactions as compared with the an anti-PD-1 antibody (Nivolumab), the native (i.e., unmodified) macrocyclic peptide-based CPI and a polymer arm linked directly to Compound Q (“PHPMA-Compound Q”) using a Promega (Madison, WI) kit for assessing PD-1/PD-L1 inhibition (Catalog number J1250) according to the manufacturer's protocol. In short, each of the compounds were serially diluted in triplicate and incubated with a co-culture of Jurkat T cells expressing human PD-1, and CHO-K1 cells expressing PD-L1 and a cell surface protein that binds T cell receptor in an antigen independent manner. Inhibition of PD1/PD-L1 interactions releases the downstream inhibitory signal and allows signaling downstream of the TCR resulting in NFAT-mediated luciferase expression, which can be quantified by fluorescence measurements.

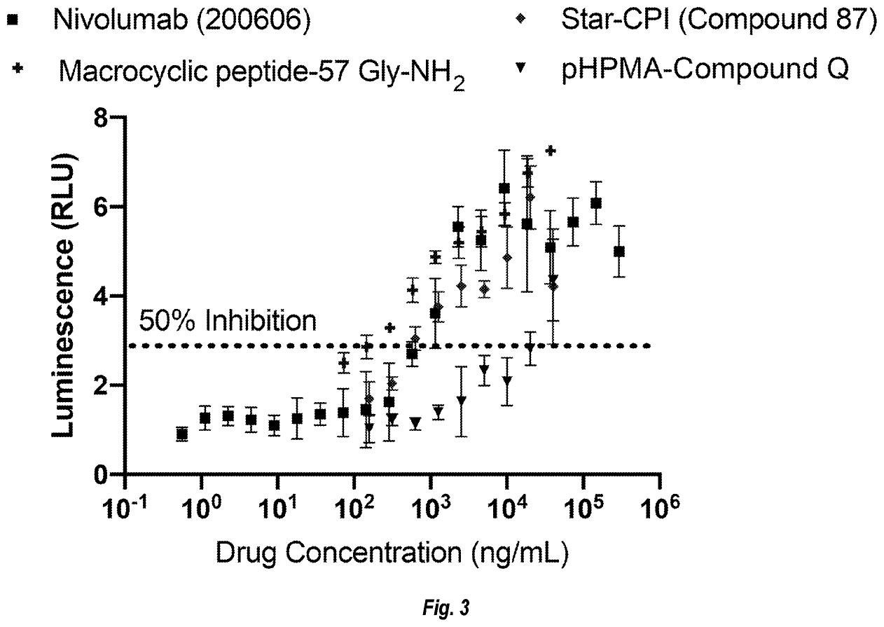

As shown in FIG. 3, Compound 87, which is multivalent, led to a nearly 100-fold increase in the potency of Compound Q as compared with the single polymer arm (PHPMA-Compound Q), which is monovalent, suggesting that Compound 87 may provide increased avidity of interaction as compared with monovalent versions of Compound Q. Importantly, these data show that the activity of Compound Q, which is a representative CPI, was preserved following conjugation to a star polymer as the surface displayed drug molecule (i.e., D3), and was as potent on a per mass basis as a FDA-approved CPI, Nivolumab.

### Example 7—Impact of Polymer Arm (A) Molecular Weight on Star Polymer Rh

The impact that polymer arm density, polymer arm molecular weight and dendrimer core generation have on the size (radius, e.g., Rg) of star polymers was investigated. Accordingly, polymer arms based on Pg-PHPMA-TT were synthesized using the same synthetic procedure as for the preparation of Compound 34 except that the monomer, chain transfer agent and initiator ratio (i.e., [M]₀:[CTA]₀:[I]₀) was adjusted to produce four HPMA-based polymers arms of varying molecular weight as summarized in Table C, below. Each of the different molecular weight HPMA-based polymers bearing an X2 linker precursor comprising a TT-activated acid was then reacted with either a PAMAM Generation G3 or G5 core with 32 or 128 amine functionalities, respectively, at different ratios of TT (X2) to amine (X1) to generate star polymers with between ˜10-30 polymers arms per star polymer (Table C). Note that the polymer arms (A) were attached to the core (O) using the same procedure as described for Compound 82, except with varying molar ratio of polymer arm and amine functionalities.

TABLE C
Star polymers comprising PAMAM cores
with different PHPMA arm lengths.

| PAMAM | | | TT/NH2 | Star polymer | | | |
|---|---|---|---|---|---|---|---|
| | # of | Arm Mn | molar | Mn | | | Rg |
| Gen. | NH2 | (kDa) | ratio | (kDa) | Mw/Mn | Arm # | (nm) |
| 3 | 32 | 10.20 | 0.2 | 118.80 | 1.18 | 11.0 | 9.6 |
| 3 | 32 | 19.20 | 0.5 | 250.80 | 1.13 | 12.7 | 13.3 |
| 3 | 32 | 50.42 | 0.4 | 1106.52 | 1.06 | 21.8 | 23.7 |
| 5 | 128 | 9.95 | 0.5 | 304.52 | 1.03 | 27.7 | 8.0 |
| 5 | 128 | 15.07 | 0.5 | 444.23 | 1.07 | 27.6 | 10.5 |
| 5 | 128 | 25.94 | 0.64 | 765.52 | 1.04 | 28.4 | 14.5 |
| 5 | 128 | 30.44 | 0.5 | 907.10 | 1.03 | 28.9 | 16.4 |
| 5 | 128 | 38.40 | 0.5 | 909.0 | 1.07 | 22.9 | 19.6 |
| 5 | 128 | 54.15 | 0.5 | 1518.53 | 1.05 | 27.5 | 23.8 |
| 5 | 128 | 70.00 | 0.5 | 1476.9 | 1.07 | 20.7 | 28.0 |
| 5 | 128 | 88.45 | 0.63 | 2005.36 | 1.05 | 22.3 | 29.2 |

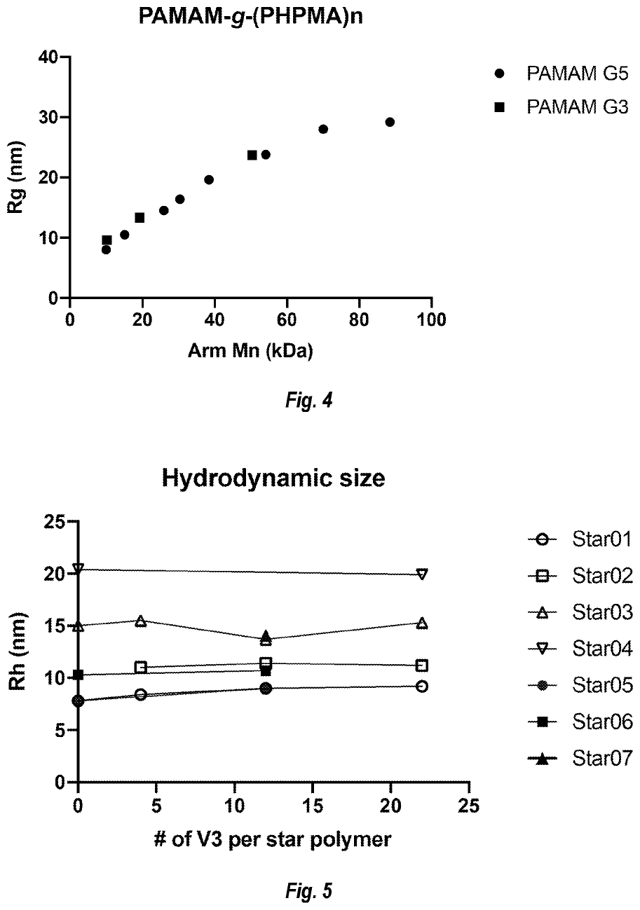

Unexpectedly, the radius of star polymers, both radius of gyration (Rg) and hydrodynamic radius (Rh) was principally dictated by the polymer arm molecular weight (FIG. 4). Separately, an HIV Env minimal immunogen, V3, was linked at different densities (4, 12 or 22 V3 peptides per star polymer) via a linker Z comprising a triazole to the star polymers of varying molecular weight and arm density (referred to as Star27 through Star07; Table 4), using the same method as described for Compound 86 to generate star polymers with varying arm length, arm number and proportion of arms linked to 03. The hydrodynamic behavior of the different star polymers is shown in FIG. 5. In brief, the data showed that increasing polymers arm length, i.e., increasing polymer arm (A) molecular weight, is associated with increased Rh, which is largely independent of the numbers of arms and proportion of those arms linked to 03.

TABLE 4
Star polymers of varying arm Mn and density displaying V3 as D3.

| | PAMAM | Pg-PHPMA-TT arm | | TT/NH2 | Star polymer properties | | |
|---|---|---|---|---|---|---|---|
| | (G5) | | Mn | molar | Mn | | Arm |
| Sample | # of NH2 | [M]₀:[CTA]₀:[I]₀ | (kDa) | ratio | (kDa) | Mw/Mn | # |
| Star01 | 128 | 120:1:0.25 | 15.0 | 0.5 | 435.5 | 1.06 | 27 |
| Star02 | 128 | 240:1:0.25 | 26.4 | 0.5 | 764.1 | 1.06 | 28 |
| Star03 | 128 | 600:1:0.25 | 54.1 | 0.5 | 1520.2 | 1.05 | 28 |
| Star04 | 128 | 1200:1:0.25 | 88.4 | 0.63 | 2512.6 | 1.08 | 28 |
| Star05 | 128 | 120:1:0.25 | 15.0 | 0.28 | 260.6 | 1.01 | 15 |
| Star06 | 128 | 240:1:0.25 | 26.4 | 0.28 | 463.2 | 1.05 | 16 |
| Star07 | 128 | 600:1:0.25 | 54.1 | 0.33 | 848.6 | 1.03 | 15 |

### Example 8—Starpolymers with an Ester-Based Core

Various branched molecules can be used as cores for generating star polymers. As an alternative to PAMAM (i.e., amide)-based cores, star polymers were produced using either generation 2, 4 or N bis(MPA), ester-based cores. TT-activated HPMA-based polymer arms (A) were reacted with bis(MPA) cores in the presence of triethylamine to generate the star polymers summarized in Table 5.

TABLE 5
Star polymers synthesized from bis(MPA) cores.

| bis(MPA) core | | Pg-PHPMA- | TT/NH2/ | Star polymer | | |
|---|---|---|---|---|---|---|
| (TFA salt) | | TT arm | TEA | properties | | |
| | # of | Mn | molar | Mn | Mw/ | Arm # |
| Generation | NH2 | (kDa) | ratio | (kDa) | Mn | (n) |
| G2 | 12 | 11.02 | 1/1/1 | 92.4 | 1.03 | 8.2 |
| G4 | 48 | 11.02 | 0.5/1/1 | 178.0 | 1.04 | 15.4 |
| G5 | 96 | 10 | 0.4/1/1 | 164.3 | 1.02 | 14.6 |
| G5 | 96 | 10 | 0.8/1/1 | 303.8 | 1.05 | 28.6 |

### Example 9—Methods for Preventing Star Polymer Cross-Linking During Manufacturing

Consistent manufacturing of uniform formulations is key to ensuring the success of any drug product for human use. Accordingly, star polymer manufacturing should ensure that star polymer compositions have uniform characteristics that are not variable between different batches.

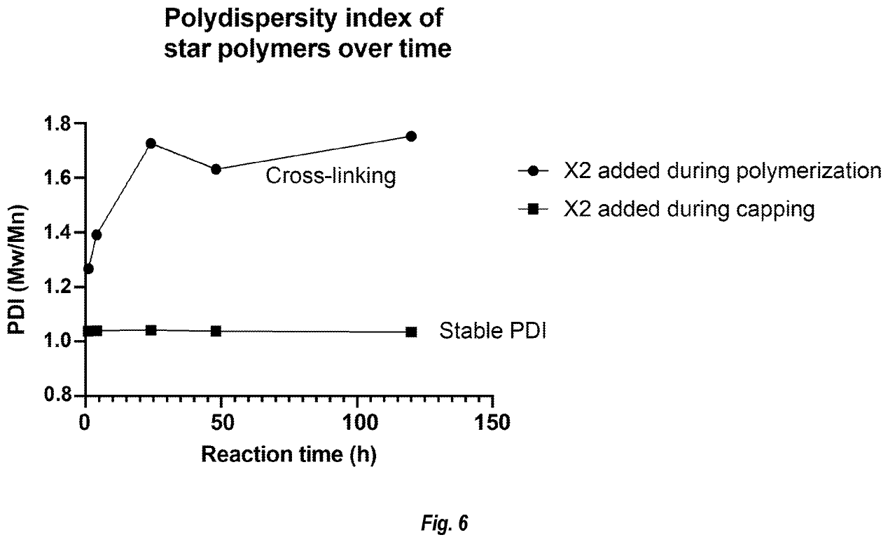

A key finding reported herein is that the process for introducing the linker precursor X2 on the star polymer can impact star polymer manufacturability. While the X2 linker precursor can be introduced on the polymer arm (A) either (i) during polymerization, i.e., by using a CTA and initiator functionalized with X2 (e.g., CTA-TT and ACVA-TT) or (ii) during the capping step, i.e., by reacting a polymer arm terminated with a CTA (e.g., PHPMA-DTB) with excess initiator functionalized with X2 (e.g., ACVA-TT), an unexpected finding reported herein is that introduction of X2 (or a reactive group for subsequent introduction of X2) during the polymerization step results in polymers arms prone to cross-linking star polymers as indicated by the high polydispersity index of star polymers produced by this route (FIG. 6). In contrast, introduction of X2 linker precursor (or a reactive group for subsequent introduction of X2) onto polymers arms during the capping step results in polymer arms that do not result in cross-linked star polymers. A non-limiting explanation for these results is that introduction of the X2 linker precursor on a polymer arm during polymerization, which is subsequently reacted with excess initiator during the capping step, results in a polymer arm impurity that is bifunctional for the linker precursor X2, i.e., the linker precursor X2 is linked to both ends of the polymer arm.

Based on these findings, several manufacturing innovations were introduced to reduce the potential for cross-linking to occur. As shown in FIG. 6, the risk of cross-linking can be eliminated by introducing the linker precursor X2 onto polymer arms during the capping step, rather than the polymerization step. However, for compositions of polymer arms that require the addition of the linker precursor X2 to the polymer arm during polymerization, two additional steps can be undertaken to reduce cross-linking, thereby improving manufacturability: (i) the concentration of the polymer arms in the reaction can be reduced and/or (ii) the time of the reaction can be reduced. Notably, it was observed that—for the synthesis of star polymers using polymer arms wherein the X2 linker precursor was introduced during the polymerization reaction—reducing the polymer arm concentration to 1 mM from 10 mM reduced the polydispersity index (PDI) of the results star polymers from about 1.7 to 1.07, indicating a marked reduction in cross-linking. Additionally, keeping the reaction time to 1 hour or less also resulted reduced PDI, indicating lower extent of cross-linking. Taken together, these results suggest that the linker precursor X2 should be introduced at any time after polymerization, e.g., during the capping step. Otherwise, if X2 must be added to the polymer arms during polymerization than the concentration of polymer arms during grafting to the core should be reduced to 1 mM or less and reaction time limited to prevent excessive cross-linking of the star polymers.

### Example 10—Methods for Improving Arm Coupling Efficiency to Star Polymers

Steric hindrance has historically prevented the efficient coupling of high densities (e.g., >10 mol %) of D2 to the arms of star polymers. Steric hindrance can also present challenges to coupling high densities of D3, especially D3 with >10,000 Dalton molecular weight, to the surface of star polymers. Therefore, it may be preferred to first attach D2 and/or D3 to polymer arms (A), and then couple these polymer arms to cores, which is a manufacturing process herein referred to as Route 1. A major challenge for Route 1 is that polymer arms bearing high densities of D2 and/or high molecular weight D3 are relatively bulky, which can impact polymer arm coupling efficiency to cores. An unexpected finding reported herein is that bulky polymer arms with high densities of drugs D2 and/or linked to moderate to higher molecular weight D3 could be more efficiently coupled to cores by introducing 4 or more ethylene oxide units onto X1 or on the linker between X1 and the core. Accordingly, the grafting efficiency, measured as mass percent conversion of polymer arms, was improved by extending the X1 linker precursor from the core using PEG13 or PEG24 (Table 6). These results show that the grafting efficiency can be improved markedly using linker precursors X1 linked to cores (O) through a PEG linker, i.e., X1 linker precursor comprising a PEG linker.

TABLE 6
Polymer arm grafting efficiency.

| | | | # of | % |
|---|---|---|---|---|
| X1 | Polymer arm | Mn | arms | Conversion |
| DBCO | Pg-poly[(HPMA)-b-(HPMA-co-Ma-b-Ala-2BXy)]-N3 | 739.4 | 17.2 | 13.8 |
| PEG13-DBCO | Pg-poly[(HPMA)-b-(HPMA-co-Ma-b-Ala-2BXy)]-N3 | 869.3 | 20.2 | 28.5 |
| PEG24-DBCO | Pg-poly[(HPMA)-b-(HPMA-co-Ma-b-Ala-2BXy)]-N3 | 812.8 | 18.6 | 67.1 |

### Example 11—Polymers with Block Architecture and/or Charged Monomers Enable Efficient Loading (i.e., High Densities) of Amphiphilic or Hydrophobic Drugs on Star Polymers

Increased density (mol %) of D2 attached to polymer arms of star polymer was generally associated with enhanced biological activity. Therefore, compositions and methods of manufacturing star polymers that enable consistent manufacturing of uniform formulations of star polymers with high densities (e.g., >10 mol %) of D2 are needed. In addition to the aforementioned challenges associated with the process for manufacturing star polymers with high densities of D2, the chemical composition of the drug (D2) can also pose challenges. Specifically, amphiphilic or hydrophobic drugs, such as small molecule drugs comprising cyclic ring structures, such as aromatic heterocycles, attached to the polymer arms of star polymers at high densities can cause aggregation of the star polymers, which can present challenges to manufacturing drug products for human use, as well as adversely alter pharmacokinetics and biodistribution in vivo when used for targeting tissues other than liver and spleen by the intravenous route.

To address this challenge, two design features were introduced that enable loading of high densities of amphiphilic or hydrophobic drug as D2 on the polymer arms of star polymers without the resulting star polymers aggregating. The two innovations were to either or both (i) use star polymers comprised of polymer arms (A) with diblock architecture wherein the amphiphilic or hydrophobic D2 is attached to the first block of the di-block copolymer and/or (ii) include charged monomers on the polymer arm (A).

It was unknown a priori what composition and magnitude of charge would be needed to fully solubilize polymer arms with high densities of amphiphilic or hydrophobic drugs linked to the polymers, or how the charged monomers would impact biological activity.

Therefore, as a model system, we first attached high densities (>10 mol %) of a representative amphiphilic or hydrophobic drug, 2BXy, which is a TLR-7/8a, to ˜40 kDa HPMA-based polymer arms (A) through a reactive monomer (E), wherein the polymer arm (A) comprised HPMA monomers as the majority hydrophilic monomer (B) and optionally included either 10 or 20 mol % charged monomers (C) comprising either negatively or positively charged functional groups.

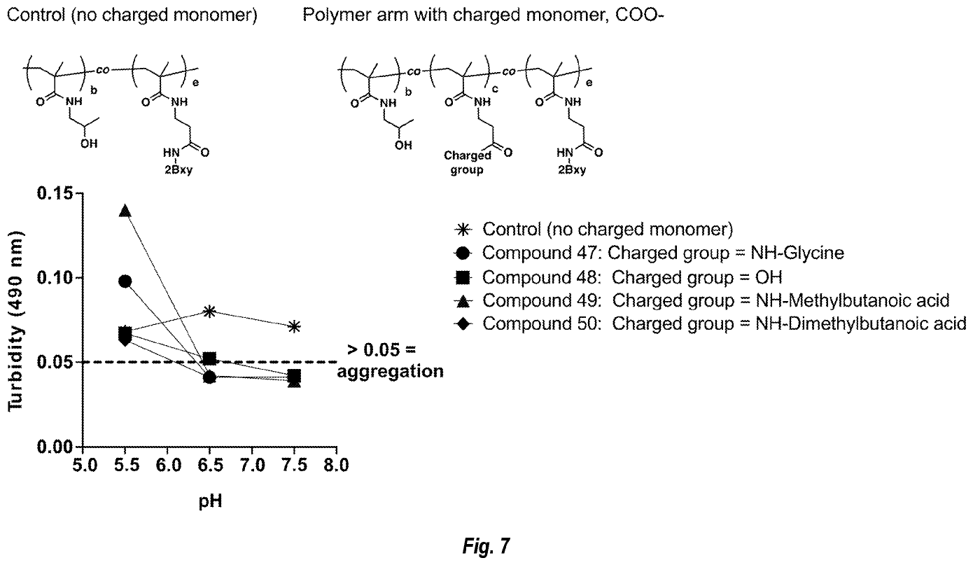

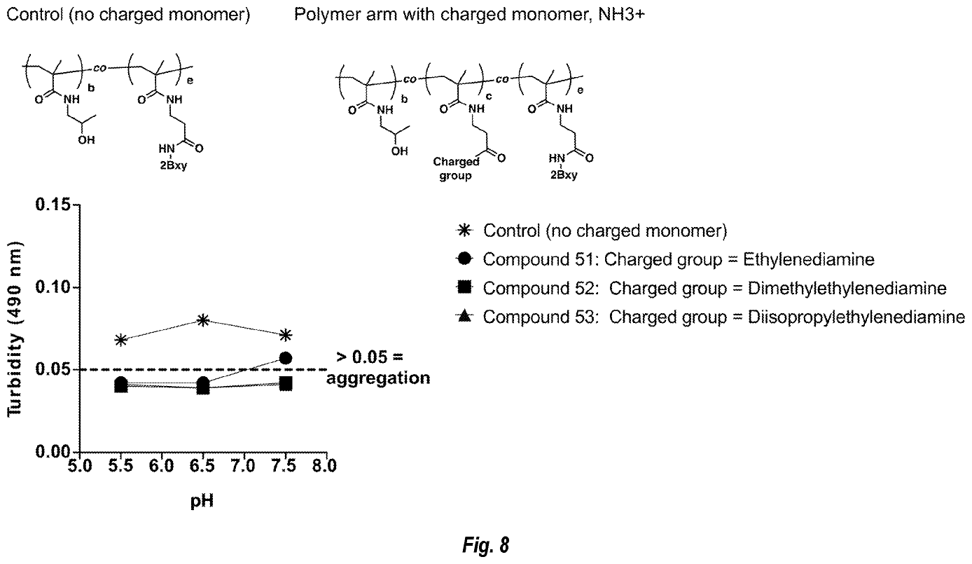

Notably, whereas the copolymer without charged monomers formed aggregates at physiologic pH, ˜pH 7.4, as indicated by turbidity measurements (FIG. 7), polymer arms (A) with negatively charged carboxylic acid groups did not form aggregates at physiologic pH. Similarly, polymer arms (A) that also included primary or tertiary amines, which can be at least partially protonated at physiologic pH, did not aggregate at physiologic pH (FIG. 8). Notably, polymer arms with ethylene diamine but not propylene diamine showed some tendency to form aggregates at physiologic pH, suggesting that C2 or higher alkyl chains, though typically no more than C6, may be preferred for alkyl-amine based charged groups (FIG. 8).

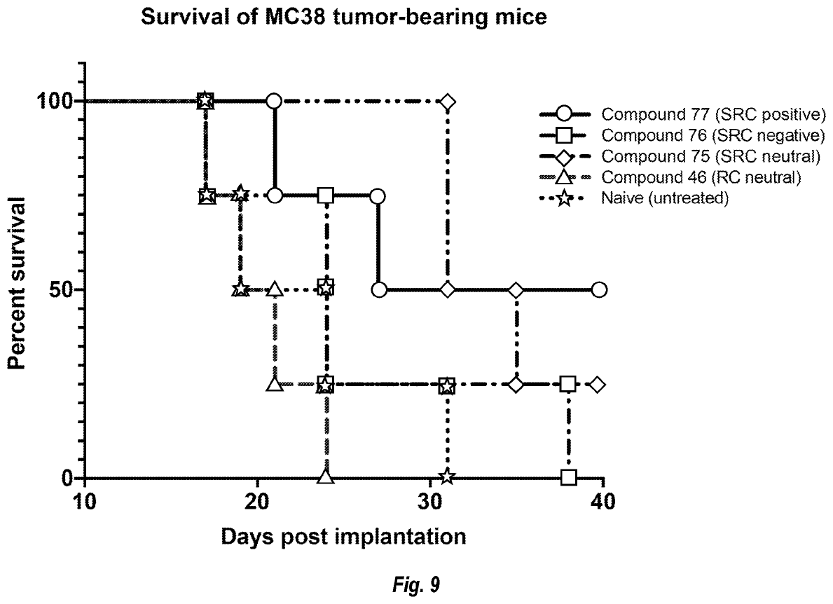

Based on these data, two different compositions of star polymers were generated with terpolymers comprised of hydrophilic monomers (HPMA), reactive monomers linked to drug (MA-b-Ala-2BXy) and charged monomers with either negative (Ma-b-Ala-COOH) or positive (Ma-b-Ala-DMEDA) functional groups (at physiologic pH). Notably, both star polymers (Compounds 76 and 77, Table 7) were stable in aqueous buffer (PBS) at physiologic pH. Importantly, preserving the small size (Rh˜10 nm) of the star polymers with high densities (˜10 mol %) of the TLR-7/8a by using high densities (˜20 mol %) of charged monomers was also associated with improved biological activity. Specifically, mice with MC38 tumors treated with the star polymers comprising TLR-7/8a and charged monomers had improved survival as compared with mice that received neutral star polymers with random coil architecture that did not include charged monomers (FIG. 9).

TABLE 7
Star polymers with polymer arms that include charged monomers and
high densitie of an amphiphilic or hydrophobic D2 (i.e., 2BXy).

| Cmpd. | | C | Mn | | Turbidity | Rh (nm) | Turbidity |
|---|---|---|---|---|---|---|---|
| # | Composition | Mol % (#) | (kDa) | PDI | at pH 7.4 | at pH 6.5 | at pH 6.5 |
| 48 | N3-poly[(HPMA-co-Ma-b-Ala-2BXy-co- | 20 (50) | 43.1 | 1.07 | 0.043 | 48.0 | Aggregate |
| | Ma-b-Ala)-Pg | | | | | | |
| 52 | N3-poly[(HPMA-co-Ma-b-Ala-2BXy-co- | 20 (50) | 46.1 | 1.10 | 0.044 | 4.1 | 0.040 |
| | Ma-b-Ala-DMEDA)-Pg | | | | | | |
| 76 | PAMAM-g-poly[(HPMA-co-Ma-b-Ala- | 20 (50) | 483.4 | 1.19 | 0.044 | | |
| | 2BXy-co-Ma-b-Ala)-Pg | | | | | | |
| 77 | PAMAM-g-poly[(HPMA-co-Ma-b-Ala- | 20 (50) | 665.6 | 1.20 | 0.052 | | |
| | 2BXy-co-Ma-b-Ala-DMEDA)-Pg | | | | | | |

Finally, star polymers with polymer arms (A) with di-block architecture were found to accommodate high densities (>10 mol %) of TLR-7/8a without forming aggregates (Table 8).

TABLE 8
Star polymers with polymer arms that have diblock architecture and high densities of an amphiphilic
or hydrophobic D2 (i.e., 2BXy) linked to reactive monomers on the first block.

| | | | | | Rh | | Rh | |
|---|---|---|---|---|---|---|---|---|
| | | | | | (nm) | Turbidity | (nm) | Turbidity |
| Cmpd. | | C | Mn | | at pH | at pH | at pH | at pH |
| # | Composition | Mol % (#) | (kDa) | PDI | 7.4 | 7.4 | 6.5 | 6.5 |
| 62 | N3-poly[(HPMA-co-Ma-b-Ala-2BXy)-b- | N.A. | 35.8 | 1.31 | 6.6 | 0.039 | 6.9 | 0.039 |
| | HPMA]-Pg | | | | | | | |
| 68 | N3-poly[(HPMA-co-Ma-b-Ala-2BXy)-b- | 5 (10) | 37 | 1.33 | 6.5 | 0.039 | 5.6 | 0.039 |
| | (HPMA-co-MA-propyl-NH₂)]-Pg | | | | | | | |
| 78 | PAMAM-g-poly[(HPMA-co-Ma-b-Ala- | N.A. | 588.2 | 1.34 | 12.9 | 0.041 | | |
| | 2BXy)-b-HPMA]-Pg | | | | | | | |
| 80 | PAMAM-g-poly[(HPMA-co-Ma-b-Ala- | 5 (10) | 372.1 | 1.35 | 12.4 | 0.043 | | |
| | 2BXy)-b-(HPMA-co-Ma-propyl-NH₂)]- | | | | | | | |
| | Pg | | | | | | | |

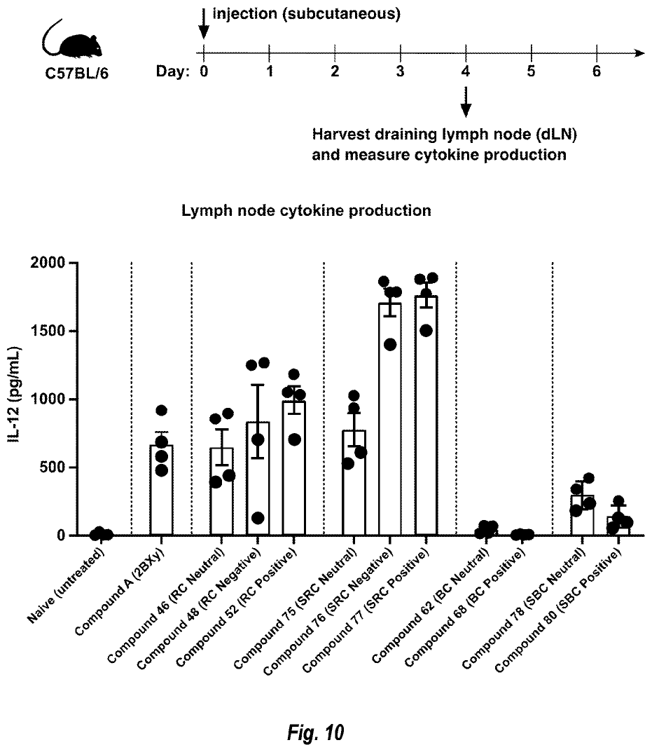

To further investigate how differences in polymer architecture and charge monomer composition impact the biological activity of star polymers comprising polymer arms with an amphiphilic or hydrophobic drug, e.g., a TLR-7/8a, linked to reactive monomers through an amide bond, we next assessed the capacity of random copolymer and diblock copolymer arms as well as star random copolymers and star diblock copolymers to induce innate immune activation in vivo. As shown schematically in the top of FIG. 10, C57Bl/6 mice were injected subcutaneously in the footpad with 25 nmol of TLR-7/8a as either the small molecule (“2BXy”) or a polymer arm-drug conjugate or star polymer-drug conjugate. Draining lymph nodes were harvested from treated animals 4 days later and cultured for 12 hours ex vivo. Lymph node culture supernatant was then assessed by ELISA for IL-12, which is a measure of innate immune activation by the TLR-7/8 agonist.

A notable and unexpected finding was that both the polymer arms and star polymers with polymer arms with random copolymer architecture, referred as RCs and SRCs led to higher magnitude immune activation as compared with polymer arms and star polymers with polymer arms with diblock architecture, referred to as DBs and SDBs (FIG. 10). A non-limiting explanation for these findings is that D2 is more accessible on SRCs than on SDBs. Though, notably, in this example, D2 is linked to the polymer arms through a relatively stable amide bond. In other studies, wherein D2 was linked to SRCs and SDBs through pH-sensitive bonds, e.g., hydrazone bonds, SRCs and SDBs, had comparable activity. These data suggest that SRCs are a more favorable architecture for attaching amphiphilic or hydrophobic D2 to polymer arms at high densities using relatively stable bonds or bonds that require enzymatic cleavage, which may otherwise be less accessible when present on the first block of diblock copolymer arms of SDBs. An additional notable finding was that the SRCs with charged monomers led to significantly increased (>2-fold) innate immune activation as compared with any of the RCs or SRC without charged monomers. These data show that star polymer carriers of a representative amphiphilic or hydrophobic drug with immunostimulatory properties, e.g., TLR-7/8a, lead to substantially higher activity as compared with the same drug molecule alone or on a single polymer arm, and that use of charged monomers to modulate hydrodynamic behavior as well as pH-responsiveness of the star polymers (e.g., star polymers of Formula V) can further improve activity of the star polymer drug carriers.

### Example 12—Efficacy of RC-diABZI with Different Polymer-Drug Linkages

The above data show how charged monomer composition and polymer architecture can be varied to impact the hydrodynamic properties as well as biological activity of star polymers carrying high densities of amphiphilic or hydrophobic drug molecules. However, the linker linking drugs, e.g., D2, to star polymers can also impact biological activity.

To assess how linker composition impacts anticancer activity of a representative amphiphilic or hydrophobic drug, a diABZI-based STING agonist was linked to polymer arms at a density of XX mol % to reactive monomers through either a stable amide bond (Compound 56), pH-sensitive hydrazone bond (Compound 94), a pH-sensitive carbohydrazone (Compound 95), a carbamate linker (i.e., PAB) linked to an enzyme (cathepsin) degradable linker (Compound 96) or a carbamate linker (i.e., PAB) linked to an enzyme (cathepsin) degradable linker linker linked to a PEG linker (Compound 97). The synthesis of Compound 94-97 and biological results are summarized below.

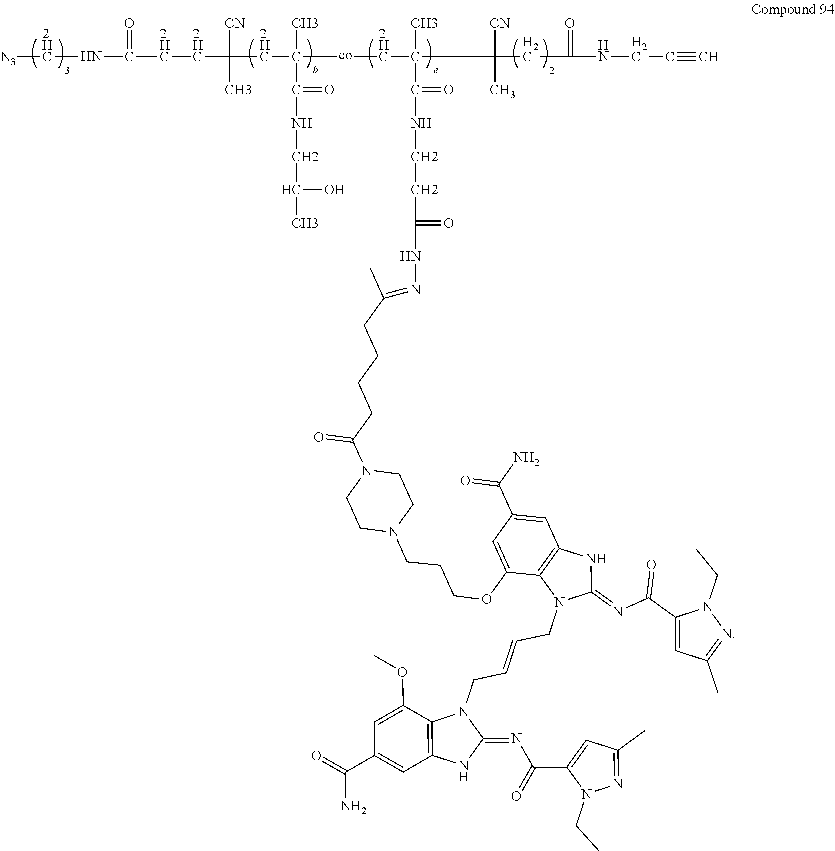

Compound 94. N3-p[(HPMA)-co-(MA-b-Ala-Hz-HA-diABZI)]-Pg was synthesized in a two-step reaction by reactive Compound 45 with hydrazine, followed by addition of diABZI-HA (Compound E). Specifically, N3-poly(HPMA-co-MA-b-Ala-TT)-Pg (Compound 45) (40 mg, 23.0 μmol TT co-monomer) were reacted with 2 equivalents of hydrazine monohydrate (CAS 7803-57-8) (2.31 mg, 46.2 μmol) in 423 μL DMSO for 20 minutes at room temperature. Excess hydrazine was then removed by dialyzing the reaction mixture in a 10 kDa MWCO regenerated cellulose dialysis tube against 1:1 DMSO/MeOH for 1 hour followed by 1 hour against pure MeOH. The polymer N3-poly(HPMA-co-MA-b-Ala-Hz)-Pg (wherein Hz=hydrazide) was then isolated by precipitation into 10× volume diethyl ether and dried to determine mass of isolated polymer (15.1 mg, 39.3% yield). The hydrazide functionalized polymer (4.1 mg, 2.4 μmol Hz) was then reacted with 1 equivalent of diABZI-HA (2.3 mg, 2.4 μmol) using 40 equivalents of acetic acid as a catalyst (5.67 mg, 90 μmol) in a total volume of 161.4 μL DMSO. The reaction was monitored using HPLC with 5-95% gradient of H₂O/ACN with a C18 Poroshell column under neutral conditions (no TFA) and stopped after 16 hours of reaction time. The polymer was purified by dialyzing the reaction mixture in a 10 kDa MWCO regenerated cellulose dialysis tube against 1:1 DMSO/MeOH for 2 hours followed by 2 hours against pure MeOH. The polymer was then precipitated into 10× volume diethyl ether, dried under vacuum and dissolved into DMSO. The concentration of diABZI in the polymer conjugate was then determined using an absorbance measurement at 320 nm in methanol with an extinction coefficient of 56,920 L/(mol·cm) (862 nmol diABZI, 35.9% yield).

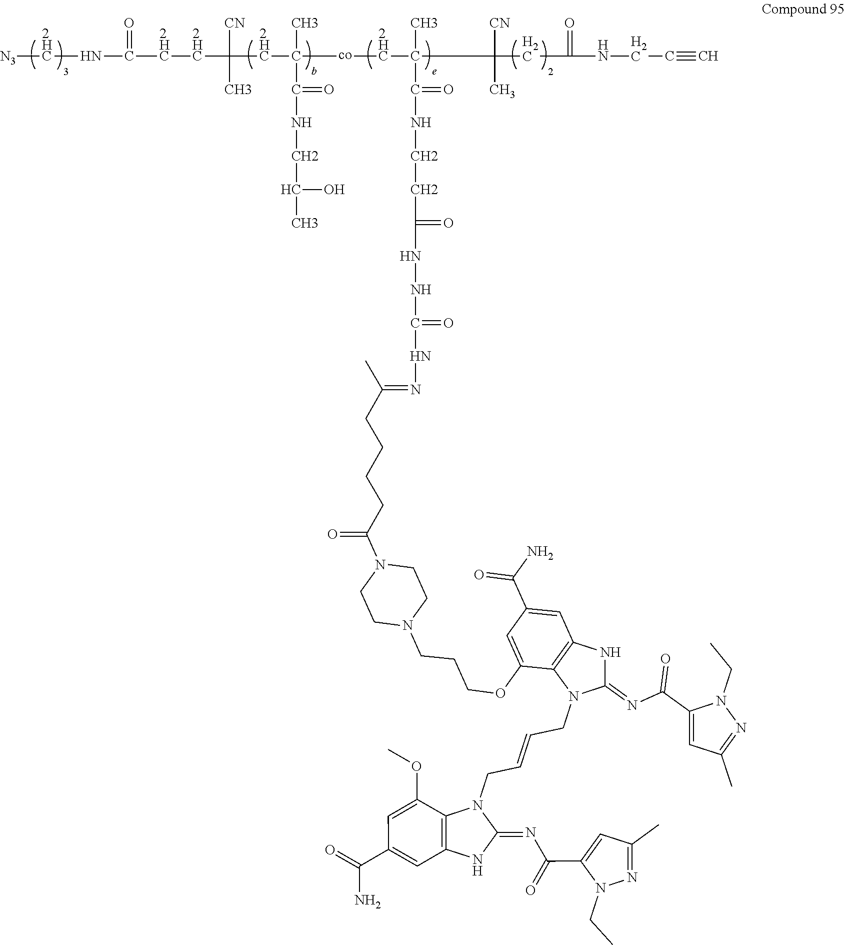

Compound 95. N3-p[(HPMA)-co-(MA-b-Ala-cHz-HA-diABZI)]-Pg was synthesized in a two-step reaction by reactive Compound 45 with carbohydrazide, followed by addition of diABZI-HA (Compound E). Specifically, N3-poly(HPMA-co-MA-b-Ala-TT)-Pg (Compound 45) (40 mg, 23.0 μmol TT co-monomer) were reacted with 2 equivalents of carbohydrazide (CAS 497-18-7) (4.15 mg, 46.2 μmol) in 607 μL DMSO for 60 minutes at room temperature. Excess carbohydrazide was then removed by dialyzing the reaction mixture in a 10 kDa MWCO regenerated cellulose dialysis tube against 1:1 DMSO/MeOH for 1.5 hour followed by 1.5 hour against 20% DMSO, followed by 1 hour dialysis against pure MeOH. The polymer N3-poly(HPMA-co-MA-b-Ala-cHz)-Pg was then isolated by precipitation into 10× volume diethyl ether and dried to determine mass of isolated polymer (22.1 mg, 56.2% yield). The carbohydrazide polymer (4.1 mg, 2.4 μmol cHz) was then reacted with 1 equivalent of diABZI-HA (2.3 mg, 2.4 μmol) using 40 equivalents of acetic acid as a catalyst (5.67 mg, 90 μmol) in a total volume of 161.4 μL DMSO. Reaction efficacy of diABZI-HA was monitored using HPLC with 5-95% gradient of H₂O/ACN with a C18 Poroshell column under neutral conditions (no TFA) and stopped after 16 hours of reaction time at room temperature. The polymer was purified by dialyzing the reaction mixture in a 10 kDa MWCO regenerated cellulose dialysis tube against 1:1 DMSO/MeOH for 2 hours followed by 2 hours against pure MeOH. The polymer was then precipitated into 10× volume diethyl ether, dried under vacuum and dissolved into DMSO. The concentration of diABZI in the polymer conjugate was then determined using an absorbance measurement at 320 nm in methanol with an extinction coefficient of 56,920 L/(mol·cm) (1176 nmol diABZI, 49% yield).

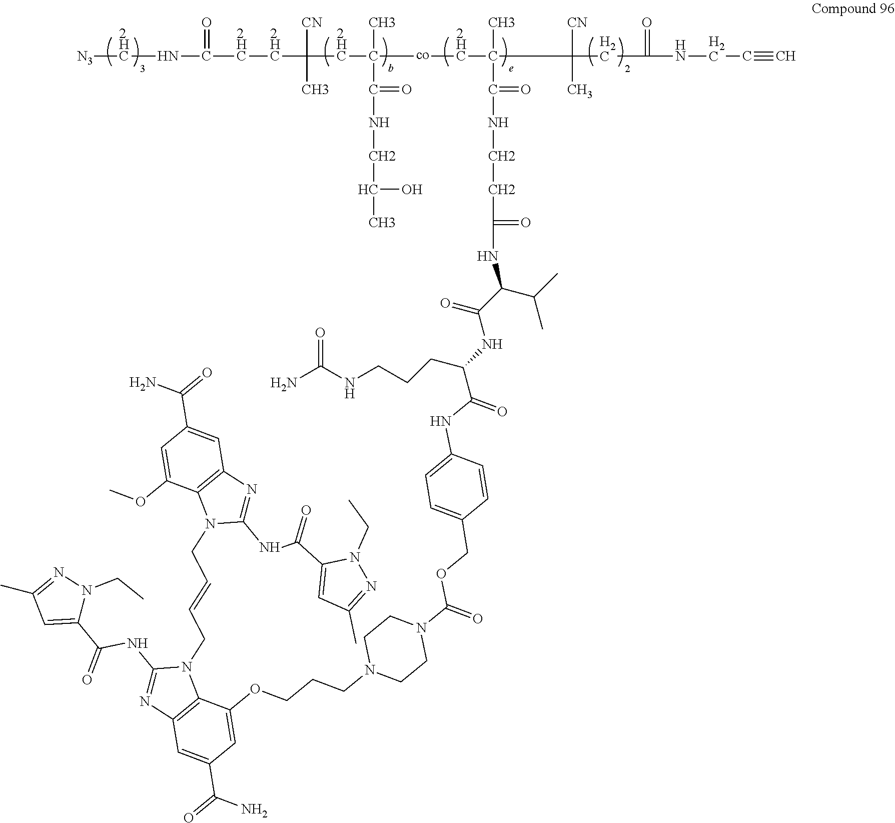

Compound 96. N3-p[(HPMA)-co-(MA-b-Ala-VZ-PAB-diABZI)]-Pg was synthesized by reacting diABZI-PAB-Cit-Val-NH₂ (Compound H) with Compound 45. Specifically, N3-poly(HPMA-co-MA-b-Ala-TT)-Pg (Compound 45) (2.75 mg, 1.6 μmol TT co-monomer) was reacted with 1 equivalent of diABZI-PAB-Cit-Val-NH2 (2.08 mg, 1.6 μmol) and 5 equivalents of triethylamine (0.8 mg, 7.9 μmol) in 142.3 μL DMSO overnight at room temperature. The reaction was monitored by HPLC and stopped after 16 hours. The polymer was purified by dialyzing the reaction mixture in a 10 kDa MWCO regenerated cellulose dialysis tube against 1:1 DMSO/MeOH for 2 hours followed by 2 hours against pure MeOH. The polymer was then precipitated into 10× volume diethyl ether, dried under vacuum, and dissolved into DMSO. The concentration of diABZI was determined using an absorbance measurement at 320 nm in methanol with an extinction coefficient of 23,822 L/(mol·cm) (1413 nmol diABZI, 88.1% yield).

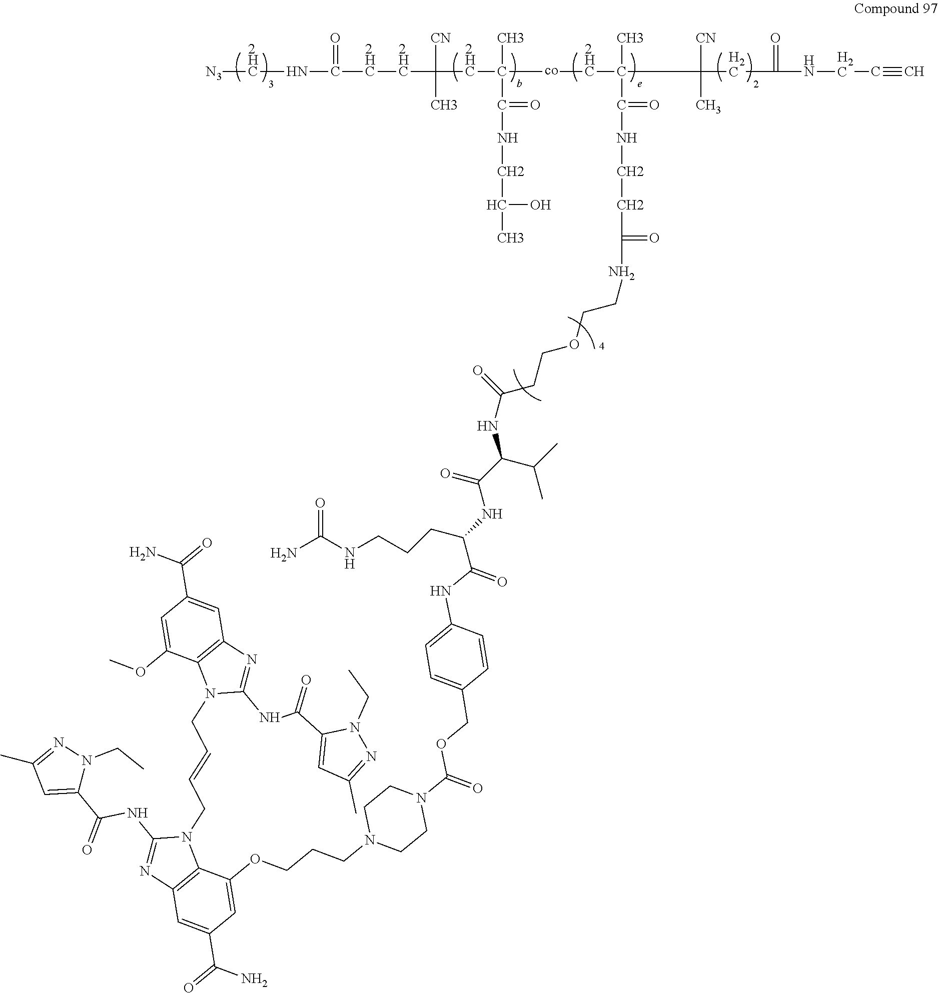

Compound 97. N3-p[(HPMA)-co-(MA-b-Ala-PEG4-VZ-PAB-diABZI)]-Pg was synthesized by reacting diABZI-PAB-Cit-Val-PEG4-NH2 (Compound J) with Compound 45. Specifically, N3-poly(HPMA-co-MA-b-Ala-TT)-Pg (Compound 45) (5 mg, 2.9 μmol TT co-monomer) was reacted with 1 equivalent of diABZI-PAB-Cit-Val-PEG4-NH2 (4.49 mg, 2.9 μmol) and 5 equivalents of triethylamine (1.46 mg, 14.4 μmol) in 294 μL DMSO overnight at room temperature. The reaction was monitored by HPLC and stopped after 16 hours. The polymer was purified by dialyzing the reaction mixture in a 10 kDa MWCO regenerated cellulose dialysis tube against 1:1 DMSO/MeOH for 2 hours followed by 2 hours against pure MeOH. The polymer was then precipitated into 10× volume diethyl ether, dried under vacuum and dissolved into DMSO. The concentration of diABZI was determined using an absorbance measurement at 320 nm in methanol with an extinction coefficient of 23,822 L/(mol·cm) (628 nmol diABZI, 21.7% yield).

To evaluate how the composition of the linker that links D2 to reactive monomers distributed along polymer arms impacts biological activity, we synthesized five different compositions of polymer arms with STINGa linked to reactive monomers using a variety of different linker compositions (sometimes referred to as “linkage”), wherein, in each case, the STINGa was linked to polymer arms at a density of 10 mol % (Table 9)

TABLE 9
Polymer arms with varying linker composition between D2 and the reactive
monomer.

| Cmpd. | | | Mn | | Rh at pH 7.4 |
|---|---|---|---|---|---|
| # | Structure | Linkage | (kDa) | PDI | (nm) |
| 56 | N3-poly(HPMA-co-MA-β-Ala-diABZl)-Pg | Amide | 62.6 | 1.09 | N/A |
| | | | | | (aggregate) |
| 94 | N3-poly(HPMA-co-MA-β-Ala-Hz-HA-diABZl)- | Hydrazone | 162.7 | 3.20 | 10.0 |
| | Pg | | | | |
| 95 | N3-poly(HPMA-co-MA-β-Ala-cHz-HA-diABZl)- | Carbohydrazone | 42.3 | 1.16 | 7.3 |
| | Pg | | | | |
| 96 | N3-poly(HPMA-co-MA-β-Ala-VZ-PAB- | Val-Cit-PAB | 49.3 | 1.38 | 26.9 |
| | diABZl)-Pg | | | | |
| 97 | N3-poly(HPMA-co-MA-β-Ala-PEG4-VZ-PAB- | PEG4-Val-Cit-PAB | 62.5 | 3.44 | 7.6 |
| | diABZl)-Pg | | | | |

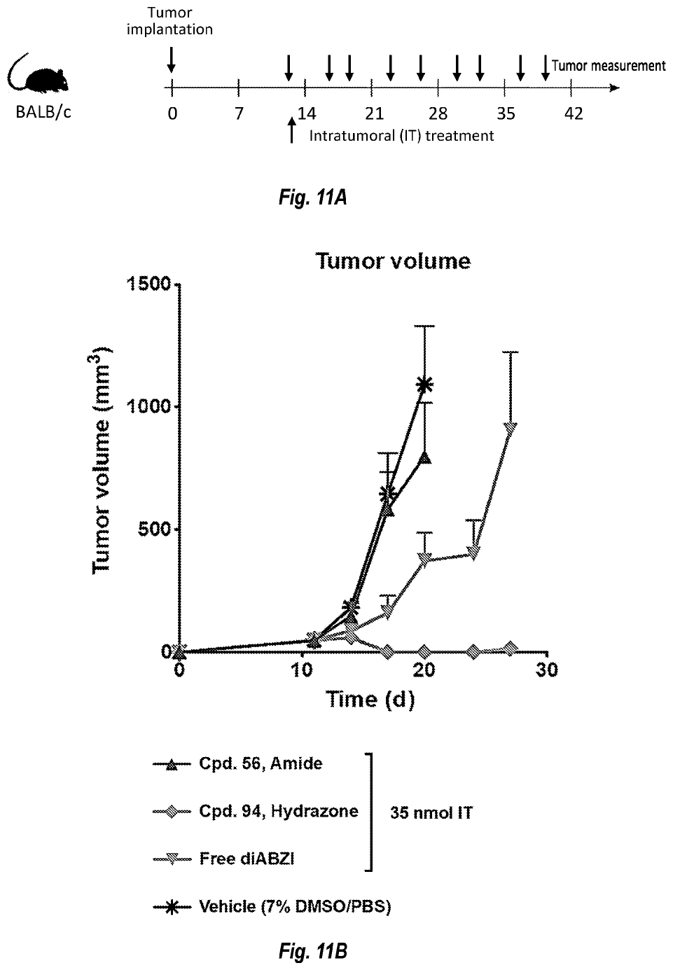

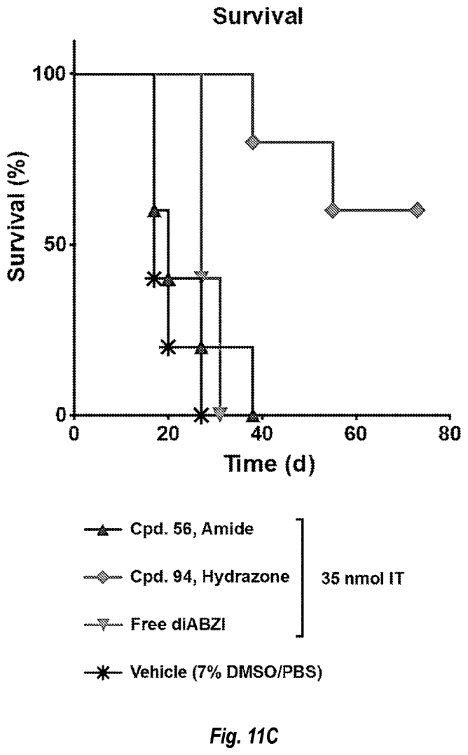

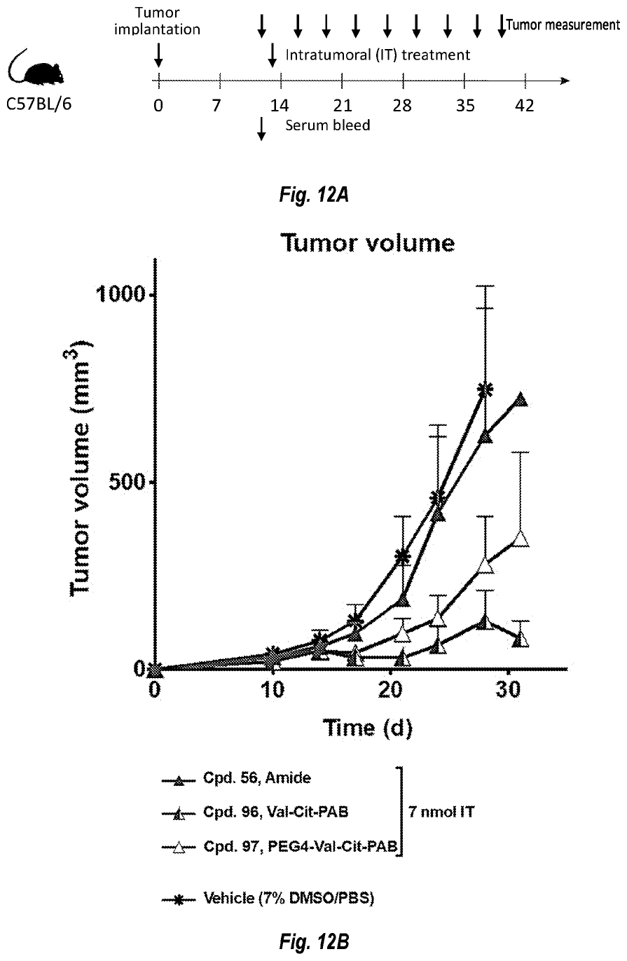

To assess the impact of linkage on efficacy in vivo, select compositions were tested in tumor-bearing mice (FIG. 11). The study design is shown in (FIG. 11A). BALB/c mice were implanted subcutaneously with 10⁵ cells of the syngeneic tumor line CT26 on day 0. Tumors were allowed to grow until all mice in the study had palpable tumors. On day 12, mice were treated with a single intratumoral (IT) injection of 35 nmol of the STINGa as either (i) Compound 56 (amide linkage); (ii) Compound 94 hydrazone (Hz) linkage; or (iii) the free STINGa. An additional group of mice was treated with the formulation vehicle (7% DMSO in PBS) as a negative control. Tumors were measured biweekly to track tumor growth after treatment.

Mice treated with the Compound 94 (hydrazone linkage) showed improved tumor control (FIG. 11B) and survival (FIG. 11C) compared to mice that were either treated with vehicle or the free STINGa. This shows that drug molecules (e.g., STINGa) conjugated to polymer arms leads to improved anti-tumor efficacy compared to free drug alone. Unexpectedly, though, mice treated with Compound 56 (amide linkage) had tumor growth and survival comparable that was comparable to the mice that received the vehicle (negative control) treatment (FIG. 11C). This is unexpected because the linkage of related PRRa immunostimulants, such as TLR-7/8a, through an amide bond to polymer arms, has been shown to result in polymer drug conjugates that are effective for promoting immune activation and tumor regression. Indeed, these results underscore the importance of linkage selection.

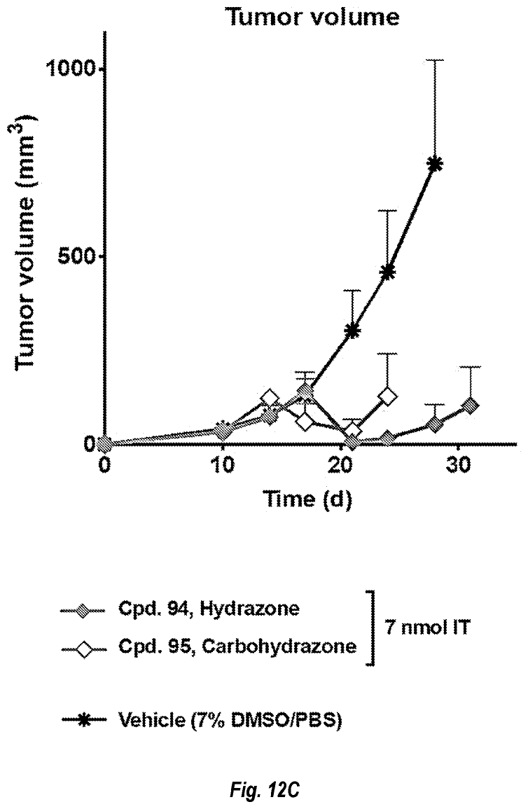

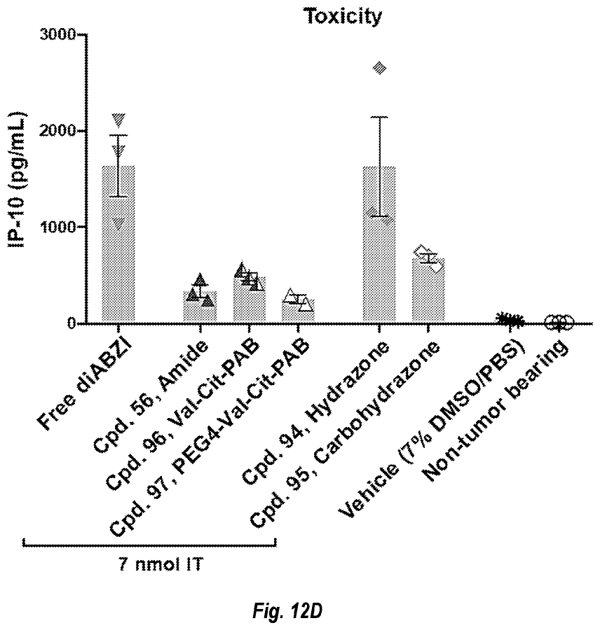

To further assess the impact of linkage on efficacy and tolerability in vivo, select compositions of the Compound listed in Table 9 were evaluated in tumor-bearing mice (FIG. 12). The study design is shown in (FIG. 12A). C57BL/6 mice were implanted subcutaneously with 10¹ cells of the syngeneic tumor line MC38 on day 0. Tumors were allowed to grow until all mice in the study had palpable tumors. On day 11, mice were treated with a single intratumoral (IT) injection of 7 nmol of STINGa (diABZI) as either Compound 56, Compound 94, Compound 95, Compound 96 or Compound 97 in PBS. As a control, a group of mice was treated with the formulation vehicle (7% DMSO in PBS). Tumors were measured biweekly to track tumor growth after treatment. Tolerability was assessed by measuring the amount of Interferon-gamma-induced protein 10 (IP-10) in the serum of animals 4 hours after intratumoral injection.

Mice treated with polymer arms linked to D2 through enzyme-degradable linkages (Compounds 96 and 97), but not a stable amide bond (Compound 56) showed improved tumor control compared with mice treated with vehicle control (FIG. 12B). Notably, the amount of IP-10 in the serum, a measure of tolerability, was lower in mice treated with Compounds 96 and 97 compared to mice treated with free STINGa (FIG. 12D).

Mice treated with polymer arms linked to D2 through pH-sensitive linkages showed tumor regression that was dependent on the exact composition of the linkage, with Compounds 94 and 95 showing improved tumor control compared to untreated mice (FIG. 12C). Though, notably, the level of serum IP-10 for free STINGa and Compound 94 (hydrazone) was similar (FIG. 12D). Interestingly, the level of serum IP-10 was lower for Compound 95 with a carbohydrazone linkage compared to either the free STINGa or the Compound 94 (hydrazone), suggesting that the linkage composition, which determines the rate of drug release, can be controlled to impact tolerability as well as efficacy.

### Example 13—Impact of D2 Density and Charge Monomer Composition on Hydrodynamic Behavior and pH-Responsiveness

As shown earlier, linking high densities of D2 comprising amphiphilic or hydrophobic drug molecules to polymer arms can lead to aggregation (e.g., Compound 56 with 10 mol % diABZI), which can be prevented by the incorporation of charged monomers that can improve solubility of polymer arms (and therefore star polymers) in aqueous solutions. Additionally, charged monomers that are pH-responsive can also be used to change the properties of the polymer arm (and therefore star polymers) in certain conditions, e.g., at reduced pH in the tumor microenvironment, which can be used to promote or prevent interactions with certain materials, such as extracellular matrix and/or cells. To evaluate the interplay between the (i) type of D2 comprising an amphiphilic or hydrophobic drug, (ii) density of D2 and (iii) charged comonomer composition on the hydrodynamic behavior and pH-responsiveness of star polymers, we synthesized a series of polymer arms with varying densities of either a hydrophobic model drug compound 1-naphthalenemethylamine (Naph) or diABZI based STINGa, charged comonomer composition, and charged monomer density (mol %), and evaluated the hydrodynamic behavior of the resulting materials in aqueous buffers at different pH.

We first assessed the impact that the density (mol %) of a hydrophobic model drug compound Naph and diABZI-based STINGa has on the hydrodynamic behavior of polymer arms. Compound 98-102 (Table 10) were synthesized following the same procedure described for Compound 56, except the density of diABZI was varied by adjusting the molar ratio of [diABZI]:[amino-2-propanol] to achieve densities of D2 (i.e., diABZI) linked to reactive monomers from about 5 mol % to about 20 mol %, with the remaining monomer units consisting of neutral hydrophilic monomers. Compound 137-142 (Table 10) were synthesized following the same procedure described for Compound 56, except the D2 was 1-naphthalenemethylamine (Naph) instead of diABZI, and the molar ratio of [Naph]:[amino-2-propanol] was varied to achieve densities of Naph from about 0 mol % to about 7 mol %, with the remaining monomer units consisting of neutral hydrophilic monomers.

TABLE 10
Polymer arms with varying mol % D2.

| | | mol | # of D2 | | | Solubility at |
|---|---|---|---|---|---|---|
| Cmpd. | | % | per polymer | Mn | | 0.5 mg/mL in 1x |
| # | D2 | D2 | arm | (kDa) | PDI | PBS at pH 7.4 |
| 98 | diABZI | 5 | 10 | 39.7 | 1.09 | Soluble |
| 99 | diABZI | 7.5 | 15 | 37.9 | 1.08 | Soluble |
| 100 | diABZI | 10 | 20 | 47.7 | 1.09 | Aggregate |
| 101 | diABZI | 15 | 30 | 57.6 | 1.07 | Aggregate |
| 102 | diABZI | 20 | 40 | 66.0 | 1.07 | Aggregate |
| 138 | Naph | 0 | 0 | 39.3 | 1.26 | Soluble |
| 139 | Naph | 1.5 | 3 | 44 | 1.28 | Soluble |
| 140 | Naph | 3 | 7 | 45.4 | 1.28 | Aggregate |
| 141 | Naph | 5 | 10 | 45.8 | 1.22 | Aggregate |
| 142 | Naph | 7 | 14 | 36.8 | 1.25 | Aggregate |

Compound 98-102 and Compound 137-142 were further characterized to show solubility in aqueous buffer at pH 7.4 by first dissolving in DMSO as a 40 mg/mL stock solution, which was then diluted to 0.5 mg/mL in 1×PBS and solubility visually assessed. As shown in Table 10, polymer arms with up to about 7.5 mol % diABZI or 1.5 mol % of Naph were soluble, whereas those with densities greater than 7.5 mol % diABZI or 1.5 mol % of Naph, i.e., greater than or equal to 10 mol % diABZI or greater than or equal to 3 mol % of Naph were insoluble and precipitated out of solution. The maximum mol % of D2 without inducing aggregation for a neutral polymer arm is determined by the hydrophobicity of D2. In this case, Naph is more hydrophobic than diABZI thus a lower mol % of Naph can be loaded to a polymer arm while maintaining good solubility in aqueous buffer.

As aggregated polymer arms are not suitable for polymers (e.g., star polymers) intended for the intravenous route of injection, unless the liver or spleen are being targeted, these data suggest that polymer arms that do not include charged monomers should have less than 1.5 mol % Naph or 7.5 mol % diABZI attached to prevent aggregation, whereas polymer arms that comprise a charged monomer comprising a charged group that is charged at physiologic pH, pH 7.4, are expected to include higher mol % of D2, e.g., greater than or equal to 10 mol % diABZI or greater than or equal to 3 mol % Naph.

We next evaluated the impact that the charged comonomer has on the hydrodynamic behavior of polymer arms with the diABZI-based STINGa linked to reactive monomers at a density of either 7.5 mol % or 10 mol % (Table 11). Compounds 102-109 were synthesized by reacting the carbonylthiazolidine-2-thione (TT) groups of Compound 45 with Compound C followed by the addition of amine molecules bearing different charged functional groups. The syntheses were performed following the same procedure described for Compound 56 except, following addition of Compound C, instead of reacting with amino-2-propanol, the polymer arm intermediates were either reacted with ethylenediamine (EDA), N,N′-dimethylethylenediamine (DMEDA), glycine (Gly), taurine, NaOH (to yield beta-alanine, b-Ala), 4-amino-2-methylbutanoic acid (Me-BA) or 4-amino-2,2-dimethylbutanoic acid (DMBA).

TABLE 11
Polymer arms with varying mol % D2 and charged monomer composition.

| Cmpd. | Mol % D2 | Charged | Mol % charged | | |
|---|---|---|---|---|---|
| # | (#) | group | monomer (#) | Mn (kDa) | PDI |
| 103 | 7.5 (15) | EDA | 22.5% (45) | 47.72 | 1.26 |
| 104 | 7.5 (15) | DMEDA | 22.5% (45) | 37.55 | 1.09 |
| 105 | 10 (20) | Gly | 20% (40) | 43.57 | 1.07 |
| 106 | 10 (20) | Taurine | 20% (40) | 42.28 | 1.12 |
| 107 | 10 (20) | b-Ala | 20% (40) | 44.11 | 1.09 |
| 108 | 10 (20) | Me-BA | 20% (40) | 44.74 | 1.06 |
| 109 | 10 (20) | DMe-BA | 20% (40) | 44.65 | 1.07 |

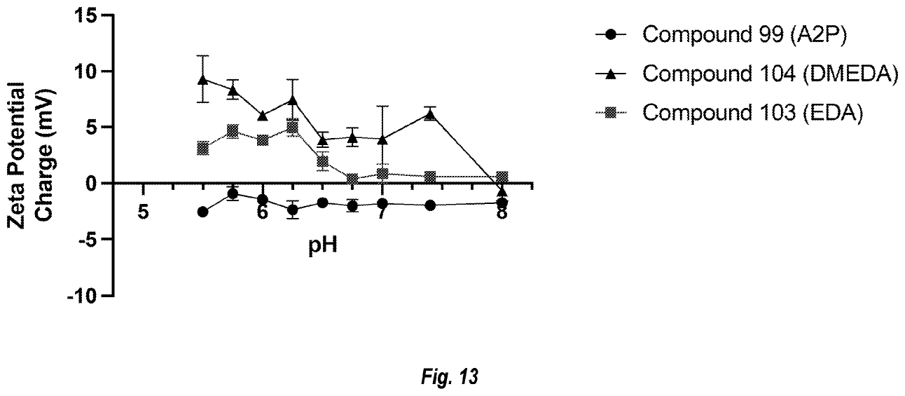

Compounds 99,103 and 104 each have 7.5 mol % diABZI, but Compounds 103 and 104 additionally comprise positively charged monomers with EDA and DMEDA groups that have lower pKa as polymers than such groups otherwise have as single molecules; as such, Compounds 103 and 104 are expected to be partially positively charged at pH 7.4 but have increased magnitude of positive charge as the pH is lowered from physiologic pH 7.4 to tumor pH, e.g., pH 6.5 or less, due to an increasing proportion of EDA and DMEDA becoming protonated. To assess pH-responsive behavior, Compounds 99, 103 and 104 were characterized using DLS to assess zeta potential in PBS buffer at a pH range from 5.5 to 8.0. For sample preparation, compounds were first dissolved in DMSO as a 40 mg/mL stock solution, then diluted to 0.5 mg/mL in 1×PBS that was titrated with either HCl or NaOH to achieve a desired pH. As shown in FIG. 13, Compound 99 remained neutral across the pH range tested, whereas both Compounds 103 and 104 showed an inverse correlation between pH and magnitude of positive charge. These results suggest that upon entry into an acidic tumor environment, Compounds 103 and 104 may became positive and “sticky,” which can enhance drug concentration and cell uptake within acidic environments, e.g., tumors.

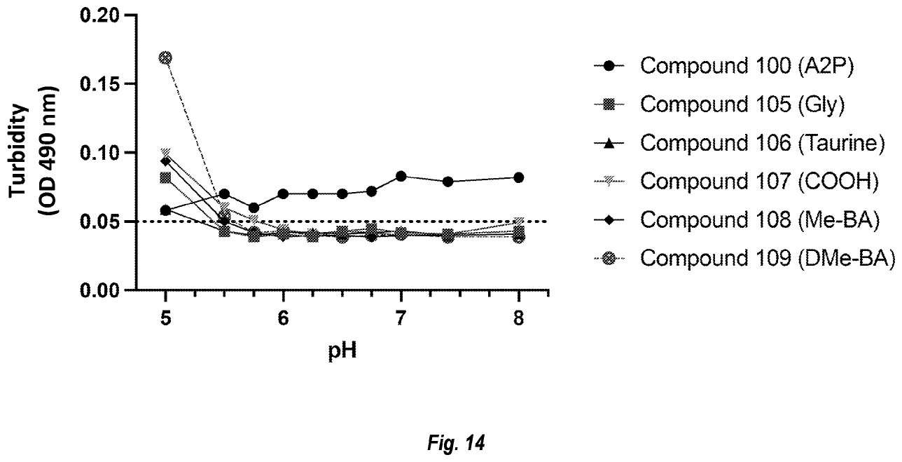

Compounds 105-109 were designed to be negatively charged and soluble in aqueous buffer at physiologic pH, pH 7.4; however, at reduced pH, e.g., within an acidic tumor environment, the conjugate base of the carboxylic acid becomes protonated leading to reduced charge as well as reduced solubility of the polymer arms, which can be observed by measuring turbidity (OD at 490 nm). The turbidity of Compounds 100 and 105-109 in PBS buffer at pH ranging from 5.0 to 8.0 was conducted by first dissolving compounds in DMSO as a 40 mg/mL stock solution, then diluted to 0.5 mg/mL in 1×PBS titrated that was titrated with either HCl or NaOH to adjust the pH. As shown in FIG. 14, Compound 100, which is not pH-responsive, remained insoluble (turbidity >0.05) across the full range of pH tested, while Compounds 105-109 transitioned from soluble to aggregates between pH˜5-6. These results show that including charged monomers on polymer arms allows for the attachment of high densities of amphiphilic or hydrophobic drug molecules without aggregation occurring in aqueous solution at about physiologic blood pH, i.e., about pH 7.4, but that the charged monomers can be tuned to be pH-responsive and become insoluble at reduced pH, e.g., tumor pH.

Thus, our results show how D2 type and density as well as charged monomer composition can be modulated to impact the hydrodynamic behavior and thus the biological activity of star polymers.

### Example 14—Impact of Charged Comonomer Density on Hydrodynamic Behavior and pH-Responsiveness

As shown in Example 13, charged monomer composition can impact the hydrodynamic behavior of polymer arms with diABZI-based STINGa linked to a reactive monomer at a density of 10 mol %. Polymer arms with DMBA charged groups demonstrated unexpected pH-responsiveness by turning from clear solution to aggregate when the buffer pH was lowered from 7.4 to 5.5. To further study the impact of charged comonomer density on the hydrodynamic behavior and pH-responsiveness of polymer arms with high density (i.e., 10 mol %) of D2 comprising amphiphilic or hydrophobic drug molecules, we synthesized a series of polymer arms sharing the structure as N3-poly[(HPMA-co-Ma-b-Ala-D2-co-Ma-b-Ala-DMBA)]-Pg, with different amphiphilic or hydrophobic drug molecules, i.e., 1-naphthalenemethylamine (Naph), TLR-7/8 agonist 2BXy, or diABZI-based STINGa, and varied the mol % of DMBA charged group, and then evaluated their hydrodynamic behavior at different pHs.

Compounds 120-125 (Table 12) were synthesized following the same procedure described for Compound 50 and 10 mol % of TLR-7/8 agonist 2BXy was attached to the reactive monomers. The density of DMBA charged group was varied by adjusting the feeding of 4-amino-2,2-dimethylbutanoic acid to achieve 0 mol % to about 20 mol %, while the remaining reactive monomer units were quenched with excess of amino-2-propanol to afford neutral hydrophilic monomers. Compounds 114-119 and Compounds 126-131 (Table 12) were generated following the same procedure described for Compounds 120-125, except the 02 was 1-naphthalenemethylamine (Naph) and diABZI-based STINGa, respectively. This synthesis protocol was further modified by skipping the 02 introduction step for Compounds 110-113 to generate drug-free polymers with 0-20 mol % of DMBA charged monomer.

TABLE 12
Polymer arms with varying D2 and mol % of DMBA composition.

| | | | # of D2 | | # of DMBA | | | Solubility |
|---|---|---|---|---|---|---|---|---|
| Cmpd. | | mol % | per polymer | mol % | per polymer | Mn | | in 1x PBS |
| # | D2 | D2 | chain | DMBA | arm | (kDa) | PDI | at pH 7.4* |
| 110 | none | 0 | 0 | 0 | 0 | 32.4 | 1.09 | |
| 111 | none | 0 | 0 | 5 | 9 | 42.1 | 1.12 | |
| 112 | none | 0 | 0 | 10 | 18 | 40.3 | 1.11 | |
| 113 | none | 0 | 0 | 20 | 37 | 53.7 | 1.18 | |
| 114 | Naph | 10 | 18 | 0 | 0 | 45.3 | 1.13 | Aggregate |
| 115 | Naph | 10 | 18 | 5 | 9 | 47.4 | 1.12 | Aggregate |
| 116 | Naph | 10 | 18 | 10 | 18 | 49.1 | 1.11 | Aggregate |
| 117 | Naph | 10 | 18 | 12.5 | 23 | 49.9 | 1.10 | Borderline |
| 118 | Naph | 10 | 18 | 15 | 28 | 50.3 | 1.10 | Borderline |
| 119 | Naph | 10 | 18 | 20 | 37 | 65.3 | 1.21 | Borderline |
| 120 | 2BXy | 10 | 18 | 0 | 0 | 81.2 | 1.53 | Aggregate |
| 121 | 2BXy | 10 | 18 | 5 | 9 | 74.8 | 1.40 | Aggregate |
| 122 | 2BXy | 10 | 18 | 10 | 18 | 87.7 | 1.45 | Soluble |
| 123 | 2BXy | 10 | 18 | 12.5 | 23 | 90.2 | 1.42 | Soluble |
| 124 | 2BXy | 10 | 18 | 15 | 28 | 94.1 | 1.40 | Soluble |
| 125 | 2BXy | 10 | 18 | 20 | 37 | 98.9 | 1.39 | Soluble |
| 126 | diABZI | 10 | 18 | 0 | 0 | 71.4 | 1.11 | Aggregate |
| 127 | diABZI | 10 | 18 | 5 | 9 | 86.6 | 1.17 | Aggregate |
| 128 | diABZI | 10 | 18 | 10 | 18 | 108.7 | 1.23 | Aggregate |
| 129 | diABZI | 10 | 18 | 12.5 | 23 | 120.4 | 1.23 | Soluble |
| 130 | diABZI | 10 | 18 | 15 | 28 | 138.8 | 1.26 | Soluble |
| 131 | diABZI | 10 | 18 | 20 | 37 | 145.5 | 1.33 | Soluble |

*Aggregate = turbidity (OD 490 nm) >0.05, borderline = turbidity (OD 490 nm) ~0.05, turbidity (OD 490 nm) <0.05.

Generally, the higher the content of charged monomer, the more soluble the polymer arm containing D2 comprising amphiphilic or hydrophobic drug molecules. The small molecule, 4-amino-2,2-dimethylbutanoic acid (DMBA), has a pKa at about 4.8. When attached to the reactive monomer units along the linear polymer chain, the pKa of DMBA is expected to increase as the immediate environment (i.e., hydrophobicity, distribution of ionizable units, and the ionization state of the neighboring units) of the charged moiety varies compared to that of the individual molecule. For applications like cancer vaccine, the star polymer drug carrier is expected to be anionic due to the deprotonation of DMBA acid groups, hence soluble at physiologic pH 7.4, but transitions to neutral aggregates in tumor microenvironment (i.e., pH 6.5).

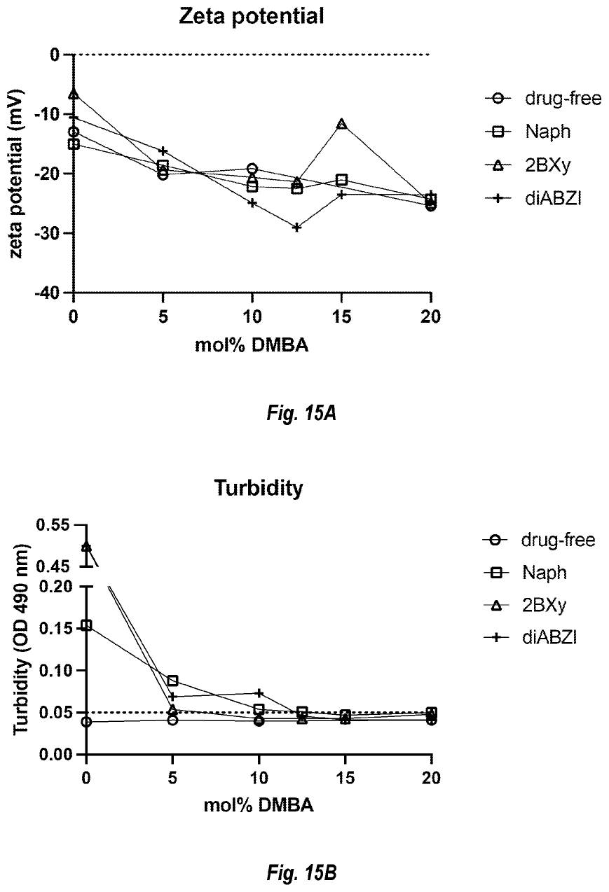

Compounds 110-131 were first characterized by UV-Vis (OD 490 nm) and DLS (dynamic light scattering) to assess their solubility and surface charge (zeta potential) in PBS buffer at physiologic pH. The sample preparation and characterization process were the same as described for Compound 109. As shown in FIG. 15, drug-free polymer arms, Compounds 110-113, with 0-20 mol % DMBA were soluble (turbidity <0.05) and negatively charged (zeta potential <−5 mV) at pH 7.4. Upon introduction of D2 comprising amphiphilic or hydrophobic drug molecules, i.e., Naph, 2BXy and diABZI, the polymer arm remained anionic across the DMBA mol % range. However, Compounds 114-119 bearing 10 mol % Naph were insoluble (turbidity >0.05) with 0-10 mol % DMBA or on the borderline (turbidity ˜0.05) with 10-20 mol % DMBA, indicating more than 20% DMBA is required to completely solubilize 10 mol % Naph.

Compounds 120-125 bearing 10 mol % 2BXy were insoluble with 0-5 mol % DMBA but became soluble when DMBA content increased to 10 mol % or higher. For Compounds 126-131 bearing 10 mol % diABZI, the solubility transition occurred at about 12.5 mol % DMBA. As explained previously, the deprotonation of DMBA groups were affected by the hydrophobic immediate environment, which was largely determined by the hydrophobicity of D2 comprising amphiphilic or hydrophobic drug molecules given the same drug density. The more hydrophobic the drug is, the fewer the deprotonation of DMBA was expected. When the total amount of anionic groups were not enough to balance the hydrophobicity of polymer arms, the polymer arms aggregated. Naph should be the most hydrophobic among all D2 drug molecules tested in this Example, as more than 20 mol % DMBA is expected to be required to completely solubilize a polymer containing 10 mol % Naph. These results also suggest that the minimal DMBA density required to solubilize polymer arms should be determined through a solubility test for different D2 drug molecules and drug densities.

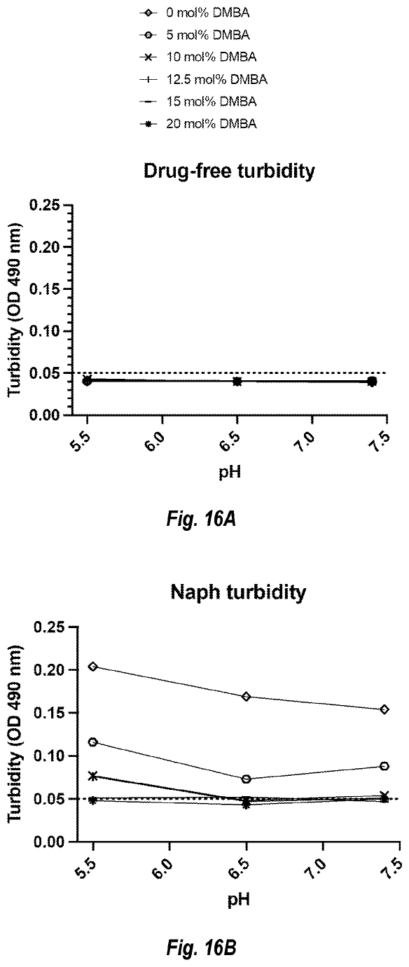

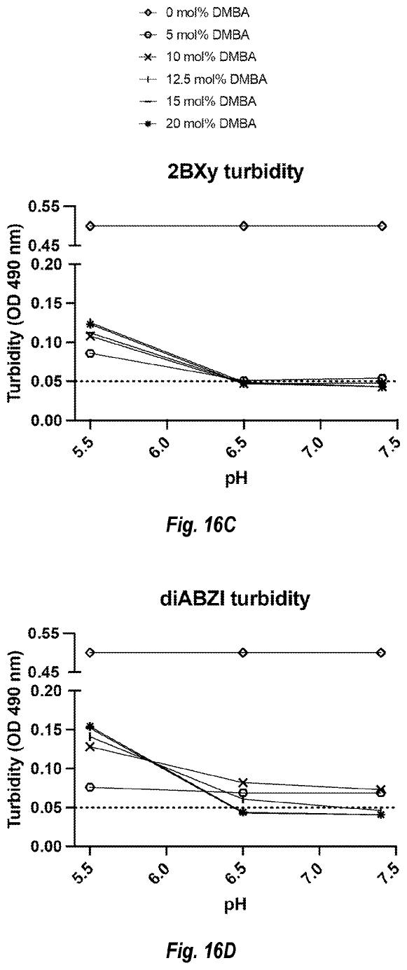

To assess the pH-responsiveness, Compounds 110-114 and Compounds 120-131 were further characterized to reveal solubility changes in PBS buffer at different pHs (5.5, 6.5 and 7.4). As shown in FIG. 16, drug-free polymer arms, Compounds 110-113, with 0-20 mol % DMBA were soluble (turbidity <0.05) throughout the pH range from 5.5 to 7.4. No aggregation was observed as the polymer arm contains no D2 molecule. In contrast, aggregation occurred for polymer arms containing 10 mol % of D2 comprising amphiphilic or hydrophobic drug molecules, i.e., 2BXy and diABZI, at lower pH as more DMBA groups becomes protonated in acid condition and the hydrophilicity of polymer arm decreases, especially for those compositions with minimal DMBA mol % (i.e., 12.5% for polymer arms with 10 mol % diABZI and about 10 mol % for polymer arms with 10 mol % 2BXy). Compounds 121-125 bearing 10 mol % 2BXy and 5-20 mol % DMBA remained the same turbidity (OD 490 nm˜0.05) at pH 6.5 which was then increased to an OD 490 nm higher than 0.07 as the media pH was lowered to 5.5, indicating that the transition pH of these polymers was between 5.5 to 6.5 and the density of DMBA charged group had no impact on solubility. Compound 120, which has same composition as Compound 121 but contains no DMBA charged group and thus insoluble in PBS buffer, exhibited high turbidity across the pH range. Compounds 126-127, that contain insoluble polymer arms with 10 mol % of diABZI and 0-5 mol % of DMBA charged group, showed the same high turbidity from pH 7.4 to 5.5. When the charged group density increased to 10 mol %, the polymer arm was still insoluble, but the turbidity increased from 0.07 at pH 7.4 to about 0.12 at pH 5.5, indicating sufficient protonation of DMBA charged groups induce further aggregation regardless of the material form (i.e., soluble, aggregated) in neutral buffer. Compounds 130-131, that contain diABZI polymer arms with more than enough charged groups (15-20 mol % of DMBA), behaved the same as Compounds 121-125, indicating the polymer arm transition pH was between 5.5 and 6.5. Interestingly, Compound 129 with the minimal charged group density (12.5 mol % of DMBA), showed a step-wise increase of turbidity—slight increase (OD 490 nm˜0.055) at pH 6.5 and then a big increase at pH 5.5, indicating a sharp transition and high transition pH (i.e., about 6.5) for compositions with precisely balanced hydrophobicity and hydrophilicity (no excess charged group).

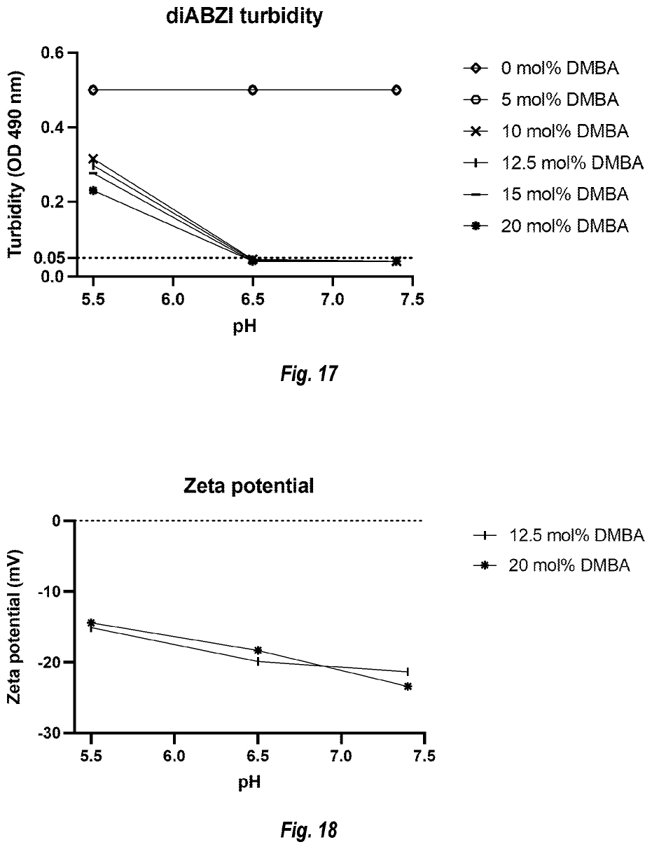

Star polymer Compounds 132-137, PAMAM-g-[PEG24-(DBCO-N3)-p(HPMA-co-MA-b-Ala-diABZI)-Pg]n, were generated through Route 1 by reacting Compounds 126-131 with Compound 72, PAMAM(G5)-g-(PEG24-DBCO)15, following the synthetic protocol described for Compound 75. Composed of linear polymer arms, the star polymers were expected to perform similarly to Compounds 126-131 with the same surface properties and pH-responsiveness. To evaluate the pH-responsiveness, the star polymers were first characterized by GPC to determine the arm # and PDI (results were shown in Table 13) and then turbidity in PBS buffer at pH 5.5 to 7.4. As shown in FIG. 17, Compounds 132-133 with 0-5 mol % DMBA charged groups were insoluble across the pH range. However, Compounds 134-137 with 10-20 mol % DMBA remained soluble at pH 7.4 and 6.5 but became insoluble at pH 5.5, indicating the pH-responsiveness of these star polymers are between 5.5 and 6.5. Unlike the insoluble original polymer arm Compound 128, Compound 134 appeared clear in PBS at pH 7.4, which could be due to the hydrophilic PAMAM dendrimer core. In addition, the zeta potential for the soluble star polymer, Compound 135 and Compound 137, increased from about −21 to about −15 mV as the result of the protonation of DMBA acid group when media pH dropped from 7.4 to 5.5 (FIG. 18).

TABLE 13
Star polymers with 10 mol % of diABZI and varying mol % of DMBA.

| | | | # of D2 | | # of DMBA | | | Solubility |
|---|---|---|---|---|---|---|---|---|
| Cmpd. | | mol % | per polymer | mol % | per polymer | Arm | | in 1x PBS |
| # | D2 | D2 | chain | DMBA | arm | #* | PDI | at pH 7.4 |
| 132 | diABZI | 10 | 18 | 0 | 0 | 5.2 | 1.27 | Aggregate |
| 133 | diABZI | 10 | 18 | 5 | 9 | 6.8 | 1.29 | Aggregate |
| 134 | diABZI | 10 | 18 | 10 | 18 | 7.3 | 1.34 | Soluble |
| 135 | diABZI | 10 | 18 | 12.5 | 23 | 6.6 | 1.38 | Soluble |
| 136 | diABZI | 10 | 18 | 15 | 28 | 6.6 | 1.44 | Soluble |
| 137 | diABZI | 10 | 18 | 20 | 37 | 8.3 | 1.40 | Soluble |

*Arm # = (Mn of star polymer − Mn of dendrimer core)/Mn of polymer arm

### Example 15—Impact of Star Nanoparticle Surface Property on Cell Uptake

Star polymers are designed to shield D2 to decrease unwanted cell uptake in blood, prolonging the circulation time. To determine (i) the impact of HPMA-based polymer in comparison with the well-known low-fouling poly(ethylene oxide) (PEG), (ii) the impact of surface D2 drug molecules, and (iii) the impact of surface charge (positive, negative or neutral) on cell uptake, drug-free star polymer Compounds 145-149 and diABZI-bearing SRCs Compounds 143-144 and 150-151 were prepared. Compounds 143-144 were prepared by first incorporating desired charge groups (i.e., DMEDA and Me-BA, respectively) to the polymer arms of Compound 58 and then coupling the polymer arms to the dye-labeled PAMAM core (Compound 154) in the same manner as Compound 150. Drug-free star polymers Compounds 145-149 were synthesized by coupling TT-PHPMA-Pg with different arm lengths (Mn of 10 kDa or 40 kDa) or PEG5 k-NHS ester to the PAMAM dendrimer using the synthetic process described for Compound 82. PAMAM-Gen 3.0 was used to target arm number s 16 and Gen 5.0 was used to target arm number >16. Polymer arms similar to Compound 96 but with a slightly lower Compound H content (6 mol %) and polymer arms similar to Compound 104 but with the D2 drug molecule diABZI (6 mol %) attached to the reactive monomers through VZ-PAB linkers were synthesized first. Compounds 150-151 were generated by coupling these polymer arms to the PAMAM core with DBCO functional groups (Compound 72) in the same manner as Compound 75.

Table 14 summarizes the composition and hydrodynamic properties of star polymers characterized using GPC-MALS, DLS and turbidity testing except for Compounds 143-144, which could not be assessed as the fluorophore excites at the detecting wavelengths. Still, these star polymers appeared as clear solution in PBS at pH 7.4. Star polymers with no fluorescent labels were allowed to react with Cyanine5 (Cy5) NHS ester (Lumiprobe, 53020) to attach three fluorophores per dendrimer core before cell uptake testing.

TABLE 14
Star polymers with varying surface properties.

| | | | | mol % | Arm | | | Solubility | |
|---|---|---|---|---|---|---|---|---|---|
| Cmpd. | | mol % | Charged | charged | Mn | Arm | | in 1x PBS | Dh |
| # | D2 | D2 | monomer | monomer | (kDa) | #* | PDI | at pH 7.4 | (nm) |
| 143 | diABZI-HA | 7.5 | DMEDA | 22.5 | 46.3 | | | soluble | |
| | (Compound E) | | | | | | | | |
| 144 | diABZI-HA | 10 | Me—BA | 20 | 51.9 | | | soluble | |
| | (Compound E) | | | | | | | | |
| 145 | none | | none | 0 | 10 | 11 | 1.12 | soluble | 9.6 |
| 146 | none | | none | 0 | 10 | 25 | 1.08 | soluble | 14.3 |
| 147 | none | | none | 0 | 40 | 10 | 1.08 | soluble | 19.7 |
| 148 | none | | none | 0 | 40 | 25 | 1.05 | soluble | 24.4 |
| 149 | none | | none | 0 | 5 | 39 | 1.22 | soluble | 17.0 |
| 150 | diABZI-PAB- | 6 | none | 0 | 53.5 | 10 | 1.16 | soluble | 14.5 |
| | Cit-Val | | | | | | | | |
| | (Compound H) | | | | | | | | |
| 151 | diABZI-PAB- | 6 | DMEDA | 20 | 67.6 | 14 | 1.33 | soluble | 48.2 |
| | Cit-Val | | | | | | | | |
| | (Compound H) | | | | | | | | |

*Arm # = (Mn of star polymer − Mn of dendrimer core)/Mn of polymer arm

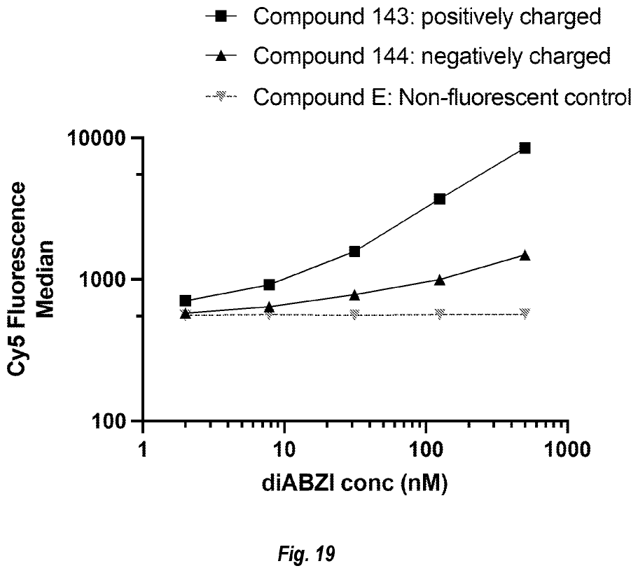

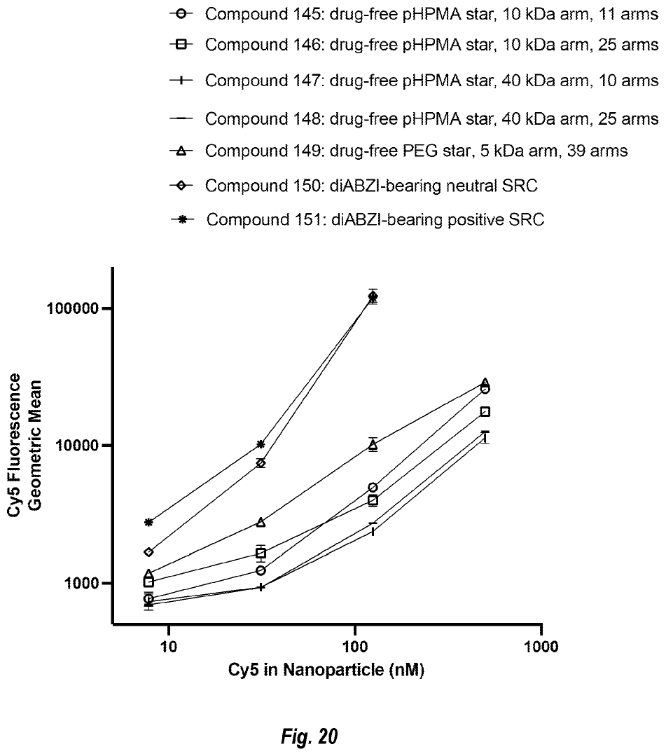

Phagocytic cell uptake of star nanoparticles was assessed using THP1-nfkb cells (Invivogen, THP-nfkb) using star polymers labeled with fluorophore and flow cytometry to assess degree of cell uptake. To assess degree of cell uptake of star polymer nanoparticles, THP1-nfkb cells were seeded to round bottom 96-well plates with 200,000 cells per well in 200 μL of cell growth media containing 10% fetal bovine serum and 1% penicillin/streptomycin. Star nanoparticles were diluted into PBS with a 4-fold dilution series and dispensed to THP1-nfkb cells (20 μL volume per well) to give a final diABZI concentration in cell growth media between 2-500 nM or final Cy5 concentration between 8-500 nM. Cells were incubated with star polymers for two hours, then prepared for flow cytometry. For flow cytometry, 100 μL of cells (from 200 μL volume) were sampled and mixed with 100 μL of PBS, centrifuged to pellet in a round bottom plate and then washed once more with FACS buffer composed of PBS with 1% fetal bovine serum to prevent non-specific inter-cell and cell nanoparticle surface interactions. After final wash, cells were fixed with a solution of 1% paraformaldehyde in PBS and resuspended in FACS buffer to prevent any efflux of nanoparticles while awaiting flow cytometry analysis. Using a flow cytometer, Cy5 fluorescence was measured on a single cell basis and median or geometric mean Cy5 fluorescence was calculated for each nanoparticle condition at the concentrations shown in FIGS. 19-20.

As shown in FIG. 19, the free drug molecule control showed minimal THP1-nfkb cell uptake. Positive SRC containing 10 mol % of diABZI, Compound 143, had higher levels (˜6-fold) of cell uptake at all tested concentration compared to its negative counterpart, Compound 144. This experiment provided evidence of a negative surface charge reducing phagocytic cell uptake of SRCs containing diABZI.

As shown in FIG. 20, all drug-free star polymers, Compounds 145-149, showed very low cell uptake across the concentrations tested. PHPMA-based star polymers showed the same or lower THP1-nfkb cell uptake compared to the PEG-based star polymer, indicating PHPMA is also low-fouling and a good candidate for drug delivery applications. In addition, PHPMA-based stars with 40 kDa arms were less likely to be taken up by THP1-nfkb cells compared to the ones with 10 kDa arm, while the arm number (30 vs 10) had little impact on the results given the same polymer arm length. It was suggested that polymers composed of HPMA hydrophilic monomers were able to suppress APC uptake hence circulate in blood for a long time and extending the hydrophilic PHPMA block length for SDBs could help nanoparticle drug carriers to further avoid unwanted cell uptake.

SRCs provide poor shielding on the drug molecules for the nanoparticles that lack the hydrophilic shell filled with the hydrophilic block (i.e., PHPMA) as SDBs do. As a results, the D2 comprising amphiphilic or hydrophobic drug molecules exposed on the particle surface are likely to interact with biomolecules circulating in blood (proteins and peptides) and receptors on certain type of cells (i.e., APCs). Compared to all drug-free star polymers, Compound 150 and Compound 151, neutral and cationic SRCs containing diABZI, strongly increased THP1-nfkb cell uptake, indicating that exposing diABZIs on nanoparticle surface promotes cell uptake. Interestingly, the positively charged and neutral particles showed the same cell uptake. In addition to the cell uptake findings for Compound 143 and Compound 144, it was clear that negative charge groups helped to decrease uptake of nanoparticles in immune cells compared to neutral or positive surfaces.

### Example 16—Impact of pH-Responsiveness on Biological Activities

The above data show negative charge groups prevent non-specific uptake by immune cells and how charged monomer (i.e., DMBA) density can be varied to impact the hydrodynamic properties and pH-responsiveness of star polymers carrying high densities of amphiphilic or hydrophobic drug molecules (i.e., Naph, 2BXy and diABZI). To study how the biological activities including (i) cellular uptake under different pH conditions and (ii) efficacy and toxicity are affected by negative surface and pH-responsiveness, SRCs containing DMBA charged groups at varying densities, PAMAM-g-[PEG24-(DBCO-N3)-p(HPMA-co-Ma-b-Ala-VZ-PAB-diABZI-co-Ma-b-Ala-DMBA)-Pg]n, were synthesized. Compound 156 was generated by coupling the polymer arm prepared in the same way as Compound 110, but contained 12.5 mol % 02 to PAMAM core with DBCO functional groups (Compound 72). Compounds 157-158 were prepared in the same way as Compounds 134-135, but the drug molecules were linked to the reactive monomers through a cathepsin-degradable VZ-PAB linker. Compound 159 was prepared the same way as Compound 150, except 10 mol % 02 was incorporated. All star polymers were analyzed using GPC-MALS, DLS and turbidity to confirm composition and physical characteristics, as shown in Table 15. For cell uptake testing, 3 molecules of Cy5 were attached to each dendrimer core and used without further characterization.

TABLE 15
Star polymers with varying mol % DMBA.

| | | | | mol % | Arm | | | Solubility | |
|---|---|---|---|---|---|---|---|---|---|
| Cmpd. | | mol % | Charged | charged | Mn | Arm | | in 1x PBS | Dh |
| # | D2 | D2 | monomer | monomer | (kDa) | #* | PDI | at pH 7.4 | (nm) |
| 156 | none | | DMBA | 12.5 | 45.6 | 9 | 1.31 | soluble | 17.1 |
| 157 | diABZI- | 10 | DMBA | 10 | 54.4 | 8 | 1.53 | soluble | 21.3 |
| | PAB-Cit-Val | | | | | | | | |
| | (Compound H) | | | | | | | | |
| 158 | diABZI- | 10 | DMBA | 12.5 | 54.8 | 6 | 1.46 | soluble | 19.1 |
| | PAB-Cit-Val | | | | | | | | |
| | (Compound H) | | | | | | | | |
| 159 | diABZI- | 10 | none | | 54.8 | 18 | 1.55 | insoluble | |
| | PAB-Cit-Val | | | | | | | | |
| | (Compound H) | | | | | | | | |

*Arm # = (Mn of star polymer − Mn of dendrimer core)/Mn of polymer arm

To evaluate the impact of the material properties in Table 15 on cellular uptake under different pH conditions, Cy5 dye-tagged materials were incubated with splenocytes and cellular uptake was assessed using flow cytometry. Briefly, C57Bl/6 mice were sacrificed and their spleens were dissected. Splenic membranes were manually disrupted and the resulting cell suspension was filtered through 70 μM filters and washed with PBS. ACK lysis buffer was added for 3 minutes before a final wash and filtration. Splenocytes were counted and resuspended in pH-adjusted media, which was prepared using HEPES (4-(2-hydroxyethyl)-1-piperazineethanesulfonic acid) and/or MES [2-(N-morpholino)ethanesulfonic acid] buffers and adjusted to pH 7.4, 6.5, or 6.0 using HCl or NaOH. The pH adjusted media was then sterile filtered through a 0.2 μm filter and stored at 4° C. Cells were plated onto 96 well V-bottom plates at a concentration of 1 E5 cells/100 μL/well. Star polymers were then added at a concentration of 200 nM Cy5 and plates incubated at 37° C. with 0% CO₂ for 2 h. Polymers were then washed away before cells were stained with a UV live/dead stain and fixed with 0.5% paraformaldehyde (PFA). Finally, cell suspensions were analyzed using flow cytometry to determine the percent of live, Cy5+ cells, in the same way as described in Example 15. In some cases, data was further analyzed by normalizing to percent uptake at pH 7.4 to better demonstrate the impact of lowering pH.

As shown in FIG. 21-a, uptake of the construct containing 12.5% DMBA without drug (Compound 156) and DMBA-free drug-bearing SRC (Compound 159) showed a negligible increase at pH 6.0. However, cells incubated with Compounds 157 and 158 containing 10 mol % diABZI and DMBA, either 10 or 12.5%, showed a large increase in uptake at pH 6.5 and 6.0 compared to uptake at pH 7.4, likely caused by the pH-induced solubility change shown in FIG. 17.

In an acidic condition (i.e., pH 6.0) mimicking the tumor microenvironment, Compounds 157 and 158 were taken up by splenocytes to the same degree as Compound 159, suggesting the absolute uptake avoidance conferred by the DMBA charge group at pH 7.4 is eliminated at pH 6.0 (FIG. 21-b). These results indicated that addition of the DMBA charge group to SRCs conferred pH-responsiveness to immune cell uptake not presented in drug-free or DMBA-free polymers. It also strongly suggested that pH-responsive SRCs containing DMBA charged monomers can be tuned to avoid immune cell uptake in circulation, reducing the likelihood of toxicity due to systemic immune activation, while enhancing immune cell uptake in the lower pH environment of the tumor, increasing the efficacy of immunotherapy treatments.

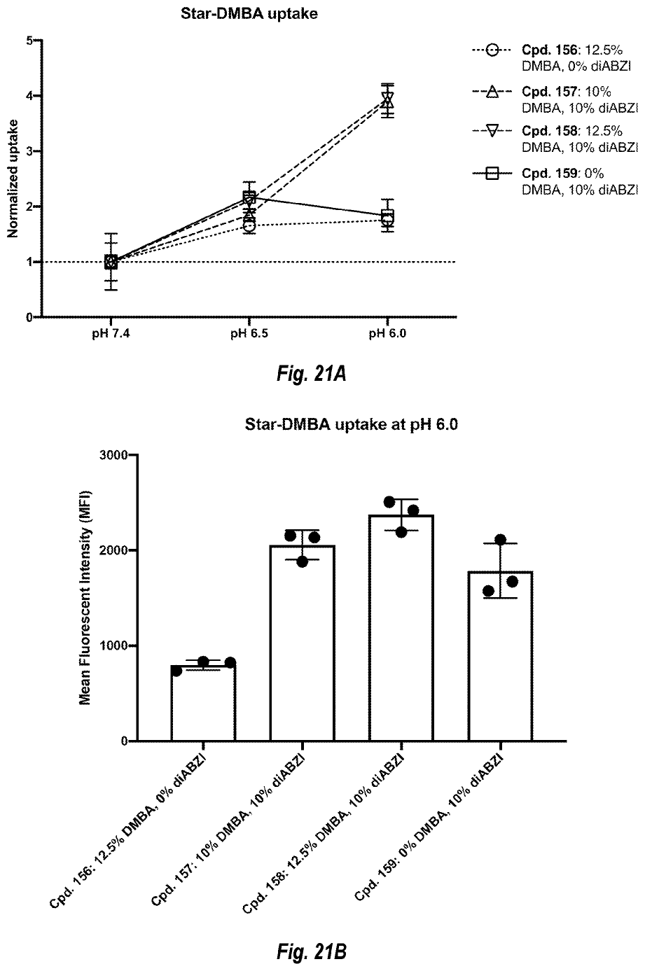

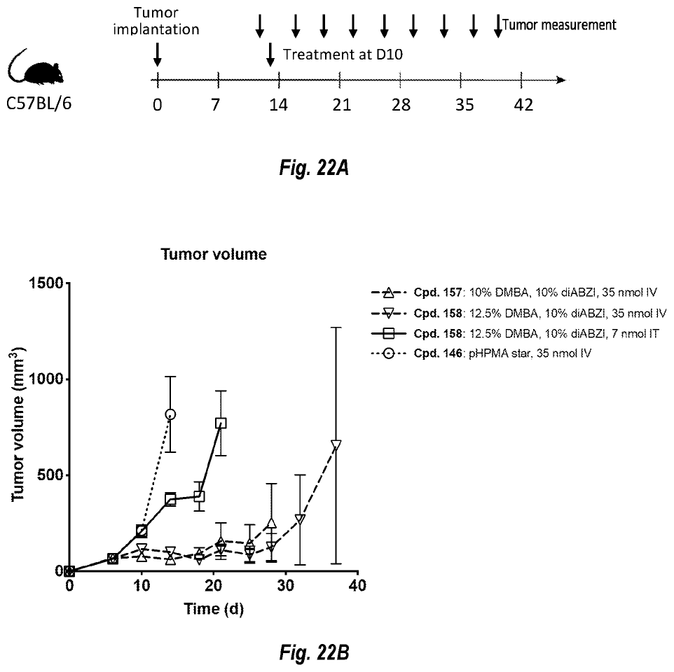

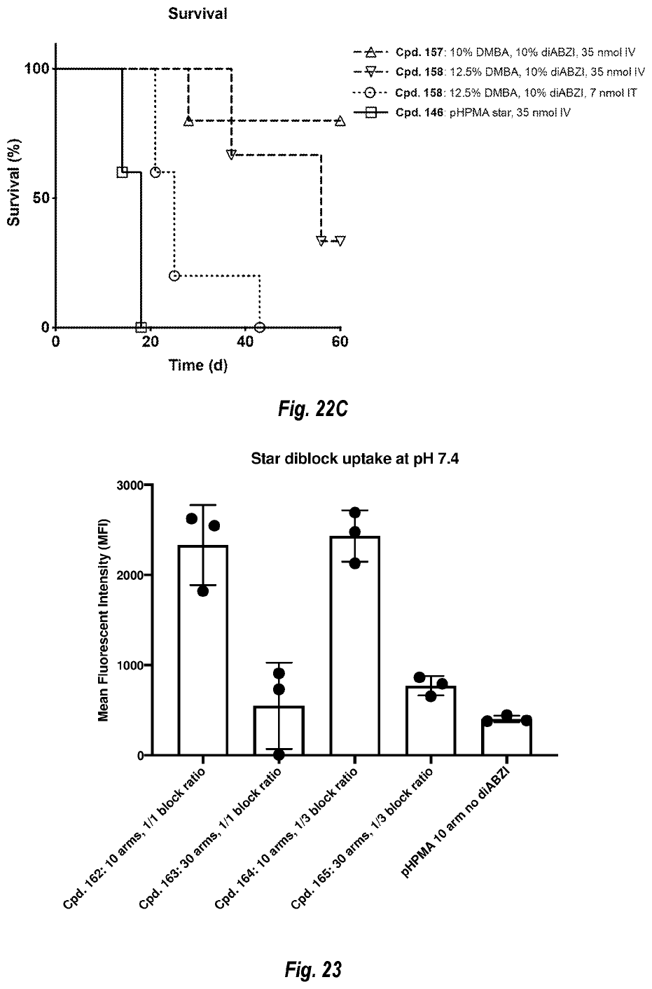

To study the pH-responsive SRCs perform on cancer treatment, Compounds 146, 157 and 158 were assessed for their efficacy in slowing tumor growth and prolonging survival in the MC38 tumor model in vivo (FIG. 22-a). The study was conducted similar to experiments in Example 12, though mice received a single treatment on day 10 of either 35 nmol diABZI intravenous (IV) of Compounds 157 and 158 in PBS or 7 nmol IT of Compound 158 in PBS. Drug-free neutral hydrophilic polymer, Compound 146, was used as the drug-free vehicle control.

As shown in FIG. 22-b, mice treated with 35 nmol diABZI by IV demonstrated improved tumor control compared to IT treatment (7 nmol) or Compound 146 control. IV treated mice also showed prolonged survival (FIG. 22-c). Both Compounds 157 and 158 dosed by IV slowed tumor growth similarly, but 10 mol % DMBA provided better long-term survival. IT treated mice showed delayed tumor growth and improved survival compared to the control but did not perform as well as IV treated mice. These results suggested that addition of the DMBA charge group did not impact the efficacy of attached diABZI molecules. Moreover, subsequent IV delivery is likely to further improve efficacy of free small molecule drugs failed to target tumor sites.

### Example 17—Impact of SDB Architecture (Arm Length and Number) on Biological Activities

Example 15 shows that addition of drug to random copolymer star polymers (SRCs) increases non-specific uptake when exposed to human THP-1 monocytes. This is attributed to the exposure of amphiphilic or hydrophobic drug molecules on the particle surface. In contrast, SDBs forms a core-shell structure for the nanoparticle drug carrier for they are synthesized from amphiphilic diblock copolymers with a hydrophobic block to accommodate drug molecules and a hydrophilic block when exposed to aqueous media. The hydrophilic block forms a hydrophilic shell to solubilize the nanoparticle and shield drug molecules, thereby improving material circulation time.

A few parameters can impact the shielding effect, e.g., arm number per star polymer and hydrophobic to hydrophilic block ratio. SDBs with fewer polymer arms and shorter hydrophilic block (higher hydrophobic to hydrophilic block ratio) are less efficient of shielding the hydrophobic core containing drug molecules with the hydrophilic moieties. To assess the impact of these parameters on non-specific immune cell uptake and anti-tumor efficacy, diblock polymer arms sharing the same hydrophobic block but with varied PHPMA hydrophilic block lengths (hydrophobic to hydrophilic block ratios=1/1 and 1/3, respectively), were first generated in the same manner as Compound 61. Compound H were then attached to the reactive monomers distributed in the hydrophobic block to afford drug-bearing diblock polymer arms (Compounds 160-161), which were then coupled to DBCO-functionalized dendrimer cores through “click” chemistry yielding SDBs with different block ratio and arm density, as depicted below. They were characterized using GPC-MALS, DLS and turbidity to reveal the composition and physical properties, as summarized in Table 16.

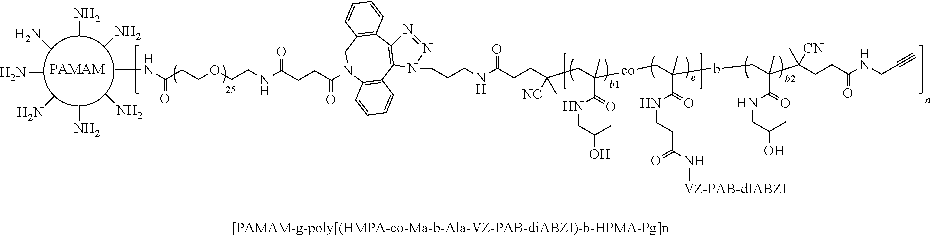

TABLE 16
SDBs with varying arm density and block ratio.

| | | D2 # | Block ratio | Arm | | | Solubility | | Zeta |
|---|---|---|---|---|---|---|---|---|---|
| Cmpd. | | per polymer | (hydrophobic/ | Mn | Arm | | in 1x PBS | Dh | potential |
| # | D2 | arm | hydrophilic) | (kDa) | #* | PDI | at PH 7.4 | (nm) | (mV) |
| 160 | diABZI- | 8 | 1/1 | 37.8 | | 1.3 | soluble | 18.2 | −3.5 |
| | PAB-Cit-Val | | | | | | | | |
| | (Compound H) | | | | | | | | |
| 161 | diABZI- | 8 | 1/3 | 79.1 | | 1.1 | soluble | 14.3 | −2.5 |
| | PAB-Cit-Val | | | | | | | | |
| | (Compound H) | | | | | | | | |
| 162 | diABZI- | 8 | 1/1 | 37.8 | 10 | 1.4 | soluble | 21.7 | −1.82 |
| | PAB-Cit-Val | | | | | | | | |
| | (Compound H) | | | | | | | | |
| 163 | diABZI- | 8 | 1/1 | 37.8 | 30 | 1.5 | soluble | 22.8 | |
| | PAB-Cit-Val | | | | | | | | |
| | (Compound H) | | | | | | | | |
| 164 | diABZI- | 8 | 1/3 | 79.1 | 10 | 1.4 | soluble | 23.8 | −1.92 |
| | PAB-Cit-Val | | | | | | | | |
| | (Compound H) | | | | | | | | |
| 165 | diABZI- | 8 | 1/3 | 79.1 | 30 | 1.3 | soluble | 29.4 | |
| | PAB-Cit-Val | | | | | | | | |
| | (Compound H) | | | | | | | | |

*Arm # = (Mn of star polymer − Mn of dendrimer core)/Mn of polymer arm

To evaluate the impact of the material properties in Table 16 on non-specific cellular uptake, Cy5-dye conjugated materials were incubated with splenocytes at pH 7.4 and cellular uptake was assessed using flow cytometry as in the example above. Cells incubated with polymers containing 30 arms (Compounds 163, 165) showed a large decrease in cell uptake compared to polymers containing 10 arms (Compounds 162, 164) (FIG. 23). However, no difference in uptake was seen between polymers with a 1/1 block ratio (Compounds 162, 163) and 1/3 block ratio (Compounds 164, 165). Polymers containing 30 arms (Compounds 163, 165) had similar levels of uptake to materials with no drug attached (Compound 147). This data suggested that star arm number has a greater impact on 02 partitioning into the core of the particles, with 30 arms providing enough shielding to reduce non-specific cell uptake to levels similar to no-drug controls. Using these principles, we can control the shielding of drug-loaded polymers to reduce immune cell uptake in systemic circulation.

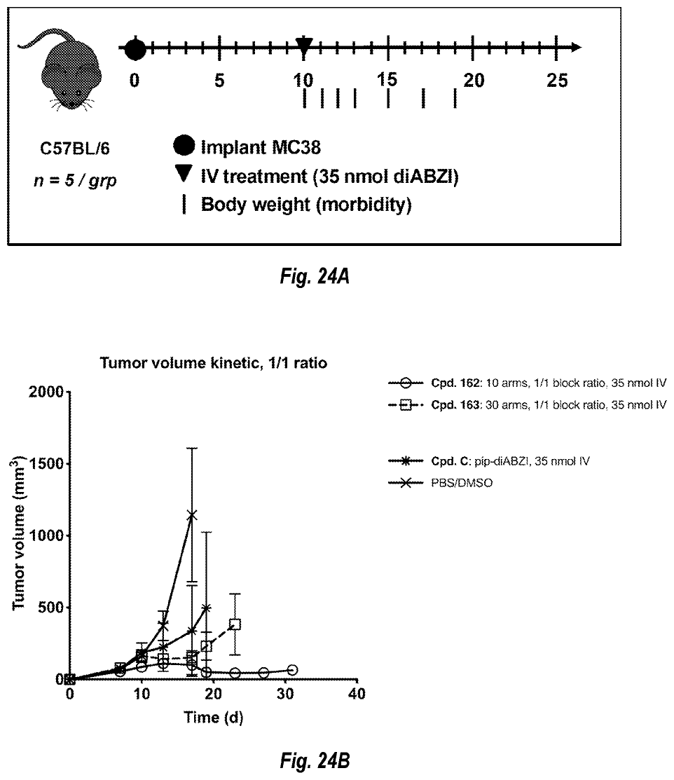

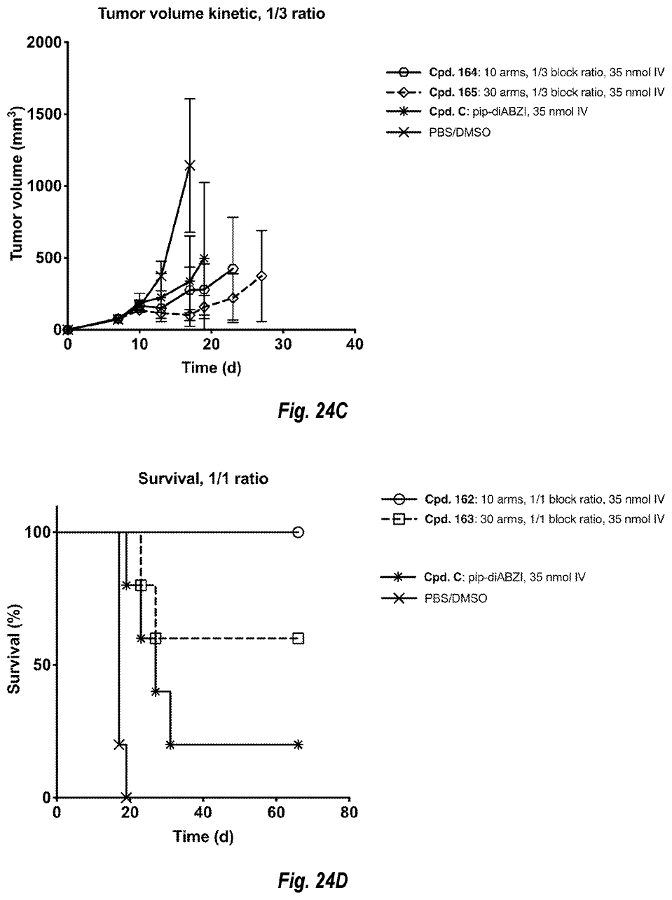

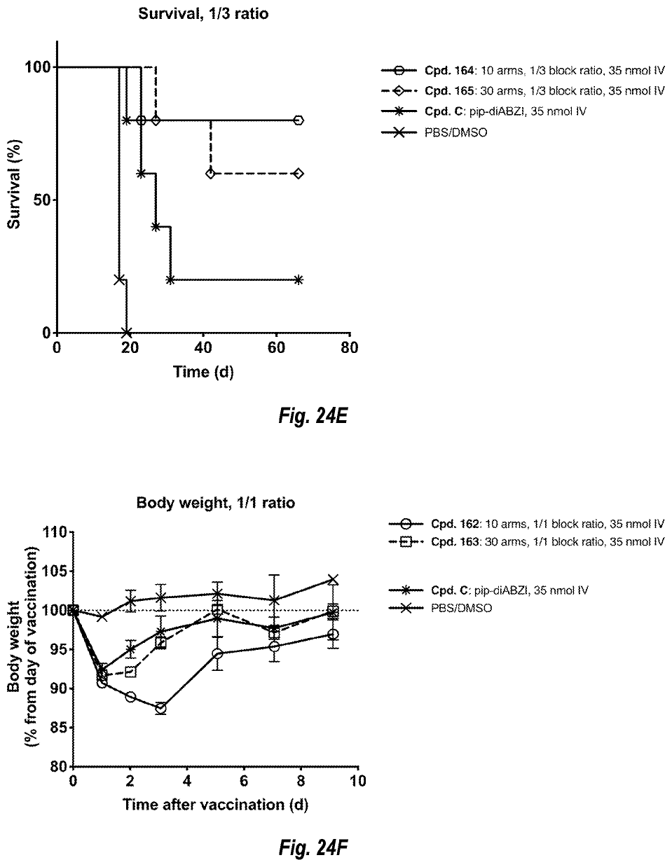

To further assess the in vivo efficacy and toxicity of materials in Compounds 162-165, mice were implanted with the MC38 tumor line and treated as described in Example 16. Mouse body weight was also assessed at the same time each day, or every other day, for 9 days after treatment (FIG. 24-a).

Mice treated with star polymer-linked diABZI, Compounds 162-165, showed improved tumor control and prolonged survival compared to PBS/DMSO formulation control and free diABZI treatment (FIGS. 24-b and c). Compounds 162-165 performed similarly to each other in terms of anti-tumor efficacy. However, mice treated with star polymers containing 30 arms (Compounds 163, 165) lost less weight after treatment and recovered the lost weight more quickly than those treated with polymers containing 10 arms (Compounds 162, 164) (FIG. 24-d). This mirrors the uptake data in FIG. 22. As weight loss is a common proxy for systemic toxicity, this data establishes a direct correlation between non-specific immune cell uptake and systemic toxicity.

### Example 18—Impact of Star Polymer Composition and Architecture on Biological Activities

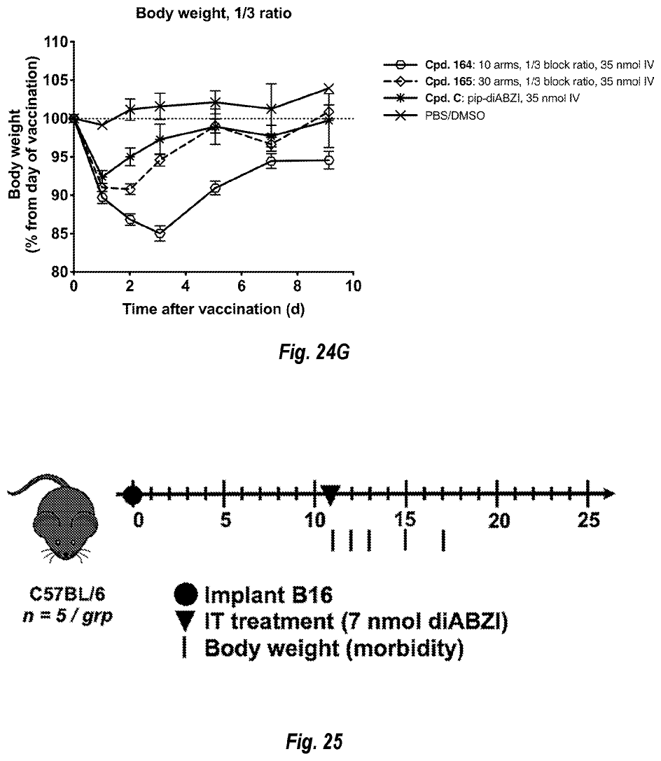

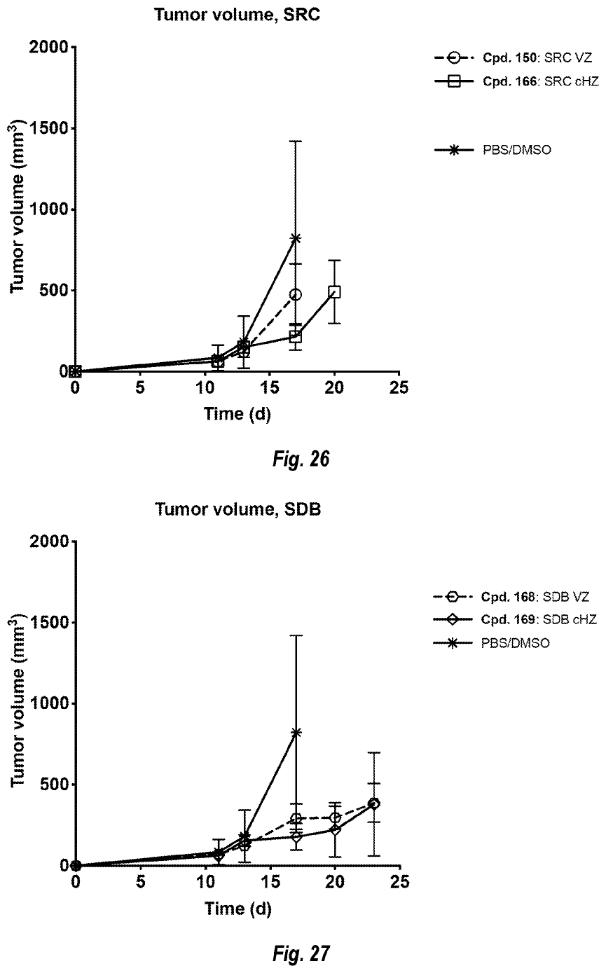

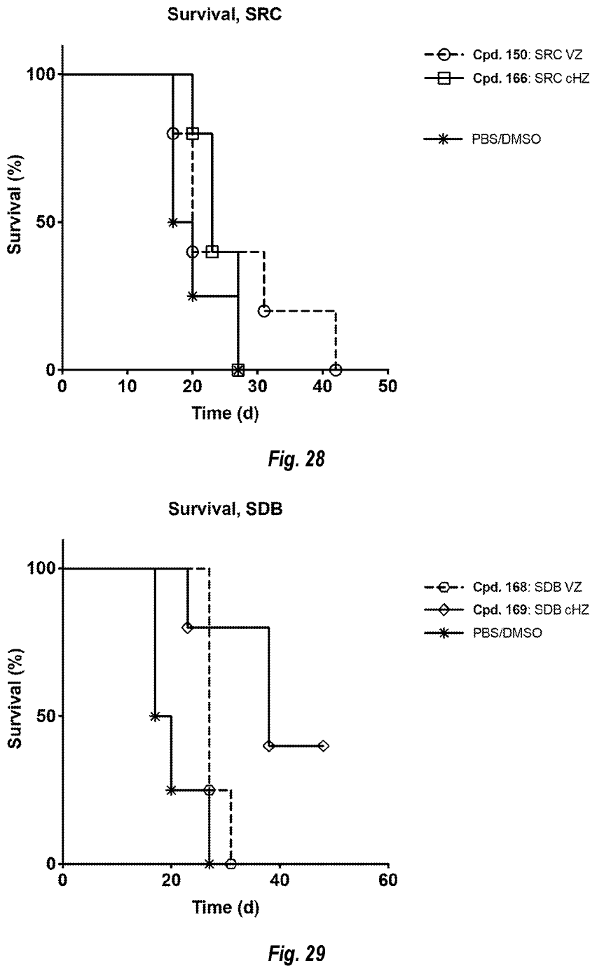

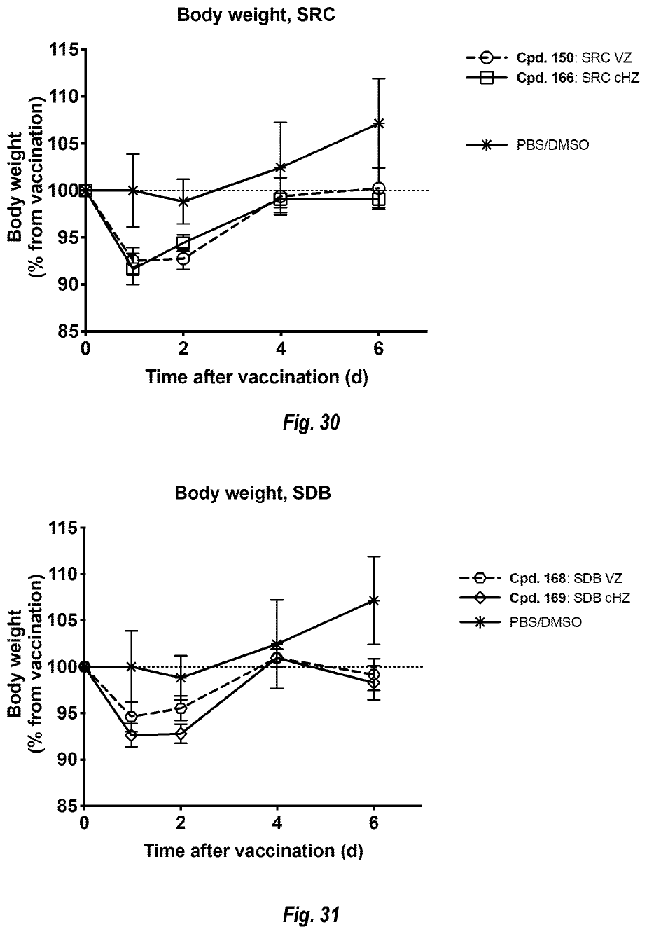

To further evaluate the in vivo efficacy and toxicity of star polymers, mice were implanted with the B16-Adpgk tumor line and treated intratumorally on day 11 as described in Example 12. As depicted in FIG. 25, C57BL/6 mice were implanted subcutaneously with B16 tumors, randomized to equal sized tumor groups and then treated as described (normalized to 7 nmol of STINGa, diABZI) on day 11 with compounds listed in Table 17. Tumor size was measured using digital calipers (FIG. 26, FIG. 27) and survival (FIG. 28, FIG. 29) were assessed up to 60 days after tumor implantation. Tumor growth curves were stopped after one mouse/group is euthanized for tumor size. Mice euthanized for reasons other than tumor size were censored. Body weight was measured at the same time on days D11-13, D15, and D17 (FIG. 30, FIG. 31). Body weight values are presented as percent of body weight on the day of vaccination.

Mice treated with star polymers with carbohydrazone linked diABZI (cHZ-diABZI) (Compound 166, 168) tended to have slower tumor growth than mice treated with star polymers with VZ-PAB linked diABZI (VZ-PAB-diABZI) (Compound 150, 169), with all diABZI-treated mice having slower growth than untreated mice (FIG. 26, FIG. 27). However, overall survival was similar to untreated control tumor bearing animals for SRC carbohydrazone linked diABZI polymer (Compound 166) and SDB VZ-PAB linked diABZI polymer (Compound 168), slightly extended for SRC with VZ-PAB linked diABZI (Compound 169) and greatly prolonged for SDB with carbohydrazone linked diABZI (Compound 169) (FIG. 28, FIG. 29). Finally, mice treated intratumorally with SRC compounds with either VZ-PAB or carbohydrazone linked diABZI (Compounds 150 and 166) lost more weight than mice treated with SDB compounds (Compounds 168 and 169) (FIG. 30, FIG. 31). Overall, the data suggests that SDB constructs (Compounds 168 and 169) have better efficacy and similar toxicity, or similar efficacy and less toxicity, than their SRC counterparts (Compounds 150 and 166).

The above data show that surface properties of star polymers impact their non-specific uptake by immune cells. Therefore, it follows that certain surface properties would be useful in circulation to avoid clearance by cells of the reticulo-endothelial system (i.e. negative surface charge) while others would be preferred at the tumor site where uptake is desired (i.e. positive or neutral surface charge).

In order to create a system where both of these properties coexist, stimuli-responsive charge groups were added to the polymer arms. The tumor microenvironment is known to be more acidic than the circulating bloodstream, making pH a suitable tumor-specific stimuli.

TABLE 17
Star polymers with different compositions and architecture.

| | | | | | | | | | | |
|---|---|---|---|---|---|---|---|---|---|---|
| | | | | Charged | | | | Solubility | | |
| | | D2 # | | monomer # | Arm | | | @ 0.5 mg/mL | | Zeta |
| Cmpd. | | per polymer | Charged | per polymer | Mn | Arm | | in 1x PBS | Dh | potential |
| # | D2 | arm | monomer | arm | (kDa) | # | PDI | at pH 7.4 | (nm) | (mV) |

| | | | | | | | | | |
|---|---|---|---|---|---|---|---|---|---|
| 150 | diABZI- | 13 | none | 53.5 | 10 | 1.16 | soluble | 14.5 | −4.3 |
| | PAB-Cit-Val | | | | | | | | |
| | (Compound H) | | | | | | | | |
| 166 | diABZI-HA | 8 | none | 66.1 | 12 | 1.35 | soluble | 13.2 | −5.3 |
| | (Compound E) | | | | | | | | |
| 168 | diABZI- | 7 | | 59.5 | 13 | 1.31 | soluble | 22.2 | −3.8 |
| | PAB-Cit-Val | | | | | | | | |
| | (Compound H) | | | | | | | | |
| 169 | diABZI-HA | 8 | | 117.8 | 13 | 1.98 | soluble | 46.8 | −2.7 |
| | (Compound E) | | | | | | | | |

### Example 19—Stability of Enzyme-Degradable Peptide Linkers

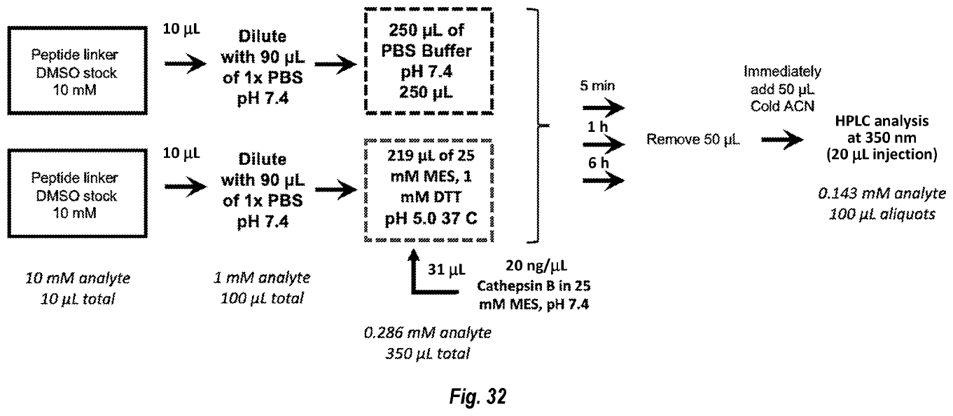

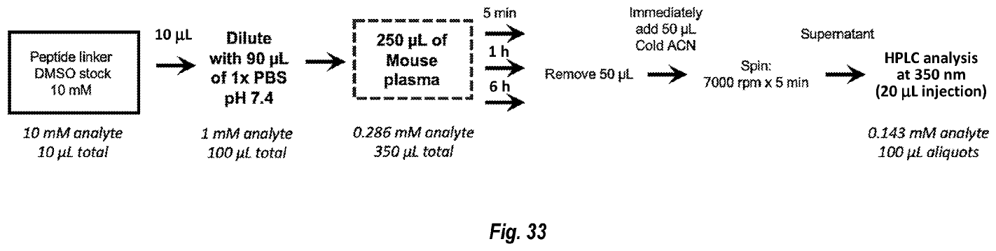

A series of peptide-based small molecules were screened in vitro to characterize their lability in human cathepsin B and mouse plasma enzymes. The screening protocol was adapted from literatures^{1,2} and is shown below in FIGS. 31 and 32. In short, a small library of peptides conjugated to 7-amino-4-methylcoumarin (AMC-peptides) were synthesized by standard solid-phase peptide synthesis (SPPS) by Genscript (Piscataway, NJ), as summarized in the Table 18. They were first dissolved in DMSO as 10 mM stock solution and then incubated in the presence of PBS buffer (Gibco, 10010-031), cathepsin B (Sino Biological, protein human recombinant), and mouse plasma (BioIVT, C57BL/6 li-hep pooled, female). Substrates of either cathepsin B or plasma enzymes (and not PBS) afforded cleavage of the peptide-AMC bond. The resultant free AMC elutes at a different time from the parent AMC-conjugated peptides on the HPLC, thereby the linker degradation was monitored and quantified over time (i.e., 5 min, 1 hr and 6 hrs). PBS buffer solution was used as the matrix for the negative control. Cleavage of cathepsin B substrate Ill (Millipore Sigma, 219392) served as the positive control.

TABLE 18
AMC-conjugated peptides

| Cmpd. # | AMC-peptide | MW |
|---|---|---|
| AH | Ac-A′VB-AMC | 472.56 |
| Al | Ac-A′SPVB-AMC | 656.75 |
| AJ | Ac-A′SK(Ac) VB-AMC | 729.84 |
| AK | Ac-A′SK(Ac) SB-AMC | 717.78 |
| AL | Ac-A′SKSB-AMC | 675.76 |
| AM | Ac-A′VnL-AMC | 500.61 |
| AN | Ac-A′SPVnL-AMC | 684.81 |
| AO | Ac-A′SK(Ac) SnL-AMC | 745.84 |
| AP | Ac-A′SKSnL-AMC | 703.81 |

A′ = beta-alanine, B: alpha-aminobutyric acid, nL: norleucine

The stability of the analyte peptide was determined by the comparing the area-under-curve (AUC) of analyte peaks at 350 nm following formula:

$$\% cleaved = ( 1 - \frac{A U C of parent molecule}{A U C of all peaks in the analysis range} ) * 100 \%$$

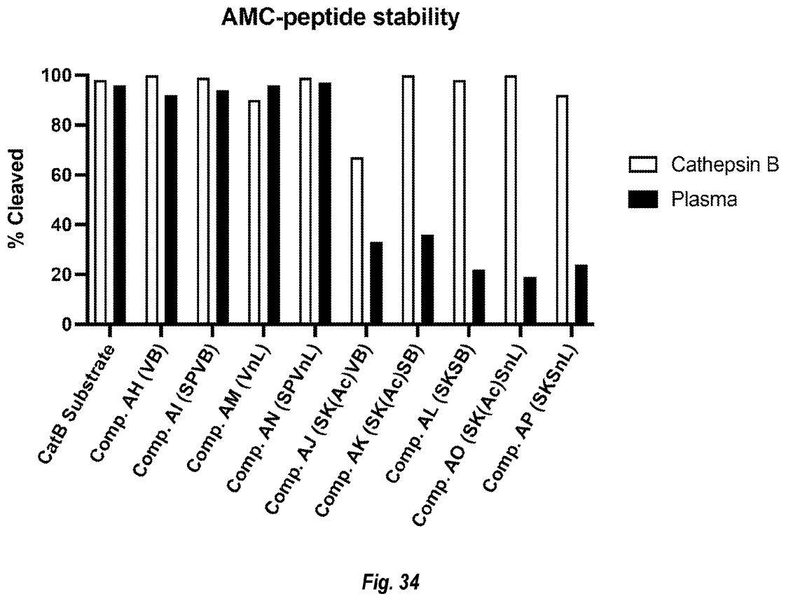

The results of this screen are shown in the FIG. 34.

A series of peptides with differential stability profiles were identified by this screen. Several of the peptides (Compound AH, AI, AM and AN) were found to be metabolized quickly in the presence of cathepsin B as well as mouse plasma. Compound AJ was relatively stable in both matrices. AMC-peptide constructs Compound AK, AL, AO and AP showed significant stability in mouse plasma, but were readily cleaved in the presence of human cathepsin.

### Additional Embodiments

In a first aspect, disclosed herein is a star polymer having the formula O[D1]-([X]-A(D2)-[Z]-[D3])n where O is a core; A is a polymer arm attached to the core; X is a linker molecule between the core and the polymer arm; Z is a linker molecule between an end of the polymer arm and D3; D1 is a drug molecule linked to the core; D2 is a drug molecule linked to reactive monomers distributed along the polymer arm; D3 is a drug molecule linked to the ends of the polymer arms; n is an integer number; [ ] denotes that the group is optional, wherein the polymer arm comprises reactive monomers, hydrophilic monomers and/or charged monomers and D2 is linked to the reactive monomers distributed along the polymer arm at a density of between 1 mol % and 80 mol %.

In certain embodiments, D2 is selected from amphiphilic or hydrophobic drug molecules, and D2 is linked to the polymer arms at a density of between about 1 mol % and about 40 mol %.

In certain embodiments, the polymer arm comprises charged monomers that are negatively charged at pH 7.4; D2 is linked to the reactive monomers distributed along the polymer arm at a density of between about 5 mol % and about 40 mol %, and the charged monomers are distributed along the polymer arm at a density of between about 10 mol % and about 60 mol %.

In certain embodiments, the charged monomers comprise carboxylic acids and/or carboxylic acid salts

In certain embodiments, the charged monomers are selected from (meth)acrylates and (meth)acrylamides having the chemical formula CH₂═CR₅—C(O)—R₄; wherein R₄ is independently selected from —OR₆, —NHR₆ or —N(CH₃)R₆; R₅ is independently selected from H or CH₃; and R₆ is selected OH (except for NHR₆ or —N(CH₃)R₆), (CH₂)ⱼCH(NH₂)COOH, (CH₂)ⱼCOOH, (CH₂)ⱼCH(CH₃)COOH, (CH₂)ⱼC(CH₃)₂COOH, CH(COOH)CHCH₂COOH, (CH₂)ⱼNH(CH₂)ⱼCOOH, (CH₂)ⱼN(CH₃)(CH₂)ⱼCOOH, (CH₂)ⱼN⁺(CH₃)₂(CH₂)ⱼCOOH, (CH₂)ⱼN⁺(CH₂—CH₃)₂(CH₂)ⱼCOOH, (CH₂)ₜ—C(O)—NH—(CH₂)ⱼCH(NH₂)COOH, (CH₂)ₜ—C(O)—NH—(CH₂)ⱼCOOH, (CH₂)ₜ—C(O)—NH—(CH₂)ⱼCH(CH₃)COOH, (CH₂)ₜ—C(O)—NH—(CH₂)ⱼC(CH₃)₂COOH, (CH₂)ₜ—C(O)—NH—CH(COOH)CHCH₂COOH, (CH₂)ₜ—C(O)—NH—(CH₂)ⱼNH(CH₂)ⱼCOOH, (CH₂)ₜ—C(O)—NH—(CH₂)ⱼN(CH₃)(CH₂)ⱼCOOH, (CH₂)ₜ—C(O)—NH—(CH₂)ⱼN⁺(CH₃)₂(CH₂)ⱼCOOH, (CH₂)ₜ—C(O)—NH—(CH₂)ⱼN⁺(CH₂—CH₃)₂(CH₂)ⱼCOOH, (CH₂CH₂O)ₜCH₂CH₂C(O)—NH—(CH₂)ⱼCH(NH₂)COOH, (CH₂CH₂O)ₜCH₂CH₂C(O)—NH—(CH₂)ⱼCOOH, (CH₂CH₂O)ₜCH₂CH₂C(O)—NH—(CH₂)ⱼCH(CH₃)COOH, (CH₂CH₂O)ₜCH₂CH₂C(O)—NH—(CH₂)ⱼC(CH₃)₂COOH, (CH₂CH₂O)ₜCH₂CH₂C(O)—NH—CH(COOH)CHCH₂COOH, (CH₂CH₂O)ₜCH₂CH₂C(O)—NH—(CH₂)ⱼNH(CH₂)ⱼCOOH, (CH₂CH₂O)ₜCH₂CH₂C(O)—NH—(CH₂)ⱼN(CH₃)(CH₂)ⱼCOOH, (CH₂CH₂O)ₜCH₂CH₂C(O)—NH—(CH₂)ⱼN⁺(CH₃)₂(CH₂)ⱼCOOH, (CH₂CH₂O)ₜCH₂CH₂C(O)—NH—(CH₂)ⱼN⁺(CH₂—CH₃)₂(CH₂)ⱼCOOH, where t and j are each an integer number of repeating units, each independently selected from between 1 to 6, such as 1, 2, 3, 4, 5 or 6. In certain specific embodiments, R₄ is independently selected from —NHR₆ or —N(CH₃)R₆; R₅ is independently selected from H or CH₃; and R₆ is selected from (CH₂)₂COOH, (CH₂)₃COOH, (CH₂)₂CH(CH₃)COOH, (CH₂)₂C(CH₃)₂COOH, (CH₂)ₜ—C(O)—NH—(CH₂)₂COOH, (CH₂)ₜ—C(O)—NH—(CH₂)₃COOH, (CH₂)ₜ—C(O)—NH—(CH₂)₂CH(CH₃)COOH or (CH₂)ₜ—C(O)—NH—(CH₂)₂C(CH₃)₂COOH, (CH₂CH₂O)ₜCH₂CH₂C(O)—(CH₂)₂COOH, (CH₂CH₂O)ₜCH₂CH₂C(O)—(CH₂)₃COOH, (CH₂CH₂O)ₜCH₂CH₂C(O)—(CH₂)₂CH(CH₃)COOH or (CH₂CH₂O)ₜCH₂CH₂C(O)—(CH₂)₂C(CH₃)₂COOH, where t is an integer number of repeating units selected from between 1 to 6, such as 1, 2, 3, 4, 5 or 6.

In certain embodiments, the carboxylic is in the form of an alkylammonium salt.

In certain embodiments, D2 is linked to reactive monomers distributed along the polymer arm at a density of between about 1 mol % and about 8 mol % or between about 3 mol % and about 7 mol % and the polymer arm comprises charged monomers that comprise a nitrogen base selected from primary amines, secondary amines, tertiary amines, aromatic amines, and nitrogen heterocycles that are distributed along the polymer arm at a density of between about 5 mol % and about 50 mol % or about 10 mol % and about 30 mol %. In certain specific embodiments, the nitrogen base is selected from groups comprising pyrrole, imidazole, pyridine, pyrimidine, pyrazine, diazepine, indole, quinoline, amino quinoline, amino pyridine, purine, pteridine, aniline, or naphthalene amine rings. In certain embodiments, the charged monomer is selected from (meth)acrylates and (meth)acrylamides with chemical formula CH₂═CR₅—C(O)—R₄ (“Formula II”), wherein R₄ is independently selected from —OR₆, —NHR₆ or —N(CH₃)R₆; R₅ is independently selected from H or CH₃; and R₆ is selected from (CH₂)ⱼ-imidazole, (CH₂)ⱼ-pyridine amine, (CH₂)ⱼ-quinoline amine, (CH₂)ⱼ-naphthalene amine, (CH₂)ⱼN(CH₃)₂, CH₂N(CH₃)₂, CH₂CH₂N(CH₃)₂, CH₂CH₂CH₂N(CH₃)₂, CH₂N(CH₂CH₃)₂, (CH₂)ⱼN(CH₂CH₃)₂, CH₂CH₂N(CH₂CH₃)₂, CH₂CH₂CH₂N(CH₂CH₃)₂, CH₂N(CH(CH₃)₂)₂, (CH₂)ⱼN((CH(CH₃)₂)₂, CH₂CH₂N((CH(CH₃)₂)₂, CH₂CH₂CH₂N(CH(CH₃)₂)₂, (CH₂)ₜ—C(O)—NH—(CH₂)ⱼ-imidazole, (CH₂)ₜ—C(O)—NH—(CH₂)ⱼ-pyridine amine, (CH₂)ₜ—C(O)—NH—(CH₂)ⱼ-quinoline amine, (CH₂)ₜ—C(O)—NH—(CH₂)ⱼ-naphthalene amine, (CH₂)ₜ—C(O)—NH—(CH₂)ⱼN(CH₃)₂, CH₂N(CH₃)₂, (CH₂)ₜ—C(O)—NH—CH₂CH₂N(CH₃)₂, (CH₂)ₜ—C(O)—NH—CH₂CH₂CH₂N(CH₃)₂, (CH₂)ₜ—C(O)—NH—CH₂N(CH₂CH₃)₂, (CH₂)ₜ—C(O)—NH—(CH₂)ⱼN(CH₂CH₃)₂, (CH₂)ₜ—C(O)—NH—CH₂CH₂N(CH₂CH₃)₂, CH₂CH₂CH₂N(CH₂CH₃)₂, CH₂N(CH(CH₃)₂)₂, (CH₂)ₜ—C(O)—NH—(CH₂)ⱼN((CH(CH₃)₂)₂, (CH₂)ₜ—C(O)—NH—CH₂CH₂N((CH(CH₃)₂)₂, (CH₂)ₜ—C(O)—NH—CH₂CH₂CH₂N(CH(CH₃)₂)₂, (CH₂CH₂O)ₜCH₂CH₂(O)—NH—(CH₂)ⱼ-imidazole, (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-quinoline amine, (CH₂CH₂O)ₜCH₂CH₂C(O)—NH—(CH₂)ⱼ-naphthalene amine, (CH₂CH₂O)ₜCH₂CH₂C(O)—NH—(CH₂)ⱼN(CH₃)₂, CH₂N(CH₃)₂, (CH₂CH₂O)ₜCH₂CH₂C(O)—NH—CH₂CH₂N(CH₃)₂, (CH₂CH₂O)ₜCH₂CH₂C(O)—NH—CH₂CH₂CH₂N(CH₃)₂, (CH₂)ₜ—C(O)—NH—CH₂N(CH₂CH₃)₂, (CH₂CH₂O)ₜCH₂CH₂C(O)—NH—(CH₂)ⱼN(CH₂CH₃)₂, (CH₂CH₂O)ₜCH₂CH₂C(O)—NH—CH₂CH₂N(CH₂CH₃)₂, CH₂CH₂CH₂N(CH₂CH₃)₂, CH₂N(CH(CH₃)₂)₂, (CH₂CH₂O)ₜCH₂CH₂C(O)—NH—(CH₂)ⱼN((CH(CH₃)₂)₂, (CH₂CH₂O)ₜCH₂CH₂C(O)—NH—CH₂CH₂N((CH(CH₃)₂)₂, or (CH₂CH₂O)ₜCH₂CH₂C(O)—NH—CH₂CH₂CH₂N(CH(CH₃)₂)₂, where t and j are each an integer number of repeating units, each independently selected from between 1 to 6, such as 1, 2, 3, 4, 5 or 6.

In certain embodiments, the amphiphilic or hydrophobic drug is selected from immunostimulants or chemotherapeutics. In certain specific embodiments, the immunostimulants are selected from pyrimidoindole or lipid-based TLR-4 agonists; adenine-, imdazoquinoline-, or benzonaphthyridine-based TLR-7, TLR-8 or TLR-7/8 agonists; xanthonoid-amidobenzimidazole-based agonists of STING; and, peptide or 3-(2,3-dihydro-1,4-benzodioxin-6-yl)-2-methylphenyl]methanol based inhibitors of PD1/PDL1.

In certain embodiments, the imidazoquinoline-based TLR-7, TLR-8 or TLR-7/8a has the structure:

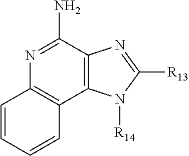

wherein R₁₃ is selected from one of hydrogen, optionally substituted lower alkyl, or optionally substituted lower alkyl ether; and R₁₄ is selected from one of optionally substituted aryalkyllamine, or optionally substituted lower alkylamine, wherein the amine provides a reactive handle for attachment to the reactive monomer either directly or via a linker.

In certain embodiments, the amidobenzimidazole-based STINGa has the following structure:

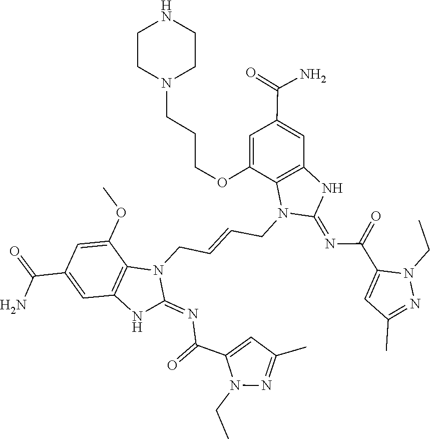 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

In certain embodiments, the chemotherapeutics are selected from alkylating agents, antibiotics, antimetabolites, topoisomerase inhibitors, mitotic inhibitors, receptor tyrosine kinase inhibitors, angiogenesis inhibitors, steroids and anti-hormonal agents.

In certain embodiments, D2 is selected from hydrophilic drug molecules and D2 is linked to the polymer arms at a density of between about 10 mol % and about 40 mol %, and the hydrophilic monomer is distributed along the backbone of the polymer arms at a density of between about 60 mol % to about 90%.

In certain embodiments, D2 is selected from hydrophilic immunostimulants or hydrophilic chemotherapeutics. In certain specific embodiments, the hydrophilic immunostimulants are selected from ssRNA-based agonists of TLR-3, hydroxy-adenine based TLR-7 agonists, oligonucleotide-based agonists of TLR-9 and/or cyclic dinucleotide-based STING agonists.

In certain embodiments, the cyclic dinucleotide-based STING agonists has the structure:

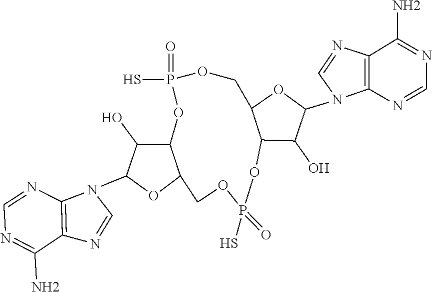 [SMILES: Nc1ncnc2c1ncn2C1OC2COP(=O)(S)OC3C(COP(=O)(S)OC2C1O)OC(n1cnc2c(N)ncnc21)C3O]

In certain embodiments, the cyclic dinucleotide-based STING agonist has R or S stereochemistry at the phosphorous stereocenter.

In a second aspect, provided herein is a star polymer of formula O[D1]-([X]-A1(D2)-b-A2-[Z]-[D3])n where O is a core; A1 and A2 collectively form a polymer arm (A) attached to the core, wherein the polymer arm comprises a first block A1 and a second block A2, which are proximal and distal to the core, respectively; X is a linker molecule between the core and the polymer arm; Z is a linker molecule between the end of the polymer arm and D3; D1 is a drug molecule linked to the core; D2 is a drug molecule linked to reactive monomers distributed along the backbone of the polymer arm; D3 is a drug molecule linked to the ends of the polymer arms; n is an integer number; [ ] denotes that the group is optional; the polymer arm comprises reactive monomers, hydrophilic monomers and/or charged monomers; and, D2 is linked to the reactive monomers distributed along the first block of the polymer arm at a density of between 1 mol % and 80 mol %.

In certain embodiments, the second block comprises charged monomers that comprise a nitrogen base selected from primary amines, secondary amines, tertiary amines, aromatic amines and nitrogen heterocycles that are distributed along the backbone of the polymer arm at a density of between about 5 mol % and about 50 mol % or about 10 mol % and about 30 mol %. In certain specific embodiments, the nitrogen base is selected from groups comprising pyrrole, imidazole, pyridine, pyrimidine, pyrazine, diazepine, indole, quinoline, amino quinoline, amino pyridine, purine, pteridine, aniline, and naphthalene amine rings.

In certain embodiments, the charged monomer is selected from (meth)acrylates and (meth)acrylamides with chemical formula CH₂═CR₅—C(O)—R₄ (“Formula II”), wherein R₄ is independently selected from —OR₆, —NHR₆ or —N(CH₃)R₆; R₅ is independently selected from H or CH₃; and R₆ is selected from (CH₂)ⱼ-imidazole, (CH₂)ⱼ-pyridine amine, (CH₂)ⱼ-quinoline amine, (CH₂)ⱼ-naphthalene amine, (CH₂)ⱼN(CH₃)₂, CH₂N(CH₃)₂, CH₂CH₂N(CH₃)₂, CH₂CH₂CH₂N(CH₃)₂, CH₂N(CH₂CH₃)₂, (CH₂)ⱼN(CH₂CH₃)₂, CH₂CH₂N(CH₂CH₃)₂, CH₂CH₂CH₂N(CH₂CH₃)₂, CH₂N(CH(CH₃)₂)₂, (CH₂)ⱼN((CH(CH₃)₂)₂, CH₂CH₂N((CH(CH₃)₂)₂, CH₂CH₂CH₂N(CH(CH₃)₂)₂, (CH₂)ₜ—C(O)—NH—(CH₂)ⱼ-imidazole, (CH₂)ₜ—C(O)—NH—(CH₂)ⱼ-pyridine amine, (CH₂)ₜ—C(O)—NH—(CH₂)ⱼ-quinoline amine, (CH₂)ₜ—C(O)—NH—(CH₂)ⱼ-naphthalene amine, (CH₂)ₜ—C(O)—NH—(CH₂)ⱼN(CH₃)₂, CH₂N(CH₃)₂, (CH₂)ₜ—C(O)—NH—CH₂CH₂N(CH₃)₂, (CH₂)ₜ—C(O)—NH—CH₂CH₂CH₂N(CH₃)₂, (CH₂)ₜ—C(O)—NH—CH₂N(CH₂CH₃)₂, (CH₂)ₜ—C(O)—NH—(CH₂)ⱼN(CH₂CH₃)₂, (CH₂)ₜ—C(O)—NH—CH₂CH₂N(CH₂CH₃)₂, CH₂CH₂CH₂N(CH₂CH₃)₂, CH₂N(CH(CH₃)₂)₂, (CH₂)ₜ—C(O)—NH—(CH₂)ⱼN((CH(CH₃)₂)₂, (CH₂)ₜ—C(O)—NH—CH₂CH₂N((CH(CH₃)₂)₂, (CH₂)ₜ—C(O)—NH—CH₂CH₂CH₂N(CH(CH₃)₂)₂, (CH₂CH₂O)ₜCH₂CH₂(O)—NH—(CH₂)ⱼ-imidazole, (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-quinoline amine, (CH₂CH₂O)ₜCH₂CH₂C(O)—NH—(CH₂)ⱼ-naphthalene amine, (CH₂CH₂O)ₜCH₂CH₂C(O)—NH—(CH₂)ⱼN(CH₃)₂, CH₂N(CH₃)₂, (CH₂CH₂O)ₜCH₂CH₂C(O)—NH—CH₂CH₂N(CH₃)₂, (CH₂CH₂O)ₜCH₂CH₂C(O)—NH—CH₂CH₂CH₂N(CH₃)₂, (CH₂)ₜ—C(O)—NH—CH₂N(CH₂CH₃)₂, (CH₂CH₂O)ₜCH₂CH₂C(O)—NH—(CH₂)ⱼN(CH₂CH₃)₂, (CH₂CH₂O)ₜCH₂CH₂C(O)—NH—CH₂CH₂N(CH₂CH₃)₂, CH₂CH₂CH₂N(CH₂CH₃)₂, CH₂N(CH(CH₃)₂)₂, (CH₂CH₂O)ₜCH₂CH₂C(O)—NH—(CH₂)ⱼN((CH(CH₃)₂)₂, (CH₂CH₂O)ₜCH₂CH₂C(O)—NH—CH₂CH₂N((CH(CH₃)₂)₂, or (CH₂CH₂O)ₜCH₂CH₂C(O)—NH—CH₂CH₂CH₂N(CH(CH₃)₂)₂, where t and j are each an integer number of repeating units, each independently selected from between 1 to 6, such as 1, 2, 3, 4, 5 or 6.

In certain embodiments, D2 is selected from amphiphilic or hydrophobic drug molecules linked to the first block of the polymer arm at a density of between about 10 mol % to about 40 mol %.

In certain embodiments, the first block is linked to the second block through a pH-sensitive bond selected from hydrazone, silyl-ether and ketal linkages.

In certain embodiments, the degree of polymerization block ratio between the first block and the second block is selected from the range of about 1:2 to about 2:1.

In certain embodiments, D2 is linked to reactive monomers selected from (meth)acrylates and (meth)acrylamides of chemical formula CH₂═CR₈—C(O)—R₇ (“Formula III”), wherein R₇ is an acryl side group comprising a linker molecule for the attachment of D2.

In certain embodiments, D2 is linked to the reactive monomers through a pH-sensitive bond selected from hydrazone, silyl ether and ketal linkages. In certain specific embodiments, the pH-sensitive bond is a carbohydrazone.

In certain embodiments, D2 is linked to reactive monomers through an enzyme degradable peptide or a sulfatase cleavable linker.

In certain embodiments, the polymer arm has a number average molecular weight between about 5 kDa to about 60 kDa, or about 10 kDa to about 40 kDa.

In certain embodiments, the core (O) has greater than 5 points of attachment for polymer arms (A).

In certain embodiments, the core (O) comprises a branched polymer or dendrimer.

In certain embodiments, the dendrimer or branched polymer that is used to form the core (O) has surface amine groups used for the attachment of polymer arms (A) either directly or via a linker X.

In certain embodiments, the core (O) is a dendrimer selected from PAMAM, bis(MPA) or lysine.

In certain embodiments, n is greater than or equal to 5.

In certain embodiments, the star polymer comprises a second polymer arm that is linked to the core through a pH-sensitive linkage selected from hydrazone, ketal and silyl ether linkages, wherein the second polymer arm comprises hydrophilic monomers and/or charged monomers, additionally wherein the second polymer arm has a number average molecular weight that is equal to or up to about 10 kDa higher than the number average molecular weight of the polymer arm.

In certain embodiments, the hydrophilic monomer is selected from acrylates, (meth)acrylates, acrylamides, (meth)acrylamides, allyl ethers, vinyl acetates, vinyl amides, substituted styrenes, amino acids, acrylonitrile, heterocyclic monomers (i.e. ethylene oxide), saccharides, phosphoesters, phosphonamides, sulfonate esters, sulfonamides, or combinations thereof.

In certain embodiments, the hydrophilic monomer is selected from (meth)acrylates or (meth)acrylamides of the chemical formula CH₂═CR₂—C(O)—R₁ (“Formula I”), wherein R₁ is independently selected from —OR₃, —NHR₃ or —N(CH₃)R₃; R₂ is independently selected from H and CH₃; and R₃ is independently selected from any neutral hydrophilic substituent, such as H (except for OR₃), CH₃, CH₂CH₃, CH₂CH₂OH, CH₂(CH₂)₂₀H, CH₂CH(OH)CH₃, CHCH₃CH₂OH or (CH₂CH₂O)ᵢH, where i is an integer number of repeating units selected from 1, 2, 3, 4, 5 or 6.

In certain embodiments, D3 is present and selected from targeting molecules or agonists of CD22.

In certain embodiments, the polymer arm is linked to the core through a triazole.

In certain embodiments, the linker X comprises between 4 and 24 ethylene oxide units.

In certain embodiments, when D3 is absent the ends of the polymer arms are capped. In certain specific embodiments, the cap is isobutyronitrile.

In a third aspect, provided herein is a process for preparing a star polymer according to any preceding claim, the process comprising: producing the polymer arm comprising reactive monomers by RAFT polymerization, reacting the polymer arm comprising the reactive monomers with D2 to link D2 to the reactive monomer, and grafting the polymer arm to the core by reacting X1 with X2 to form the linker X, which links the polymer arm to the core.

In certain embodiments, X1 comprises a strained alkyne and X2 comprises an azide.

In certain embodiments, the strained alkyne is linked to the core via a linker comprising between 4 and 24 ethylene oxide units.

In a fourth aspect, provided herein is a star polymer having the formula O[D1]-([X]-A[(D2)]-[Z]-D3)n where 0 is a core; A is a polymer arm attached to the core; X is a linker molecule between the core and the polymer arm; Z is a linker molecule between an end of the polymer arm and D3; D1 is a drug molecule linked to the core; D2 is a drug molecule linked to reactive monomers distributed along the backbone of the polymer arm; D3 is a drug molecule linked to the ends of the polymer arms; n is an integer number; [ ] denotes that the group is optional, wherein the polymer arm comprises reactive monomers, hydrophilic monomers and/or charged monomers, the polymer arm has a number average molecular weight between about 5 kDa to about 60 kDa, or about 10 kDa to about 40 kDa, and n is greater than or equal to 5.

In certain embodiments, D3 is selected from peptide-based CPIs. In certain specific embodiments, the peptide-based CPI has the structure:

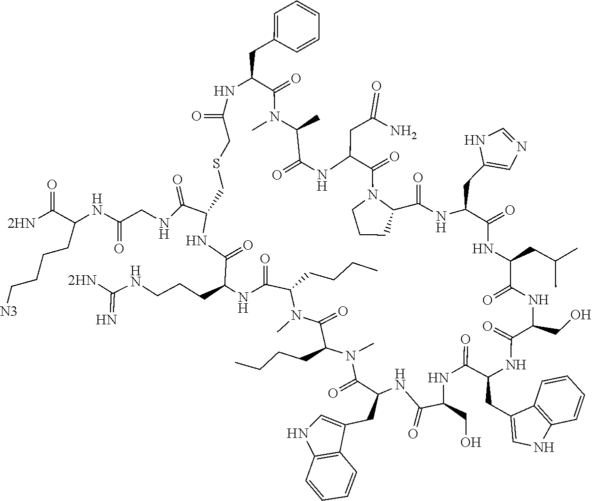

wherein the azide provides a reactive handle for attachment to a polymer arm either directly or via a linker.

In a fifth aspect, provided herein is a use of the star polymer of any of the first, second or fourth embodiments as a medicament.

In a sixth aspect, provided herein is a pharmaceutical composition comprising the star polymer of any of the first, second or fourth embodiments and a pharmaceutically acceptable carrier. In certain embodiments of the sixth aspect, the pharmaceutical composition is for use in the treatment or prophylaxis of cancer. In certain embodiments of the sixth aspect, the pharmaceutical composition is used in the treatment or prophylaxis of cancer.

In a seventh aspect, provided herein is a use of the pharmaceutical composition of the sixth aspect for the treatment or prophylaxis of cancer.

In an eighth aspect, provided herein is method of treating cancer in a subject in need of treatment, the method comprising administering the pharmaceutical composition of the sixth aspect to the subject.

In a ninth aspect, provided herein is a use of the star polymer of any of the first, second or fourth embodiments in the preparation of a medicament for the treatment or prophylaxis of cancer.

The star polymer may be administered by intravenous, intratumor, intramuscular or subcutaneous routes of administration.

The cancer to be treated may be selected from hematological tumors, such as leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia; solid tumors, such as sarcomas and carcinomas, including fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers (including adenocarcinoma, a bronchiolaveolar carcinoma, a large cell carcinoma, or a small cell carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma); skin cancer, such as a basal cell carcinoma, a squamous cell carcinoma, a Kaposi's sarcoma, or a melanoma; and, premalignant conditions, such as variants of carcinoma in situ, or vulvar intraepithelial neoplasia, cervical intraepithelial neoplasia, or vaginal intraepithelial neoplasia.

In some embodiments, of the star polymer provided herein, the maximum drug density on a hydrophilic polymer arm without inducing aggregation depends on the hydrophobic nature of D2.

In some embodiments, of the star polymer provided herein, the charge groups are selected to impart pH-induced hydrodynamic behavior changes from physiologic pH 7.4 to tumor pH.

In some embodiments, of the star polymer provided herein, the transition pH of polymers with D2 and charge groups depends on the charge group native pKa.

In some embodiments, of the star polymer provided herein, the positive charge groups significantly enhance non-specific immune cell uptake and systemic toxicity of polymers with D2.

In some embodiments, of the star polymer provided herein, the negative charge groups solubilize polymers with D2 in physiologic pH 7.4 and avoid non-specific immune cell uptake in blood circulation while inducing aggregation in tumor microenvironment at acidic pH to increase the efficacy of immunotherapy treatments.

In some embodiments, the star polymers comprising HPMA-based hydrophilic blocks provide sufficient shielding of D2 on the first block, elongating circulation in blood and higher enrichment in tumor.

In some embodiments, the star polymers comprising higher density of polymer arms with a PHPMA hydrophilic block provide higher shielding of D2 on the first block, therefore less non-specific immune cell uptake and lower systemic toxicity.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A star polymer having the formula O[D1]-([X]-A(D2)-[Z]-[D3])ₙ where O is a core; each A is a polymer arm attached to the core; each X is a linker molecule between the core and the polymer arm; each Z is a linker molecule between an end of the polymer arm and D3; D1 is a drug molecule linked to the core; each D2 is a drug molecule linked to reactive monomers distributed along the backbone of the polymer arm; each D3 is a drug molecule linked to the ends of the polymer arms; n is an integer from 5 to 60; wherein each A, X, Z, D2 and D3 may be the same or different; [ ] denotes that the group is optional; wherein the polymer arm, A, comprises reactive monomers, hydrophilic monomers, charged monomers, or any combination thereof, and D2 is linked to the reactive monomers distributed along the polymer arm at a density of between 1 mol % and 80 mol %.

2. The star polymer of claim 1, wherein each D2 is independently selected from amphiphilic or hydrophobic drug molecules, and D2 is linked to the polymer arms at a density of between about 1 mol % and about 40 mol %, or between about 5 mol % and 20 mol %, or between about 7.5 mol % and 15 mol %.

3. The star polymer of claim 1 or 2, wherein the polymer arm comprises charged monomers that are negatively charged at pH 7.4.

4. The star polymer of any one of claims 1 to 3, wherein the charged monomers are distributed along the polymer arm at a density of between about 0.125 to 2.0 times the density at which D2 is linked to reactive monomers distributed along the backbone of the polymer arm.

5. The star polymer of any one of claims 1 to 4, wherein the charged monomers comprise carboxylic acids and/or carboxylic acid salts.

6. The star polymer of any one of claims 1 to 5, wherein the charged monomer comprises beta-alanine, butanoic acid, methyl butanoic acid, dimethylbutanoic acid, 3,3′-((2-(6-aminohexanamido)propane-1,3-diyl)bis(oxy))dipropionic acid, or 13-(6-aminohexanamido)-6,20-bis((2-carboxyethoxy)methyl)-8,18-dioxo-4,11,15,22-tetraoxa-7,19-diazapentacosanedioic acid.

7. The star polymer of any one of claims 1 to 6, wherein the charged monomers are selected from (meth)acrylates and (meth)acrylamides having the chemical formula CH₂═CR₅—C(O)—R₄; wherein R₄ is independently selected from —OR₆, —NHR₆ or —N(CH₃)R₆; R₅ is independently selected from H or CH₃; and R₆ is selected from OH (except for NHR₆ or —N(CH₃)R₆), (CH₂)ⱼCH(NH₂)COOH, (CH₂)ⱼCOOH, (CH₂)ⱼCH(CH₃)COOH, (CH₂)ⱼC(CH₃)₂COOH, CH(COOH)CHCH₂COOH, (CH₂)ⱼNH(CH₂)ⱼCOOH, (CH₂)ⱼN(CH₃)(CH₂)ⱼCOOH, (CH₂)ⱼN⁺(CH₃)₂(CH₂)ⱼCOOH, (CH₂)ⱼN⁺(CH₂—CH₃)₂(CH₂)ⱼCOOH, (CH₂)ₜ—C(O)—NH—(CH₂)ⱼCH(NH₂)COOH, (CH₂)ₜ—C(O)—NH—(CH₂)ⱼCOOH, (CH₂)ₜ—C(O)—NH—(CH₂)ⱼCH(CH₃)COOH, (CH₂)ₜ—C(O)—NH—(CH₂)ⱼC(CH₃)₂COOH, (CH₂)ₜ—C(O)—NH—CH(COOH)CHCH₂COOH, (CH₂)ₜ—C(O)—NH—(CH₂)ⱼNH(CH₂)ⱼCOOH, (CH₂)ₜ—C(O)—NH—(CH₂)ⱼN(CH₃)(CH₂)ⱼCOOH, (CH₂)ₜ—C(O)—NH—(CH₂)ⱼN⁺(CH₃)₂(CH₂)ⱼCOOH, (CH₂)ₜ—C(O)—NH—(CH₂)ⱼN⁺(CH₂—CH₃)₂(CH₂)ⱼCOOH, (CH₂CH₂O)ₜCH₂CH₂C(O)—NH—(CH₂)ⱼCH(NH₂)COOH, (CH₂CH₂O)ₜCH₂CH₂C(O)—NH—(CH₂)ⱼCOOH, (CH₂CH₂O)ₜCH₂CH₂C(O)—NH—(CH₂)ⱼCH(CH₃)COOH, (CH₂CH₂O)ₜCH₂CH₂C(O)—NH—(CH₂)ⱼC(CH₃)₂COOH, (CH₂CH₂O)ₜCH₂CH₂C(O)—NH—CH(COOH)CHCH₂COOH, (CH₂CH₂O)ₜCH₂CH₂C(O)—NH—(CH₂)ⱼNH(CH₂)ⱼCOOH, (CH₂CH₂O)ₜCH₂CH₂C(O)—NH—(CH₂)ⱼN(CH₃)(CH₂)ⱼCOOH, (CH₂CH₂O)ₜCH₂CH₂C(O)—NH—(CH₂)ⱼN⁺(CH₃)₂(CH₂)ⱼCOOH, (CH₂CH₂O)ₜCH₂CH₂C(O)—NH—(CH₂)ⱼN⁺(CH₂—CH₃)₂(CH₂)ⱼCOOH, wherein t and j are each an integer number of repeating units, each independently selected from between 1 to 6, such as 1, 2, 3, 4, 5 or 6.

8. The star polymer of claim 7, wherein R₄ is independently selected from —NHR₆ or —N(CH₃)R₆; R₅ is independently selected from H or CH₃; and R₆ is selected from (CH₂)₂COOH, (CH₂)₃COOH, (CH₂)₂CH(CH₃)COOH, (CH₂)₂C(CH₃)₂COOH, (CH₂)ₜ—C(O)—NH—(CH₂)₂COOH, (CH₂)ₜ—C(O)—NH—(CH₂)₃COOH, (CH₂)ₜ—C(O)—NH—(CH₂)₂CH(CH₃)COOH or (CH₂)ₜ—C(O)—NH—(CH₂)₂C(CH₃)₂COOH, (CH₂CH₂O)ₜCH₂CH₂C(O)—(CH₂)₂COOH, (CH₂CH₂O)ₜCH₂CH₂C(O)—(CH₂)₃COOH, (CH₂CH₂O)ₜCH₂CH₂C(O)—(CH₂)₂CH(CH₃)COOH or (CH₂CH₂O)ₜCH₂CH₂C(O)—(CH₂)₂C(CH₃)₂COOH, wherein t is an integer number of repeating units selected from between 1 to 6, such as 1, 2, 3, 4, 5 or 6.

9. The star polymer of any one of claims 5 to 8, wherein the carboxylic acid is in the form of an alkylammonium salt.

10. The star polymer of any one of claims 1 to 9, wherein D2 is linked to reactive monomers distributed along the polymer arm at a density of between about 1 mol % and about 8 mol % or between about 3 mol % and about 7 mol % and the polymer arm comprises charged monomers that comprise a nitrogen base selected from primary amines, secondary amines, tertiary amines, aromatic amines, and nitrogen heterocycles that are distributed along the polymer arm at a density of between about 3 mol % and about 30 mol % or about 5 mol % and about 20 mol %.

11. The star polymer of claim 10, wherein the nitrogen base is selected from groups comprising pyrrole, imidazole, pyridine, pyrimidine, pyrazine, diazepine, indole, quinoline, amino quinoline, amino pyridine, purine, pteridine, aniline, or naphthalene amine rings.

12. The star polymer of any one of claims 10 to 11, wherein the charged monomer is selected from (meth)acrylates and (meth)acrylamides with chemical formula CH₂═CR₅—C(O)—R₄ (“Formula II”), wherein R₄ is independently selected from —OR₆, —NHR₆ or —N(CH₃)R₆; R₅ is independently selected from H or CH₃; and R₆ is selected from (CH₂)ⱼ-imidazole, (CH₂)ⱼ-pyridine amine, (CH₂)ⱼ-quinoline amine, (CH₂)ⱼ-naphthalene amine, (CH₂)ⱼN(CH₃)₂, CH₂N(CH₃)₂, CH₂CH₂N(CH₃)₂, CH₂CH₂CH₂N(CH₃)₂, CH₂N(CH₂CH₃)₂, (CH₂)ⱼN(CH₂CH₃)₂, CH₂CH₂N(CH₂CH₃)₂, CH₂CH₂CH₂N(CH₂CH₃)₂, CH₂N(CH(CH₃)₂)₂, (CH₂)ⱼN((CH(CH₃)₂)₂, CH₂CH₂N((CH(CH₃)₂)₂, CH₂CH₂CH₂N(CH(CH₃)₂)₂, (CH₂)ₜ—C(O)—NH—(CH₂)ⱼ-imidazole, (CH₂)ₜ—C(O)—NH—(CH₂)ⱼ-pyridine amine, (CH₂)ₜ—C(O)—NH—(CH₂)ⱼ-quinoline amine, (CH₂)ₜ—C(O)—NH—(CH₂)ⱼ-naphthalene amine, (CH₂)ₜ—C(O)—NH—(CH₂)ⱼN(CH₃)₂, CH₂N(CH₃)₂, (CH₂)ₜ—C(O)—NH—CH₂CH₂N(CH₃)₂, (CH₂)ₜ—C(O)—NH—CH₂CH₂CH₂N(CH₃)₂, (CH₂)ₜ—C(O)—NH—CH₂N(CH₂CH₃)₂, (CH₂)ₜ—C(O)—NH—(CH₂)ⱼN(CH₂CH₃)₂, (CH₂)ₜ—C(O)—NH—CH₂CH₂N(CH₂CH₃)₂, CH₂CH₂CH₂N(CH₂CH₃)₂, CH₂N(CH(CH₃)₂)₂, (CH₂)ₜ—C(O)—NH—(CH₂)ⱼN((CH(CH₃)₂)₂, (CH₂)ₜ—C(O)—NH—CH₂CH₂N((CH(CH₃)₂)₂, (CH₂)ₜ—C(O)—NH—CH₂CH₂CH₂N(CH(CH₃)₂)₂, (CH₂CH₂O)ₜCH₂CH₂(O)—NH—(CH₂)ⱼ-imidazole, (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-quinoline amine, (CH₂CH₂O)ₜCH₂CH₂C(O)—NH—(CH₂)ⱼ-naphthalene amine, (CH₂CH₂O)ₜCH₂CH₂C(O)—NH—(CH₂)ⱼN(CH₃)₂, CH₂N(CH₃)₂, (CH₂CH₂O)ₜCH₂CH₂C(O)—NH—CH₂CH₂N(CH₃)₂, (CH₂CH₂O)ₜCH₂CH₂C(O)—NH—CH₂CH₂CH₂N(CH₃)₂, (CH₂)ₜ—C(O)—NH—CH₂N(CH₂CH₃)₂, (CH₂CH₂O)ₜCH₂CH₂C(O)—NH—(CH₂)ⱼN(CH₂CH₃)₂, (CH₂CH₂O)ₜCH₂CH₂C(O)—NH—CH₂CH₂N(CH₂CH₃)₂, CH₂CH₂CH₂N(CH₂CH₃)₂, CH₂N(CH(CH₃)₂)₂, (CH₂CH₂O)ₜCH₂CH₂C(O)—NH—(CH₂)ⱼN((CH(CH₃)₂)₂, (CH₂CH₂O)ₜCH₂CH₂C(O)—NH—CH₂CH₂N((CH(CH₃)₂)₂, or (CH₂CH₂O)ₜCH₂CH₂C(O)—NH—CH₂CH₂CH₂N(CH(CH₃)₂)₂, wherein t and j are each an integer number of repeating units, each independently selected from between 1 to 6, such as 1, 2, 3, 4, 5 or 6.

13. The star polymer of any one of claims 2 to 12, wherein the amphiphilic or hydrophobic drug molecule is selected from immunostimulants or chemotherapeutics.

14. The star polymer of claim 13, wherein the immunostimulants are selected from pyrimidoindole or lipid-based TLR-4 agonists; adenine-, imdazoquinoline-, or benzonaphthyridine-based TLR-7, TLR-8 or TLR-7/8 agonists; xanthonoid-, amidobenzimidazole-based agonists of STING; and, peptide or 3-(2,3-dihydro-1,4-benzodioxin-6-yl)-2-methylphenyl]methanol based inhibitors of PD1/PDL1.

15. The star polymer of claim 14, wherein the imidazoquinoline-based TLR-7, TLR-8 or TLR-7/8a has the structure: 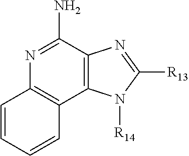
wherein R₁₃ is selected from one of hydrogen, optionally substituted lower alkyl, or optionally substituted lower alkyl ether; and R₁₄ is selected from one of optionally substituted arylalkylamine, or optionally substituted lower alkylamine, wherein the amine provides a reactive handle for attachment to the reactive monomer either directly or via a linker.

16. The star polymer of claim 14, wherein the amidobenzimidazole-based STINGa has the following structure:

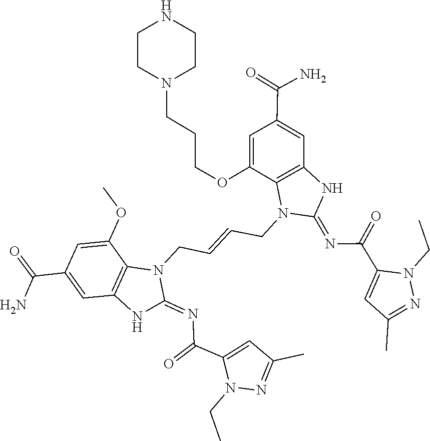 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

17. The star polymer of claim 13, wherein the chemotherapeutics are selected from alkylating agents, antibiotics, antimetabolites, topoisomerase inhibitors, mitotic inhibitors, receptor tyrosine kinase inhibitors, angiogenesis inhibitors, steroids and anti-hormonal agents.

18. The star polymer of claim 1, wherein each D2 is independently selected from hydrophilic drug molecules and D2 is linked to the polymer arms at a density of between about 1 mol % and about 40 mol %, and the hydrophilic monomer is distributed along the polymer arms at a density of between about 60 mol % to about 99 mol %.

19. The star polymer of claim 18, wherein each D2 is independently selected from hydrophilic immunostimulants or hydrophilic chemotherapeutics.

20. The star polymer of claim 19, wherein the hydrophilic immunostimulants are selected from ssRNA-based agonists of TLR-3, hydroxy-adenine based TLR-7 agonists, oligonucleotide-based agonists of TLR-9 and/or cyclic dinucleotide-based STING agonists.

21. The star polymer of claim 20, wherein the cyclic dinucleotide-based STING agonists has the structure:

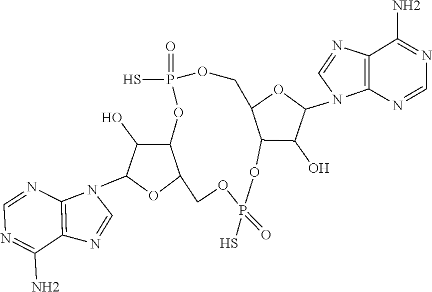 [SMILES: Nc1ncnc2c1ncn2C1OC2COP(=O)(S)OC3C(COP(=O)(S)OC2C1O)OC(n1cnc2c(N)ncnc21)C3O]

22. The star polymer of claim 21, wherein the cyclic dinucleotide-based STING agonist has R or S stereochemistry at the phosphorous stereocenter.

23. A star polymer of formula O[D1]-([X]-A1(D2)-b-A2-[Z]-[D3])n where O is a core; A1 and A2 collectively form a polymer arm (A) attached to the core, wherein each polymer arm comprises a first block A1 and a second block A2, which are proximal and distal to the core, respectively; each X is a linker molecule between the core and the polymer arm; each Z is a linker molecule between the end of the polymer arm and D3; D1 is a drug molecule linked to the core; each D2 is a drug molecule linked to reactive monomers distributed along the backbone of the polymer arm; each D3 is a drug molecule linked to the ends of the polymer arms; n is an integer number from 5 to 60; wherein each A, A1, A2, X, Z, D2 and D3 may be the same or different; [ ] denotes that the group is optional; the polymer arm comprises reactive monomers, hydrophilic monomers, charged monomers, or any combination thereof; and, D2 is linked to the reactive monomers distributed along the first block of the polymer arm at a density of between 1 mol % and 80 mol %.

24. The star polymer of claim 23, wherein the second block comprises charged monomers that comprise a nitrogen base selected from primary amines, secondary amines, tertiary amines, aromatic amines and nitrogen heterocycles that are distributed along the backbone of the polymer arm at a density of between about 3 mol % and about 30 mol % or about 5 mol % and about 20 mol %.

25. The star polymer of claim 24, wherein the nitrogen base is selected from groups comprising pyrrole, imidazole, pyridine, pyrimidine, pyrazine, diazepine, indole, quinoline, amino quinoline, amino pyridine, purine, pteridine, aniline, and naphthalene amine rings.

26. The star polymer of claim 24 or 25, wherein the charged monomer is selected from (meth)acrylates and (meth)acrylamides with chemical formula CH₂═CR₅—C(O)—R₄ (“Formula II”), wherein R₄ is independently selected from —OR₆, —NHR₆ or —N(CH₃)R₆; R₅ is independently selected from H or CH₃; and R₆ is selected from (CH₂)ⱼ-imidazole, (CH₂)ⱼ-pyridine amine, (CH₂)ⱼ-quinoline amine, (CH₂)ⱼ-naphthalene amine, (CH₂)ⱼN(CH₃)₂, CH₂N(CH₃)₂, CH₂CH₂N(CH₃)₂, CH₂CH₂CH₂N(CH₃)₂, CH₂N(CH₂CH₃)₂, (CH₂)ⱼN(CH₂CH₃)₂, CH₂CH₂N(CH₂CH₃)₂, CH₂CH₂CH₂N(CH₂CH₃)₂, CH₂N(CH(CH₃)₂)₂, (CH₂)ⱼN((CH(CH₃)₂)₂, CH₂CH₂N((CH(CH₃)₂)₂, CH₂CH₂CH₂N(CH(CH₃)₂)₂, (CH₂)ₜ—C(O)—NH—(CH₂)ⱼ-imidazole, (CH₂)ₜ—C(O)—NH—(CH₂)ⱼ-pyridine amine, (CH₂)ₜ—C(O)—NH—(CH₂)ⱼ-quinoline amine, (CH₂)ₜ—C(O)—NH—(CH₂)ⱼ-naphthalene amine, (CH₂)ₜ—C(O)—NH—(CH₂)ⱼN(CH₃)₂, CH₂N(CH₃)₂, (CH₂)ₜ—C(O)—NH—CH₂CH₂N(CH₃)₂, (CH₂)ₜ—C(O)—NH—CH₂CH₂CH₂N(CH₃)₂, (CH₂)ₜ—C(O)—NH—CH₂N(CH₂CH₃)₂, (CH₂)ₜ—C(O)—NH—(CH₂)ⱼN(CH₂CH₃)₂, (CH₂)ₜ—C(O)—NH—CH₂CH₂N(CH₂CH₃)₂, CH₂CH₂CH₂N(CH₂CH₃)₂, CH₂N(CH(CH₃)₂)₂, (CH₂)ₜ—C(O)—NH—(CH₂)ⱼN((CH(CH₃)₂)₂, (CH₂)ₜ—C(O)—NH—CH₂CH₂N((CH(CH₃)₂)₂, (CH₂)ₜ—C(O)—NH—CH₂CH₂CH₂N(CH(CH₃)₂)₂, (CH₂CH₂O)ₜCH₂CH₂(O)—NH—(CH₂)ⱼ-imidazole, (CH₂CH₂O)ₜCH₂CH₂C(O)—NH—(CH₂)ⱼ-pyridine amine, (CH₂CH₂O)ₜCH₂CH₂C(O)—NH—(CH₂)ⱼ-quinoline amine, (CH₂CH₂O)ₜCH₂CH₂C(O)—NH—(CH₂)ⱼ-naphthalene amine, (CH₂CH₂O)ₜCH₂CH₂C(O)—NH—(CH₂)ⱼN(CH₃)₂, CH₂N(CH₃)₂, (CH₂CH₂O)ₜCH₂CH₂C(O)—NH—CH₂CH₂N(CH₃)₂, (CH₂CH₂O)ₜCH₂CH₂C(O)—NH—CH₂CH₂CH₂N(CH₃)₂, (CH₂)ₜ—C(O)—NH—CH₂N(CH₂CH₃)₂, (CH₂CH₂O)ₜCH₂CH₂C(O)—NH—(CH₂)ⱼN(CH₂CH₃)₂, (CH₂CH₂O)ₜCH₂CH₂C(O)—NH—CH₂CH₂N(CH₂CH₃)₂, CH₂CH₂CH₂N(CH₂CH₃)₂, CH₂N(CH(CH₃)₂)₂, (CH₂CH₂O)ₜCH₂CH₂C(O)—NH—(CH₂)ⱼN((CH(CH₃)₂)₂, (CH₂CH₂O)ₜCH₂CH₂C(O)—NH—CH₂CH₂N((CH(CH₃)₂)₂, or (CH₂CH₂O)ₜCH₂CH₂C(O)—NH—CH₂CH₂CH₂N(CH(CH₃)₂)₂, wherein t and j are each an integer number of repeating units, each independently selected from between 1 to 6, such as 1, 2, 3, 4, 5 or 6.

27. The star polymer of any of claim 23 to 26, wherein each D2 is independently selected from amphiphilic or hydrophobic drug molecules linked to the first block of the polymer arm at a density of between about 1 mol % to about 80 mol %, or between about 5 mol % to about 40 mol %, or between about 10 mol % to about 30 mol %.

28. The star polymer of any one of claims 23 to 27, wherein the first block is linked to the second block through a pH-sensitive bond selected from hydrazone, silyl-ether and ketal linkages.

29. The star polymer of any one of claims 23 to 28, wherein the degree of polymerization block ratio of the first block to the second block is about 1:5 to about 2:1.

30. The star polymer of any one of claims 1 to 29, wherein D2 is linked to reactive monomers selected from (meth)acrylates and (meth)acrylamides of chemical formula CH₂═CR₈—C(O)—R₇ (“Formula III”), wherein R₇ is an acryl side group comprising a linker molecule for the attachment of D2.

31. The star polymer of any one of claims 1 to 29, wherein D2 is linked to the reactive monomers through a pH-sensitive bond selected from hydrazone, silyl ether and ketal linkages.

32. The star polymer of claim 31, wherein the pH-sensitive bond is a carbohydrazone.

33. The star polymer of any one of claims 1 to 29, wherein D2 is linked to reactive monomers through an enzyme degradable peptide or a sulfatase cleavable linker.

34. The star polymer of any one of claims 1 to 33, wherein each polymer arm independently has a number average molecular weight between about 5 kDa to about 60 kDa, or about 15 kDa to about 50 kDa or about 20 kDa to 40 kDa or about 25 to about 35 kDa.

35. The star polymer of any one of claims 1 to 34, wherein the core (O) has greater than 5 points of attachment for polymer arms (A).

36. The star polymer of any one of claims 1 to 35, wherein the core (O) comprises a branched polymer or dendrimer.

37. The star polymer of any one of claims 1 to 36, wherein the dendrimer or branched polymer that is used to form the core (O) has surface amine groups used for the attachment of polymer arms (A) either directly or via a linker X.

38. The star polymer of any one of claims 1 to 37, wherein the core (O) is a dendrimer selected from PAMAM, bis(MPA), or poly(L-lysine) (PLL).

39. The star polymer of any one of claims 1 to 38, wherein n is greater than or equal to 5 and less than or equal to 60, or n is greater than or equal to 10 and less than or equal to 45, or n is greater than or equal to 20 and less than or equal to 35.

40. The star polymer of any one of claims 1 to 39 comprising a second polymer arm that is linked to the core through an amide linker or pH-sensitive linkage selected from hydrazone, ketal and silyl ether linkages, wherein the second polymer arm comprises hydrophilic monomers, charged monomers, or any combination thereof, additionally wherein the second polymer arm has a number average molecular weight that is equal to or higher than the number average molecular weight of first the polymer arm.

41. The star polymer of claim 40, wherein the polymer arm is 5% to 80% of the polymer arms, and the second polymer arm is 20% to 95% of the polymer arms, or wherein the polymer arm, A, is 50% to 80% of the polymer arms, and the second polymer arm is 20% to 50% of the polymer arms.

42. The star polymer of any one of claims 1 to 41, wherein the hydrophilic monomer is selected from acrylates, (meth)acrylates, acrylamides, (meth)acrylamides, allyl ethers, vinyl acetates, vinyl amides, substituted styrenes, amino acids, acrylonitrile, heterocyclic monomers, saccharides, phosphoesters, phosphonamides, sulfonate esters, sulfonamides, or combinations thereof.

43. The star polymer of claim 42, wherein the hydrophilic monomer is selected from (meth)acrylates or (meth)acrylamides of the chemical formula CH₂═CR₂—C(O)—R₁ (“Formula I”), wherein R₁ is independently selected from —OR₃, —NHR₃ or —N(CH₃)R₃; R₂ is independently selected from H and CH₃; and R₃ is independently selected from a neutral hydrophilic substituent, such as H (except for OR₃), CH₃, CH₂CH₃, CH₂CH₂OH, CH₂(CH₂)₂OH, CH₂CH(OH)CH₃, CHCH₃CH₂OH or (CH₂CH₂O)ᵢH, where i is an integer number of repeating units selected from 1, 2, 3, 4, 5 or 6.

44. The star polymer of any one of claims 1 to 43, wherein each D3 is independently selected from targeting molecules.

45. The star polymer of any one of claims 1 to 44, wherein X comprises a triazole, or wherein X comprises between 4 and 24 ethylene oxide units, or wherein X comprises an enzyme degradable linker.

46. The star polymer of claim 45, wherein Z comprises a triazole, or wherein Z comprises an enzyme degradable linker.

47. The star polymer of any one of claims 1 to 46, wherein enzyme degradable linker comprises single amino acids, or dipeptides, tripeptides, or tetrapeptides, or combinations thereof.

48. The star polymer of any one of claims 1 to 47, wherein when D3 is absent and the ends of the polymer arms are capped.

49. The star polymer of claim 48, wherein the cap is isobutyronitrile.

50. The star polymer of any one of claims 1 to 49, wherein n is an integer from 20 to 35 and each A, X, and Z is the same.

51. The star polymer of any one of claims 1 to 49, wherein n is an integer from 20 to 35 and each A, X, and Z are chosen to provide at least two different combinations of polymer arm and linkers.

52. The star polymer of any one of claims 1 to 51, wherein the density of charged monomers with a single charged functional group is selected based on the density of attached drug molecule according to Table 1.

53. The star polymer of claim 52, wherein the density of amphiphilic or hydrophobic drug molecules linked to reactive monomers is about 7 mol % to about 15 mol %; and wherein the charged monomers comprise about 5 mol % to about 23 mol % of the monomers in the star polymer.

54. The star polymer of any one of claims 1 to 51, wherein the density of charged monomers with two charged functional groups is selected based on the density of attached drug molecule according to Table 2.

55. The star polymer of claim 54, wherein the density of amphiphilic or hydrophobic drug molecules linked to reactive monomers is about 7 mol % to about 15 mol %; and wherein the bifunctional charged monomers comprises about 3 mol % to about 11 mol % of the monomers in the star polymer.

56. The star polymer of any one of claims 1 to 51, wherein the density of charged monomers with three or four charged functional groups is selected based on the density of attached drug molecule according to Table 3.

57. The star polymer of claim 56, wherein the density of amphiphilic or hydrophobic drug molecules linked to reactive monomers is about 7 mol % to about 15 mol %; and the trifunctional or tetrafunctional charged monomers comprise about 1 mol % to about 6 mol % of the monomers in the star polymer.

58. A process for preparing a star polymer according to any one of claims 1 to 57, the process comprising: producing the polymer arm comprising reactive monomers by RAFT polymerization, reacting the polymer arm comprising the reactive monomers with D2 to link D2 to the reactive monomer, and grafting the polymer arm to the core by reacting X1 with X2 to form the linker X, which links the polymer arm to the core.

59. The process according to claim 58, wherein X1 comprises a strained alkyne and X2 comprises an azide.

60. The process according to claim 59, wherein the strained alkyne is linked to the core via a linker comprising between 4 and 24 ethylene oxide units.

61. A star polymer having the formula O[D1]-([X]-A-[Z]-D3)n where O is a core; each A is a polymer arm attached to the core; each X is a linker molecule between the core and the polymer arm; each Z is a linker molecule between an end of the polymer arm and D3; D1 is a drug molecule linked to the core; each D3 is a drug molecule linked to the ends of the polymer arms; n is an integer number from 1 to 60; wherein each A, X, Z, and D3 may be the same or different; [ ] denotes that the group is optional, wherein the polymer arm comprises reactive monomers, hydrophilic monomers, charged monomers, or any combination thereof, the polymer arm has a number average molecular weight between about 5 kDa to about 60 kDa, or about 15 kDa to about 50 kDa, or about 20 kDa to about 40 kDa.

62. The star polymer of any one of claims 1 to 57 or 61, wherein D3 is selected from peptide-based CPIs.

63. The star polymer of claim 62, wherein the peptide-based CPI has the structure: 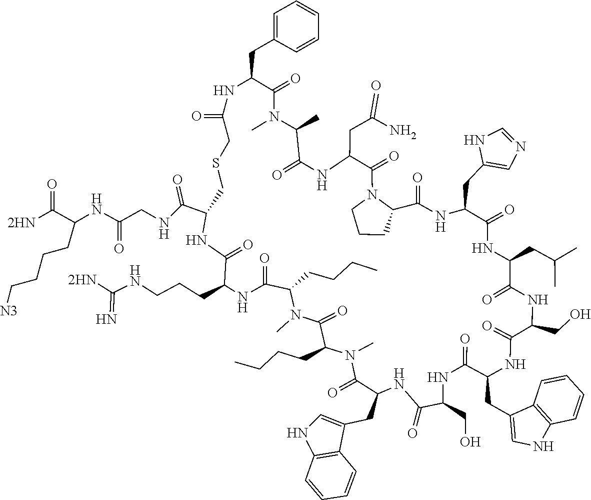
wherein the azide provides a reactive handle for attachment to a polymer arm either directly or via a linker.

64. Use of the star polymer of any one of claims 1 to 63 as a medicament.

65. A pharmaceutical composition comprising the star polymer of any one of claims 1 to 63 and a pharmaceutically acceptable carrier.

66. The pharmaceutical composition of claim 65 for use in the treatment or prophylaxis of cancer.

67. The pharmaceutical composition of claim 65 when used in the treatment or prophylaxis of cancer.

68. Use of the pharmaceutical composition of claim 65 for the treatment or prophylaxis of cancer.

69. A method of treating cancer in a subject in need of treatment, the method comprising administering the pharmaceutical composition of claim 65 to the subject.

70. Use of the star polymer of any one of claims 1 to 63 in the preparation of a medicament for the treatment or prophylaxis of cancer.

71. The pharmaceutical composition of any one of claims 65 to 67, the use of claim 68 or the method of claim 69 wherein the star polymer is administered by intravenous, intratumoral, intramuscular or subcutaneous routes of administration.

72. The pharmaceutical composition of any one of claims 65 to 67, the use of claim 68, the method of claim 69 or the use of claim 70 wherein the cancer is selected from hematological tumors, such as leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia; solid tumors, such as sarcomas and carcinomas, including fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers (including adenocarcinoma, a bronchiolaveolar carcinoma, a large cell carcinoma, or a small cell carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma); skin cancer, such as a basal cell carcinoma, a squamous cell carcinoma, a Kaposi's sarcoma, or a melanoma; and, premalignant conditions, such as variants of carcinoma in situ, or vulvar intraepithelial neoplasia, cervical intraepithelial neoplasia, or vaginal intraepithelial neoplasia.
